(19) United States
(12) Patent Application Publication
(10) Pub. No.: US 2023/0287001 A1
(43) Pub. Date: Sep. 14, 2023

(54) Organic Semiconducting Compounds

(71) Applicant: Raynergy Tek Incorporation, Hsinchu (TW)

(72) Inventors: Graham MORSE, SOUTHAMPTON (GB); Kane HEARD, SOUTHAMPTON (GB); William MITCHELL, CHANDLER'S FORD (GB); Michal KROMPIEC, SOUTHAMPTON (GB); Mansoor D'LAVARI, SOUTHAMPTON (GB); Agnieszka PRON, EASTLEIGH (GB)

(21) Appl. No.: 18/128,783

(22) Filed: Mar. 30, 2023

Related U.S. Application Data
(63) Continuation of application No. 16/646,846, filed on Mar. 12, 2020, now abandoned.

(30) Foreign Application Priority Data
Sep. 13, 2017 (EP) 17190866.8
Nov. 14, 2017 (EP) 17201478.9

(51) Int. Cl. C07D 495/22; C07D 409/10; C07D 495/04; H10K 30/30; H10K 85/10; H10K 85/60

(52) U.S. Cl. CPC C07D 495/22; C07D 409/10; C07D 495/04; H10K 30/30; H10K 85/113; H10K 85/623; H10K 85/655; H10K 85/656; H10K 85/6576

(57) ABSTRACT

The invention relates to novel organic semiconducting compounds containing a polycyclic unit, to methods for their preparation and educts or intermediates used therein, to compositions, polymer blends and formulations containing them, to the use of the compounds, compositions and polymer blends as organic semiconductors in, or for the preparation of, organic electronic (OE) devices, especially organic photovoltaic (OPV) devices, perovskite-based solar cell (PSC) devices, organic photodetectors (OPD), organic field effect transistors (OFET) and organic light emitting diodes (OLED), and to OE, OPV, PSC, OPD, OFET and OLED devices comprising these compounds, compositions or polymer blends.

### TECHNICAL FIELD

The invention relates to novel organic semiconducting compounds containing a polycyclic unit, to methods for their preparation and educts or intermediates used therein, to compositions, polymer blends and formulations containing them, to the use of the compounds, compositions and polymer blends as organic semiconductors in, or for the preparation of, organic electronic (OE) devices, especially organic photovoltaic (OPV) devices, perovskite-based solar cell (PSC) devices, organic photodetectors (OPD), organic field effect transistors (OFET) and organic light emitting diodes (OLED), and to OE, OPV, PSC, OPD, OFET and OLED devices comprising these compounds, compositions or polymer blends.

### BACKGROUND

In recent years, there has been development of organic semiconducting (OSC) materials in order to produce more versatile, lower cost electronic devices. Such materials find application in a wide range of devices or apparatus, including organic field effect transistors (OFETs), organic light emitting diodes (OLEDs), organic photodetectors (OPDs), organic photovoltaic (OPV) cells, perovskite-based solar cell (PSC) devices, sensors, memory elements and logic circuits to name just a few. The organic semiconducting materials are typically present in the electronic device in the form of a thin layer, for example of between 50 and 300 nm thickness.

One particular area of importance is organic photovoltaics (OPV). Conjugated polymers have found use in OPVs as they allow devices to be manufactured by solution-processing techniques such as spin casting, dip coating or ink jet printing. Solution processing can be carried out cheaper and on a larger scale compared to the evaporative techniques used to make inorganic thin film devices. Currently, polymer based photovoltaic devices are achieving efficiencies above 10%.

Another particular area of importance are OFETs. The performance of OFET devices is principally based upon the charge carrier mobility of the semiconducting material and the current on/off ratio, so the ideal semiconductor should have a low conductivity in the off state, combined with high charge carrier mobility (>1×10⁻³ cm² V⁻¹ s⁻¹). In addition, it is important that the semiconducting material is stable to oxidation i.e. it has a high ionisation potential, as oxidation leads to reduced device performance. Further requirements for the semiconducting material are good processability, especially for large-scale production of thin layers and desired patterns, and high stability, film uniformity and integrity of the organic semiconductor layer.

Organic photodetectors (OPDs) are a further particular area of importance, for which conjugated light-absorbing polymers offer the hope of allowing efficient devices to be produced by solution-processing technologies, such as spin casting, dip coating or ink jet printing, to name a few only.

The photosensitive layer in an OPV or OPD device is usually composed of at least two materials, a p-type semiconductor, which is typically a conjugated polymer, an oligomer or a defined molecular unit, and an n-type semiconductor, which is typically a fullerene or substituted fullerene, graphene, a metal oxide, or quantum dots.

However, the OSC materials disclosed in prior art for use in OE devices have several drawbacks. They are often difficult to synthesis or purify (fullerenes), and/or do not absorb light strongly in the near infrared spectrum >700 nm. In addition, other OSC materials do not often form a favourable morphology and/or donor phase miscibility for use in organic photovoltaics or organic photodetectors.

Therefore there is still a need for OSC materials for use in OE devices like OPVs, OPDs and OFETs, which have advantageous properties, in particular good processability, high solubility in organic solvents, good structural organization and film-forming properties. In addition, the OSC materials should be easy to synthesize, especially by methods suitable for mass production. For use in OPV cells, the OSC materials should especially have a low bandgap, which enables improved light harvesting by the photoactive layer and can lead to higher cell efficiencies, high stability and long lifetime. For use in OFETs the OSC materials should especially have high charge-carrier mobility, high on/off ratio in transistor devices, high oxidative stability and long lifetime.

It was an aim of the present invention to provide new OSC compounds, especially n-type OSCs, which can overcome the drawbacks of the OSCs from prior art, and which provide one or more of the above-mentioned advantageous properties, especially easy synthesis by methods suitable for mass production, good processability, high stability, long lifetime in OE devices, good solubility in organic solvents, high charge carrier mobility, and a low bandgap. Another aim of the invention was to extend the pool of OSC materials and n-type OSCs available to the expert. Other aims of the present invention are immediately evident to the expert from the following detailed description.

The inventors of the present invention have found that one or more of the above aims can be achieved by providing compounds as disclosed and claimed hereinafter. These compounds are small molecules comprising a polycyclic electron donating core and two terminal electron-withdrawing groups R^{T1} and R^{T2}, wherein the polycyclic core contains one or more groups which interrupt conjugation throughout the molecule, so that the terminal groups R^{T1} and R^{T2} are not in conjugation with each other.

Conjugation as defined in PAC, 1994, 66, 1077 (Glossary of terms used in physical organic chemistry (IUPAC Recommendations 1994)) on page 1099: In the original meaning a conjugated system is a molecular entity whose structure may be represented as a system of alternating single and multiple bonds: e.g. CH2═CH—CH═CH2, CH2═CH—C≡N. In such systems, conjugation is the interaction of one p-orbital with another across an intervening σ-bond in such structures. (In appropriate molecular entities d-orbitals may be involved.) The term is also extended to the analogous interaction involving a p-orbital containing an unshared electron pair, e.g.: Cl—CH═CH2. For example in the compound having a polycyclic core of the structure (1) as shown below, the terminal group R^{T1} is not in conjugation with the terminal group R^{T2}, due to the presence of the two thieno[2,3-b]thiophene groups which interrupt a possible conjugation throughout the molecule, while in a compound of the structure (2) as shown below, the terminal group R^{T1} is not in conjugation with the terminal group R^{T2}, due to one of these groups being located not in 2-position but in 3-position of the thiophene ring.

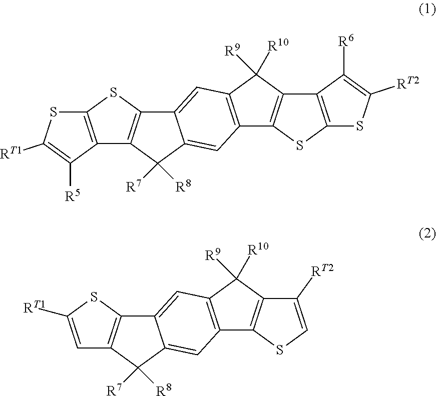

It has been found that such compounds can be used as n-type OSCs which show advantageous properties as described above and below.

### SUMMARY

The invention relates to a compound of formula I

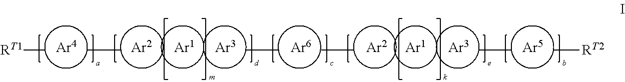

wherein the individual radicals, independently of each other and on each occurrence identically or differently, have the following meanings

Ar¹ is selected from the group consisting of the following formulae

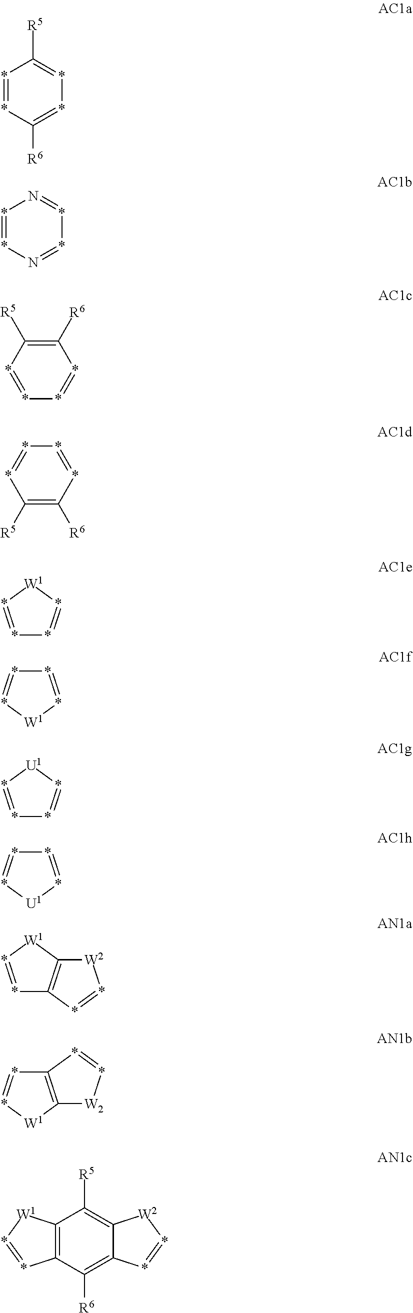

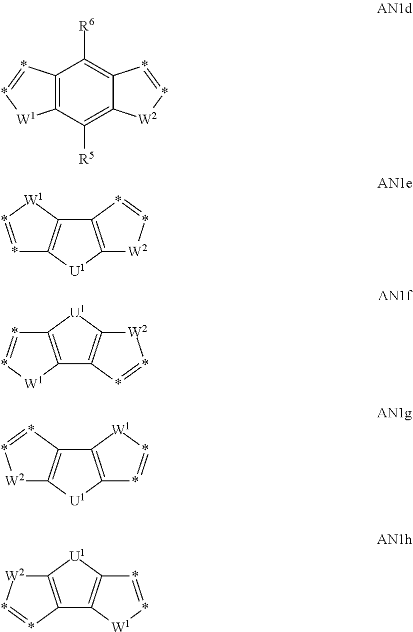

wherein a group

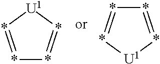

is not adjacent to another group

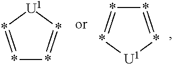

Ar² is selected from the group consisting of the following formulae

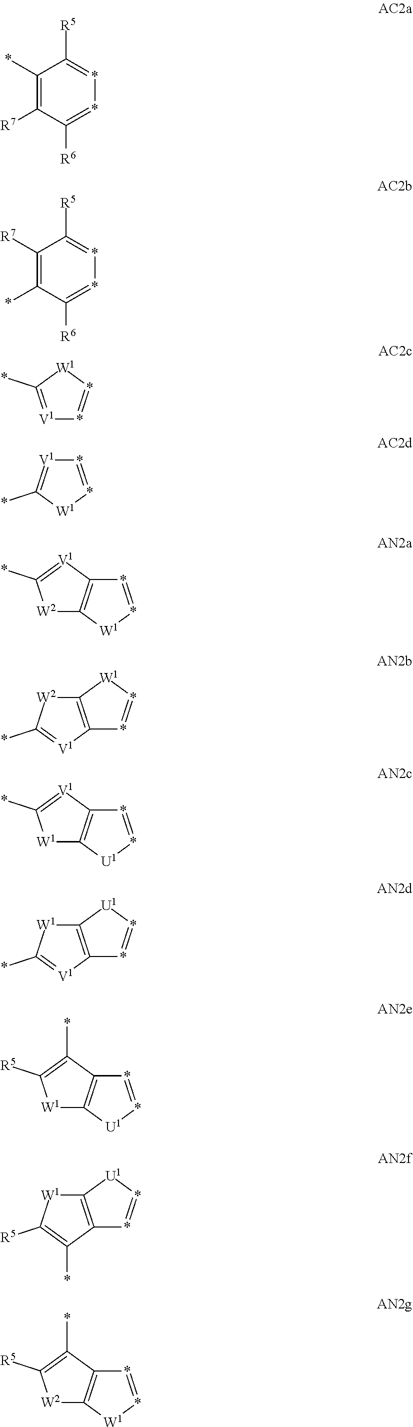

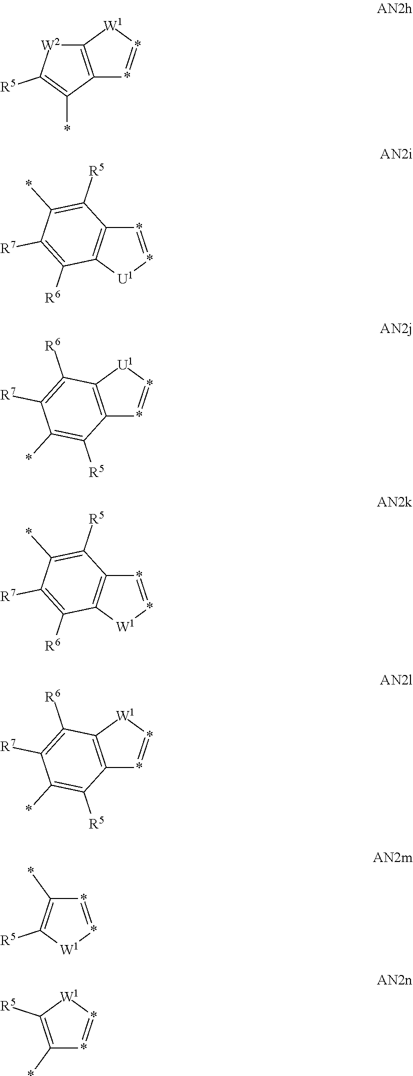

Ar³ is selected from the group consisting of the following formulae

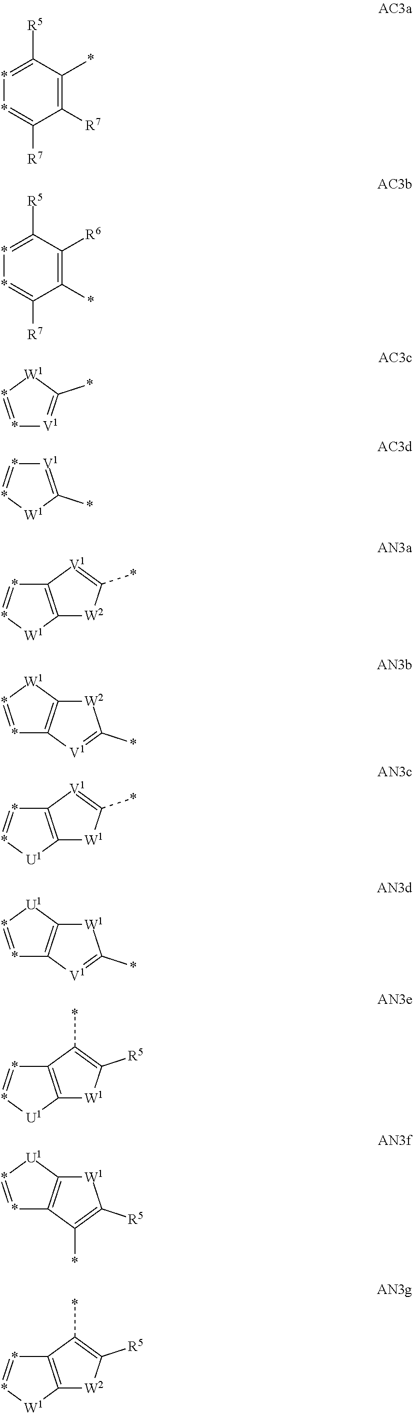

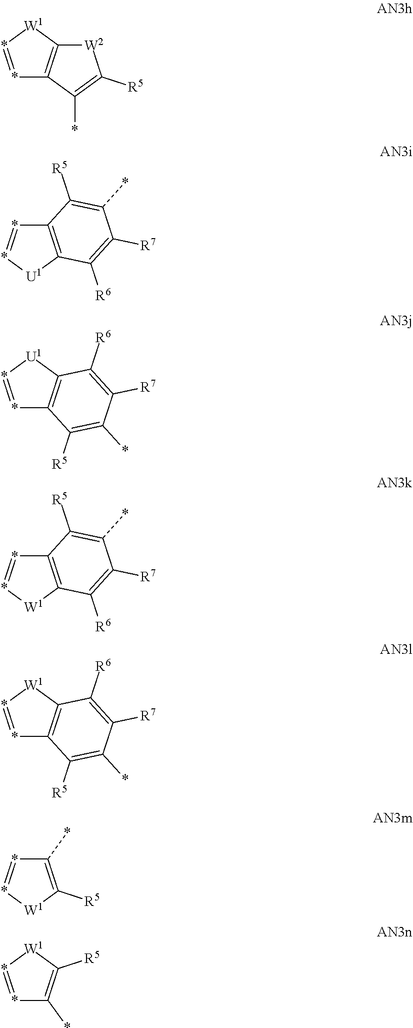

Ar⁴, Ar⁵, Ar⁶ arylene or heteroarylene that has from 5 to 20 ring atoms, is mono- or polycyclic, optionally contains fused rings, and is unsubstituted or substituted by one or more identical or different groups R¹ or L, or CY¹═CY² or —C═C—,
   one or more of Ar⁴, Ar⁵ and Ar⁶ may also be selected from the following groups or their mirror images

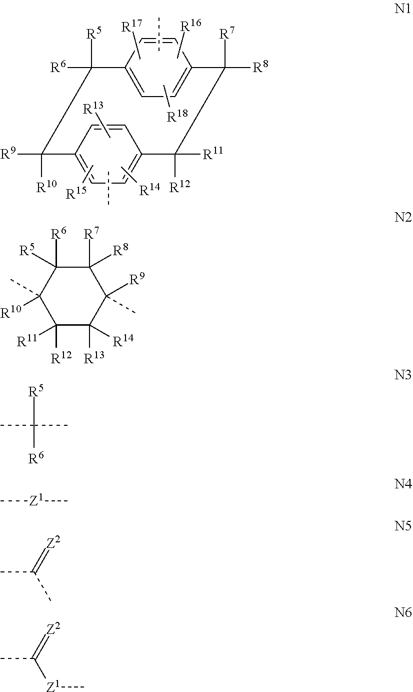

U¹ CR¹R², SiR¹R², GeR¹R², NR¹, C═O or C═CR¹R²,
V¹ CR⁵ or N,
W¹, W² S, O, Se or C═O,
Z¹ —O—, —S—,
Z² ═O, ═S, ═CR¹R², ═NR¹,
R¹⁻¹⁷ R^{W}, H, F, Cl, CN, or straight-chain, branched or cyclic alkyl with 1 to 30, preferably 1 to 20, C atoms, in which one or more CH₂ groups are optionally replaced by —O—, —S—, —C(═O), —C(═S)—, —C(═O)—O—, —O—C(═O)—, —NR⁰—, —SiR⁰R⁰⁰—, —CF₂—, —CR⁰═CR⁰⁰—, —CY¹═CY²— or —C═C— in such a manner that 0 and/or S atoms are not linked directly to one another, and in which one or more H atoms are optionally replaced by F, Cl, Br, I or CN, and in which one or more CH₂ or CHs groups are optionally replaced by a cationic or anionic group, or aryl, heteroaryl, arylalkyl, heteroarylalkyl, aryloxy or heteroaryloxy, wherein each of the aforementioned cyclic groups has 5 to 20 ring atoms, is mono- or polycyclic, does optionally contain fused rings, and is unsubstituted or substituted by one or more identical or different groups L,
   and the pair of R¹ and R², together with the C, Si or Ge atom to which they are attached, may also form a spiro group with 5 to 20 ring atoms which is mono- or polycyclic, does optionally contain fused rings, and is unsubstituted or substituted by one or more identical or different groups L,
R^{W} an electron withdrawing group, which preferably has one of the meanings given for an electron withdrawing group R^{T1},
   Y¹, Y²H, F, Cl or CN,
L F, Cl, Br, I, —NO₂, —CN, —NC, —NCO, —NCS, —OCN, —SCN, R⁰, OR⁰, SR⁰, —C(═O)X°, —C(═O)R⁰, —C(═O)—OR⁰, —O—C(═O)—R⁰, —NH₂, —NHR⁰, —NR⁰R⁰⁰, —C(═O)NHR⁰, —C(═O)NR⁰R⁰⁰, —SO₃R⁰, —SO₂R⁰, —OH, —NO₂, —CF₃, —SF₅, or optionally substituted silyl, or carbyl or hydrocarbyl with 1 to 30, preferably 1 to 20 C atoms that is optionally substituted and optionally comprises one or more hetero atoms, preferably F, —CN, R⁰, —OR⁰, —SR⁰, —C(═O)—R⁰, —C(═O)—OR⁰, —O—C(═O)—R⁰, —O—C(═O)—OR⁰, —C(═O)—NHR⁰, or —C(═O)—NR⁰R⁰⁰,
R⁰, R⁰⁰H or straight-chain or branched alkyl with 1 to 20, preferably 1 to 12, C atoms that is optionally fluorinated,
X⁰ halogen, preferably F or Cl,
R^{T1}, R^{T2} H, a carbyl or hydrocarbyl group with 1 to 30 C atoms that is optionally substituted by one or more groups L and optionally comprises one or more hetero atoms,
a, b, c 0 or an integer from 1 to 10, preferably 0, 1, 2, 3, 4 or 5,
d, e 0, 1, 2 or 3,
k 0 or an integer from 1 to 10, preferably 0, 1, 2, 3, 4, 5, 6 or 7,
m 0 or an integer from 1 to 10, preferably 0, 1, 2, 3, 4, 5, 6 or 7,

wherein a+b+c+d+e≥1, and

at least one of R^{T1} and R^{T2} is an electron withdrawing group, and

wherein the compound contains at least one moiety selected from the group consisting of AN1a, AN1b, AN1c, AN1d, AN1e, AN1f, AN1g, AN1h, AN2a, AN2b, AN2c, AN2d, AN2e, AN2f, AN2g, AN2h, AN2i, AN2j, AN2k, AN2l, AN2m, AN2n, AN3a, AN3b, AN3c, AN3d, AN3e, AN3f, AN3g, AN3h, AN3f, AN3f, AN3k, AN3l, AN3m, AN3n, N1, N2, N3, N4, N5, N6.

The compound of formula I preferably has a molecular weight≤5000 g/mol.

The invention further relates to novel synthesis methods for preparing compounds of formula I, and novel intermediates used therein.

The invention further relates to the use of compounds of formula I as semiconductor, preferably as electron acceptor or n-type semiconductor, preferably in a semiconducting material, an electronic or optoelectronic device, or a component of an electronic or optoelectronic device.

The invention further relates to the use of compounds of formula I as dyes or pigments.

The invention further relates to a composition comprising one or more compounds of formula I, and further comprising one or more compounds having one or more of a semiconducting, hole or electron transport, hole or electron blocking, insulating, binding, electrically conducting, photoconducting, photoactive or light emitting property.

The invention further relates to a composition comprising one or more compounds of formula I, and further comprising a binder, preferably an electrically inert binder, very preferably an electrically inert polymeric binder.

The invention further relates to a composition comprising a compound of formula I, and further comprising one or more electron donors or p-type semiconductors, preferably selected from conjugated polymers.

The invention further relates to a composition comprising one or more n-type semiconductors, at least one of which is a compound of formula I, and further comprising one or more p-type semiconductors.

The invention further relates to a composition comprising one or more n-type semiconductors, at least one of which is a compound of formula I, and at least one other of which is a fullerene or fullerene derivative, and further comprising one or more p-type semiconductors, preferably selected from conjugated polymers.

The invention further relates to a bulk heterojunction (BHJ) formed from a composition comprising a compound of formula I as electron acceptor or n-type semiconductor, and one or more compounds which are electron donor or p-type semiconductors, and are preferably selected from conjugated polymers.

The invention further relates to the use of a compound of formula I or a composition as described above and below, as semiconducting, charge transporting, electrically conducting, photoconducting, photoactive or light emitting material.

The invention further relates to the use of a compound of formula I or a composition as described above and below, in an electronic or optoelectronic device, or in a component of such a device or in an assembly comprising such a device.

The invention further relates to a semiconducting, charge transporting, electrically conducting, photoconducting, photoactive or light emitting material, comprising a compound of formula I or a composition as described above and below.

The invention further relates to an electronic or optoelectronic device, or a component thereof, or an assembly comprising it, which comprises a compound of formula I or a composition as described above and below.

The invention further relates to an electronic or optoelectronic device, or a component thereof, or an assembly comprising it, which comprises a semiconducting, charge transporting, electrically conducting, photoconducting or light emitting material as described above and below.

The invention further relates to a formulation comprising one or more compounds of formula I, or comprising a composition or semiconducting material as described above and below, and further comprising one or more solvents, preferably selected from organic solvents.

The invention further relates to the use of a formulation as described above and below for the preparation of an electronic or optoelectronic device or a component thereof.

The invention further relates to an electronic or optoelectronic device or a component thereof, which is obtained through the use of a formulation as described above and below.

The electronic or optoelectronic device includes, without limitation, organic field effect transistors (OFET), organic thin film transistors (OTFT), organic light emitting diodes (OLED), organic light emitting transistors (OLET), organic light emitting electrochemical cell (OLEC), organic photovoltaic devices (OPV), organic photodetectors (OPD), organic solar cells, dye-sensitized solar cells (DSSC), organic photoelectrochemical cells (OPEC), perovskite-based solar cells (PSCs), laser diodes, Schottky diodes, photoconductors, photodetectors and thermoelectric devices.

Preferred devices are OFETs, OTFTs, OPVs, PSCs, OPDs and OLEDs, in particular OPDs and BHJ OPVs or inverted BHJ OPVs.

The component of the electronic or optoelectronic device includes, without limitation, charge injection layers, charge transport layers, interlayers, planarising layers, antistatic films, printed polarizers, polymer electrolyte membranes (PEM), conducting substrates and conducting patterns.

The assembly comprising an electronic or optoelectronic device includes, without limitation, integrated circuits (IC), radio frequency identification (RFID) tags, security markings, security devices, flat panel displays, backlights of flat panel displays, electrophotographic devices, electrophotographic recording devices, organic memory devices, sensor devices, biosensors and biochips.

In addition the compounds of formula I and compositions as described above and below can be used as electrode materials in batteries, or in components or devices for detecting and discriminating DNA sequences.

### Terms and Definitions

Above and below, subformulae named “AN”, “ARN”, “CN” and “N”, such as AN1a, AN1-1, ARN1, CNaa, N1 etc., are used to denote a group interrupting conjugation, whereas subformulae named “AC”, “ARC” and “CC”, such as AC1a, AC1-1, ARC1, CCaa etc., are used to denote a group enabling conjugation. Above and below, groups interrupting conjugation will also be shortly referred to as “non-conjugating groups”, and groups enabling conjugation will also be shortly referred to as “conjugating groups”

As used herein, the term “polymer” will be understood to mean a molecule of high relative molecular mass, the structure of which essentially comprises multiple repetitions of units derived, actually or conceptually, from molecules of low relative molecular mass (Pure Appl. Chem., 1996, 68, 2291). The term “oligomer” will be understood to mean a molecule of intermediate relative molecular mass, the structure of which essentially comprises a small plurality of units derived, actually or conceptually, from molecules of lower relative molecular mass (Pure Appl. Chem., 1996, 68, 2291). In a preferred meaning as used herein present invention a polymer will be understood to mean a compound having >1, i.e. at least 2 repeat units, preferably ≥5 repeat units, and an oligomer will be understood to mean a compound with >1 and <10, preferably <5, repeat units.

Further, as used herein, the term “polymer” will be understood to mean a molecule that encompasses a backbone (also referred to as “main chain”) of one or more distinct types of repeat units (the smallest constitutional unit of the molecule) and is inclusive of the commonly known terms “oligomer”, “copolymer”, “homopolymer”, “random polymer” and the like.

Further, it will be understood that the term polymer is inclusive of, in addition to the polymer itself, residues from initiators, catalysts and other elements attendant to the synthesis of such a polymer, where such residues are understood as not being covalently incorporated thereto. Further, such residues and other elements, while normally removed during post polymerization purification processes, are typically mixed or co-mingled with the polymer such that they generally remain with the polymer when it is transferred between vessels or between solvents or dispersion media.

As used herein, in a formula showing a polymer or a repeat unit, like for example a unit of formula I or a polymer of formula III or IV or their subformulae, an asterisk (*) will be understood to mean a chemical linkage to an adjacent unit or to a terminal group in the polymer backbone. In a ring, like for example a benzene or thiophene ring, an asterisk (*) will be understood to mean a C atom that is fused to an adjacent ring.

As used herein, the terms “repeat unit”, “repeating unit” and “monomeric unit” are used interchangeably and will be understood to mean the constitutional repeating unit (CRU), which is the smallest constitutional unit the repetition of which constitutes a regular macromolecule, a regular oligomer molecule, a regular block or a regular chain (Pure Appl. Chem., 1996, 68, 2291). As further used herein, the term “unit” will be understood to mean a structural unit which can be a repeating unit on its own, or can together with other units form a constitutional repeating unit.

As used herein, a “terminal group” will be understood to mean a group that terminates a polymer backbone. The expression “in terminal position in the backbone” will be understood to mean a divalent unit or repeat unit that is linked at one side to such a terminal group and at the other side to another repeat unit. Such terminal groups include endcap groups, or reactive groups that are attached to a monomer forming the polymer backbone which did not participate in the polymerisation reaction, like for example a group having the meaning of R⁵ or R⁶ as defined below.

As used herein, the term “endcap group” will be understood to mean a group that is attached to, or replacing, a terminal group of the polymer backbone. The endcap group can be introduced into the polymer by an endcapping process. Endcapping can be carried out for example by reacting the terminal groups of the polymer backbone with a monofunctional compound (“endcapper”) like for example an alkyl- or arylhalide, an alkyl- or arylstannane or an alkyl- or arylboronate. The endcapper can be added for example after the polymerisation reaction. Alternatively the endcapper can be added in situ to the reaction mixture before or during the polymerisation reaction. In situ addition of an endcapper can also be used to terminate the polymerisation reaction and thus control the molecular weight of the forming polymer. Typical endcap groups are for example H, phenyl and lower alkyl.

As used herein, the term “small molecule” will be understood to mean a monomeric compound which typically does not contain a reactive group by which it can be reacted to form a polymer, and which is designated to be used in monomeric form. In contrast thereto, the term “monomer” unless stated otherwise will be understood to mean a monomeric compound that carries one or more reactive functional groups by which it can be reacted to form a polymer.

As used herein, the terms “donor” or “donating” and “acceptor” or “accepting” will be understood to mean an electron donor or electron acceptor, respectively. “Electron donor” will be understood to mean a chemical entity that donates electrons to another compound or another group of atoms of a compound. “Electron acceptor” will be understood to mean a chemical entity that accepts electrons transferred to it from another compound or another group of atoms of a compound. See also International Union of Pure and Applied Chemistry, Compendium of Chemical Technology, Gold Book, Version 2.3.2, 19. Aug. 2012, pages 477 and 480.

As used herein, the term “n-type” or “n-type semiconductor” will be understood to mean an extrinsic semiconductor in which the conduction electron density is in excess of the mobile hole density, and the term “p-type”or “p-type semiconductor” will be understood to mean an extrinsic semiconductor in which mobile hole density is in excess of the conduction electron density (see also, J. Thewlis, Concise Dictionary of Physics, Pergamon Press, Oxford, 1973).

As used herein, the term “leaving group” will be understood to mean an atom or group (which may be charged or uncharged) that becomes detached from an atom in what is considered to be the residual or main part of the molecule taking part in a specified reaction (see also Pure Appl. Chem., 1994, 66, 1134).

As used herein, unless stated otherwise the molecular weight is given as the number average molecular weight Mₙ or weight average molecular weight M_{W}, which is determined by gel permeation chromatography (GPC) against polystyrene standards in eluent solvents such as tetrahydrofuran, trichloromethane (TCM, chloroform), chlorobenzene or 1,2,4-trichloro-benzene. Unless stated otherwise, chlorobenzene is used as solvent. The degree of polymerization, also referred to as total number of repeat units, n, will be understood to mean the number average degree of polymerization given as n=Mₙ/M_{U}, wherein Mₙ is the number average molecular weight and M_{U} is the molecular weight of the single repeat unit, see J. M. G. Cowie, Polymers: Chemistry & Physics of Modern Materials, Blackie, Glasgow, 1991.

As used herein, the term “carbyl group” will be understood to mean any monovalent or multivalent organic moiety which comprises at least one carbon atom either without any non-carbon atoms (like for example —C═C—), or optionally combined with at least one non-carbon atom such as B, N, O, S, P, Si, Se, As, Te or Ge (for example carbonyl etc.).

As used herein, the term “hydrocarbyl group” will be understood to mean a carbyl group that does additionally contain one or more H atoms and optionally contains one or more hetero atoms like for example B, N, O, S, P, Si, Se, As, Te or Ge.

As used herein, the term “hetero atom” will be understood to mean an atom in an organic compound that is not a H- or C-atom, and preferably will be understood to mean B, N, O, S, P, Si, Se, Sn, As, Te or Ge.

A carbyl or hydrocarbyl group comprising a chain of 3 or more C atoms may be straight-chain, branched and/or cyclic, and may include spiro-connected and/or fused rings.

Preferred carbyl and hydrocarbyl groups include alkyl, alkoxy, thioalkyl, alkylcarbonyl, alkoxycarbonyl, alkylcarbonyloxy and alkoxycarbonyloxy, each of which is optionally substituted and has 1 to 40, preferably 1 to 25, very preferably 1 to 18 C atoms, furthermore optionally substituted aryl or aryloxy having 6 to 40, preferably 6 to 25 C atoms, furthermore alkylaryloxy, arylcarbonyl, aryloxycarbonyl, arylcarbonyloxy and aryloxycarbonyloxy, each of which is optionally substituted and has 6 to 40, preferably 7 to 40 C atoms, wherein all these groups do optionally contain one or more hetero atoms, preferably selected from B, N, O, S, P, Si, Se, As, Te and Ge.

Further preferred carbyl and hydrocarbyl group include for example: a C₁-C₄₀ alkyl group, a C₁-C₄₀ fluoroalkyl group, a C₁-C₄₀ alkoxy or oxaalkyl group, a C₂-C₄₀ alkenyl group, a C₂-C₄₀ alkynyl group, a C₃-C₄₀ allyl group, a C₄-C₄₀ alkylidenyl group, a C₄-C₄₀ polyenyl group, a C₂-C₄₀ ketone group, a C₂-C₄₀ ester group, a C₆-C₁₈ aryl group, a C₆-C₄₀ alkylaryl group, a C₆-C₄₀ arylalkyl group, a C₄-C₄₀ cycloalkyl group, a C₄-C₄₀ cycloalkenyl group, and the like. Preferred among the foregoing groups are a C₁-C₂₀ alkyl group, a C₁-C₂₀ fluoroalkyl group, a C₂-C₂₀ alkenyl group, a C₂-C₂₀ alkynyl group, a C₃-C₂₀ ally) group, a C₄-C₂₀ alkylidenyl group, a C₂-C₂₀ ketone group, a C₂-C₂₀ ester group, a C₆-C₁₂ aryl group, and a C₄-C₂₀ polyenyl group, respectively.

Also included are combinations of groups having carbon atoms and groups having hetero atoms, like e.g. an alkynyl group, preferably ethynyl, that is substituted with a silyl group, preferably a trialkylsilyl group.

The carbyl or hydrocarbyl group may be an acyclic group or a cyclic group. Where the carbyl or hydrocarbyl group is an acyclic group, it may be straight-chain or branched. Where the carbyl or hydrocarbyl group is a cyclic group, it may be a non-aromatic carbocyclic or heterocyclic group, or an aryl or heteroaryl group.

A non-aromatic carbocyclic group as referred to above and below is saturated or unsaturated and preferably has 4 to 30 ring C atoms. A non-aromatic heterocyclic group as referred to above and below preferably has 4 to 30 ring C atoms, wherein one or more of the C ring atoms are optionally replaced by a hetero atom, preferably selected from N, O, P, S, Si and Se, or by a —S(O)— or —S(O)₂— group. The non-aromatic carbo- and heterocyclic groups are mono- or polycyclic, may also contain fused rings, preferably contain 1, 2, 3 or 4 fused or unfused rings, and are optionally substituted with one or more groups L, wherein

L is selected from F, Cl, Br, I, —CN, —NC, —NCO, —NCS, —OCN, —SCN, —R⁰, —OR⁰, —SR⁰, —C(═O)X°, —C(═O)R⁰, —C(═O)—OR⁰, —O—C(═O)—R⁰, —NH₂, —NHR⁰, —NR⁰R⁰⁰, —C(═O)NHR⁰, —C(═O)NR⁰R⁰⁰, —SO₃R⁰, —SO₂R⁰, —OH, —NO₂, —CF₃, —SF₅, or optionally substituted silyl, or carbyl or hydrocarbyl with 1 to 30, preferably 1 to 20 C atoms that is optionally substituted and optionally comprises one or more hetero atoms, wherein X⁰ is halogen, preferably F or Cl, and R⁰, R⁰⁰ denote H or straight-chain or branched alkyl with 1 to 24, preferably 1 to 12 C atoms that is optionally fluorinated, preferably L is a C₁₋₂₄ alkyl chain selected from formulae SUB1-6 as defined below that is optionally fluorinated.

Preferably L is selected from F, Cl, —CN, —R⁰, —OR⁰, —SR⁰, —C(═O)—R⁰, —C(═O)—OR⁰, —O—C(═O)—R⁰, —O—C(═O)—OR⁰, —C(═O)—NHR⁰ and —C(═O)—NR⁰R⁰⁰.

Further preferably L is selected from F or alkyl, alkoxy, oxaalkyl, thioalkyl, fluoroalkyl, fluoroalkoxy, alkylcarbonyl, alkoxycarbonyl, with 1 to 12 C atoms, or alkenyl or alkynyl with 2 to 12 C atoms or a C₁₋₂₄ alkyl chain selected from formulae SUB1-6 as defined below that is optionally fluorinated.

Preferred non-aromatic carbocyclic or heterocyclic groups are tetrahydrofuran, indane, pyran, pyrrolidine, piperidine, cyclopentane, cyclohexane, cycloheptane, cyclopentanone, cyclohexanone, dihydro-furan-2-one, tetrahydro-pyran-2-one and oxepan-2-one.

An aryl group as referred to above and below preferably has 4 to 30 ring C atoms, is mono- or polycyclic and may also contain fused rings, preferably contains 1, 2, 3 or 4 fused or unfused rings, and is optionally substituted with one or more groups L as defined above.

A heteroaryl group as referred to above and below preferably has 4 to 30 ring C atoms, wherein one or more of the C ring atoms are replaced by a hetero atom, preferably selected from N, O, S, Si and Se, is mono- or polycyclic and may also contain fused rings, preferably contains 1, 2, 3 or 4 fused or unfused rings, and is optionally substituted with one or more groups L as defined above.

An arylalkyl or heteroarylalkyl group as referred to above and below preferably denotes —(CH₂)ₐ-aryl or —(CH₂)ₐ-heteroaryl, wherein a is an integer from 1 to 6, preferably 1, and “aryl” and “heteroaryl” have the meanings given above and below. A preferred arylalkyl group is benzyl which is optionally substituted by L.

As used herein, “arylene” will be understood to mean a divalent aryl group, and “heteroarylene” will be understood to mean a divalent heteroaryl group, including all preferred meanings of aryl and heteroaryl as given above and below.

Preferred aryl and heteroaryl groups are phenyl in which, in addition, one or more CH groups may be replaced by N, naphthalene, thiophene, selenophene, thienothiophene, dithienothiophene, fluorene and oxazole, all of which can be unsubstituted, mono- or polysubstituted with L as defined above. Very preferred aryl and heteroaryl groups are selected from pyrrole, preferably N-pyrrole, furan, pyridine, preferably 2- or 3-pyridine, pyrimidine, pyridazine, pyrazine, triazole, tetrazole, pyrazole, imidazole, isothiazole, thiazole, thiadiazole, isoxazole, oxazole, oxadiazole, thiophene, preferably 2-thiophene or 2,5-dithiophene-2′,5′-diyl, selenophene, preferably 2-selenophene, thieno[3,2-b]thiophene, thieno[2,3-b]thiophene, furo[3,2-b]furan, furo[2,3-b]furan, seleno[3,2-b]selenophene, seleno[2,3-b]selenophene, thieno[3,2-b]selenophene, thieno[3,2-b]furan, indole, isoindole, benzo[b]furan, benzo[b]thiophene, benzo[1,2-b;4,5-b′]dithiophene, benzo[2,1-b;3,4-b′]dithiophene, quinole, 2 methylquinole, isoquinole, quinoxaline, quinazoline, benzotriazole, benzimidazole, benzothiazole, benzisothiazole, benzisoxazole, benzoxadiazole, benzoxazole, benzothiadiazole, 4H-cyclopenta[2,1-b;3,4-b′]dithiophene, 7H-3,4-dithia-7-sila-cyclopenta[a]pentalene, all of which can be unsubstituted, mono- or polysubstituted with L as defined above. Further examples of aryl and heteroaryl groups are those selected from the groups shown hereinafter.

An alkyl group or an alkoxy group, i.e., where the terminal CH₂ group is replaced by —O—, can be straight-chain or branched. Particularly preferred straight-chain groups have 2, 3, 4, 5, 6, 7, 8, 12 or 16 carbon atoms and accordingly denote preferably ethyl, propyl, butyl, pentyl, hexyl, heptyl, octyl, dodecyl or hexadecyl, ethoxy, propoxy, butoxy, pentoxy, hexoxy, heptoxy, octoxy, dodecoxy or hexadecoxy, furthermore methyl, nonyl, decyl, undecyl, tridecyl, tetradecyl, pentadecyl, nonoxy, decoxy, undecoxy, tridecoxy or tetradecoxy, for example.

An alkenyl group, i.e., wherein one or more CH₂ groups are replaced by —CH═CH— can be straight-chain or branched. It is preferably straight-chain, has 2 to 10 C atoms and accordingly is preferably vinyl, prop-1-, or prop-2-enyl, but-1-, 2- or but-3-enyl, pent-1-, 2-, 3- or pent-4-enyl, hex-1-, 2-, 3-, 4- or hex-5-enyl, hept-1-, 2-, 3-, 4-, 5- or hept-6-enyl, oct-1-, 2-, 3-, 4-, 5-, 6- or oct-7-enyl, non-1-, 2-, 3-, 4-, 5-, 6-, 7- or non-8-enyl, dec-1-, 2-, 3-, 4-, 5-, 6-, 7-, 8- or dec-9-enyl.

Especially preferred alkenyl groups are C₂-C₇-1E-alkenyl, C₄-C₇-3E-alkenyl, C₅-C₇-4-alkenyl, C₆-C₇-5-alkenyl and C₇-6-alkenyl, in particular C₂-C₇-1E-alkenyl, C₄-C₇-3E-alkenyl and C₅-C₇-4-alkenyl. Examples for particularly preferred alkenyl groups are vinyl, 1E-propenyl, 1E-butenyl, 1E-pentenyl, 1E-hexenyl, 1E-heptenyl, 3-butenyl, 3E-pentenyl, 3E-hexenyl, 3E-heptenyl, 4-pentenyl, 4Z-hexenyl, 4E-hexenyl, 4Z-heptenyl, 5-hexenyl, 6-heptenyl and the like. Groups having up to 5 C atoms are generally preferred.

An oxaalkyl group, i.e., where one CH₂ group is replaced by —O—, can be straight-chain. Particularly preferred straight-chain groups are 2-oxapropyl (=methoxymethyl), 2-(=ethoxymethyl) or 3-oxabutyl (=2-methoxyethyl), 2-, 3-, or 4-oxapentyl, 2-, 3-, 4-, or 5-oxahexyl, 2-, 3-, 4-, 5-, or 6-oxaheptyl, 2-, 3-, 4-, 5-, 6- or 7-oxaoctyl, 2-, 3-, 4-, 5-, 6-, 7- or 8-oxanonyl or 2-, 3-, 4-, 5-, 6-, 7-, 8- or 9-oxadecyl, for example.

In an alkyl group wherein one CH₂ group is replaced by —O— and one CH₂ group is replaced by —C(O)—, these radicals are preferably neighboured. Accordingly these radicals together form a carbonyloxy group —C(O)—O— or an oxycarbonyl group —O—C(O)—. Preferably this group is straight-chain and has 2 to 6 C atoms. It is accordingly preferably acetyloxy, propionyloxy, butyryloxy, pentanoyloxy, hexanoyloxy, acetyloxymethyl, propionyloxymethyl, butyryloxymethyl, pentanoyloxymethyl, 2-acetyloxyethyl, 2-propionyloxyethyl, 2-butyryloxyethyl, 3-acetyloxypropyl, 3-propionyloxypropyl, 4-acetyloxybutyl, methoxycarbonyl, ethoxycarbonyl, propoxycarbonyl, butoxycarbonyl, pentoxycarbonyl, methoxycarbonylmethyl, ethoxycarbonylmethyl, propoxycarbonylmethyl, butoxycarbonylmethyl, 2-(methoxycarbonyl)ethyl, 2-(ethoxycarbonyl)ethyl, 2-(propoxycarbonyl)ethyl, 3-(methoxycarbonyl)propyl, 3-(ethoxycarbonyl)propyl, 4-(methoxycarbonyl)-butyl.

An alkyl group wherein two or more CH₂ groups are replaced by —O— and/or —C(O)O— can be straight-chain or branched. It is preferably straight-chain and has 3 to 12 C atoms. Accordingly, it is preferably bis-carboxy-methyl, 2,2-bis-carboxy-ethyl, 3,3-bis-carboxy-propyl, 4,4-bis-carboxy-butyl, 5,5-bis-carboxy-pentyl, 6,6-bis-carboxy-hexyl, 7,7-bis-carboxy-heptyl, 8,8-bis-carboxy-octyl, 9,9-bis-carboxy-nonyl, 10,10-bis-carboxy-decyl, bis-(methoxycarbonyl)-methyl, 2,2-bis-(methoxycarbonyl)-ethyl, 3,3-bis-(methoxycarbonyl)-propyl, 4,4-bis-(methoxycarbonyl)-butyl, 5,5-bis-(methoxycarbonyl)-pentyl, 6,6-bis-(methoxycarbonyl)-hexyl, 7,7-bis-(methoxycarbonyl)-heptyl, 8,8-bis-(methoxycarbonyl)-octyl, bis-(ethoxycarbonyl)-methyl, 2,2-bis-(ethoxycarbonyl)-ethyl, 3,3-bis-(ethoxycarbonyl)-propyl, 4,4-bis-(ethoxycarbonyl)-butyl, 5,5-bis-(ethoxycarbonyl)-hexyl.

A thioalkyl group, i.e., where one CH₂ group is replaced by —S—, is preferably straight-chain thiomethyl (—SCH₃), 1-thioethyl (—SCH₂CH₃), 1-thiopropyl (=—SCH₂CH₂CH₃), 1-(thiobutyl), 1-(thiopentyl), 1-(thiohexyl), 1-(thioheptyl), 1-(thiooctyl), 1-(thiononyl), 1-(thiodecyl), 1-(thioundecyl) or 1-(thiododecyl), wherein preferably the CH₂ group adjacent to the sp² hybridised vinyl carbon atom is replaced.

A fluoroalkyl group can either be perfluoroalkyl CᵢF₂ᵢ₊₁, wherein i is an integer from 1 to 15, in particular CF₃, C₂F₅, C₃F₇, C₄F₉, C₅F₁₁, C₆F₁₃, C₇F₁₅ or C₈F₁₇, very preferably C₆F₁₃, or partially fluorinated alkyl, preferably with 1 to 15 C atoms, in particular 1,1-difluoroalkyl, all of the aforementioned being straight-chain or branched.

Preferably “fluoroalkyl” means a partially fluorinated (i.e. not perfluorinated) alkyl group.

Alkyl, alkoxy, alkenyl, oxaalkyl, thioalkyl, carbonyl and carbonyloxy groups can be achiral or chiral groups. Particularly preferred chiral groups are 2-butyl (=1-methylpropyl), 2-methylbutyl, 2-methylpentyl, 3-methylpentyl, 2-ethylhexyl, 2-butyloctyl, 2-hexyldecyl, 2-octyldodecyl, 3,7-dimethyloctyl, 3,7,11-trimethyldodecyl, 2-propylpentyl, in particular 2-methylbutyl, 2 methylbutoxy, 2-methylpentoxy, 3-methyl-pentoxy, 2-ethyl-hexoxy, 2-butyloctoxyo, 2-hexyldecoxy, 2-octyldodecoxy, 3,7-dimethyloctoxy, 3,7,11-trimethyldodecoxy, 1-methylhexoxy, 2-octyloxy, 2-oxa-3-methylbutyl, 3-oxa-4-methyl-pentyl, 4-methylhexyl, 2-hexyl, 2-octyl, 2-nonyl, 2-decyl, 2-dodecyl, 6-methoxy-octoxy, 6-methyloctoxy, 6-methyloctanoyloxy, 5 methylheptyloxy-carbonyl, 2-methylbutyryloxy, 3-methylvaleroyloxy, 4-methylhexanoyloxy, 2-chloro-propionyloxy, 2-chloro-3-methylbutyryloxy, 2-chloro-4-methyl-valeryl-oxy, 2-chloro-3-methylvaleryloxy, 2-methyl-3-oxapentyl, 2-methyl-3-oxa-hexyl, 1-methoxypropyl-2-oxy, 1-ethoxypropyl-2-oxy, 1-propoxypropyl-2-oxy, 1-butoxypropyl-2-oxy, 2-fluorooctyloxy, 2 fluorodecyloxy, 1,1,1-trifluoro-2-octyloxy, 1,1,1-trifluoro-2-octyl, 2-fluoromethyloctyloxy for example. Very preferred are 2-methylbutyl, 2-ethylhexyl, 2-butyloctyl, 2-hexyldecyl, 2-octyldodecyl, 3,7-dimethyloctyl, 3,7,11-trimethyldodecyl, 2-hexyl, 2-octyl, 2-octyloxy, 1,1,1-trifluoro-2-hexyl, 1,1,1-trifluoro-2-octyl and 1,1,1-trifluoro-2-octyloxy.

Preferred achiral branched groups are isopropyl, isobutyl (=methylpropyl), isopentyl (=3-methylbutyl), tert. butyl, isopropoxy, 2-methyl-propoxy and 3-methylbutoxy.

In a preferred embodiment, the substituents on an aryl or heteroaryl ring are independently of each other selected from primary, secondary or tertiary alkyl, alkoxy, oxaalkyl, thioalkyl, alkylcarbonyl or alkoxycarbonyl with 1 to 30 C atoms, wherein one or more H atoms are optionally replaced by F, or aryl, aryloxy, heteroaryl or heteroaryloxy that is optionally alkylated, alkoxylated, alkylthiolated or esterified and has 4 to 30 ring atoms. Further preferred substituents are selected from the group consisting of the following formulae

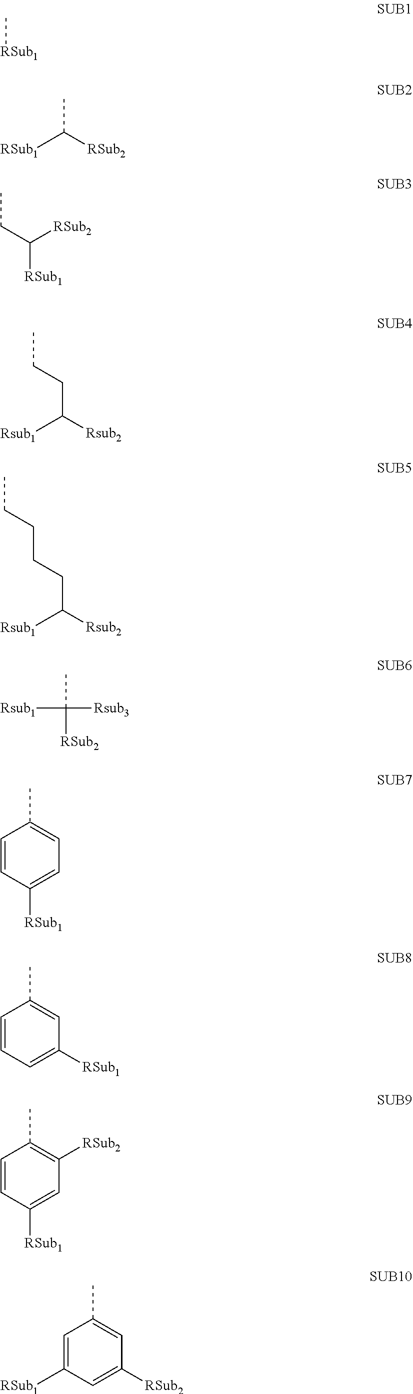

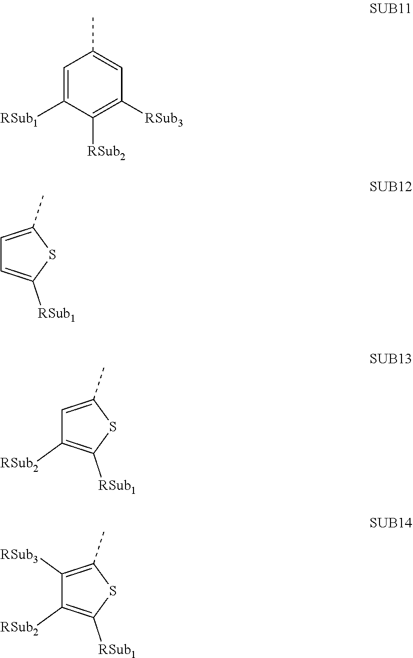

wherein RSub₁₋₃ denotes L as defined above and below and where at least one, preferably all groups RSub₁₋₃ are selected from alkyl, alkoxy, oxaalkyl, thioalkyl, alkylcarbonyl or alkoxycarbonyl with 1 to 24 C atoms, preferably 1 to 20 C atoms, that is optionally fluorinated, and wherein the dashed line denotes the link to the ring to which these groups are attached. Very preferred among these substituents are those wherein all RSub₁₋₃ subgroups are identical.

As used herein, if an aryl(oxy) or heteroaryl(oxy) group is “alkylated or alkoxylated”, this means that it is substituted with one or more alkyl or alkoxy groups having from 1 to 24 C-atoms and being straight-chain or branched and wherein one or more H atoms are optionally substituted by an F atom.

Above and below, Y¹ and Y² are independently of each other H, F, Cl or CN.

As used herein, —CO—, —C(═O)— and —C(O)— will be understood to mean a carbonyl group, i.e. a group having the structure

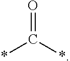

As used herein, C═CR¹R² will be understood to mean a group having the structure

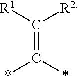

As used herein, “halogen” includes F, Cl, Br or I, preferably F, Cl or Br. A halogen atom that represents a substituent on a ring or chain is preferably F or Cl, very preferably F. A halogen atom that represents a reactive group in a monomer or an intermediate is preferably Br or I.

Above and below, the term “mirror image” means a moiety that can be obtained from another moiety by flipping it vertically or horizontally across an external symmetry plane or a symmetry plane extending through the moiety. For example the moiety

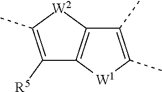

also includes the mirror images

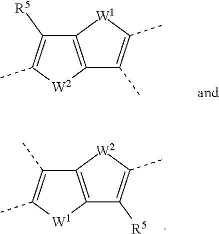

### DETAILED DESCRIPTION

The compounds of the present invention are easy to synthesize and exhibit advantageous properties. They show good processability for the device manufacture process, high solubility in organic solvents, and are especially suitable for large scale production using solution processing methods.

The compounds of formula I are especially suitable as (electron) acceptor or n-type semiconductor, and for the preparation of blends of n-type and p-type semiconductors which are suitable for use in OPD or BHJ OPV devices.

The compounds of formula I are further suitable to replace the fullerene compounds that have hitherto been used as n-type semiconductor in OPV or OPD devices.

Besides, the compounds of formula I show the following advantageous properties:
i) Substitution in positions R¹⁻¹⁷ and/or Ar¹⁻⁶ for example with solubilising groups enables greater light stability of the bulk heterojunction.
ii) Substitution in positions R¹⁻¹⁷ and/or Ar¹⁻⁶ for example with solubilising groups enables greater stability towards light illumination of the bulk heterojunction through mediation of the crystallisation and/or phase separation kinetic, thus stabilising the initial equilibrium thermodynamics in the BHJ.
iii) Substitution in positions R¹⁻¹⁷ and/or Ar¹⁻⁶ for example with solubilising groups enables greater thermal stability of the bulk heterojunction through mediation of the crystallisation and/or phase separation kinetic, thus stabilising the initial equilibrium thermodynamics in the BHJ.
iv) Compared to previously disclosed n-type OSCs for OPV/OPD application, the compounds of formula I provide the advantage that they enable further optimization of the HOMO and LUMO levels of the polycyclic unit through substitution, careful selection of the Ar¹⁻⁶ units, and inclusion of non-conjugated moieties can give improved light absorption.
v) Further optimization of the HOMO and LUMO levels of the polycyclic unit in formula I through substitution and/or careful selection of the Ar¹⁻⁶ units and inclusion of non-conjugated moieties can increase the open circuit potential (V_{oc}).
vi) When using the compounds as n-type OSC in a composition with a p-type OSC in the photoactive layer of an OPV or OPD, additional fine-tuning of the HOMO and LUMO levels of the polycyclic unit in formula I, for example through substitution and/or careful selection of the Ar¹⁻⁶ units and inclusion of non-conjugated moieties can reduce the energy loss in the electron transfer process between the n-type acceptor and the p-type donor material in the photoactive layer.
vii) Substitution in positions R¹⁻¹⁷ and/or Ar¹⁻⁶ can enable higher solubility in non-halogenated solvents due to the increased number of solubilising groups.

The synthesis of the compounds of formula I can be achieved based on methods that are known to the skilled person and described in the literature, as will be further illustrated herein.

Preferred conjugating groups Ar¹ of formula AC1a-AC1h are selected from the following formulae

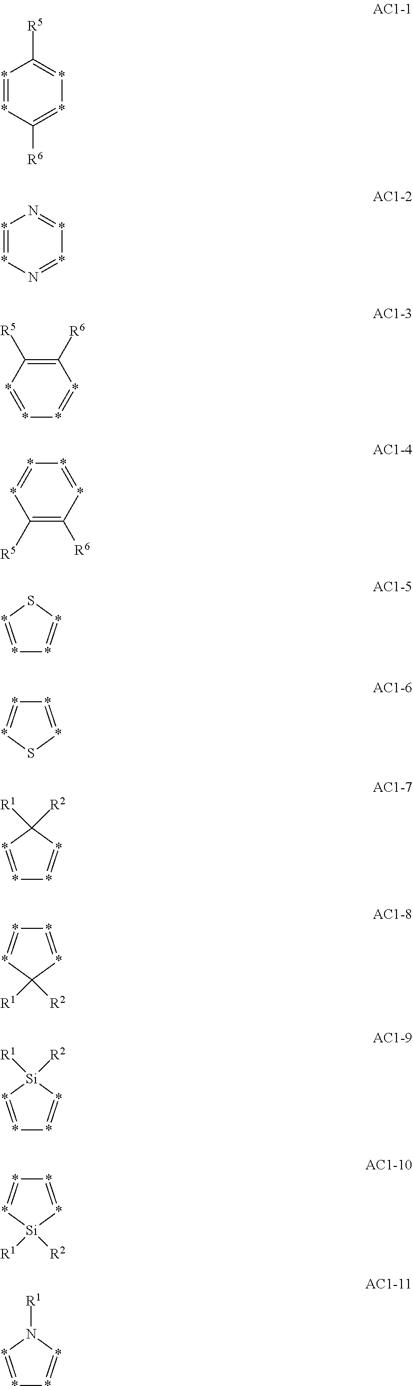

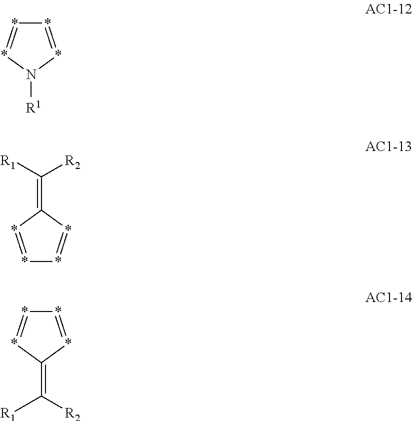

Preferred non-conjugating groups Art of formula AN1a-AN1h are selected from the following formulae

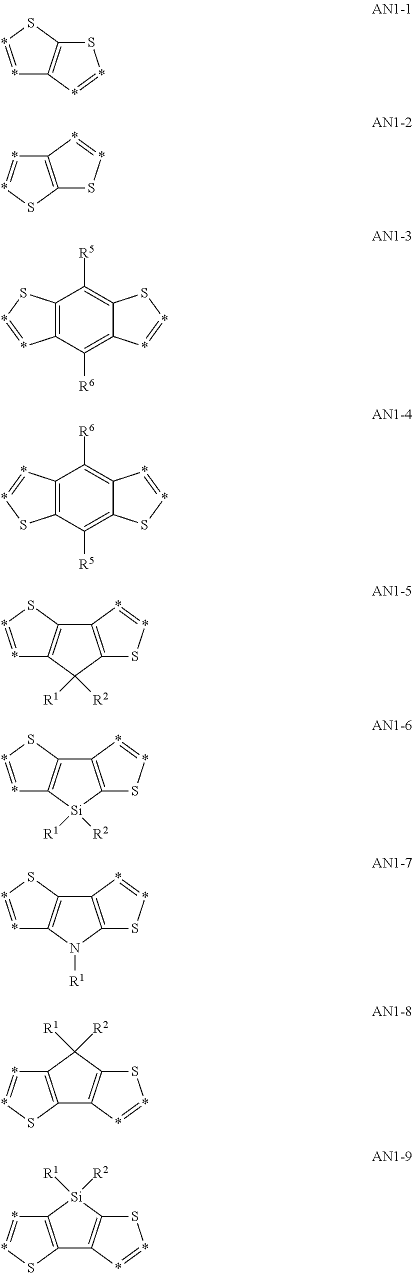

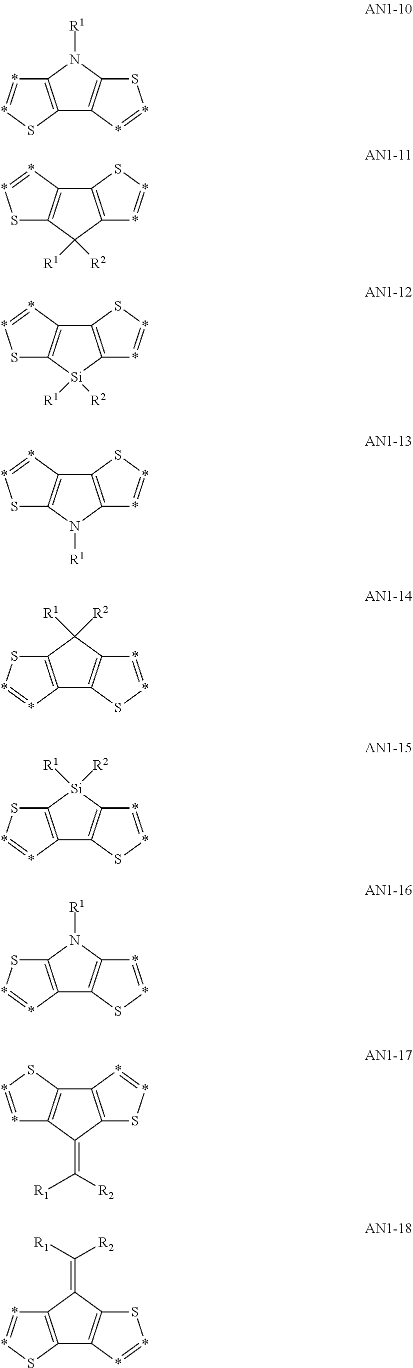

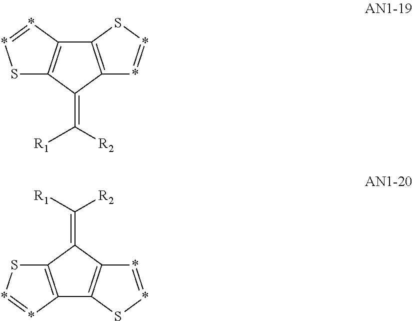

wherein R¹⁻⁶ are as defined above and below.

Preferred conjugating groups Are of formulae AC2a-AC2d are selected from the following formulae

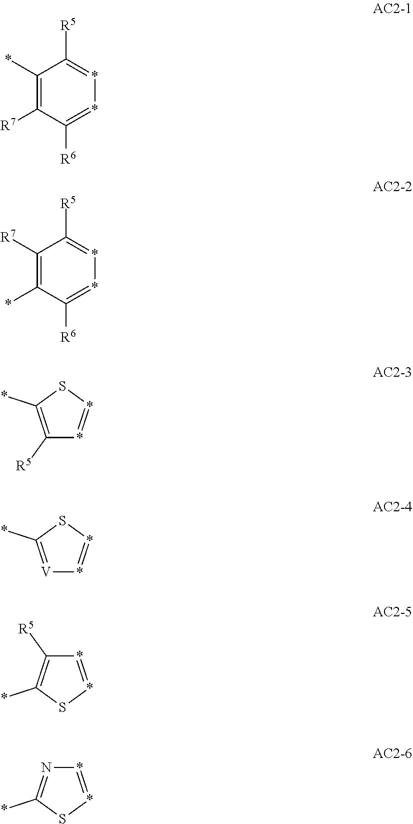

Preferred non-conjugating groups Are of formulae AN2a-AN2n are selected from the following formulae

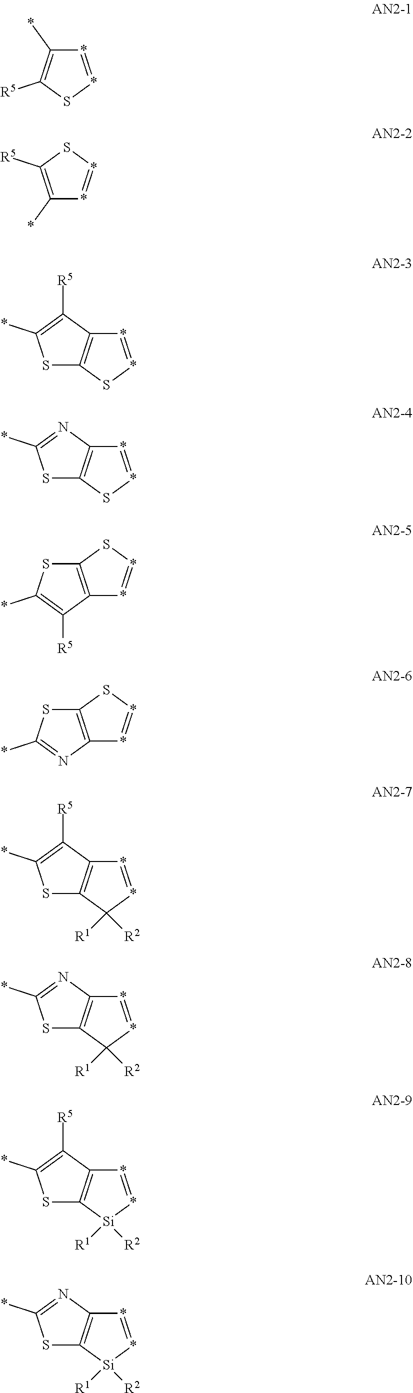

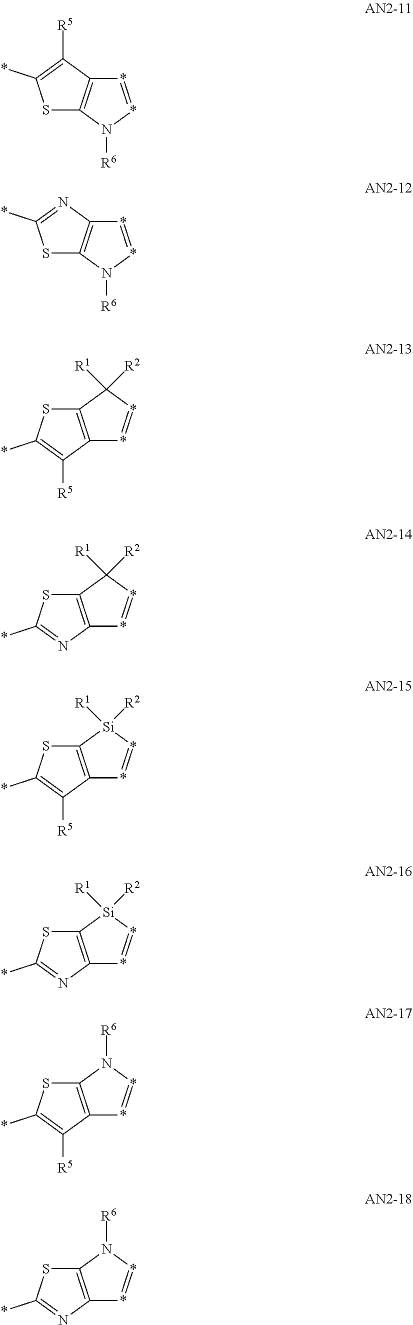

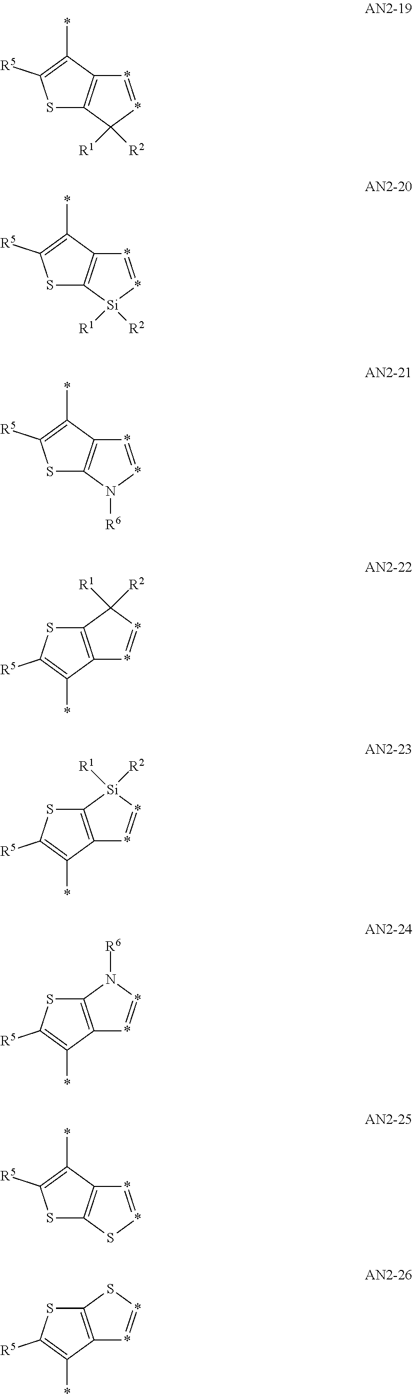

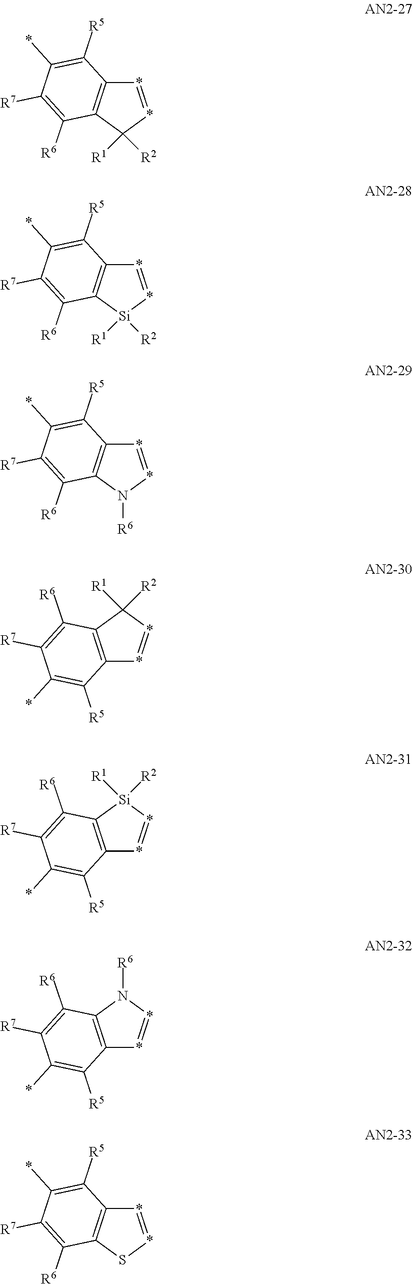

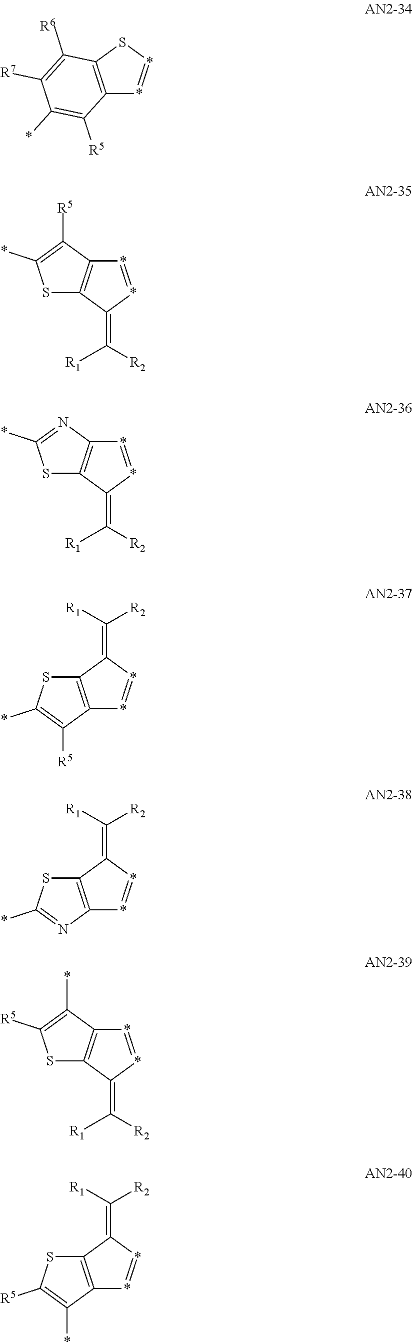

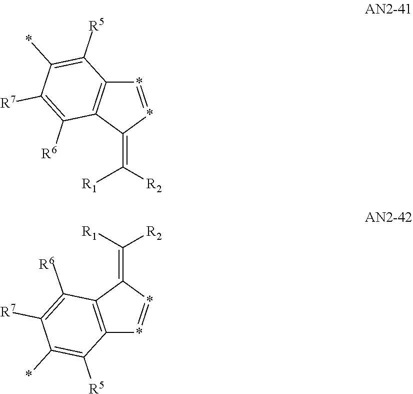

wherein R¹⁻⁷ are as defined above and below.

Preferred conjugating groups Ar³ of formulae AC3a-AC3d are selected from the following formulae

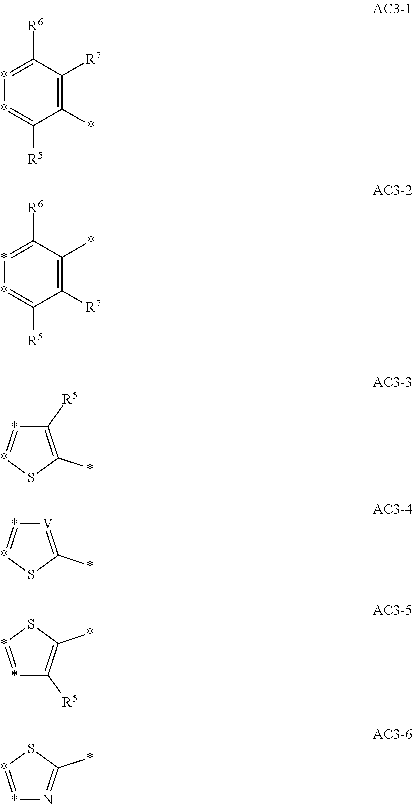

Preferred non-conjugating groups Ar³ of formulae AN3a-AN3n are selected from the following formulae

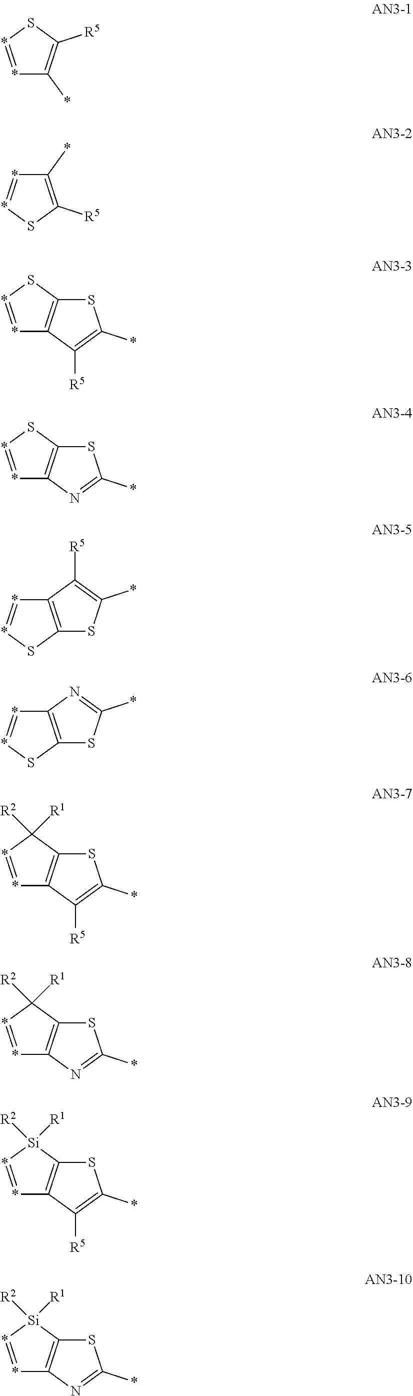

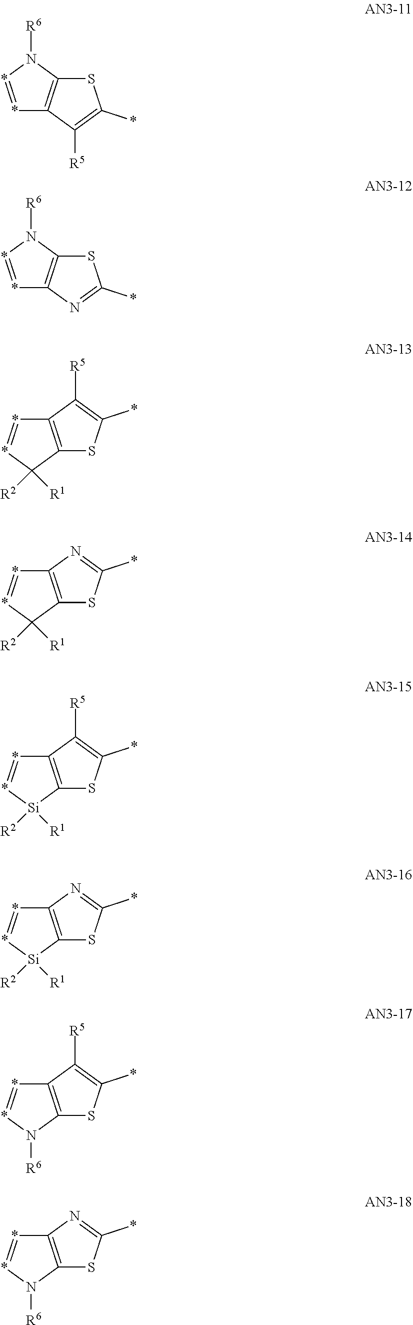

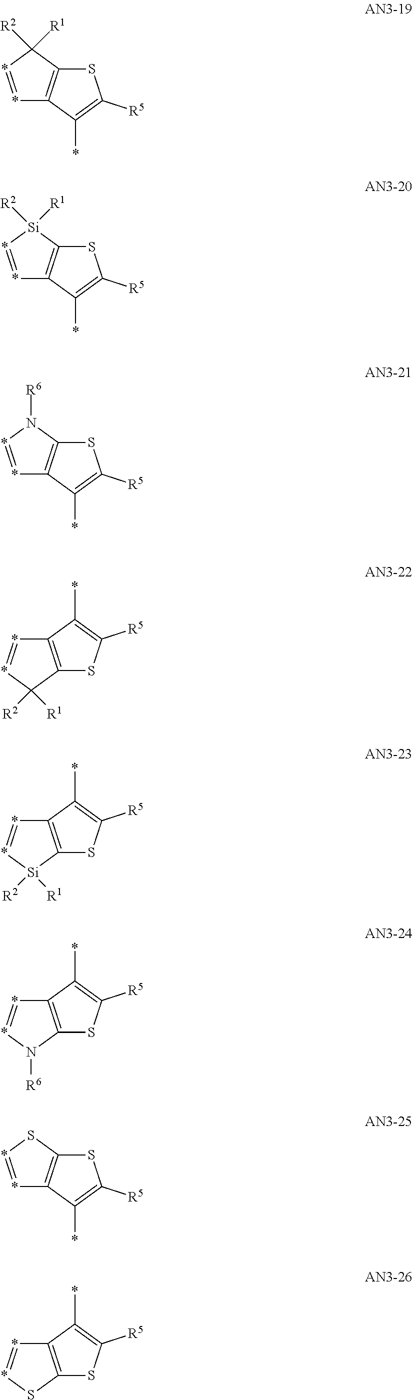

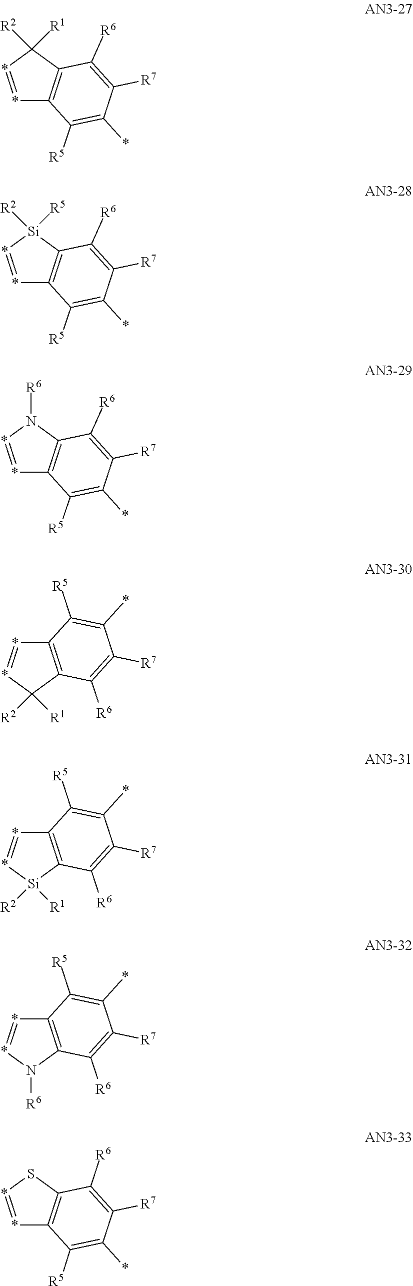

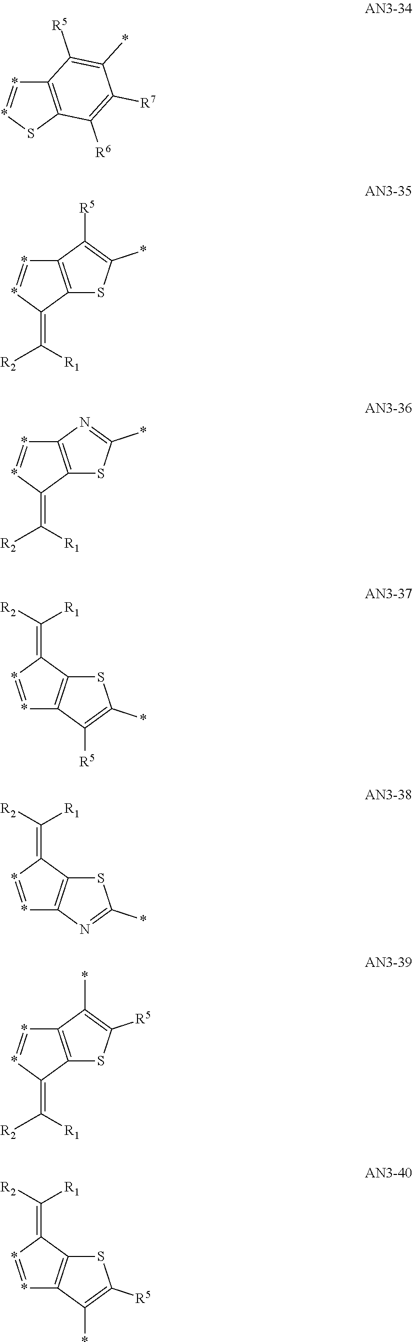

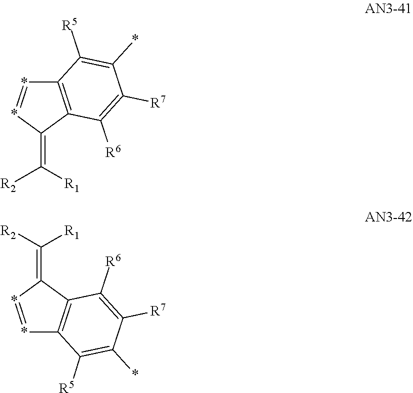

wherein R¹⁻⁷ are as defined above and below.

Preferred conjugating groups Ar⁴, Ar⁵ and Ar⁶ in formula I are selected from the following formulae and their mirror images.

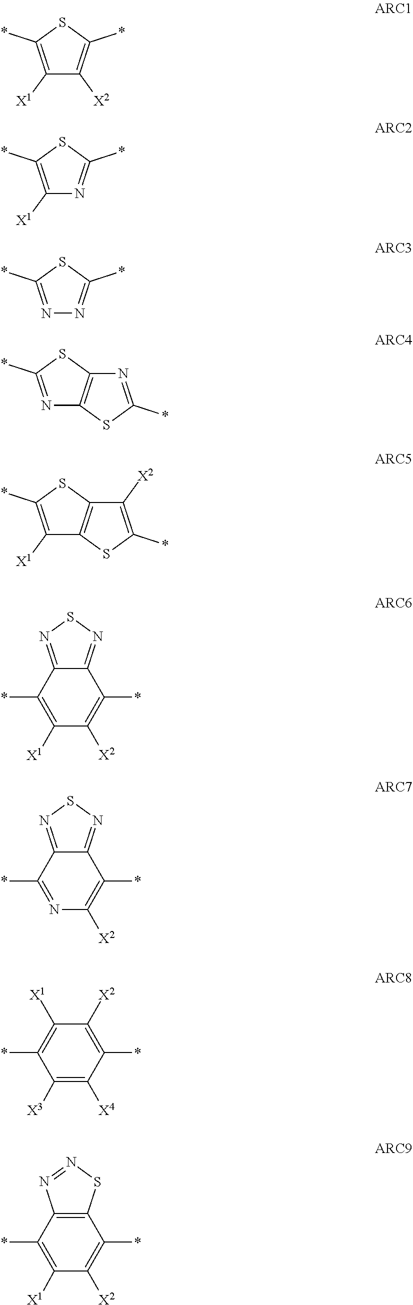

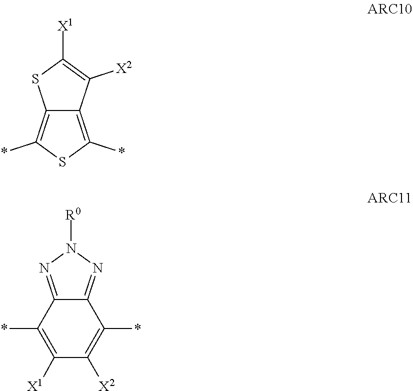

Preferred non-conjugating groups Ar⁴, Ar⁵ and Ar⁶ in formula I are selected from the following formulae and their mirror images.

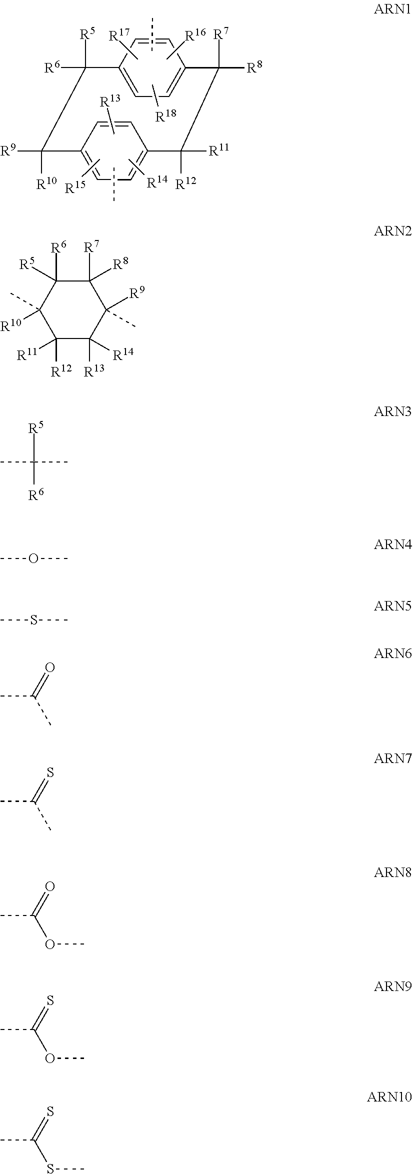

wherein X¹, X², X³ and X⁴ have one of the meanings given for R⁵ above and below, and preferably denote H, F, Cl, —CN, —R⁰, —OR⁰ or —C(═O)OR⁰. Preferred formulae ARC1, ARC2, ARC5, ARC6, ARC7, ARC8, ARC9, ARC10 and ARC11 are those containing at least one, preferably one, two or four substituents X¹⁻⁴ selected from F and Cl, very preferably F.

Preferably the compound of formula I comprises one or more conjugating groups selected from the following formulae

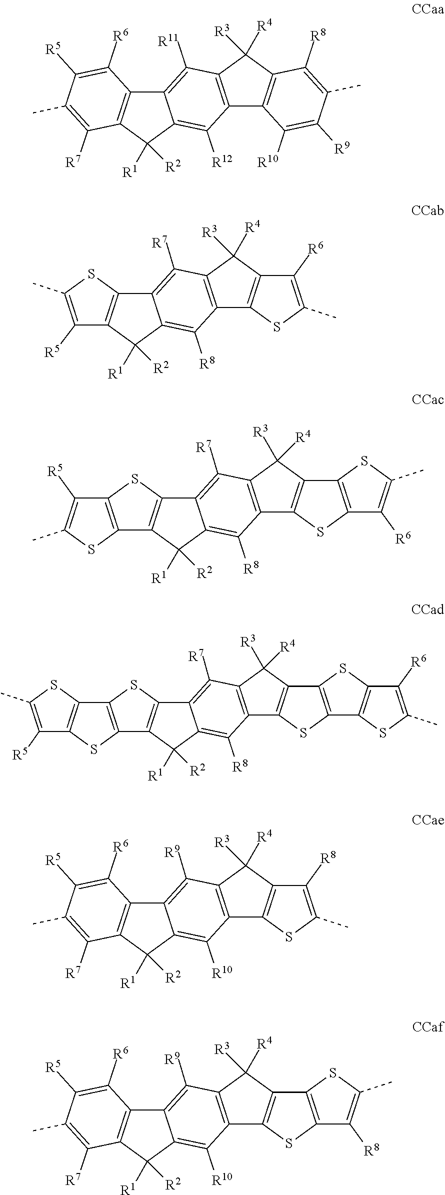

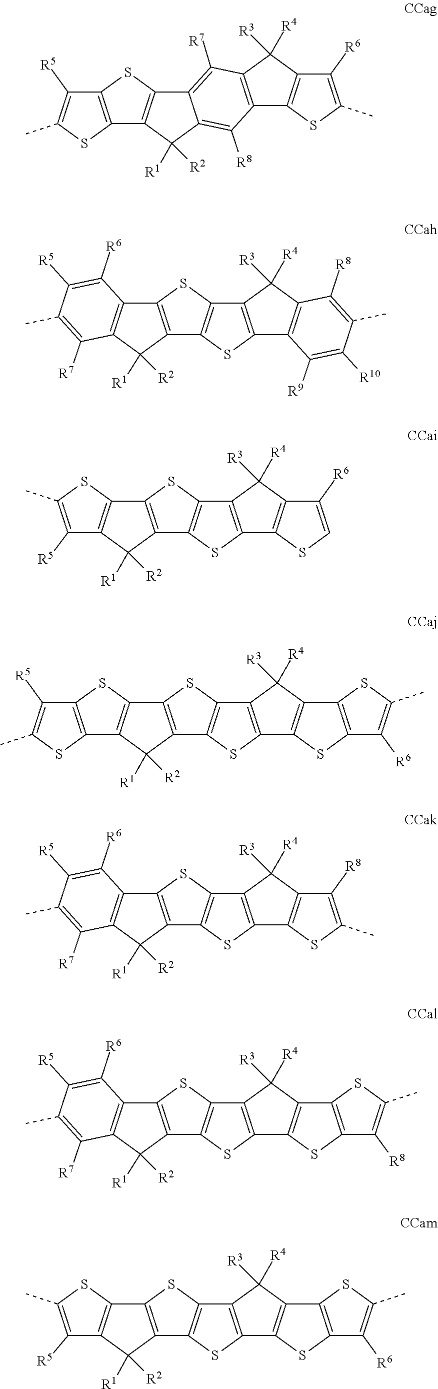

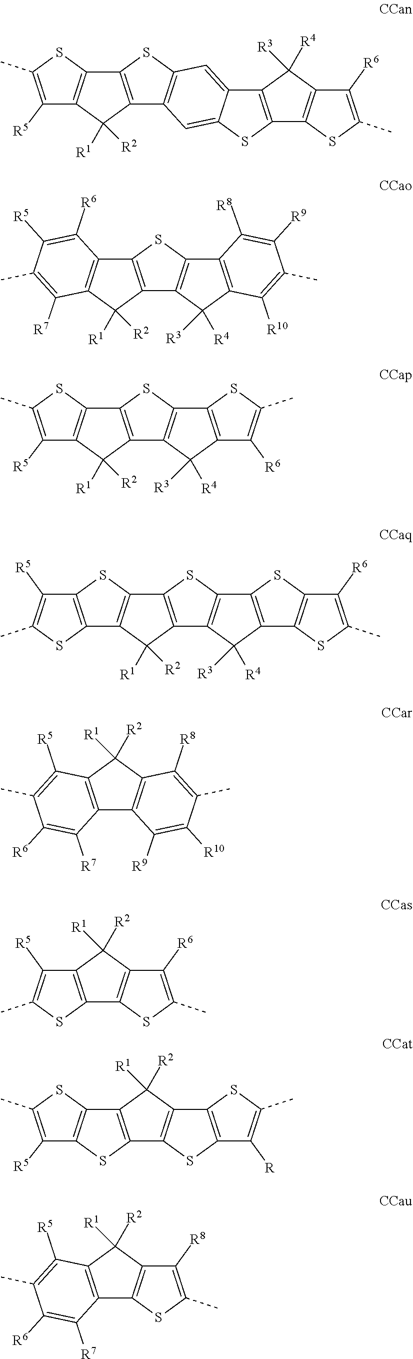

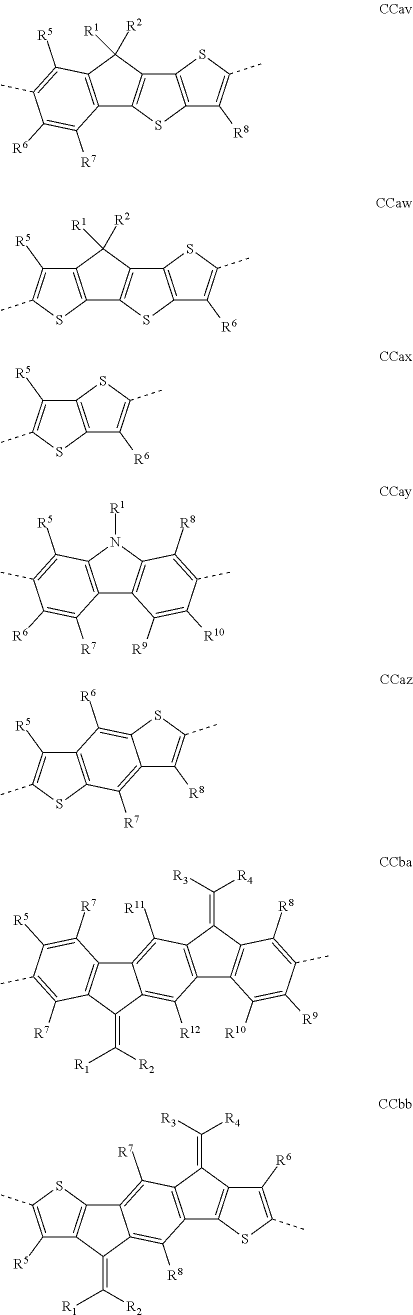

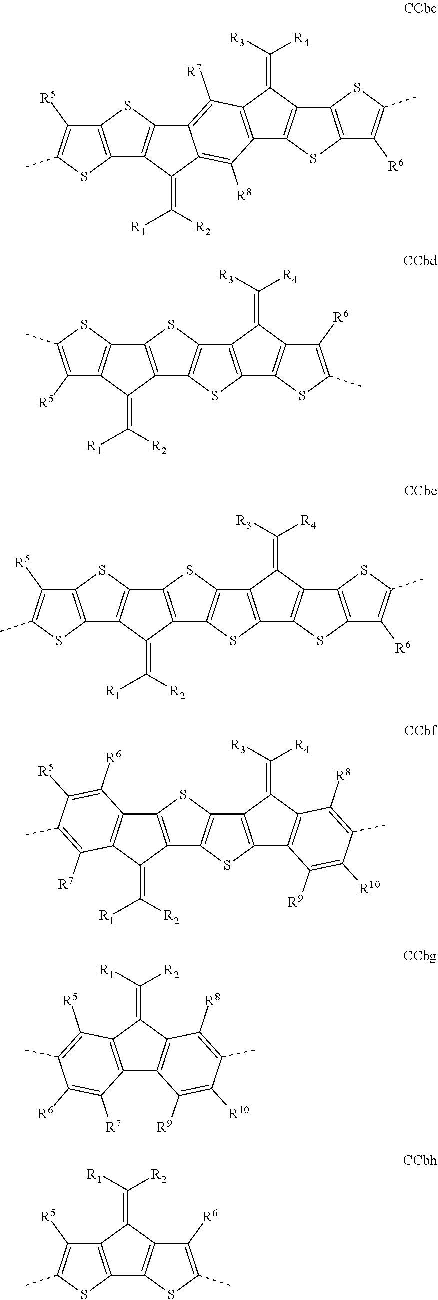

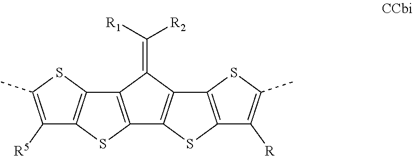

Further preferably the compound of formula I comprises one or more non-conjugating groups selected from the following formulae

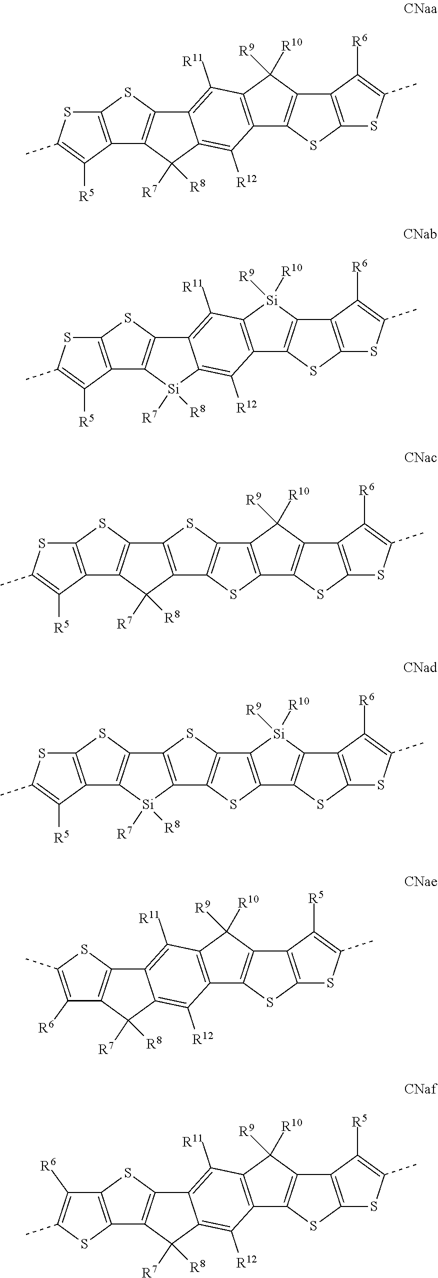

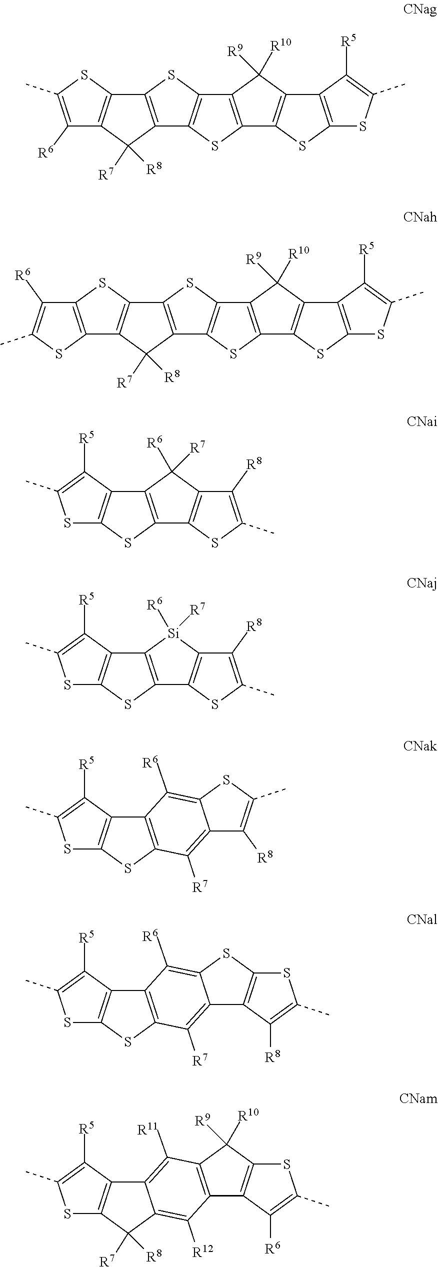

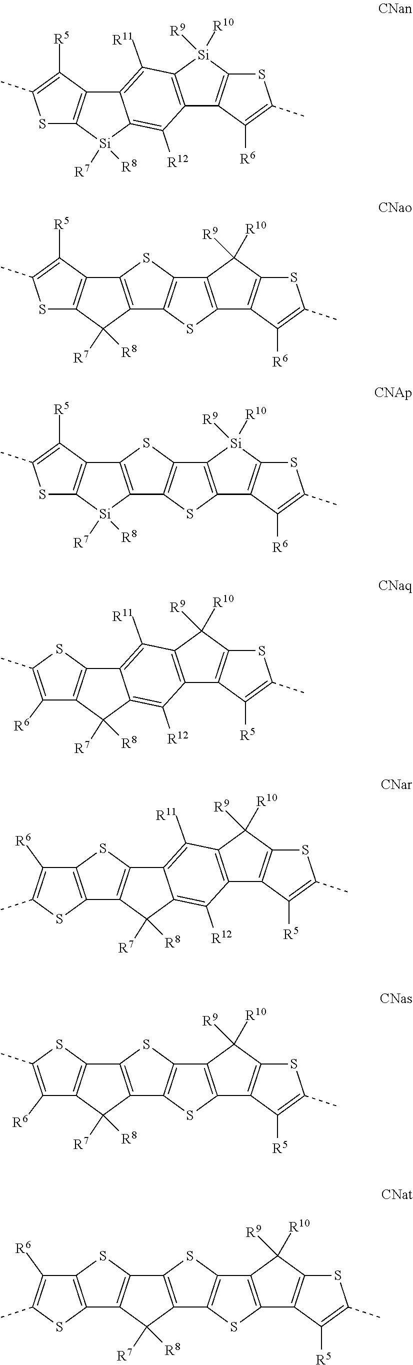

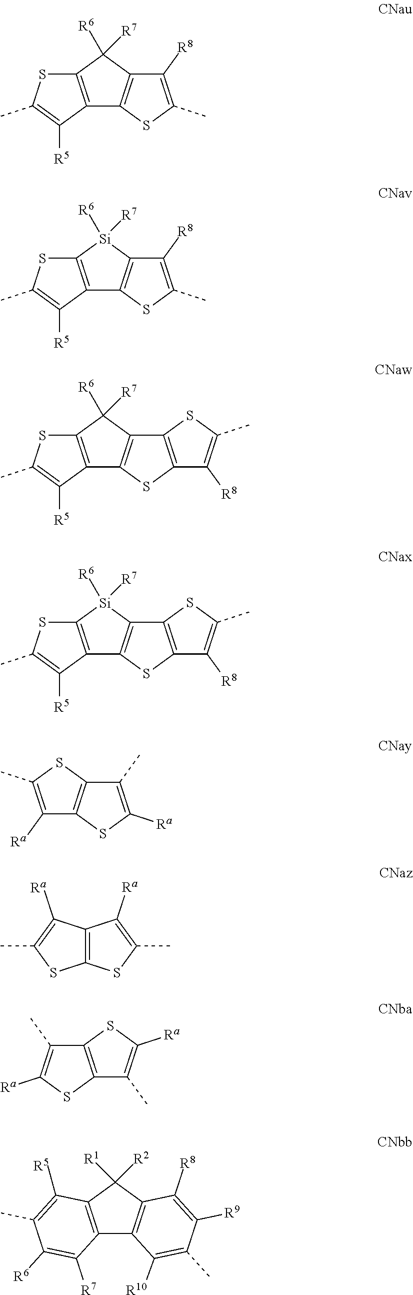

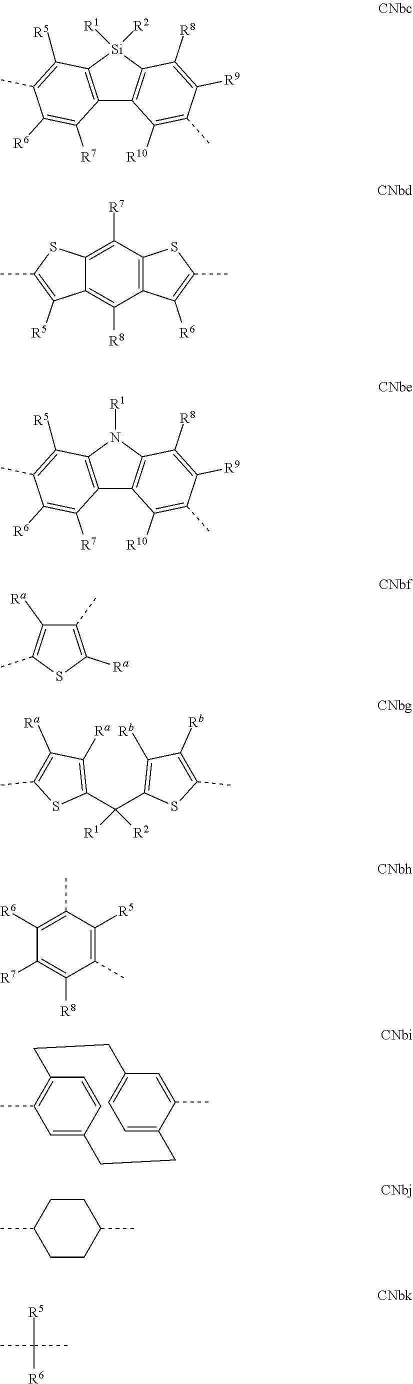

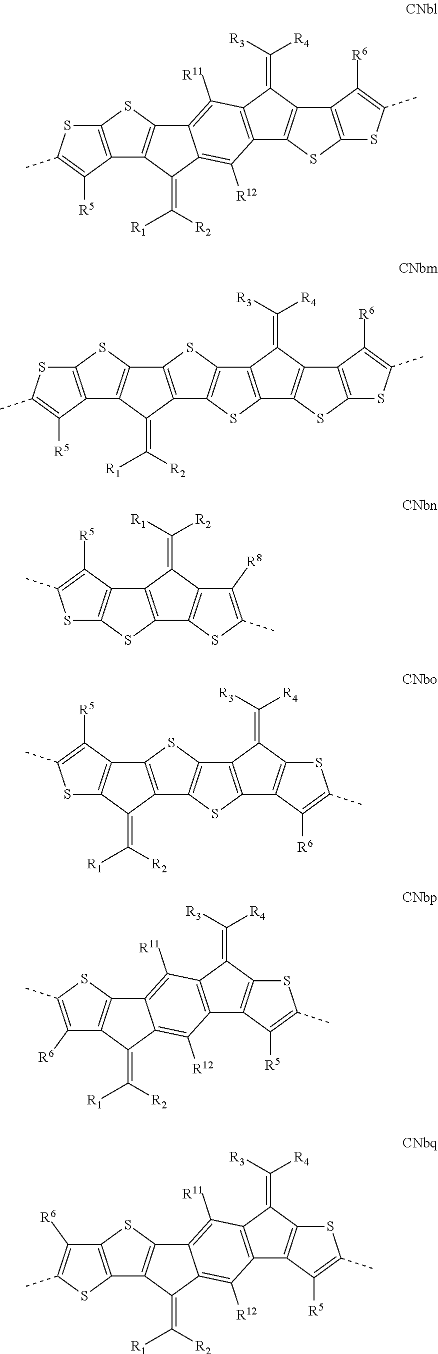

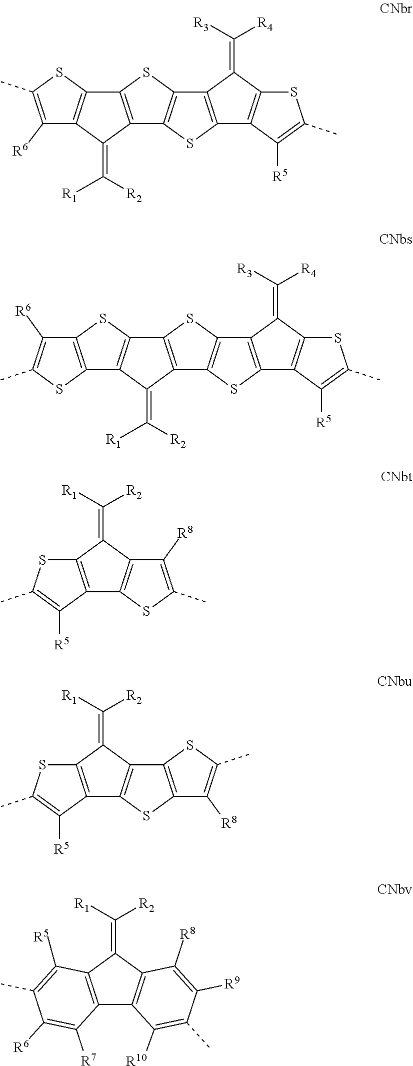

wherein R¹⁻¹² have the meanings given above and below, and R^{a} and R^{b} are, independently of each other and on each occurrence identically or differently, H, F, Cl or 01-12 alkyl or alkoxy, preferably H, F, Cl or CH₃.

Very preferably the compound of formula I comprises one or more non-conjugating groups selected from formulae CNaa to CNbv above.

Preferably the groups R^{W}, R^{T1} and R^{T2} in formula I are selected from H, F, Cl, Br, —NO₂, —CN, —CF₃, R*, —CF₂—R*, —O—R*, —S—R*, —SO₂—R*, —SO₃—R*, —C(═O)—H, —C(═O)—R*, —C(═S)—R*, —C(═O)—CF₂—R*, —C(═O)—OR*, —C(═S)—OR*, —O—C(═O)—R*, —O—C(═S)—R*, —C(═O)—SR*, —S—C(═O)—R*, —C(═O)NR*R**, —NR*—C(═O)—R*, —NHR*, —NR*R**, —CR*═CR*R**, —C═C—R*, —C═C—SiR*R**R***, —SiR*R**R***, —CH═CH(CN), —CH═C(CN)₂, —C(CN)═C(CN)₂, —CH═C(CN)(R^{a}), CH═C(CN)—C(═O)—OR*, —CH═C(CO—OR*)₂, —CH═C(CO—NR*R**)₂, and the group consisting of the following formulae

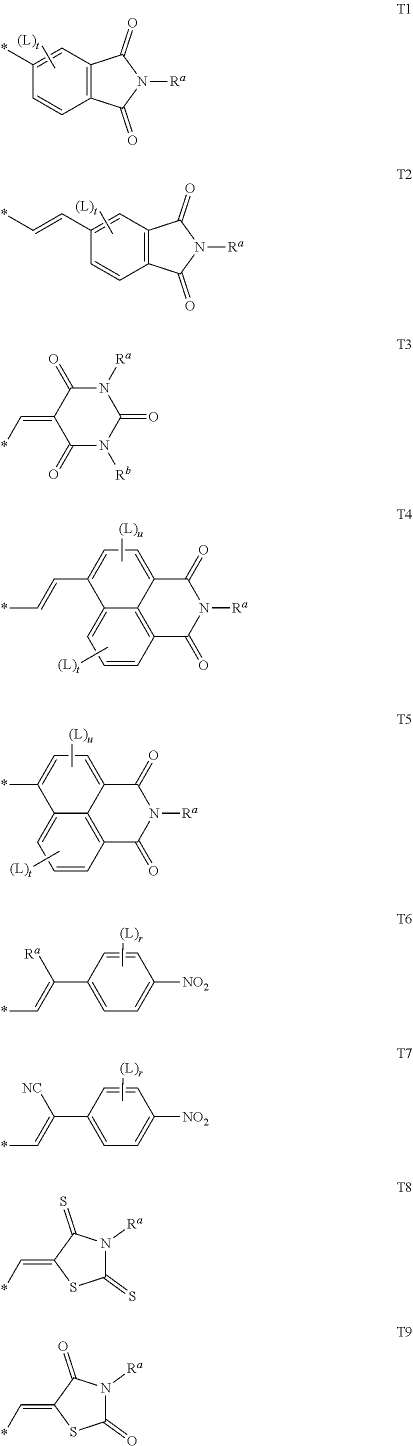

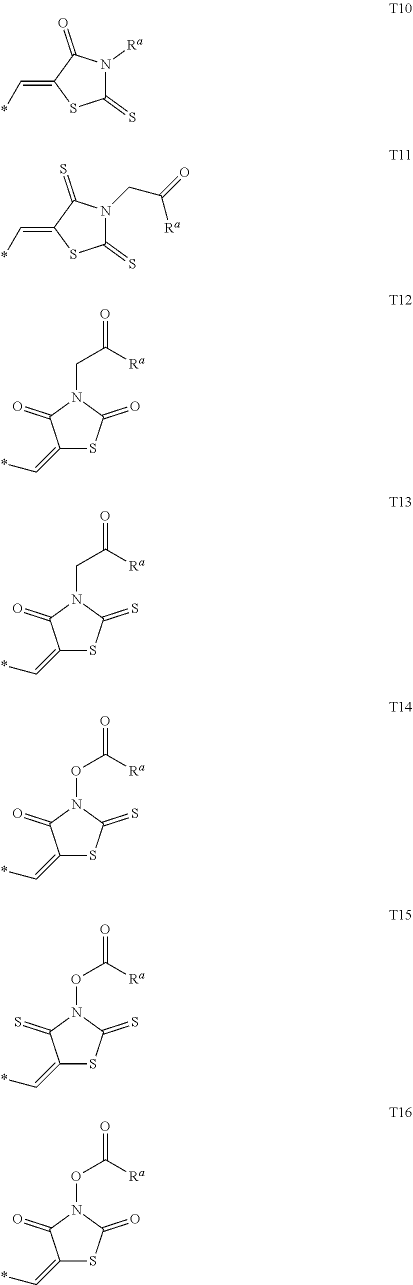

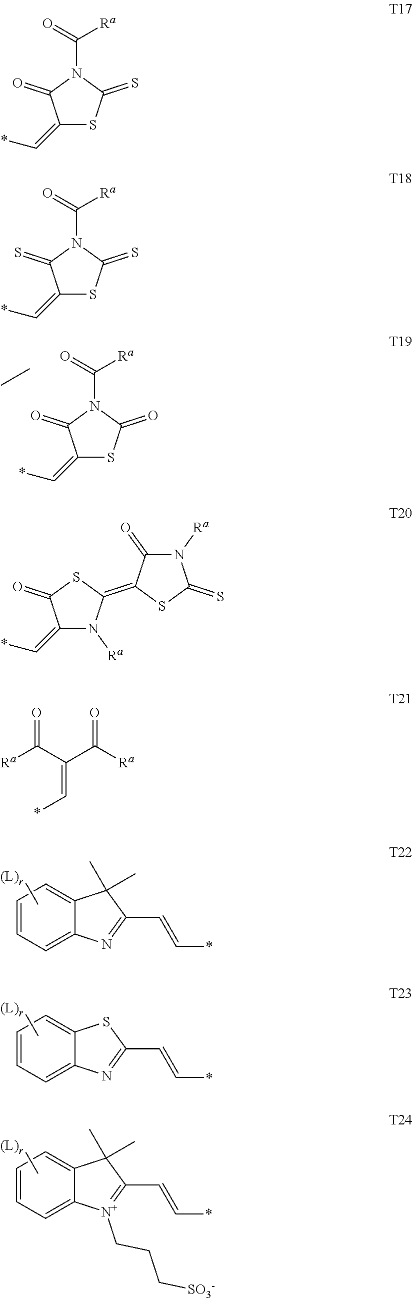

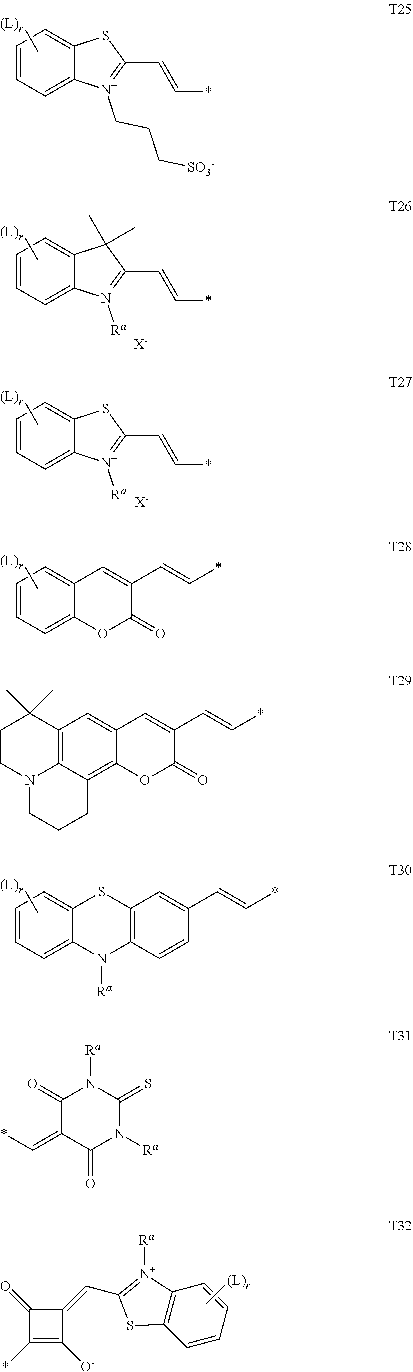

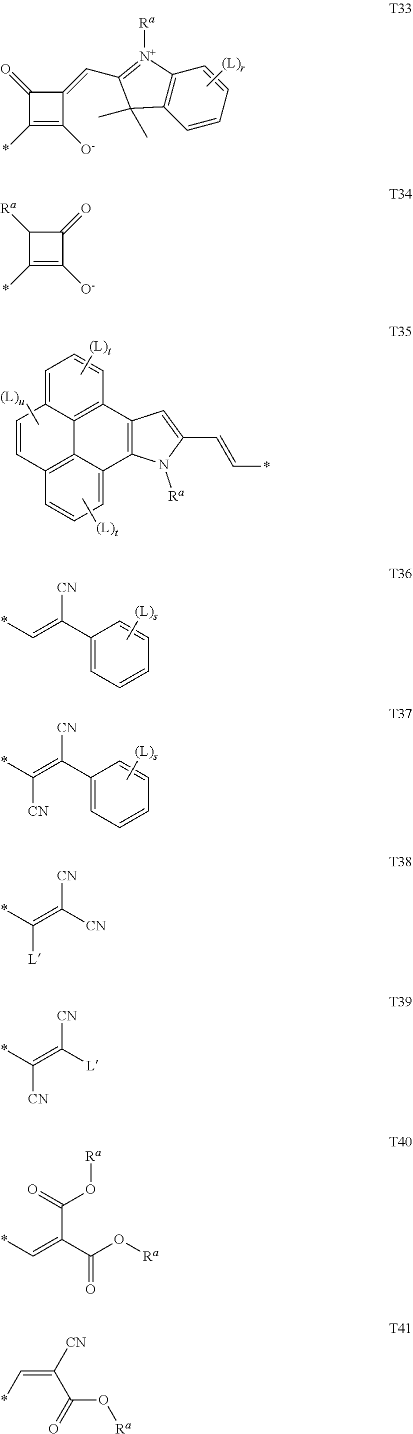

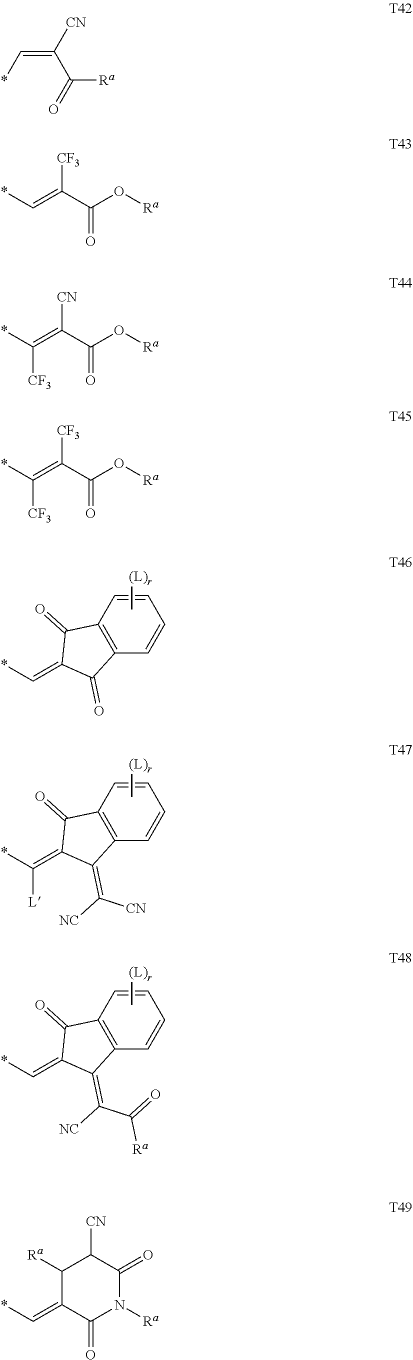

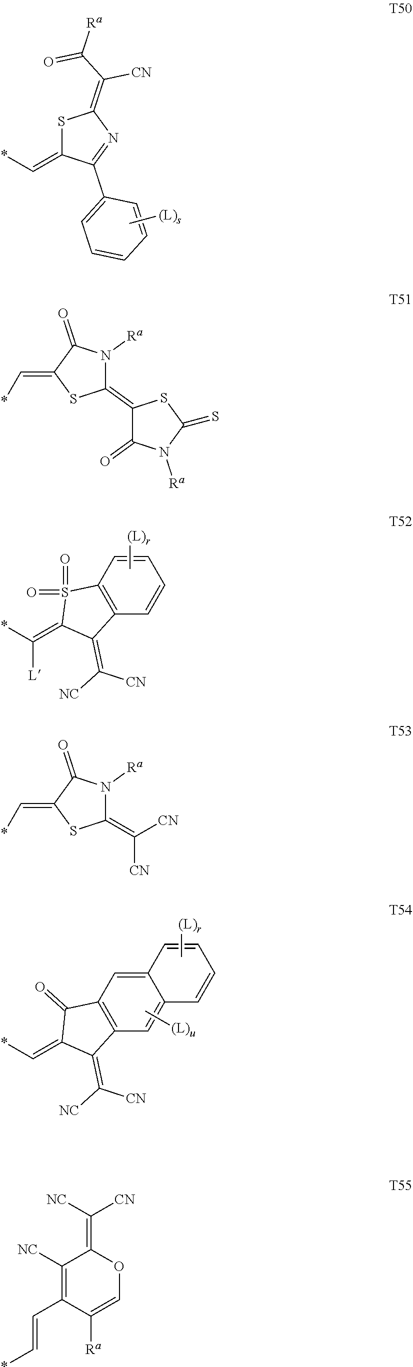

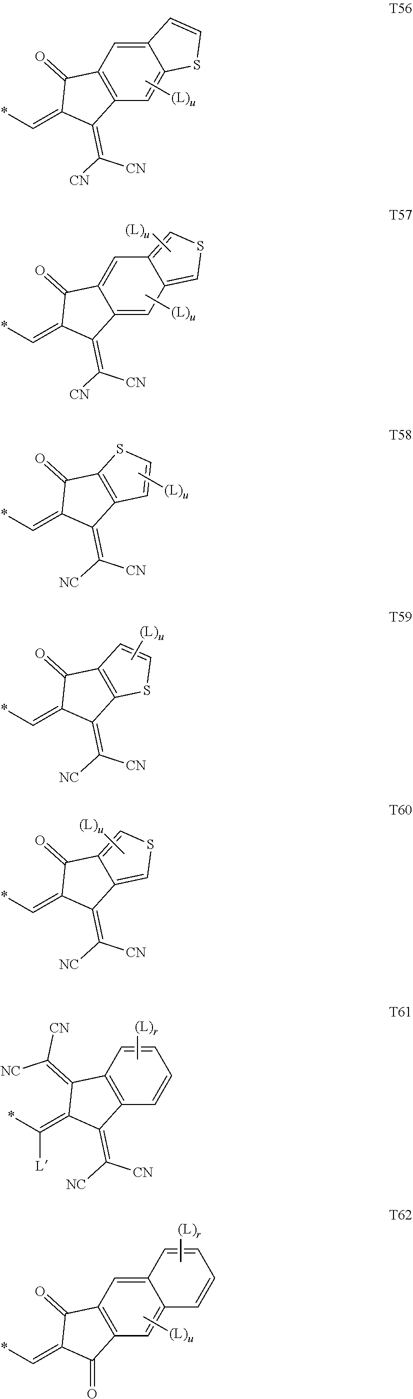

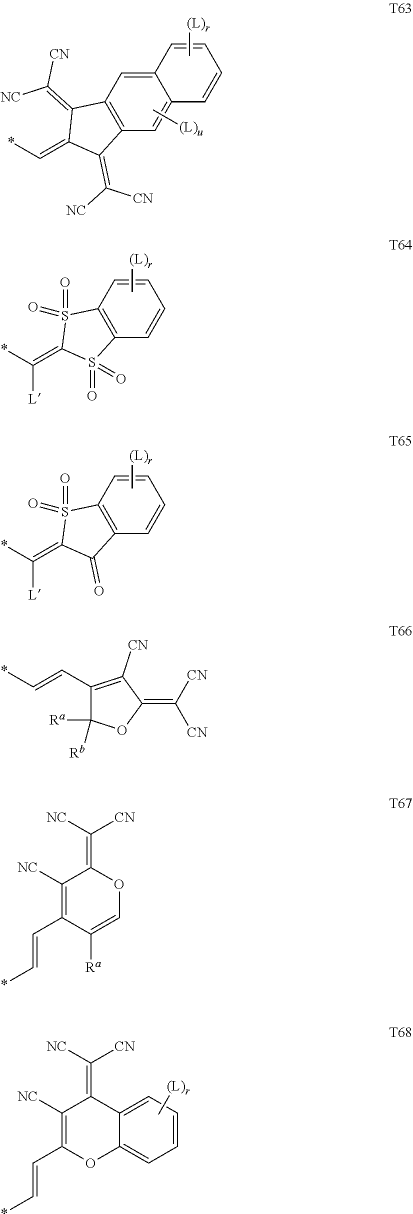

wherein the individual radicals, independently of each other and on each occurrence identically or differently, have the following meanings
R^{a}, R^{b} aryl or heteroaryl, each having from 4 to 30 ring atoms, optionally containing fused rings and being unsubstituted or substituted with one or more groups L, or one of the meanings given for L,
R*, R**, R*** alkyl with 1 to 20 C atoms which is straight-chain, branched or cyclic, and is unsubstituted, or substituted with one or more F or Cl atoms or CN groups, or perfluorinated, and in which one or more C atoms are optionally replaced by —O—, S—, —C(═O)—, —C(═S)—, —SiR⁰R⁰⁰—, —NR⁰R⁰⁰—, —CHR⁰═CR⁰⁰— or —C≡C— such that O- and/or S-atoms are not directly linked to each other,
L F, Cl, Br, I, —NO₂, —CN, —NC, —NCO, —NCS, —OCN, —SCN, R⁰, OR⁰, SR⁰, —C(═O)X°, —C(═O)R⁰, —C(═O)—OR⁰, —O—C(═O)—R⁰, —NH₂, —NHR⁰, —NR⁰R⁰⁰, —C(═O)NHR⁰, —C(═O)NR⁰R⁰⁰, —SO₃R⁰, —SO₂R⁰, —OH, —NO₂, —CF₃, —SF₅, or optionally substituted silyl, or carbyl or hydrocarbyl with 1 to 30, preferably 1 to 20 C atoms that is optionally substituted and optionally comprises one or more hetero atoms, preferably F, —CN, R⁰, —OR⁰, —SR⁰, —C(═O)—R⁰, —C(═O)—OR⁰, —O—C(═O)—R⁰, —O—C(═O)—OR⁰, —C(═O)—NHR⁰, —C(═O)—NR⁰R⁰⁰
L′ H or one of the meanings of L,
R⁰, R⁰⁰H or straight-chain or branched alkyl with 1 to 20, preferably 1 to 12 C atoms that is optionally fluorinated,
Y¹, Y²H, F, Cl or —CN,
X⁰ halogen, preferably F or Cl,
r 0, 1, 2, 3 or 4,
s 0, 1, 2, 3, 4 or 5,
t 0, 1, 2 or 3,
u 0, 1 or 2,
   and wherein at least one of R^{T1} and R^{T2} denotes an electron withdrawing group.

Preferred compounds of formula I are those wherein both of R^{T1} and R^{T2} denote an electron withdrawing group.

Preferred electron withdrawing groups R^{W}, R^{T1} and R^{T2} are selected from —CN, —C(═O)—OR*, —C(═S)—OR*, —CH═CH(CN), —CH═C(CN)₂, —C(CN)═C(CN)₂, —CH═C(CN)(R^{a}), CH═C(CN)—C(═O)—OR*, —CH═C(CO—OR*)₂, and formulae T1-T68.

Very preferred groups R^{W}, R^{T1} and R^{T2} are selected from the following formulae

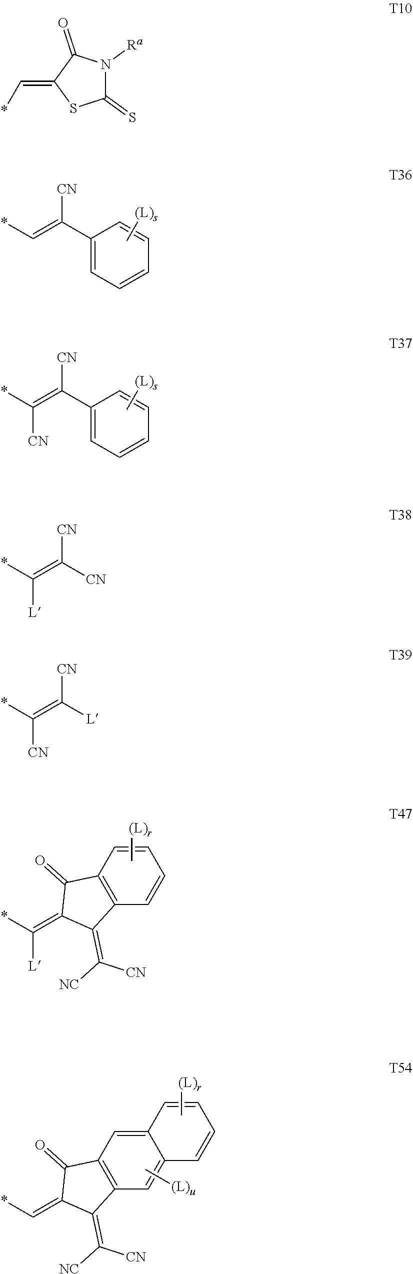

wherein L, L′, R^{a}, r and s have the meanings given above and below, and L′ is H or has one of the meanings given for L. Preferably in these formulae L′ is H. Further preferably in these formulae r is 0, 1 or 2 and u is 0.

The above formulae T1-T68 are meant to also include their respective E- or Z-stereoisomer with respect to the C═C bond in α-position to the adjacent group Ar⁴ or Ar⁵, thus for example the group

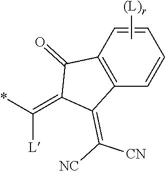

may also denote

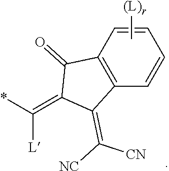

In the compounds of formula I and Ia preferably R¹⁻⁴ are different from H.

In a preferred embodiment of the present invention, R¹⁻⁴ in formula I are selected from F, Cl or straight-chain or branched alkyl, alkoxy, sulfanylalkyl, sulfonylalkyl, alkylcarbonyl, alkoxycarbonyl and alkylcarbonyloxy, each having 1 to 20 C atoms and being unsubstituted or substituted by one or more F atoms.

In another preferred embodiment of the present invention, R¹⁻⁴ in formula I are selected from mono- or poylcyclic aryl or heteroaryl, each of which is optionally substituted with one or more groups L as defined in formula I and has 4 to 30 ring atoms, and wherein two or more rings may be fused to each other or connected with each other by a covalent bond.

In a preferred embodiment of the present invention, R⁵⁻¹⁷ in formula I are H.

In another preferred embodiment of the present invention, at least one of R⁵⁻¹⁷ in formula I is different from H.

In a preferred embodiment of the present invention, R⁵⁻¹⁷ in formula I, when being different from H, are selected from F, Cl or straight-chain or branched alkyl, alkoxy, sulfanylalkyl, sulfonylalkyl, alkylcarbonyl, alkoxycarbonyl and alkylcarbonyloxy, each having 1 to 20 C atoms and being unsubstituted or substituted by one or more F atoms, without being perfluorinated.

In another preferred embodiment of the present invention, R⁵⁻¹⁷ in formula I, when being different from H, are selected from aryl or heteroaryl, each of which is optionally substituted with one or more groups R^{S} as defined in formula I and has 4 to 30 ring atoms.

Preferred aryl and heteroaryl groups R¹⁻¹⁷ are selected from the following formulae

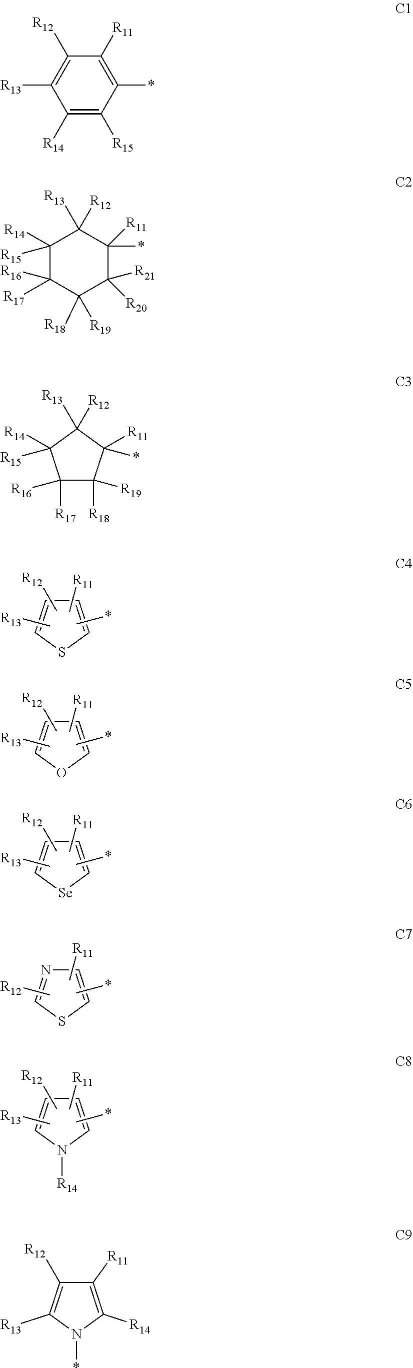

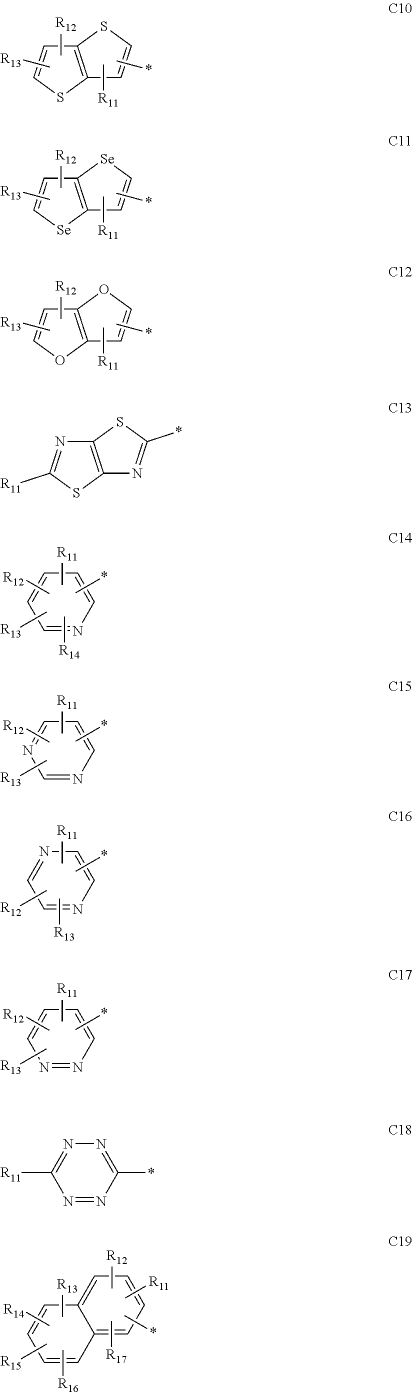

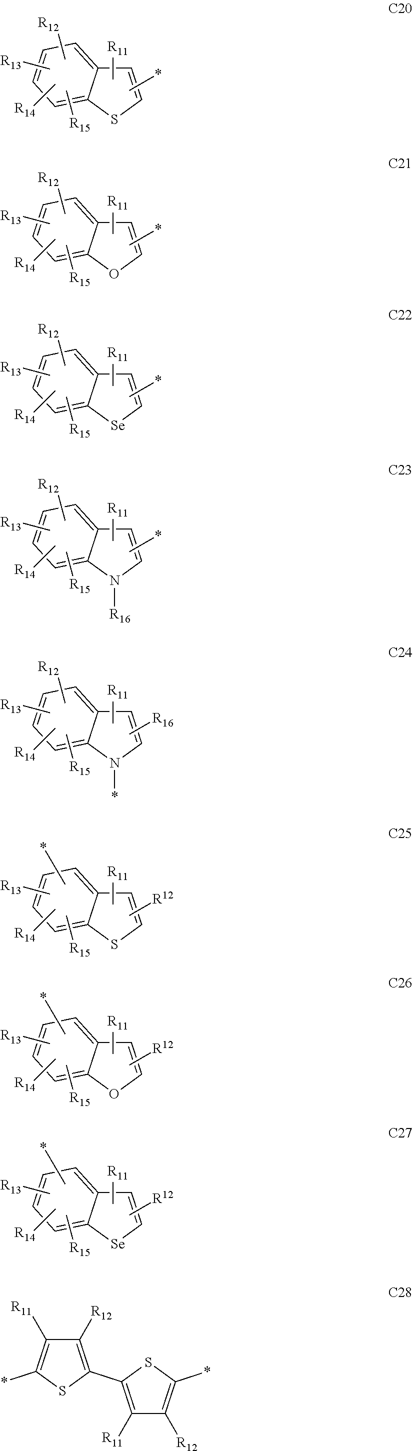

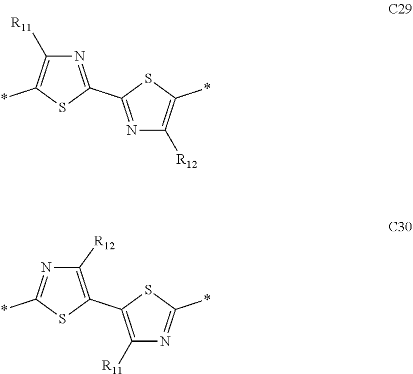

wherein R¹¹⁻¹⁷, independently of each other, and on each occurrence identically or differently, have one of the meanings given in formula I or one of its preferred meanings as given above and below.

Very preferred aryl and heteroaryl groups R¹⁻¹⁷ are selected from the following formulae

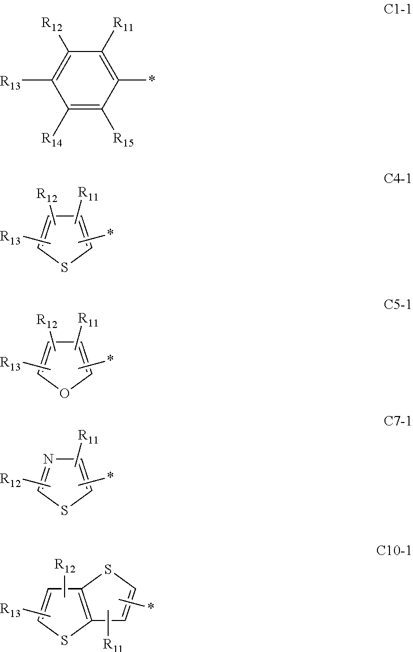

wherein R¹¹⁻¹⁵ are as defined above. Most preferably R₁-R₁₀ are selected from SUB7-SUB14.

In another preferred embodiment one or more of R¹⁻¹⁷ denote a straight-chain, branched or cyclic alkyl group with 1 to 50, preferably 2 to 50, very preferably 2 to 30, more preferably 2 to 24, most preferably 2 to 16 C atoms, in which one or more CH₂ or CHs groups are replaced by a cationic or anionic group.

The cationic group is preferably selected from the group consisting of phosphonium, sulfonium, ammonium, uronium, thiouronium, guanidinium or heterocyclic cations such as imidazolium, pyridinium, pyrrolidinium, triazolium, morpholinium or piperidinium cation.

Preferred cationic groups are selected from the group consisting of tetraalkylammonium, tetraalkylphosphonium, N-alkylpyridinium, N,N-dialkylpyrrolidinium, 1,3-dialkylimidazolium, wherein “alkyl” preferably denotes a straight-chain or branched alkyl group with 1 to 12 C atoms and very preferably is selected from formulae SUB1-6.

Further preferred cationic groups are selected from the group consisting of the following formulae

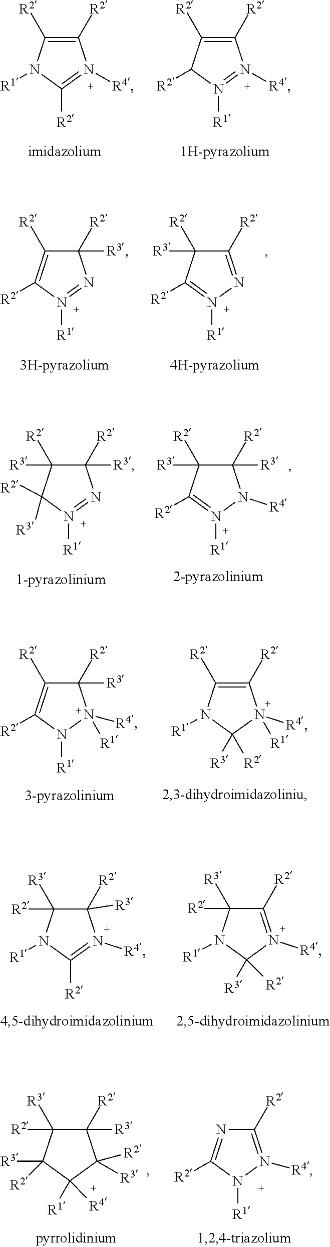

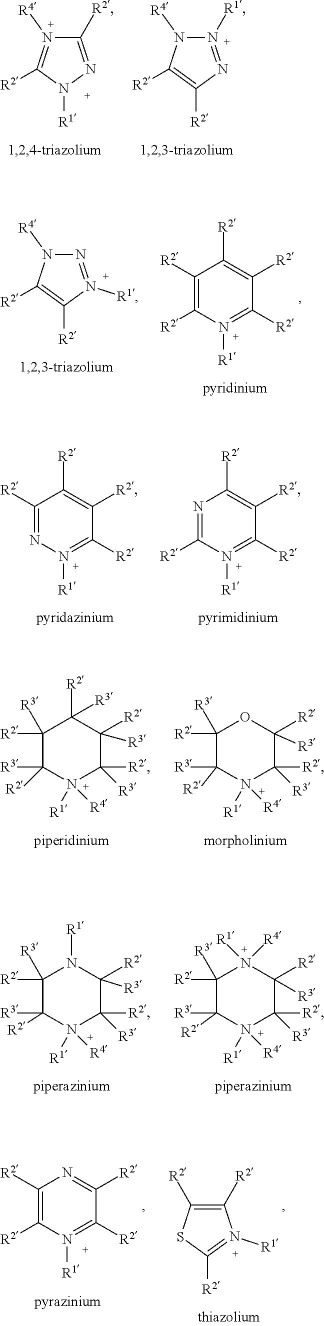

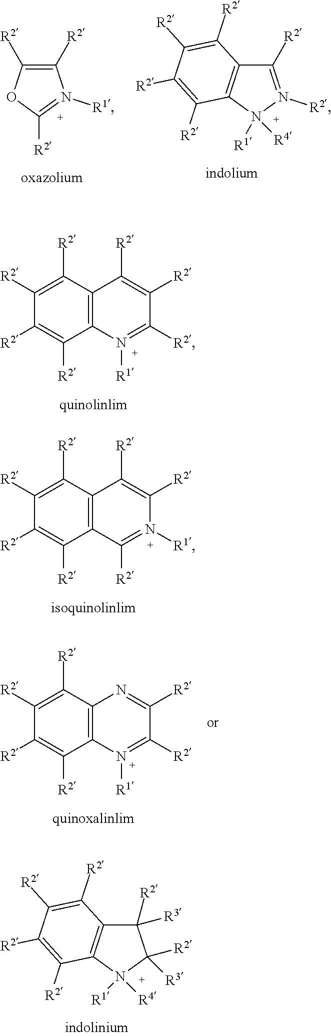

wherein R^{1′}, R^{2′}, R^{3′} and R^{4′} denote, independently of each other, H, a straight-chain or branched alkyl group with 1 to 12 C atoms or non-aromatic carbo- or heterocyclic group or an aryl or heteroaryl group, each of the aforementioned groups having 3 to 20, preferably 5 to 15, ring atoms, being mono- or polycyclic, and optionally being substituted by one or more identical or different substituents L as defined above, or denote a link to the respective group R¹⁻¹⁰.

In the above cationic groups of the above-mentioned formulae any one of the groups R^{1′}, R^{2′}, R^{3′} and R^{4′} (if they replace a CH₃ group) can denote a link to the respective group R¹⁻¹⁰, or two neighbored groups R^{1′}, R^{2′}, R^{3′} or R^{4′} (if they replace a CH₂ group) can denote a link to the respective group R¹.

The anionic group is preferably selected from the group consisting of borate, imide, phosphate, sulfonate, sulfate, succinate, naphthenate or carboxylate, very preferably from phosphate, sulfonate or carboxylate.

In a preferred embodiment of the present invention the groups R^{T1} and R^{T2} in formula I are selected from alkyl with 1 to 16 C atoms which is straight-chain, branched or cyclic, and is unsubstituted, substituted with one or more F or Cl atoms or CN groups, or perfluorinated, and in which one or more C atoms are optionally replaced by —O—, —S—, —C(O)—, —C(S)—, —SiR⁰R⁰⁰—, —NR⁰R⁰⁰—, —CHR⁰═CR⁰⁰— or —C═C— such that O- and/or S-atoms are not directly linked to each other.

In a preferred embodiment the compound of formula I consists of a conjugating group selected from formulae CCaa to CCbi, one or more non-conjugating groups Ar⁴, Ar⁵ or Ar⁶ selected from formulae N1 to N6, preferably from formulae ARN1-ARN10, and electron-withdrawing groups R^{T1} and R^{T2}, preferably selected from formulae T1-T68, very preferably from formulae T10, T36-T39, T47 and T54.

In another preferred embodiment the compound of formula I consists of a non-conjugating group selected from formulae CNaa to CNaz, and CNba to CNbv, one or more conjugating groups Ar⁴, Ar⁵ or Ar⁶ selected from formulae ARC1 to ARC11, and electron-withdrawing groups R^{T1} and R^{T2}, preferably selected from formulae T1-T68, very preferably from formulae T10, T36-T39, T47 and T54.

Further preferred compounds of formula I are selected from the following preferred embodiments or any combination thereof:
the compound comprises one or more conjugating groups Ar¹ selected from formulae AC1a to AC1h, preferably selected from formulae AC1-1 to AC1-14,
the compound comprises one or more non-conjugating groups Ar¹ selected from formulae AN1a to AN1h, preferably selected from formulae AN1-1 to AN1-20,
the compound comprises one or more conjugating groups Ar² selected from formulae AC2a to AC2d, preferably selected from formulae AC2-1 to AC2-6,
the compound comprises one or more non-conjugating groups Ar² selected from formulae AN2a to AN2n, preferably selected from formulae AN1-1 to AN2-42,
the compound comprises one or more conjugating groups Ar³ selected from formulae AC3a to AC3d, preferably selected from formulae AC3-1 to AC3-6,
the compound comprises one or more non-conjugating groups Ar³ selected from formulae AN3a to AN3n, preferably selected from formulae AN3-1 to AN3-42,
the compound comprises one or more conjugating groups Ar⁴, Ar⁵ or Ar⁶ selected from formulae ARC1 to ARC11,
the compound comprises one or more non-conjugating groups Ar⁴, Ar⁵ or Ar⁶ selected from formulae N1 to N6,
the compound comprises one or more non-conjugating groups Ar⁴, Ar⁵ or Ar⁶ selected from formulae ARN1 to ARN10,
the compound comprises one or more conjugating groups selected from formulae CCaa to CCbi,
the compound comprises one or more non-conjugating groups selected from formulae CNaa to CNaz and formulae CNba to CNbv,
the compound consists of a conjugating group selected from formulae CCaa to CCbi, one or more non-conjugating groups Ar⁴, Ar⁵ or Ar⁶ selected from formulae N1 to N6, preferably from formulae ARN1-ARN10, and electron-withdrawing groups R^{T1} and R^{T2}, preferably selected from formulae T1-T68, very preferably from formulae T10, T36-T39, T47 and T54,
the compound consists of a non-conjugating group selected from formulae CNaa to CNaz, and CNba to CNbv, one or more conjugating groups Ar⁴, Ar⁵ or Ar⁶ selected from formulae ARC1 to ARC11, and
electron-withdrawing groups R^{T1} and R^{T2}, preferably selected from formulae T1-T68, very preferably from formulae T10, T36-T39, T47 and T54,
W¹ is S or Se, preferably S,
U¹ is CR¹R² or SiR¹R²,
U¹ is CR¹R²,
V¹ is CR⁵
V¹ is N,
m=k=0,
m=k=1,
m is 1 and k is 0 or m is 0 and k is 1,
a=b=1 or 2, preferably 1,
a=b=0,
c=0,
c=1,
d=e=1,
d=1 and c=e=0
m=1, c=e=0, and a=b=0,
m=1, c=e=0, and a and b denote independently of each other 0, 1 or 2,
m=2, c=e=0, and a=b=0,
m=2, c=e=0, and a and b denote independently of each other 0, 1 or 2,
m is different from 0, preferably 1 or 2, and d is different from 0, preferably 1, 2, 3, 4, 5, 6, 7 or 8,
k is different from 0, preferably 1 or 2, and e is different from 0, preferably 1, 2, 3, 4, 5, 6, 7 or 8,
in one or both of Ar⁴ and Ar⁵ all substituents X¹⁻⁴ are H,
in one or both of Ar⁴ and Ar⁵ at least one, preferably one or two of X¹⁻⁴ are different from H,
Ar⁴ and Ar⁵ denote thiophene, thiazole, thieno[3,2-b]thiophene,
thiazolo[5,4-d]thiazole, benzene, 2,1,3-benzothiadiazole, 1,2,3-benzothiadiazole, thieno[3,4-b]thiophene, benzotriazole, thiadiazole[3,4-c]pyridine or vinyl, which are substituted by X¹, X², X³ and X⁴ as defined above,
Ar⁴ and Ar⁵ denote thiophene, thiazole, thieno[3,2-b]thiophene, thiazolo[5,4-d]thiazole, benzene, 2,1,3-benzothiadiazole, 1,2,3-benzothiadiazole, thieno[3,4-b]thiophene, benzotriazole, thiadiazole[3,4-c]pyridine or vinyl, wherein X¹, X², X³ and X⁴ are H,
Ar⁴ and Ar⁵ denote thiophene, thiazole, thieno[3,2-b]thiophene, thiazolothiazole, benzene, 2,1,3-benzothiadiazole, 1,2,3-benzothiadiazole, thieno[3,4-b]thiophene, benzotriazole, thiadiazole[3,4-c]pyridine or vinyl, wherein one or more of X¹, X², X³ and X⁴ are different from H,
R¹ and R² are different from H,
R¹ and R², when being different from H, are each independently selected from F, Cl or straight-chain or branched alkyl, alkoxy, sulfanylalkyl, sulfonylalkyl, alkylcarbonyl, alkoxycarbonyl and alkylcarbonyloxy, each having 1 to 20 C atoms and being unsubstituted or substituted by one or more F atoms, or alkyl or alkoxy having 1 to 12 C atoms that is optionally fluorinated, more preferably from formulae SUB1-SUB6 above,
R¹ and R², when being different from H, and are each independently selected from phenyl that is substituted, preferably in 4-position, or in 2,4-positions, or in 2,4,6-positions or in 3,5-positions, with alkyl or alkoxy having 1 to 20 C atoms, preferably 1 to 16 C atoms, very preferably 4-alkylphenyl wherein alkyl is C1-16 alkyl, most preferably 4-methylphenyl, 4-hexylphenyl, 4-octylphenyl or 4-dodecylphenyl, or 4-alkoxyphenyl wherein alkoxy is C1-16 alkoxy, most preferably 4 hexyloxyphenyl, 4-octyloxyphenyl or 4-dodecyloxyphenyl or 2,4-dialkylphenyl wherein alkyl is C1-16 alkyl, most preferably 2,4-dihexylphenyl or 2,4-dioctylphenyl or 2,4-didecylphenyl, or 2,4-dialkoxyphenyl wherein alkoxy is C1-16 alkoxy, most preferably 2,4-dihexyloxyphenyl or 2,4-dioctyloxyphenyl or 2,4-didecyloxyphenyl or 3,5-dialkylphenyl wherein alkyl is C1-16 alkyl, most preferably 3,5-dihexylphenyl or 3,5-dioctylphenyl or 3,5-didecylphenyl, or 3,5-dialkoxyphenyl wherein alkoxy is C1-16 alkoxy, most preferably 3,5-dihexyloxyphenyl or 3,5-dioctyloxyphenyl or 3,5-didecyloxyphenyl, or 2,4,6-trialkylphenyl wherein alkyl is C1-16 alkyl, most preferably 2,4,6 trihexylphenyl or 2,4,6-trioctylphenyl or 2,4,6-trialkoxyphenyl wherein alkoxy is C1-16 alkoxy, most preferably 2,4,6-trihexyloxyphenyl or 2,4,6-trioctyloxyphenyl or 4-thioalkylphenyl wherein thioalkyl is C1-16 thioalkyl, most preferably 4-thiohexylphenyl, 4-thiooctylphenyl or 4-thiododecylphenyl, or 2,4-dithioalkylphenyl wherein thioalkyl is C1-16 thioalkyl, most preferably 2,4-dithiohexylphenyl or 2,4-dithiooctylphenyl, or 3,5-dithioalkylphenyl wherein thioalkyl is C1-16 thioalkyl, most preferably 3,5-dithiohexylphenyl or 3,5-dithiooctylphenyl, or 2,4,6-trithioalkylphenyl wherein thioalkyl is C1-16 thioalkyl, most preferably 2,4,6-trithiohexylphenyl or 2,4,6-trithiooctylphenyl, or from thiophene that is optionally substituted, preferably in 5-position, 4,5-positions or 3,5-positions, with alkyl, alkoxy or thioalkyl having 1 to 16 C atoms, most preferably from formulae SUB7-SUB18 above,
R³ and R⁴ are H,
R³ and R⁴ are different from H,
R³ and R⁴, when being different from H, are each independently selected from F, Cl or straight-chain or branched alkyl, alkoxy, sulfanylalkyl, sulfonylalkyl, alkylcarbonyl, alkoxycarbonyl and alkylcarbonyloxy, each having 1 to 20 C atoms and being unsubstituted or substituted by one or more F atoms, without being perfluorinated, or alkyl or alkoxy having 1 to 12 C atoms that is optionally fluorinated, more preferably from formulae SUB1-SUB6 above,
R³ and R⁴ are different from H, and are each independently selected from phenyl that is substituted, preferably in 4-position, or in 2,4-positions, or in 2,4,6-positions or in 3,5-positions, with alkyl or alkoxy having 1 to 20 C atoms, preferably 1 to 16 C atoms, very preferably 4-alkylphenyl wherein alkyl is C1-16 alkyl, most preferably 4-methylphenyl, 4-hexylphenyl, 4-octylphenyl or 4-dodecylphenyl, or 4-alkoxyphenyl wherein alkoxy is C1-16 alkoxy, most preferably 4-hexyloxyphenyl, 4-octyloxyphenyl or 4-dodecyloxyphenyl or 2,4-dialkylphenyl wherein alkyl is C1-16 alkyl, most preferably 2,4-dihexylphenyl or 2,4-dioctylphenyl or 2,4-didecylphenyl, or 2,4-dialkoxyphenyl wherein alkoxy is C1-16 alkoxy, most preferably 2,4-dihexyloxyphenyl or 2,4-dioctyloxyphenyl or 2,4-didecyloxyphenyl, or 3,5-dialkylphenyl wherein alkyl is C1-16 alkyl, most preferably 3,5-dihexylphenyl or 3,5-dioctylphenyl or 3,5-didecylphenyl, or 3,5-dialkoxyphenyl wherein alkoxy is C1-16 alkoxy, most preferably 3,5-dihexyloxyphenyl or 3,5-dioctyloxyphenyl or 3,5-didecyloxyphenyl, or 2,4,6-trialkylphenyl wherein alkyl is C1-16 alkyl, most preferably 2,4,6-trihexylphenyl or 2,4,6-trioctylphenyl or 2,4,6-tialkoxyphenyl wherein alkoxy is C1-16 alkoxy, most preferably 2,4,6-trihexyloxyphenyl or 2,4,6-trioctyloxyphenyl or 4-thioalkylphenyl wherein thioalkyl is C1-16 thioalkyl, most preferably 4-thiohexylphenyl, 4-thiooctylphenyl or 4-thiododecylphenyl, or 2,4-dithioalkylphenyl wherein thioalkyl is C1-16 thioalkyl, most preferably 2,4-dithiohexylphenyl or 2,4-dithiooctylphenyl, or 3,5-dithioalkylphenyl wherein thioalkyl is C1-16 thioalkyl, most preferably 3,5-dithiohexylphenyl or 3,5-dithiooctylphenyl, or 2,4,6-trithioalkylphenyl wherein thioalkyl is C1-16 thioalkyl, most preferably 2,4,6-trithiohexylphenyl or 2,4,6-trithiooctylphenyl, or from thiophene that is optionally substituted, preferably in 5-position, 4,5-positions or 3,5-positions, with alkyl, alkoxy or thioalkyl having 1 to 16 C atoms, more preferably from formulae SUB7-SUB18 above, most preferably from subformulae SUB14-SUB18,
L′ is H,
L, L′ denote F, Cl, Br, I, —CN, —NO₂, or alkyl or alkoxy with 1 to 16 C atoms that is optionally fluorinated,
r is 2 and L is F, Cl, Br, I —CN, —NO₂, or alkyl or alkoxy with 1 to 16 C atoms that is optionally fluorinated,
r is 1 and L is F, Cl, Br, I, —CN, —NO₂, or alkyl or alkoxy with 1 to 16 C atoms that is optionally fluorinated,
r is 4 and L is F, Cl, Br, I, —CN, —NO₂, or alkyl or alkoxy with 1 to 16 C atoms that is optionally fluorinated,
u=r=0,
u is 2 and L is F, Cl, Br, I —CN, —NO₂, or alkyl or alkoxy with 1 to 16 C atoms that is optionally fluorinated,
u is 1 and L is F, Cl, Br, I, —CN, —NO₂, or alkyl or alkoxy with 1 to 16 C atoms that is optionally fluorinated,
R³⁻¹⁷, when being different from H, are selected from F, Cl or straight-chain or branched alkyl, alkoxy, sulfanylalkyl, sulfonylalkyl, alkylcarbonyl, alkoxycarbonyl and alkylcarbonyloxy, each having 1 to 24 C atoms and being unsubstituted or substituted by one or more F atoms, without being perfluorinated, preferably from F, or alkyl or alkoxy having 1 to 16 C atoms that is optionally fluorinated, very preferably SUB1-SUB6 as defined above,
R^{T1} and R^{T2} are different from H and are both an electron withdrawing group,
R^{W} has one of the meanings given for R^{T1},
one or more of X¹⁻⁴ denote F or Cl, preferably F.

Preferred compounds of formula I are selected of formula IA

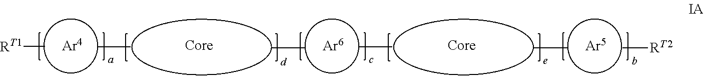

wherein Ar⁴, Ar⁵, Ar⁶, a, b, c, d, e, R^{T1}, R^{T2} have the meanings given above, and “core” is a group independently of each other and on each occurrence identically or differently is selected from formulae CCaa to CCbi or CNaa to CNbv as given above and wherein the compound contains at least one moiety selected from the group consisting of AN1a, AN1b, AN1c, AN1d, AN1e, AN1f, AN1g, AN1h, AN2a, AN2b, AN2c, AN2d, AN2e, AN2f, AN2g, AN2h, AN2i, AN2j, AN2k, AN2l, AN2m, AN2n, AN3a, AN3b, AN3c, AN3d, AN3e, AN3f, AN3g, AN3h, AN3i, AN3j, AN3k, AN3l, AN3m, AN3n, N1, N2, N3, N4, N5, N6 or their subformulae.

Preferred are compounds of formula IA selected from the following groups or any combination thereof:

a) Compounds of formula IA wherein c and e are 0, d is 1, one or both of a and b are different from 0 and preferably denote 0, 1 or 2, Ar⁴ and Ar⁵ are selected from formula ARC1-ARC11 or ARN1-ARN10 and “core” is selected from formulae CNaa-CNbv.

b) Compounds of formula IA wherein a, b, c, e are 0, d is 1 and “core” is selected from formulae CNaa-CNbe.

c) Compounds of formula IA wherein c and e are 0, d is 1, one or both of a and b are different from 0 and preferably denote 0, 1 or 2, Ar⁴ and Ar⁵ are selected from formula ARC1-ARC11 or ARN1-ARN10 with one or more selected from formula ARN1-ARN10, and “core” is selected from formulae CCaa-CCbi.

d) Compounds of formula IA wherein c is different from 0, preferably is 1 and Ar⁶ is selected from N1-N6 and “core” is selected from formulae CCaa-CCbi or CNaa-CNbv.

e) Compounds selected from above groups a-d, wherein R^{T1} and R^{T2} are selected from formulae T10, T36, T37, T38, T39, T47 and T54.

f) Compounds selected from above groups a-e, wherein R¹, R², R³ and R⁴ are selected from alkyl or alkoxy having 1 to 16 C atoms that is optionally fluorinated.

g) Compounds selected from above groups a-f, wherein R¹, R², R³ and R⁴ are selected from phenyl that is optionally substituted, preferably in 4-position, 3,4,5-positions or 3,5-positions, with alkyl, alkoxy or thioalkyl having 1 to 16 C atoms.

Another embodiment of the invention relates to a composition comprising a compound of formula I, and further comprising one or more electron donors or p-type semiconductors, preferably selected from conjugated polymers.

Preferably the conjugated polymer used in the said composition comprises at least one electron donating unit (“donor unit”) and at least one electron accepting unit (“acceptor unit”), and optionally at least one spacer unit separating a donor unit from an acceptor unit,

wherein each donor and acceptor units is directly connected to another donor or acceptor unit or to a spacer unit, and wherein all of the donor, acceptor and spacer units are selected from arylene or heteroarylene that has from 5 to 20 ring atoms, is mono- or polycyclic, optionally contains fused rings, are is unsubstituted or substituted by one or more identical or different groups L as defined above.

Preferably the spacer units, if present, are located between the donor and acceptor units such that a donor unit and an acceptor unit are not directly connected to each other.

Preferred conjugated polymers comprise, very preferably consist of, one or more units of the formula U1 and one or more units of the formula U2

-(D-Sp)- U1

-(A-Sp)- U2

wherein D denotes a donor unit, A denotes an acceptor unit and Sp denotes a spacer unit, all of which are selected from arylene or heteroarylene that has from 5 to 20 ring atoms, is mono- or polycyclic, optionally contains fused rings, are is unsubstituted or substituted by one or more identical or different groups L as defined above.

Very preferred are polymers of formula Pi and Pii

-[(D-Sp)ₓ-(A-Sp)_{y}]ₙ— Pi

-[(D-A)ₓ-(A-Sp)_{y}]ₙ— Pii

wherein A, D and Sp are as defined in formula U1 and U2, x denotes the molar fraction of the units (D-Sp) or (D-A), y denotes the molar fraction of the units (A-Sp), x and y are each, independently of one another >0 and <1, with x+y=1, and n is an integer >1.

In the polymers of formula P1 and P2 and their subformulae, x and y are preferably from 0.1 to 0.9, very preferably from 0.25 to 0.75, most preferably from 0.4 to 0.6.

Preferred donor units or units D are selected from the following formulae

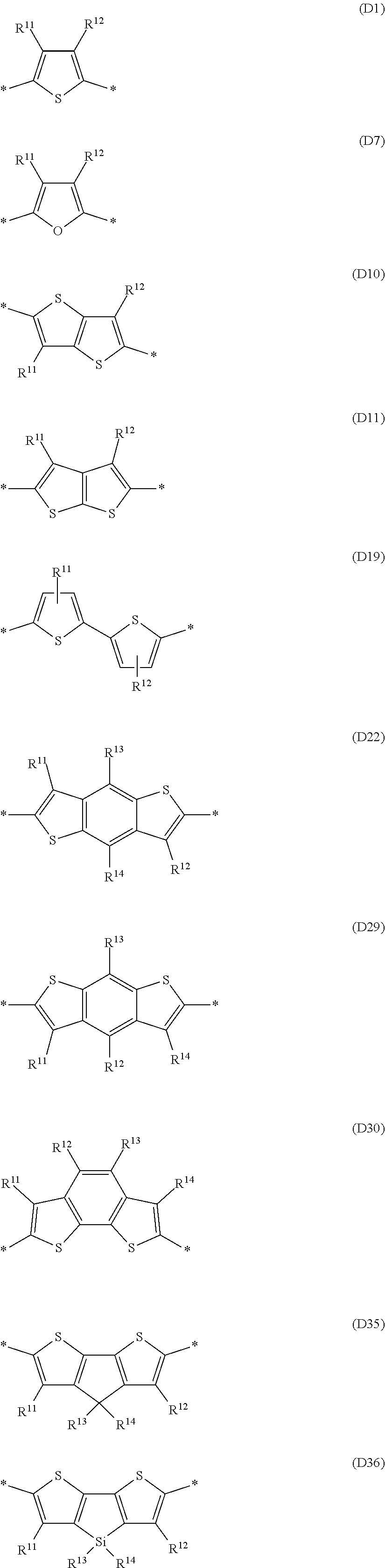

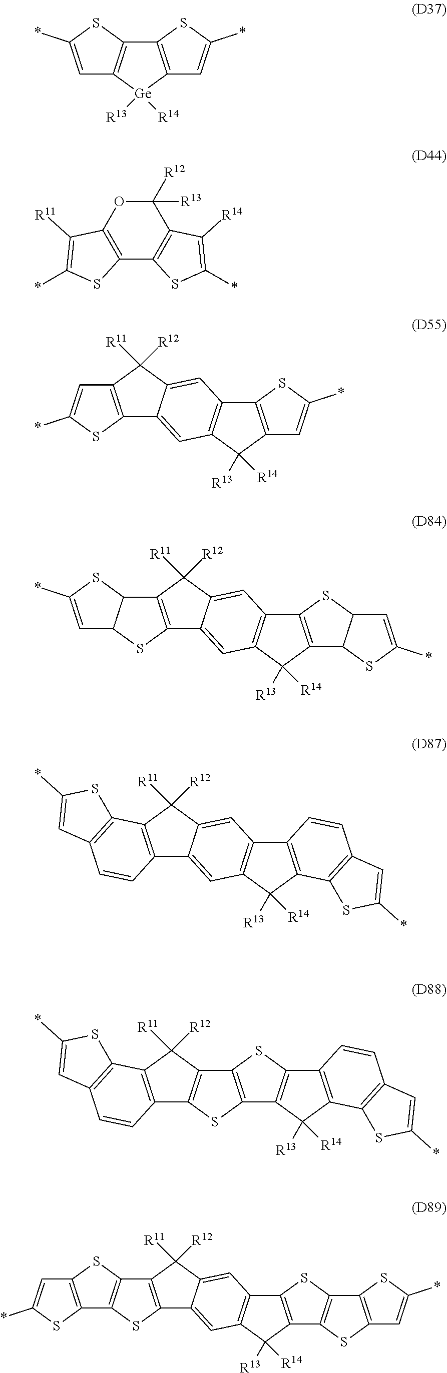

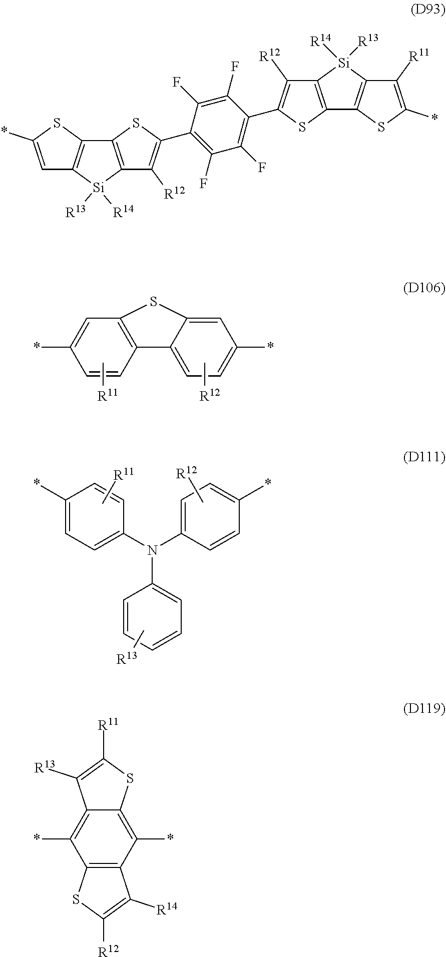

wherein R¹¹, R¹², R¹³, R¹⁴ independently of each other denote H or have one of the meanings of L as defined above.

Preferred acceptor units or units A are selected from the following formulae

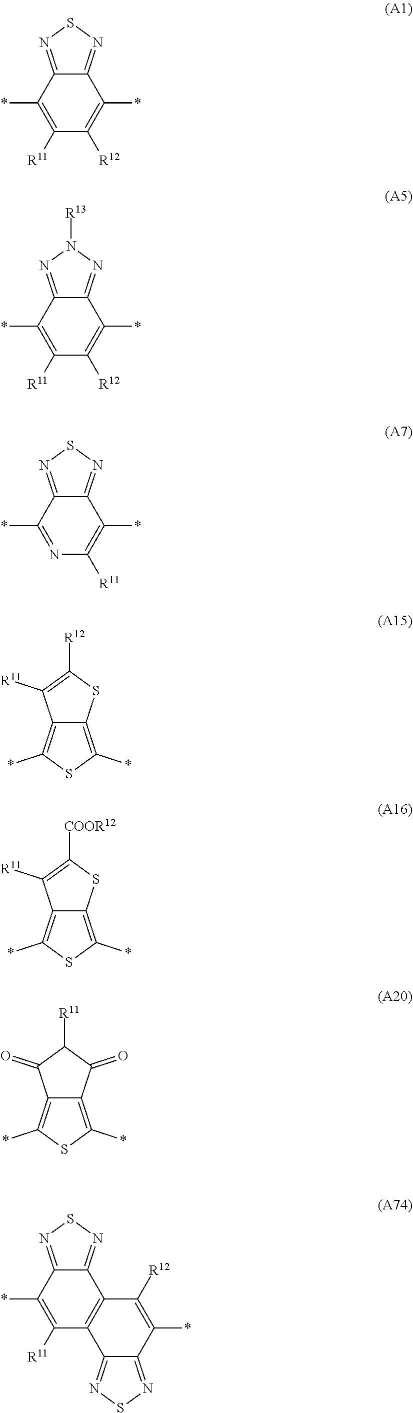

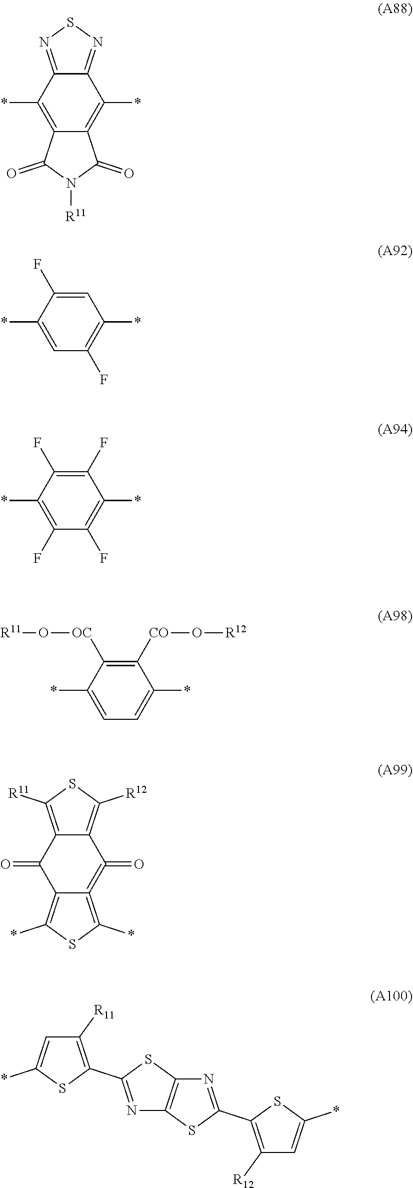

wherein R¹¹, R¹², R¹³, R¹⁴ independently of each other denote H or have one of the meanings of L as defined above.

Preferred spacer units or units Sp are selected from the following formulae

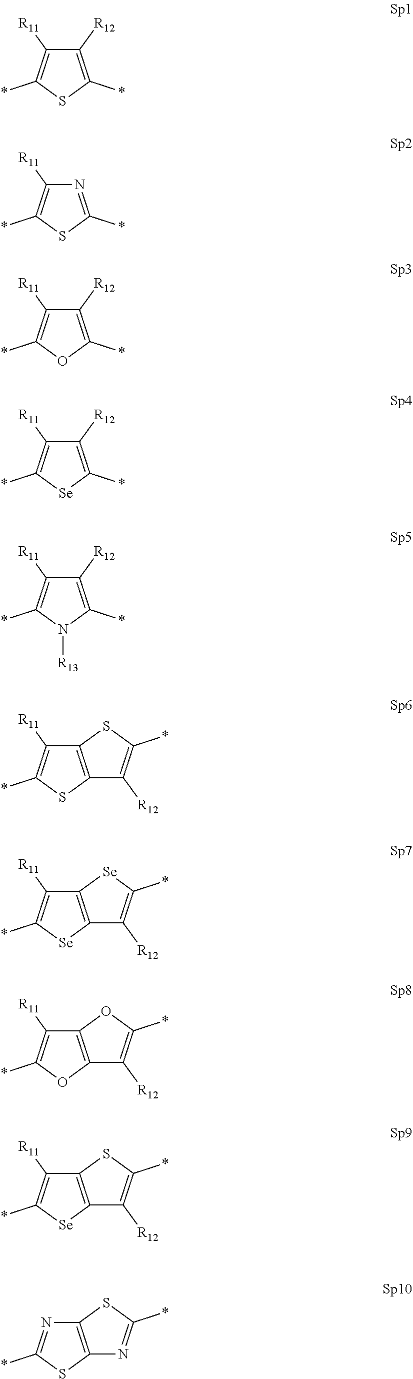

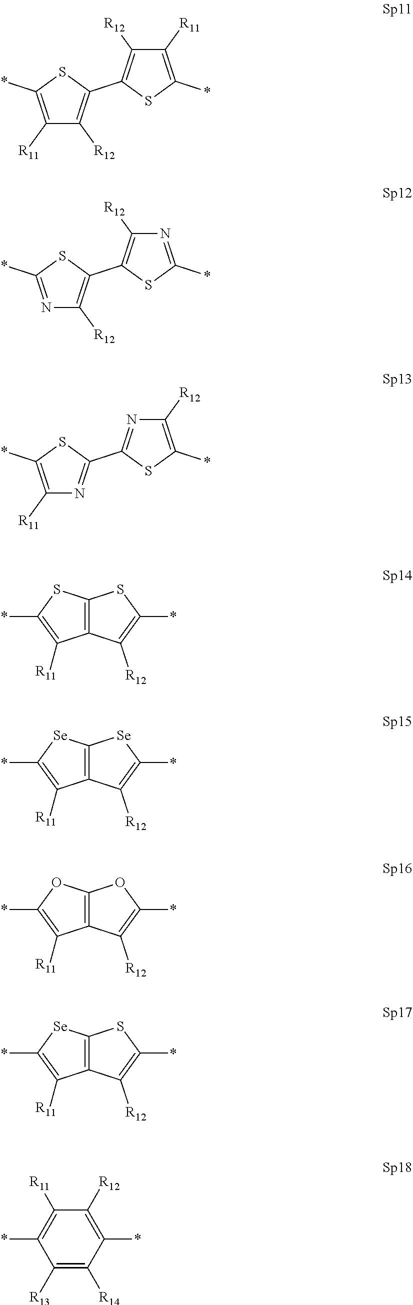

wherein R¹¹, R¹², R¹³, R¹⁴ independently of each other denote H or have one of the meanings of L as defined above.

In the formulae Sp1 to Sp17 preferably R¹¹ and R¹² are H. In formula Sp18 preferably R¹¹⁻¹⁴ are H or F.

Preferably the conjugated polymer contains, preferably consists of
a) one or more donor units selected from the group consisting of the formulae D1, D7, D10, D11, D19, D22, D29, D30, D35, D36, D37, D44, D55, D84, D87, D88, D89, D93, D106, D111, D119, D140, D141, D146, and D147 and/or
b) one or more acceptor units selected from the group consisting of the formulae A1, A5, A7, A15, A16, A20, A74, A88, A92, A94 and A98, A99, A100 and
c) optionally one or more spacer units selected from the group consisting of the formulae Sp1-Sp18, very preferably of the formulae Sp1, Sp6, Sp11 and Sp14,
   wherein the spacer units, if present, are preferably located between the donor and acceptor units such that a donor unit and an acceptor unit are not directly connected to each other.

In a second preferred embodiment the compound of formula I is a conjugated polymer that comprises, preferably consists of one or more, preferably one, two, three or four, distinct repeating units D, and one or more, preferably one, two or three, distinct repeating units A.

Preferably the conjugated polymer according to this second preferred embodiment contains from one to six, very preferably one, two, three or four distinct units D and from one to six, very preferably one, two, three or four distinct units A, wherein d1, d2, d3, d4, d5 and d6 denote the molar ratio of each distinct unit D, and a1, a2, a3, a4, a5 and a6 denote the molar ratio of each distinct unit A, and

each of d1, d2, d3, d4, d5 and d6 is from 0 to 0.6, and d1+d2+d3+d4+d5+d6 is from 0.2 to 0.8, preferably from 0.3 to 0.7, and

each of a1, a2, a3, a4, a5 and a6 is from 0 to 0.6, and a1+a2+a3+a4+a5+d6 is from 0.2 to 0.8, preferably from 0.3 to 0.7, and

d1+d2+d3+d4+d5+d6+a1+a2+a3+a4+a5+a6 is from 0.8 to 1, preferably 1.

Preferably the conjugated polymer according to this second preferred embodiment contains, preferably consists of
a) one or more donor units selected from the group consisting of the formulae D1, D7, D10, D11, D19, D22, D29, D30, D35, D36, D37, D44, D55, D84, D87, D88, D89, D93, D106, D111, D119, D140, D141, D146, and D147 and/or
b) one or more acceptor units selected from the group consisting of the formulae A1, A5, A7, A15, A16, A20, A74, A88, A92, A94, A98, A99 and A100.

In the above conjugated polymers, like those of formula P and its subformulae, the total number of repeating units n is preferably from 2 to 10,000. The total number of repeating units n is preferably ≥5, very preferably ≥10, most preferably ≥50, and preferably ≤500, very preferably ≤1,000, most preferably ≤2,000, including any combination of the aforementioned lower and upper limits of n.

The conjugated polymers are preferably statistical or random copolymers.

Very preferred conjugated polymers comprise one or more of the following subformulae as one or more repeating units

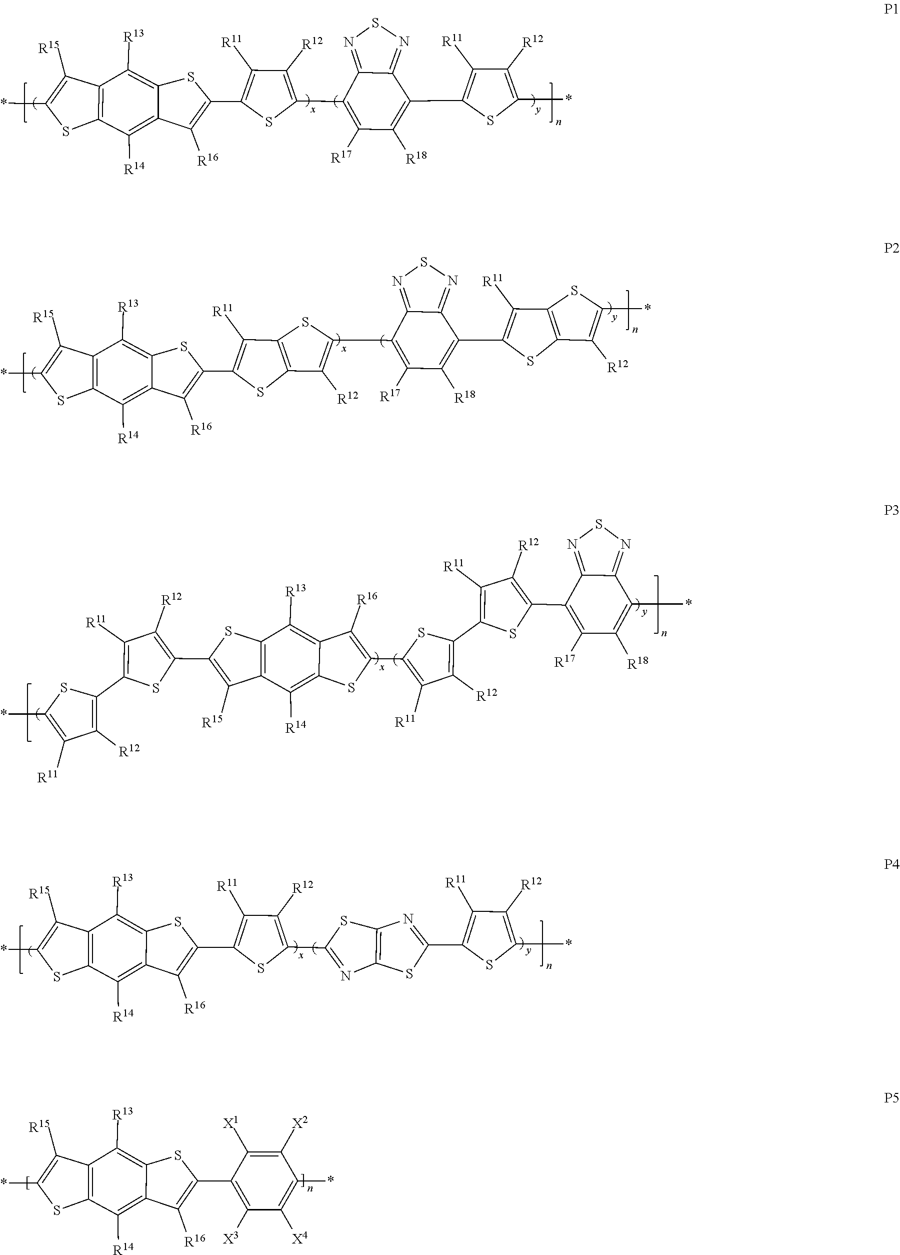

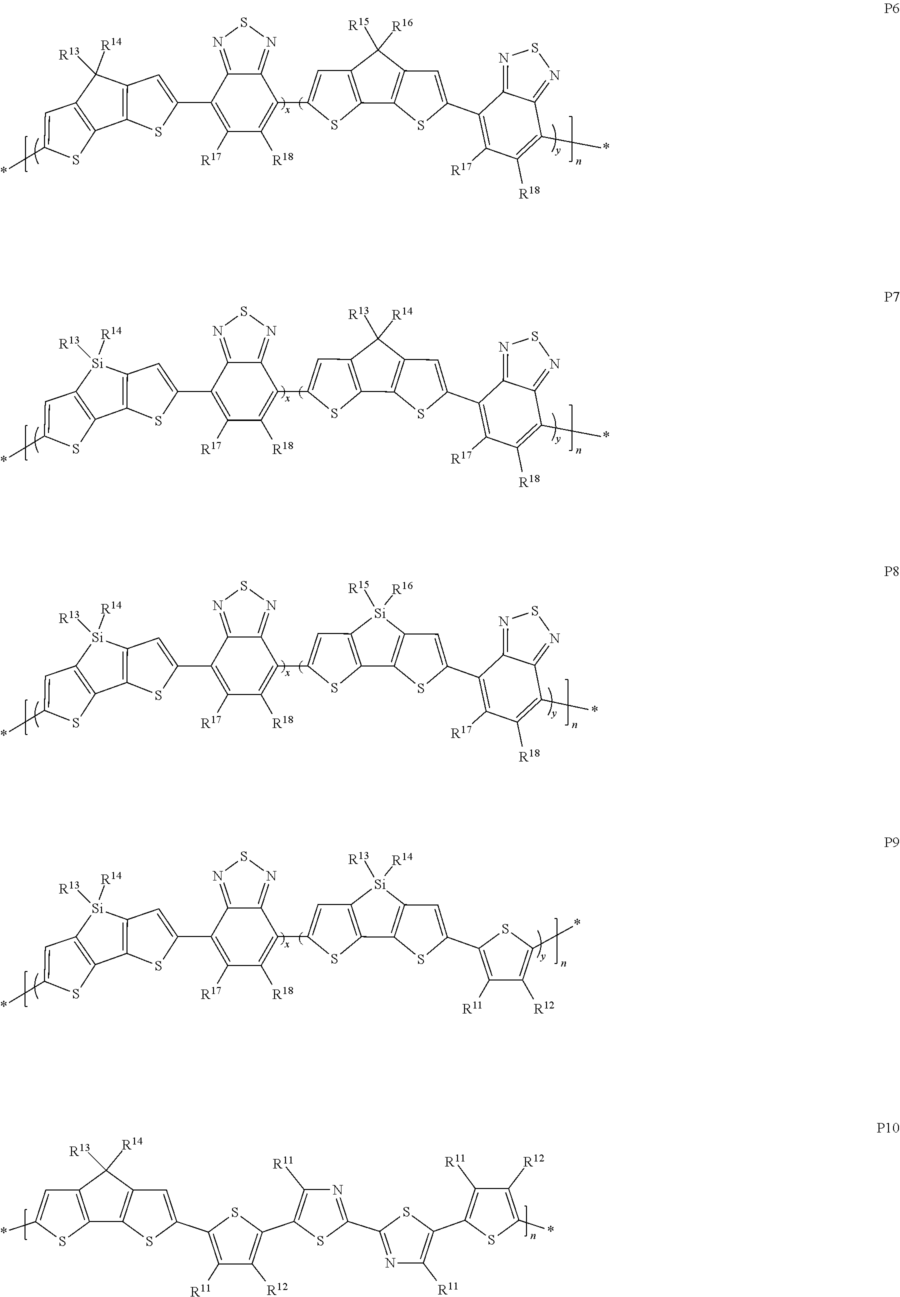

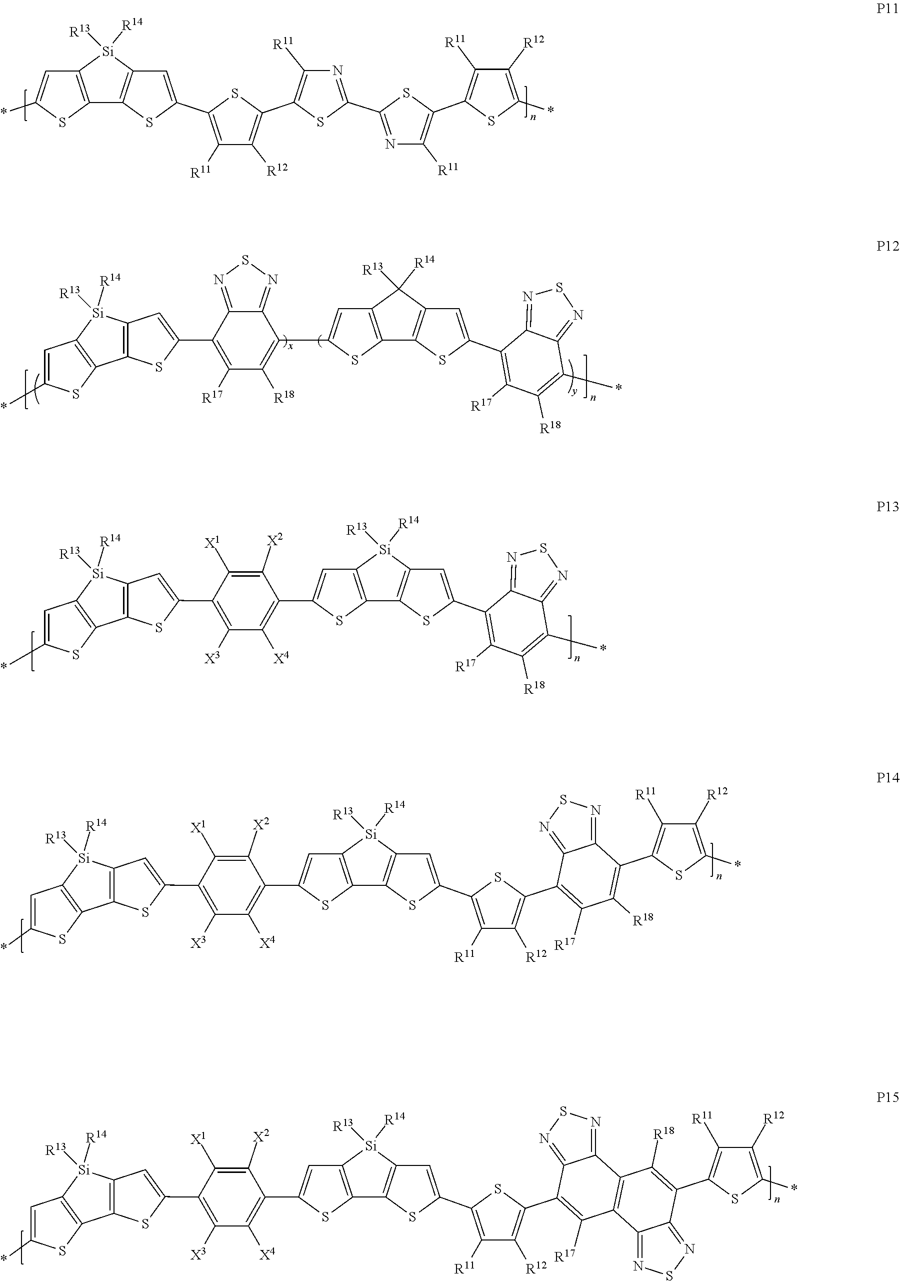

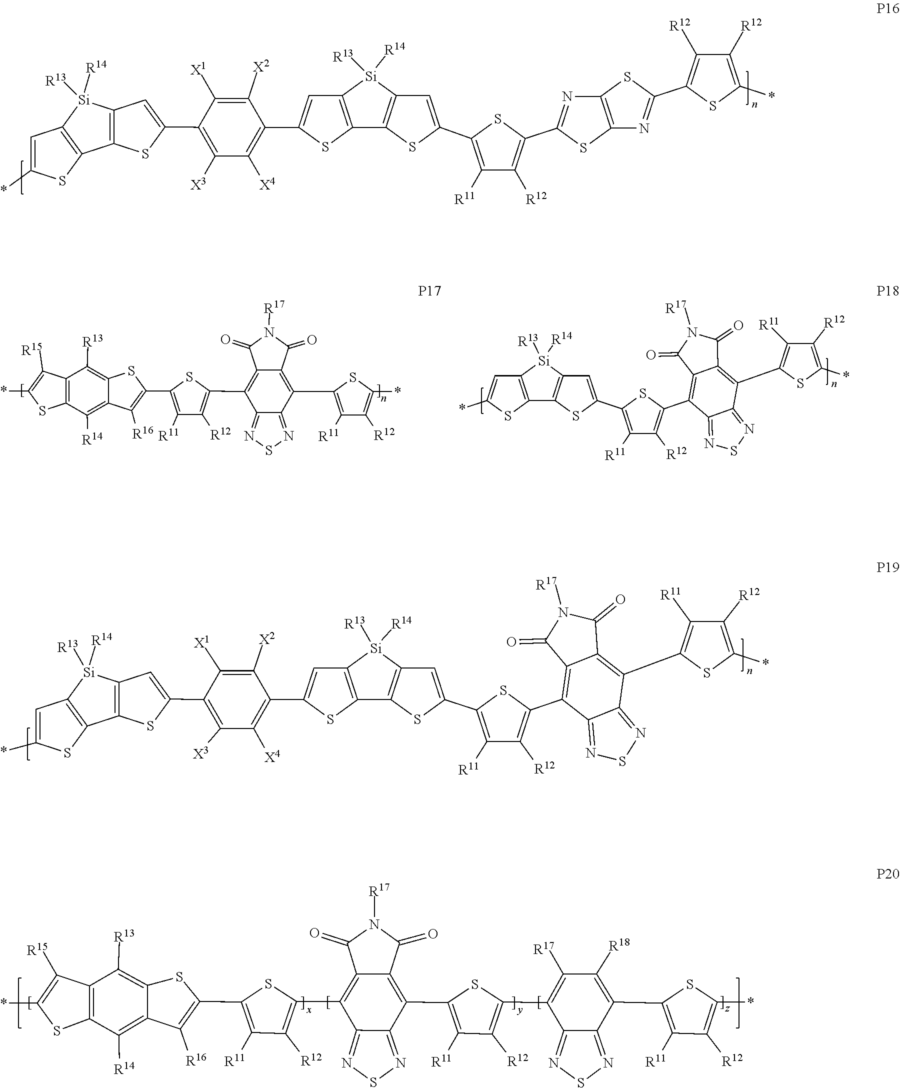

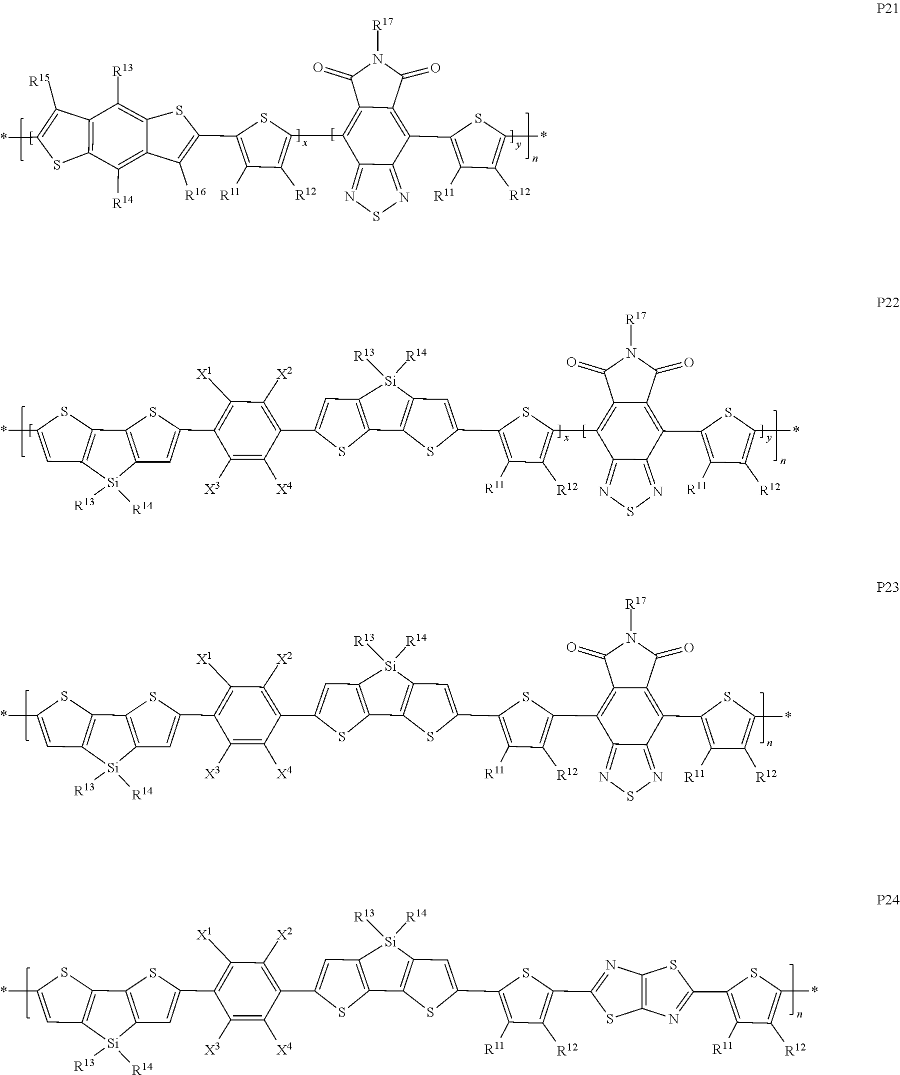

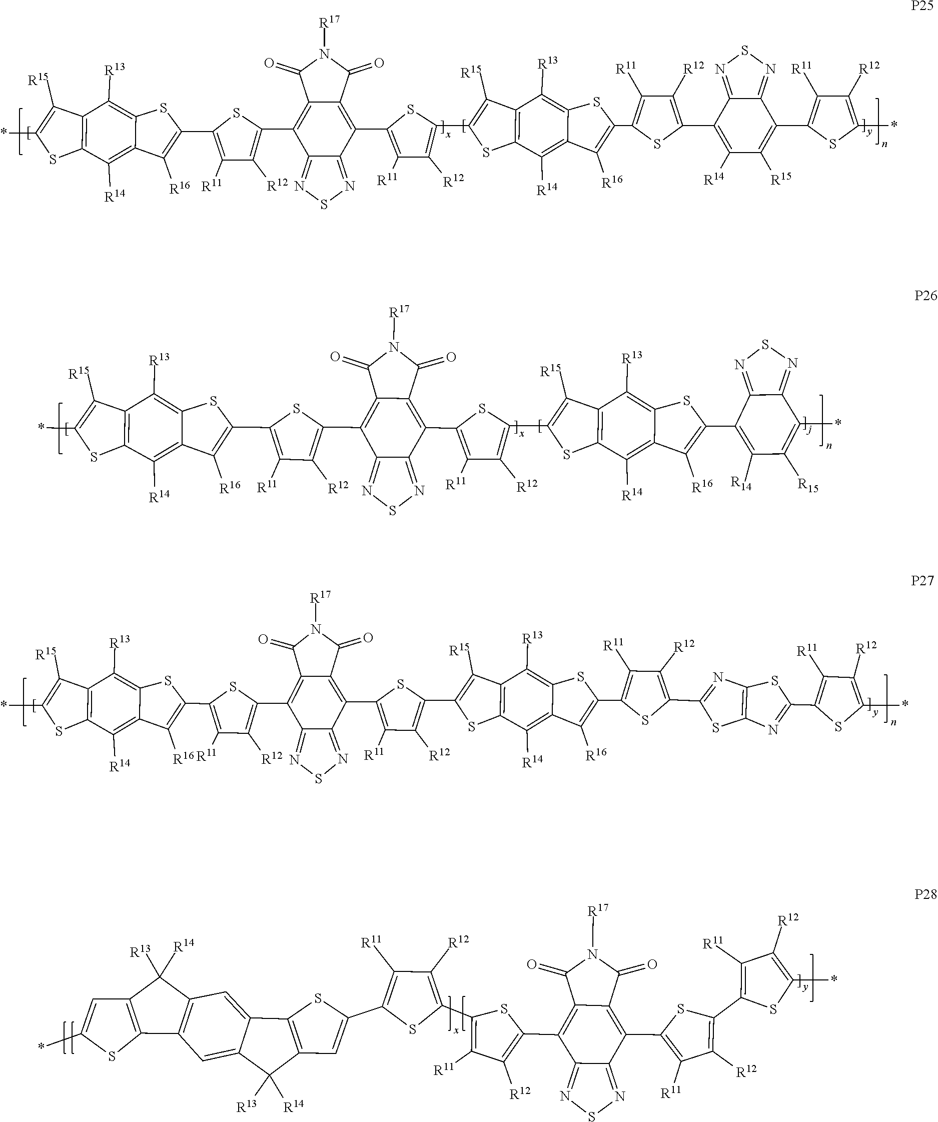

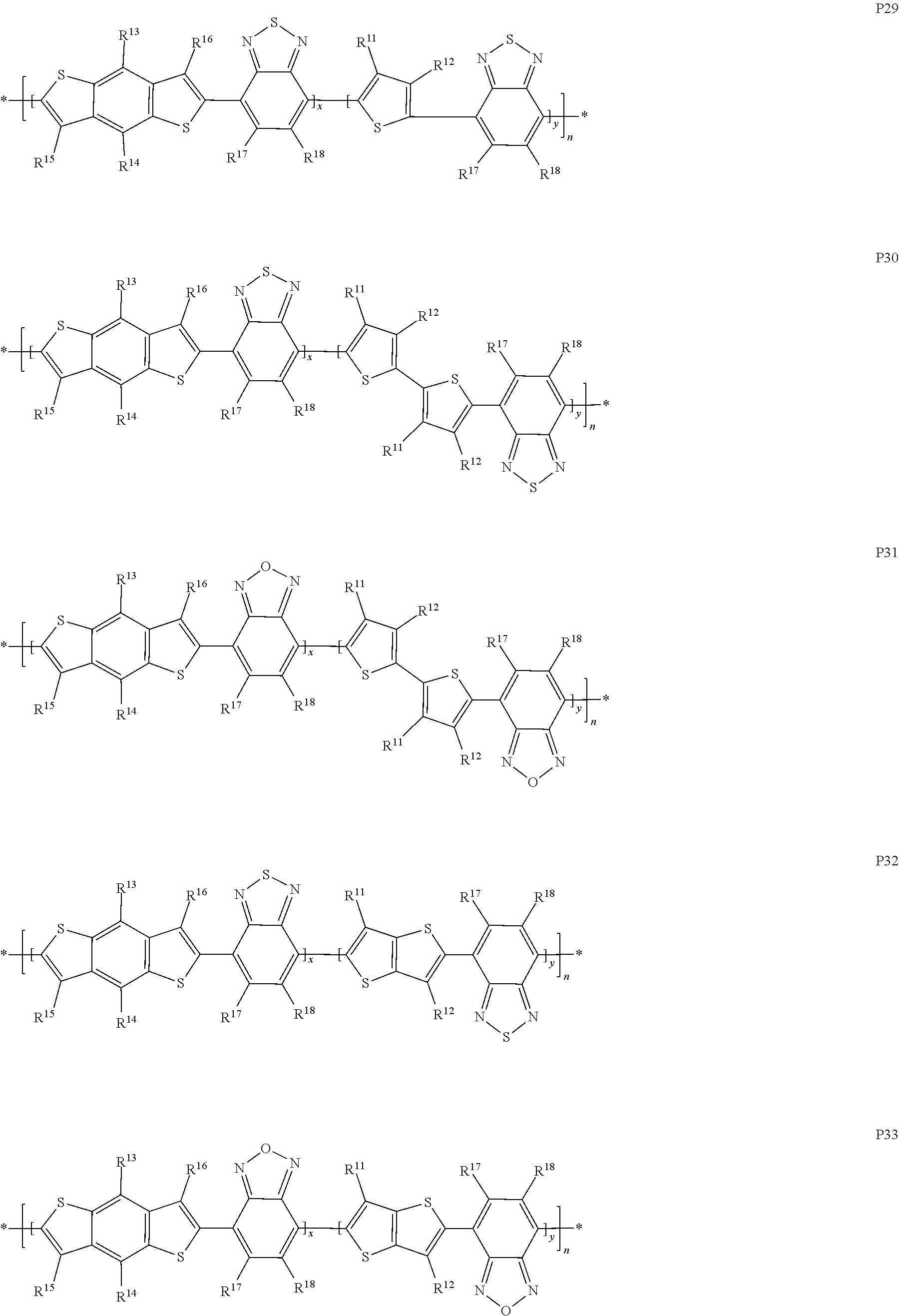

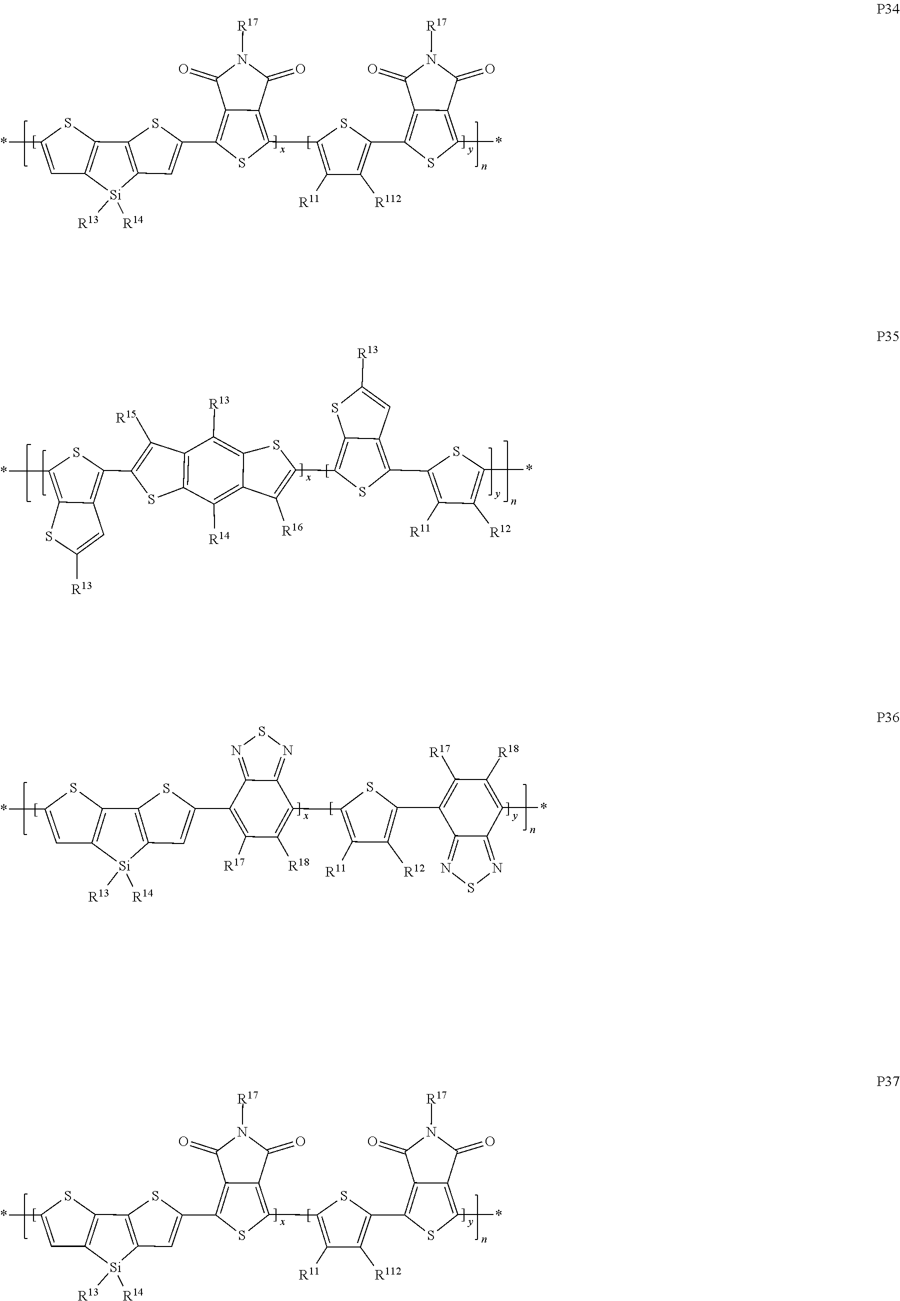

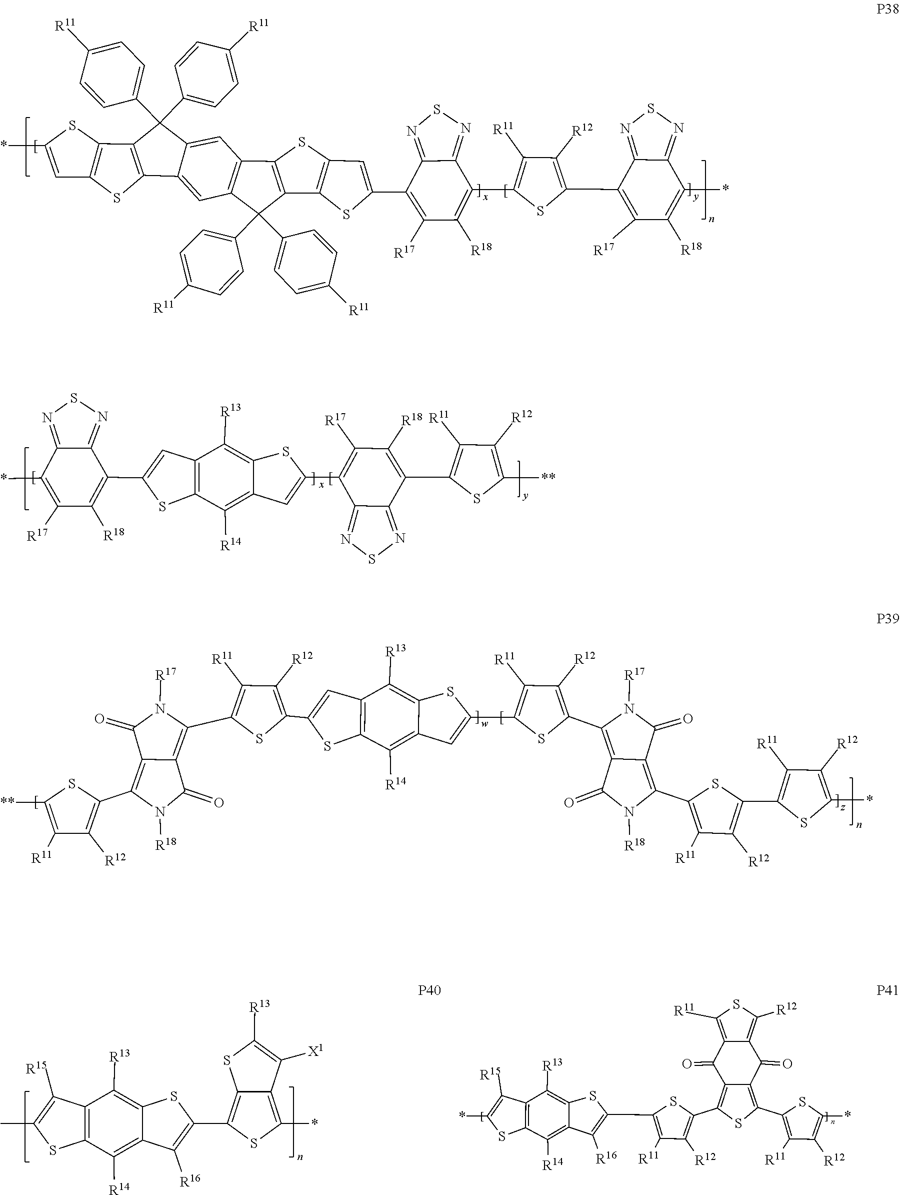

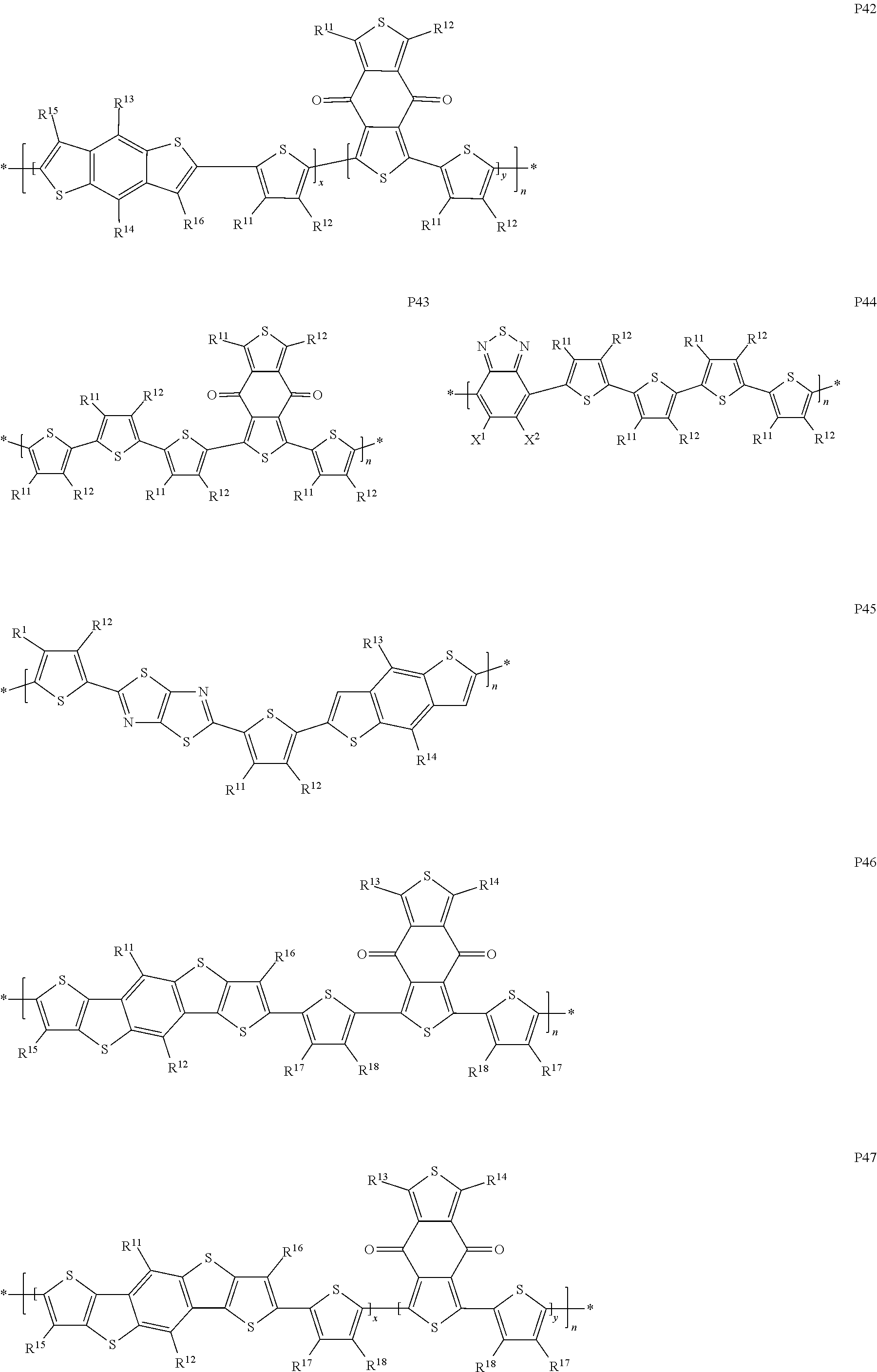

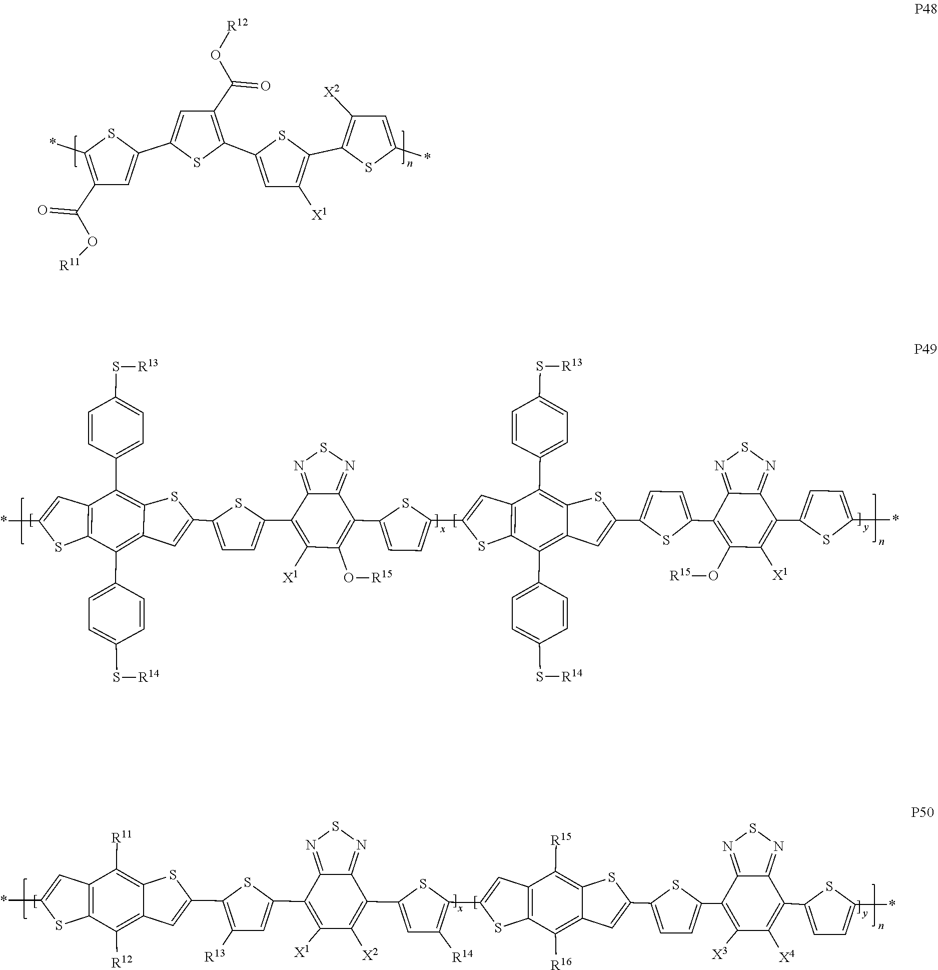

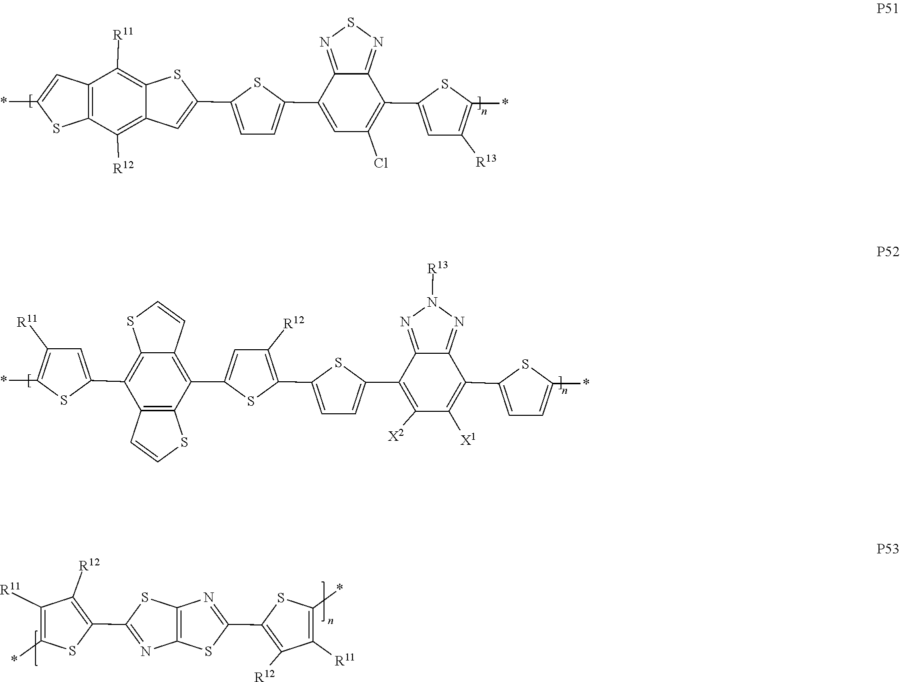

wherein R¹¹⁻¹⁷, x, y and n are as defined above, w and z have one of the meanings given for y, x+y+w+z=1, R¹⁸ and R¹⁹ have one of the meanings given for R^{T1}, and X¹, X², X³ and X⁴ denote H, F or Cl.

In the polymers of formulae P1-P53, x, y, w and z are preferably from 0.1 to 0.9, very preferably from 0.3 to 0.7, most preferably from 0.4 to 0.6.

In the formulae P1-P53 preferably one or more of X¹, X², X³ and X⁴ denote F, very preferably all of X¹, X², X³ and X⁴ denote F or X¹ and X² denote H and X³ and X⁴ denote F.

In the formulae P1-P53, preferably R¹¹ and R¹² are H. Further preferably R¹¹ and R¹², when being different from H, denote straight-chain or branched alkyl with 1 to 30, preferably 1 to 20, C atoms that is optionally fluorinated.

In the formulae P1-P53, preferably R¹⁵ and R¹⁶ are H, and R¹³ and R¹⁴ are different from H.

In the formulae P1-P53, preferably R¹³, R¹⁴, R¹⁵ and R¹⁶, when being different from H, are selected from the following groups:
the group consisting of straight-chain or branched alkyl, alkoxy or sulfanylalkyl with 1 to 30, preferably 1 to 20, C atoms that is optionally fluorinated,
the group consisting of straight-chain or branched alkylcarbonyl or alkylcarbonyloxy with 2 to 30, preferably 2 to 20, C atoms, that is optionally fluorinated.

In the formulae P1-P53, preferably R¹⁷ and R¹⁸, when being different from H, are selected from the following groups:
the group consisting of straight-chain or branched alkyl, alkoxy or sulfanylalkyl with 1 to 30, preferably 1 to 20, C atoms that is optionally fluorinated,
the group consisting of straight-chain or branched alkylcarbonyl or alkylcarbonyloxy with 2 to 30, preferably 2 to 20, C atoms, that is optionally fluorinated.
the group consisting of F and Cl.

Further preferred are conjugated polymers selected of formula PT

R³¹-chain-R³² PT

wherein “chain” denotes a polymer chain selected of formula Pi, Pii or P1-P53, and R³¹ and R³² have independently of each other one of the meanings of R^{T1} as defined above, or denote, independently of each other, H, F, Br, Cl, I, —CH₂Cl, —CHO, —CR′═CR″₂, —SiR′R″R″′, —SiR′X′X″, —SiR′R″X′, —SnR′R″R″′, —BR′R″, —B(OR′)(OR″), —B(OH)₂, —O—SO₂—R′, —C═CH, —C═C—SiR′₃, —ZnX′ or an endcap group, X′ and X″ denote halogen, R′, R″ and R″′ have independently of each other one of the meanings of R⁰ given in formula 1, and preferably denote alkyl with 1 to 12 C atoms, and two of R′, R″ and R″′ may also form a cyclosilyl, cyclostannyl, cycloborane or cycloboronate group with 2 to 20 C atoms together with the respective hetero atom to which they are attached.

Preferred endcap groups R³¹ and R³² are H, C1-20 alkyl, or optionally substituted 06-12 aryl or 02-10 heteroaryl, very preferably H, phenyl or thiophene.

The compounds of formula I and the conjugated polymers of formula P and PT can be synthesized according to or in analogy to methods that are known to the skilled person and are described in the literature. Other methods of preparation can be taken from the examples.

For example, the compounds of the present invention can be suitably prepared by aryl-aryl coupling reactions, such as Yamamoto coupling, Suzuki coupling, Stille coupling, Sonogashira coupling, Heck coupling or Buchwald coupling. The educts can be prepared according to methods which are known to the person skilled in the art.

Preferred aryl-aryl coupling methods used in the synthesis methods as described above and below are Yamamoto coupling, Kumada coupling, Negishi coupling, Suzuki coupling, Stille coupling, Sonogashira coupling, Heck coupling, C—H activation coupling, Ullmann coupling or Buchwald coupling. Especially preferred are Suzuki coupling, Negishi coupling, Stille coupling and Yamamoto coupling. Suzuki coupling is described for example in WO 00/53656 A1. Negishi coupling is described for example in J. Chem. Soc., Chem. Commun., 1977, 683-684. Yamamoto coupling is described in for example in T. Yamamoto et al., Prog. Polym. Sci., 1993, 17, 1153-1205, or WO 2004/022626 A1. Stille coupling is described for example in Z. Bao et al., J. Am. Chem. Soc., 1995, 117, 12426-12435 and C—H activation is described for example in M. Leclerc et al, Angew. Chem. Int. Ed., 2012, 51, 2068-2071. For example, when using Yamamoto coupling, educts having two reactive halide groups are preferably used. When using Suzuki coupling, educts having two reactive boronic acid or boronic acid ester groups or two reactive halide groups are preferably used. When using Stille coupling, edcuts having two reactive stannane groups or two reactive halide groups are preferably used. When using Negishi coupling, educts having two reactive organozinc groups or two reactive halide groups are preferably used.

Preferred catalysts, especially for Suzuki, Negishi or Stille coupling, are selected from Pd(0) complexes or Pd(II) salts. Preferred Pd(0) complexes are those bearing at least one phosphine ligand such as Pd(Ph₃P)₄. Another preferred phosphine ligand is tris(ortho-tolyl)phosphine, i.e. Pd(o-Tol₃P)₄. Preferred Pd(II) salts include palladium acetate, i.e. Pd(OAc)₂. Alternatively the Pd(0) complex can be prepared by mixing a Pd(0) dibenzylideneacetone complex, for example tris(dibenzyl-ideneacetone)dipalladium(0), bis(dibenzylideneacetone)palladium(0), or Pd(II) salts e.g. palladium acetate, with a phosphine ligand, for example triphenylphosphine, tris(ortho-tolyl)phosphine or tri(tert-butyl)phosphine. Suzuki coupling is performed in the presence of a base, for example sodium carbonate, potassium carbonate, cesium carbonate, lithium hydroxide, potassium phosphate or an organic base such as tetraethylammonium carbonate or tetraethylammonium hydroxide. Yamamoto coupling employs a Ni(0) complex, for example bis(1,5-cyclooctadienyl) nickel(0).

As alternatives to halogens as described above, leaving groups of formula —O—SO₂Z⁰ can be used wherein Z° is an alkyl or aryl group, preferably C₁₋₁₀ alkyl or C₆₋₁₂ aryl. Particular examples of such leaving groups are tosylate, mesylate and triflate.

Especially suitable and preferred synthesis methods of the compounds of formula I and its subformulae are illustrated in the synthesis schemes shown hereinafter.

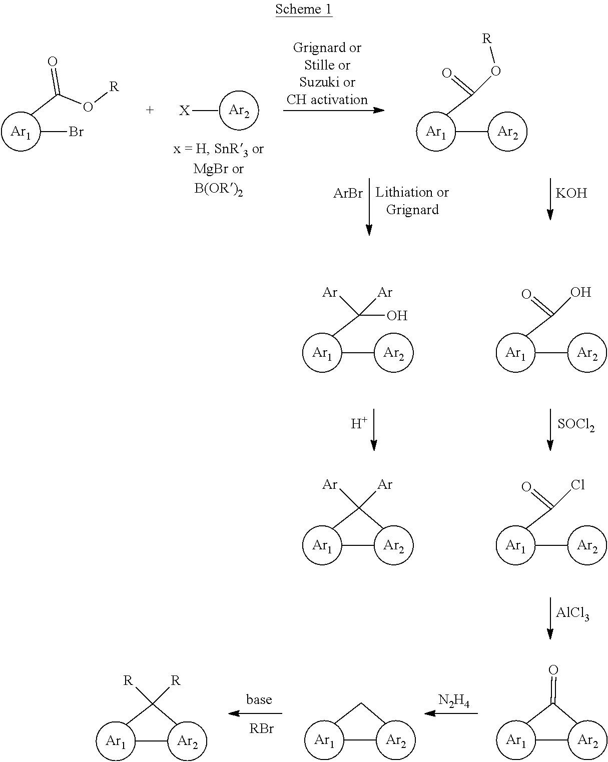

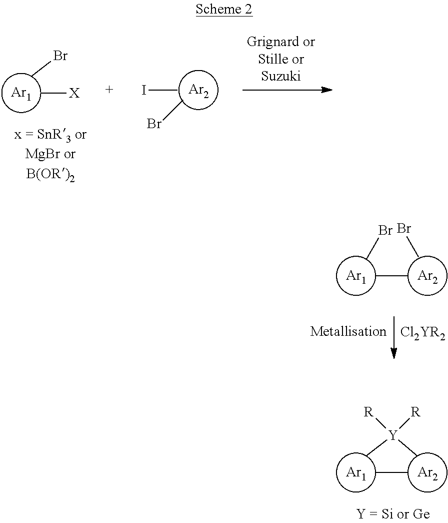

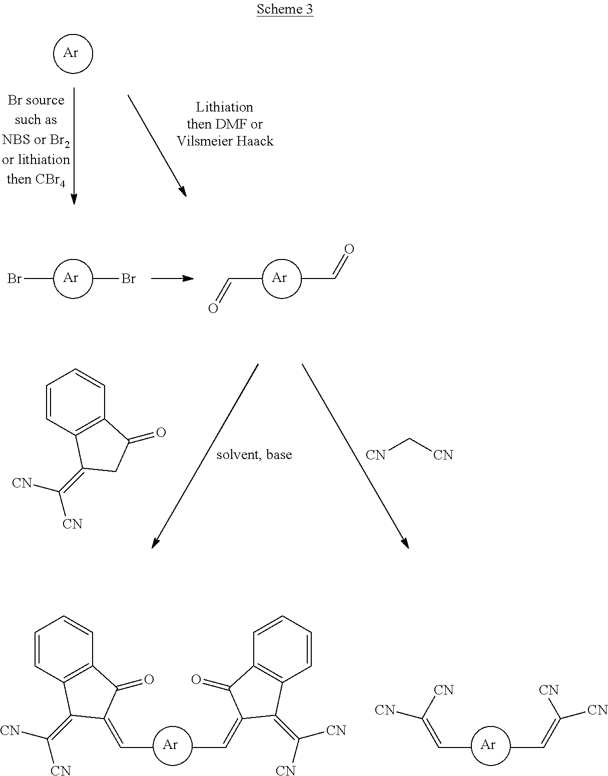

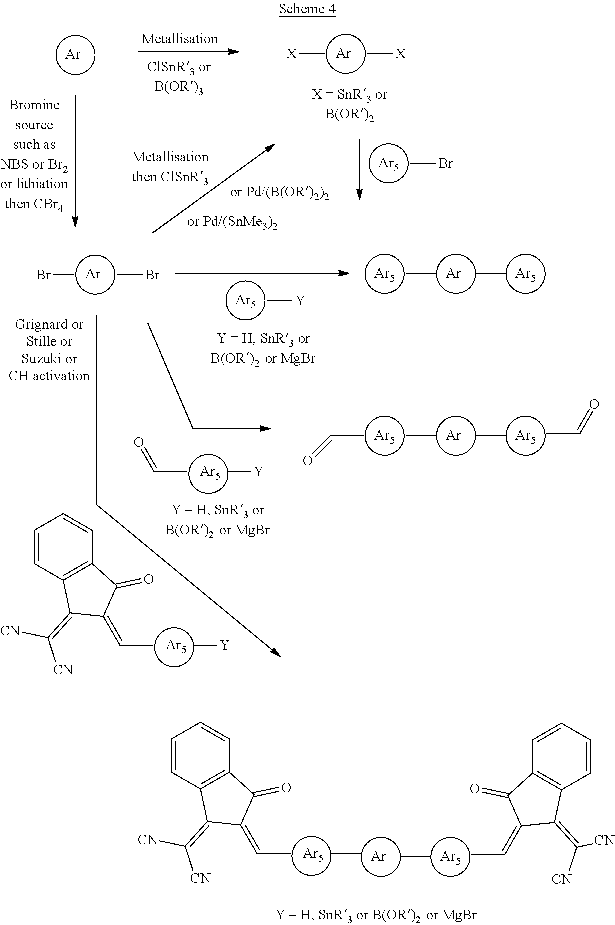

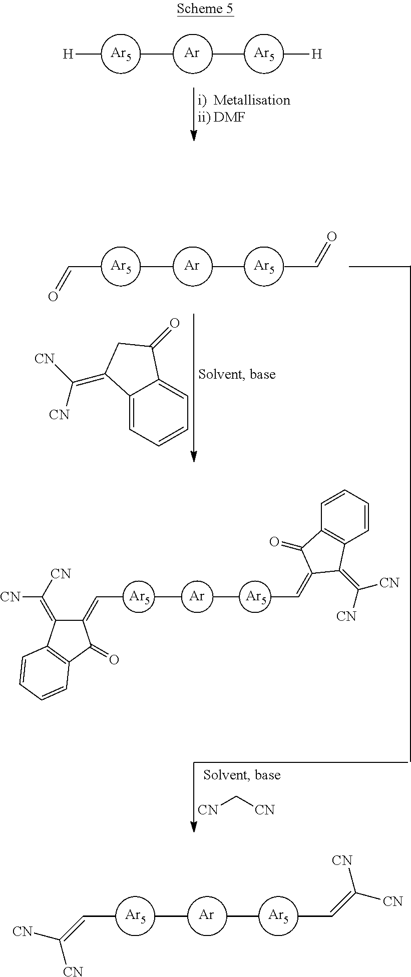

Novel methods of preparing compounds of formula I as described above and below are another aspect of the invention.

The compounds of formula I can also be used in compositions, for example together with monomeric or polymeric compounds having charge-transport, semiconducting, electrically conducting, photo-conducting and/or light emitting semiconducting properties, or for example with compounds having hole blocking or electron blocking properties for use as interlayers or charge blocking layers in OLEDs or PSCs.

Thus, another aspect of the invention relates to a composition comprising one or more compounds of formula I and one or more small molecule compounds and/or polymers having one or more of a charge-transport, semiconducting, electrically conducting, photoconducting, hole blocking and electron blocking property.

These compositions blends can be prepared by conventional methods that are described in prior art and known to the skilled person. Typically the compounds and/or polymers are mixed with each other or dissolved in suitable solvents and the solutions combined.

Another aspect of the invention relates to a formulation comprising one or more compounds of formula I or compositions as described above and below and one or more organic solvents.

Preferred solvents are aliphatic hydrocarbons, chlorinated hydrocarbons, aromatic hydrocarbons, ketones, ethers and mixtures thereof. Additional solvents which can be used include 1,2,4-trimethylbenzene, 1,2,3,4-tetra-methyl benzene, pentylbenzene, mesitylene, cumene, cymene, cyclohexylbenzene, diethylbenzene, tetralin, decalin, 2,6-lutidine, 2-fluoro-m-xylene, 3-fluoro-o-xylene, 2-chlorobenzotrifluoride, N,N-dimethylformamide, 2-chloro-6-fluorotoluene, 2-fluoroanisole, anisole, 2,3-dimethylpyrazine, 4-fluoroanisole, 3-fluoroanisole, 3-trifluoro-methylanisole, 2-methylanisole, phenetol, 4-methylanisole, 3-methylanisole, 4-fluoro-3-methylanisole, 2-fluorobenzonitrile, 4-fluoroveratrol, 2,6-dimethylanisole, 3-fluorobenzo-nitrile, 2,5-dimethylanisole, 2,4-dimethylanisole, benzonitrile, 3,5-dimethyl-anisole, N,N-dimethylaniline, ethyl benzoate, 1-fluoro-3,5-dimethoxy-benzene, 1 methylnaphthalene, N-methylpyrrolidinone, 3-fluorobenzo-trifluoride, benzotrifluoride, dioxane, trifluoromethoxy-benzene, 4-fluorobenzotrifluoride, 3-fluoropyridine, toluene, 2-fluoro-toluene, 2-fluorobenzotrifluoride, 3-fluorotoluene, 4-isopropylbiphenyl, phenyl ether, pyridine, 4-fluorotoluene, 2,5-difluorotoluene, 1-chloro-2,4-difluorobenzene, 2-fluoropyridine, 3-chlorofluoro-benzene, 1-chloro-2,5-difluorobenzene, 4-chlorofluorobenzene, chloro-benzene, o-dichlorobenzene, 2-chlorofluorobenzene, p-xylene, m-xylene, o-xylene or mixture of o-, m-, and p-isomers. Solvents with relatively low polarity are generally preferred. For inkjet printing solvents and solvent mixtures with high boiling temperatures are preferred. For spin coating alkylated benzenes like xylene and toluene are preferred.

Examples of especially preferred solvents include, without limitation, dichloromethane, trichloromethane, chlorobenzene, o-dichlorobenzene, tetrahydrofuran, anisole, 2,4-dimethylanisole, 1-methylnaphthalene, morpholine, toluene, o-xylene, m-xylene, p-xylene, 1,4-dioxane, acetone, methylethylketone, 1,2-dichloroethane, 1,1,1-trichloroethane, 1,1,2,2-tetrachloroethane, ethyl acetate, n-butyl acetate, N,N-dimethylformamide, dimethylacetamide, dimethylsulfoxide, 1,5-dimethyltetraline, propiophenone, acetophenone, tetraline, 2-methylthiophene, 3 methylthiophene, decaline, indane, methyl benzoate, ethyl benzoate, mesitylene and/or mixtures thereof.

The concentration of the compounds or polymers in the solution is preferably 0.1 to 10% by weight, more preferably 0.5 to 5% by weight. Optionally, the solution also comprises one or more binders to adjust the rheological properties, as described for example in WO 2005/055248 A1.

After the appropriate mixing and ageing, solutions are evaluated as one of the following categories: complete solution, borderline solution or insoluble. The contour line is drawn to outline the solubility parameter-hydrogen bonding limits dividing solubility and insolubility. ‘Complete’ solvents falling within the solubility area can be chosen from literature values such as published in “Crowley, J. D., Teague, G. S. Jr and Lowe, J. W. Jr., Journal of Paint Technology, 1966, 38 (496), 296”. Solvent blends may also be used and can be identified as described in “Solvents, W. H. Ellis, Federation of Societies for Coatings Technology, p 9-10, 1986”. Such a procedure may lead to a blend of ‘non’ solvents that will both dissolve the compounds of the present invention, although it is desirable to have at least one true solvent in a blend.

The compounds of formula I can also be used in patterned OSC layers in the devices as described above and below. For applications in modern microelectronics it is generally desirable to generate small structures or patterns to reduce cost (more devices/unit area), and power consumption. Patterning of thin layers comprising a compound according to the present invention can be carried out for example by photolithography, electron beam lithography or laser patterning.

For use as thin layers in electronic or electrooptical devices the compounds, compositions or formulations of the present invention may be deposited by any suitable method. Liquid coating of devices is more desirable than vacuum deposition techniques. Solution deposition methods are especially preferred. The formulations of the present invention enable the use of a number of liquid coating techniques. Preferred deposition techniques include, without limitation, dip coating, spin coating, ink jet printing, nozzle printing, letter-press printing, screen printing, gravure printing, doctor blade coating, roller printing, reverse-roller printing, offset lithography printing, dry offset lithography printing, flexographic printing, web printing, spray coating, curtain coating, brush coating, slot dye coating or pad printing.

Ink jet printing is particularly preferred when high resolution layers and devices needs to be prepared. Selected formulations of the present invention may be applied to prefabricated device substrates by ink jet printing or microdispensing. Preferably industrial piezoelectric print heads such as but not limited to those supplied by Aprion, Hitachi-Koki, InkJet Technology, On Target Technology, Picojet, Spectra, Trident, Xaar may be used to apply the organic semiconductor layer to a substrate. Additionally semi-industrial heads such as those manufactured by Brother, Epson, Konica, Seiko Instruments Toshiba TEC or single nozzle microdispensers such as those produced by Microdrop and Microfab may be used.

In order to be applied by ink jet printing or microdispensing, the compounds or polymers should be first dissolved in a suitable solvent. Solvents must fulfil the requirements stated above and must not have any detrimental effect on the chosen print head. Additionally, solvents should have boiling points >100° C., preferably >140° C. and m ore preferably >150° C. in order to prevent operability problems caused by the solution drying out inside the print head. Apart from the solvents mentioned above, suitable solvents include substituted and non-substituted xylene derivatives, di-C₁₋₂-alkyl formamide, substituted and non-substituted anisoles and other phenol-ether derivatives, substituted heterocycles such as substituted pyridines, pyrazines, pyrimidines, pyrrolidinones, substituted and non-substituted N,N-di-C₁₋₂-alkylanilines and other fluorinated or chlorinated aromatics.

A preferred solvent for depositing a compound of formula I by ink jet printing comprises a benzene derivative which has a benzene ring substituted by one or more substituents wherein the total number of carbon atoms among the one or more substituents is at least three. For example, the benzene derivative may be substituted with a propyl group or three methyl groups, in either case there being at least three carbon atoms in total. Such a solvent enables an ink jet fluid to be formed comprising the solvent with the compound or polymer, which reduces or prevents clogging of the jets and separation of the components during spraying. The solvent(s) may include those selected from the following list of examples: dodecylbenzene, 1-methyl-4-tert-butylbenzene, terpineol, limonene, isodurene, terpinolene, cymene, diethylbenzene. The solvent may be a solvent mixture, that is a combination of two or more solvents, each solvent preferably having a boiling point >100° C., more preferably >140° C. Such solvent(s) also enhance film formation in the layer deposited and reduce defects in the layer.

The ink jet fluid (that is mixture of solvent, binder and semiconducting compound) preferably has a viscosity at 20° C. of 1-100 mPa s, more preferably 1-50 mPa s and most preferably 1-30 mPa s.

The compositions and formulations according to the present invention can additionally comprise one or more further components or additives selected for example from surface-active compounds, lubricating agents, wetting agents, dispersing agents, hydrophobing agents, adhesive agents, flow improvers, defoaming agents, deaerators, diluents which may be reactive or non-reactive, auxiliaries, colourants, dyes or pigments, sensitizers, stabilizers, nanoparticles or inhibitors.

The compounds according to the present invention are useful as charge transport, semiconducting, electrically conducting, photoconducting or light emitting materials in optical, electrooptical, electronic, electroluminescent or photoluminescent components or devices. In these devices, the compounds of the present invention are typically applied as thin layers or films.

Thus, the present invention also provides the use of the semiconducting compound or composition or layer in an electronic device. The compound or composition may be used as a high mobility semiconducting material in various devices and apparatus. The compound or composition may be used, for example, in the form of a semiconducting layer or film. Accordingly, in another aspect, the present invention provides a semiconducting layer for use in an electronic device, the layer comprising a compound or composition according to the invention. The layer or film may be less than about 30 microns. For various electronic device applications, the thickness may be less than about 1 micron thick. The layer may be deposited, for example on a part of an electronic device, by any of the aforementioned solution coating or printing techniques.

The invention additionally provides an electronic device comprising compound or composition or organic semiconducting layer according to the present invention. Especially preferred devices are OFETs, TFTs, ICs, logic circuits, capacitors, RFID tags, OLEDs, OLETs, OPEDs, OPVs, PSCs, OPDs, solar cells, laser diodes, photoconductors, photodetectors, electrophotographic devices, electrophotographic recording devices, organic memory devices, sensor devices, charge injection layers, Schottky diodes, planarising layers, antistatic films, conducting substrates and conducting patterns.

Especially preferred electronic device are OFETs, OLEDs, OPV, PSC and OPD devices, in particular OPD, PSC and bulk heterojunction (BHJ) OPV devices. In an OFET, for example, the active semiconductor channel between the drain and source may comprise the compound or composition of the invention. As another example, in an OLED device, the charge (hole or electron) injection or transport layer may comprise the compound or composition of the invention.

For use in the photoactive layer of OPV or OPD devices the compounds according to the present invention are preferably used in a composition that comprises or contains, more preferably consists of, one or more p-type (electron donor) semiconductors and one or more n-type (electron acceptor) semiconductors.

The n-type semiconductor is for example constituted by a compound of formula I.

The p-type semiconductor is preferably a conjugated polymer as defined above.

The composition can also comprise a compound of formula I as n-type semiconductor, a p-type semiconductor like a conjugated polymer, and a second n-type semiconductor, which is preferably a fullerene or substituted fullerene.

The fullerene is for example an indene-C₆₀-fullerene bisaduct like ICBA, or a (6,6)-phenyl-butyric acid methyl ester derivatized methano C₆₀ fullerene, also known as “PCBM-C₆₀” or “C₆₀PCBM”, as disclosed for example in G. Yu, J. Gao, J. C. Hummelen, F. Wudl, A. J. Heeger, Science 1995, Vol. 270, p. 1789 ff and having the structure shown below, or structural analogous compounds with e.g. a C₆₁ fullerene group, a C₇₀ fullerene group, or a C₇₁ fullerene group, or an organic polymer (see for example Coakley, K. M. and McGehee, M. D. Chem. Mater. 2004, 16, 4533).

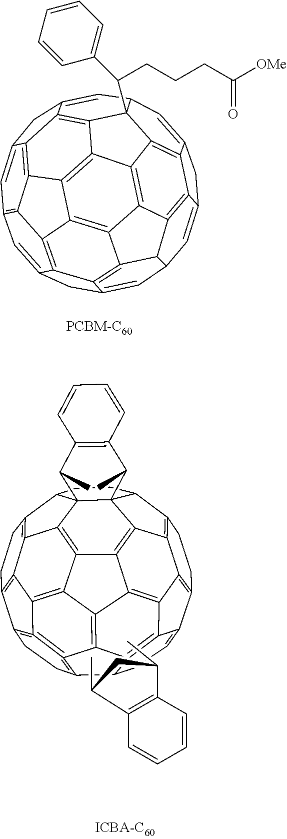

Preferably the compound according to the present invention is blended with an additional n-type semiconductor such as a fullerene or substituted fullerene of formula Full-I to form the active layer in an OPV or OPD device

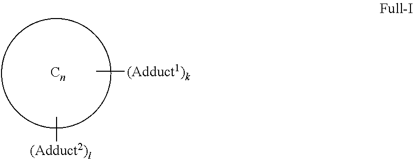

wherein
Cₙ denotes a fullerene composed of n carbon atoms, optionally with one or more atoms trapped inside,
Adduct¹ is a primary adduct appended to the fullerene Cₙ with any connectivity,
Adduct² is a secondary adduct, or a combination of secondary adducts, appended to the fullerene Cₙ with any connectivity,
k is an integer ≥1, and
I is 0, an integer ≥1, or a non-integer >0.

In the formula Full-I and its subformulae, k preferably denotes 1, 2, 3 or, 4, very preferably 1 or 2.

The fullerene Cₙ in formula Full-I and its subformulae may be composed of any number n of carbon atoms Preferably, in the compounds of formula Full-I and its subformulae the number of carbon atoms n of which the fullerene Cₙ is composed is 60, 70, 76, 78, 82, 84, 90, 94 or 96, very preferably 60 or 70.

The fullerene Cₙ in formula Full-I and its subformulae is preferably selected from carbon based fullerenes, endohedral fullerenes, or mixtures thereof, very preferably from carbon based fullerenes.

Suitable and preferred carbon based fullerenes include, without limitation, (C_{60-Ih})[5,6]fullerene, (C_{70-D5h})[5,6]fullerene, (C_{76-D2*})[5,6]fullerene, (C_{84-D2*})[5,6]fullerene, (C_{84-D2d})[5,6]fullerene, or a mixture of two or more of the aforementioned carbon based fullerenes.

The endohedral fullerenes are preferably metallofullerenes. Suitable and preferred metallofullerenes include, without limitation, La@C₆₀, La@C₈₂, Y@C₈₂, Sc₃N@C₈₀, Y₃N@C₈₀, Sc₃C₂@C₈₀ or a mixture of two or more of the aforementioned metallofullerenes.

Preferably the fullerene Cₙ is substituted at a [6,6] and/or [5,6] bond, preferably substituted on at least one [6,6] bond.

Primary and secondary adduct, named “Adduct” in formula Full-I and its subformulae, is preferably selected from the following formulae

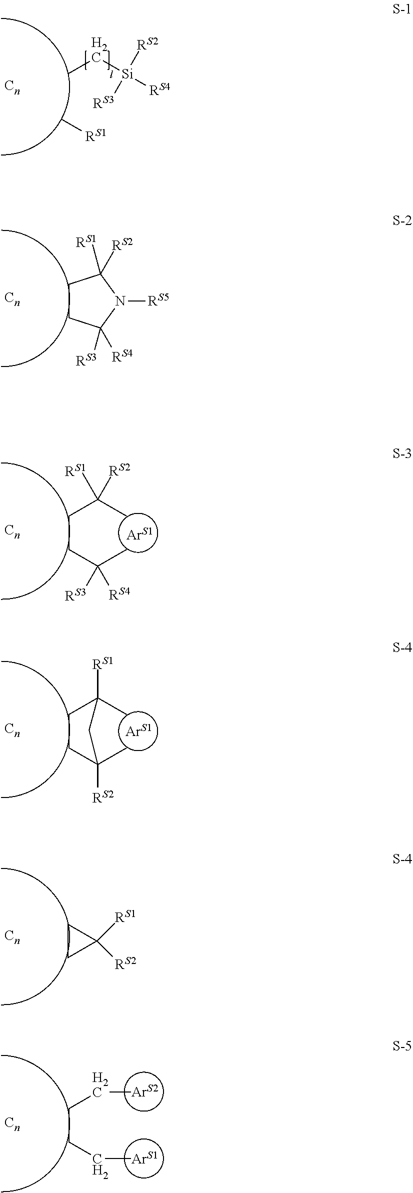

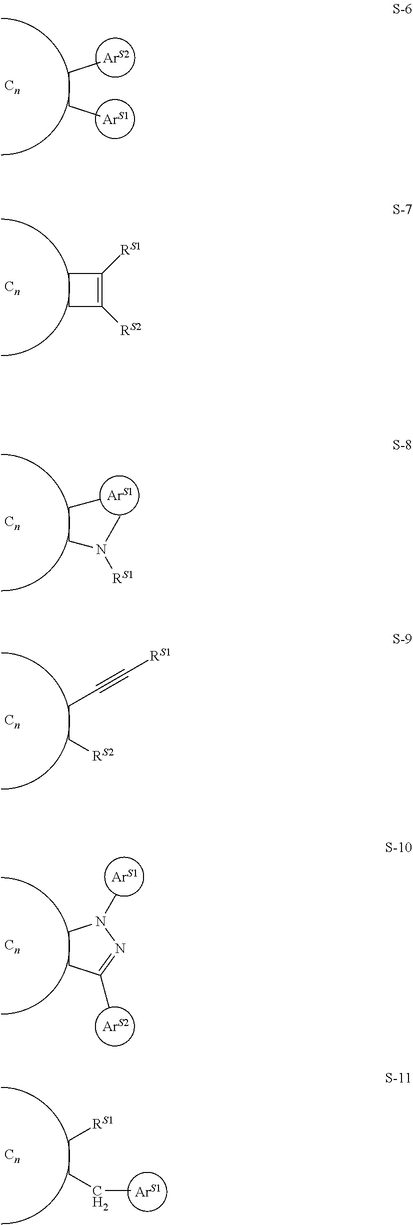

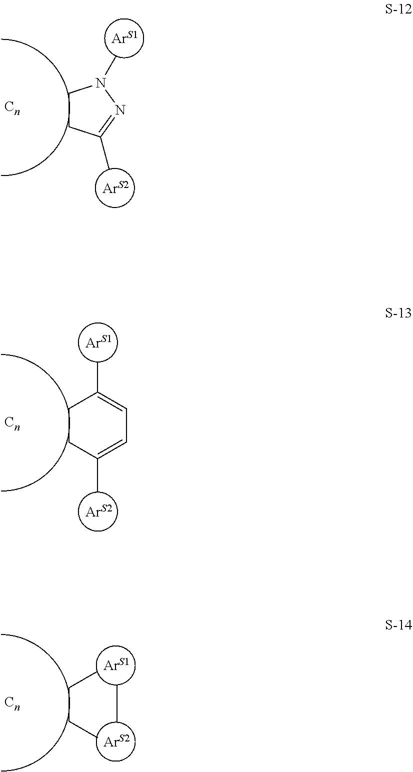

wherein
Ar^{S1}, Ar^{S2} denote, independently of each other, an aryl or heteroaryl group with 5 to 20, preferably 5 to 15, ring atoms, which is mono- or polycyclic, and which is optionally substituted by one or more identical or different substituents having one of the meanings of L as defined above and below.
   R^{S1}, R^{S2}, R^{S3}, R^{S4} and R^{S5} independently of each other denote H, CN or have one of the meanings of L as defined above and below.

Preferred compounds of formula Full-I are selected from the following subformulae:

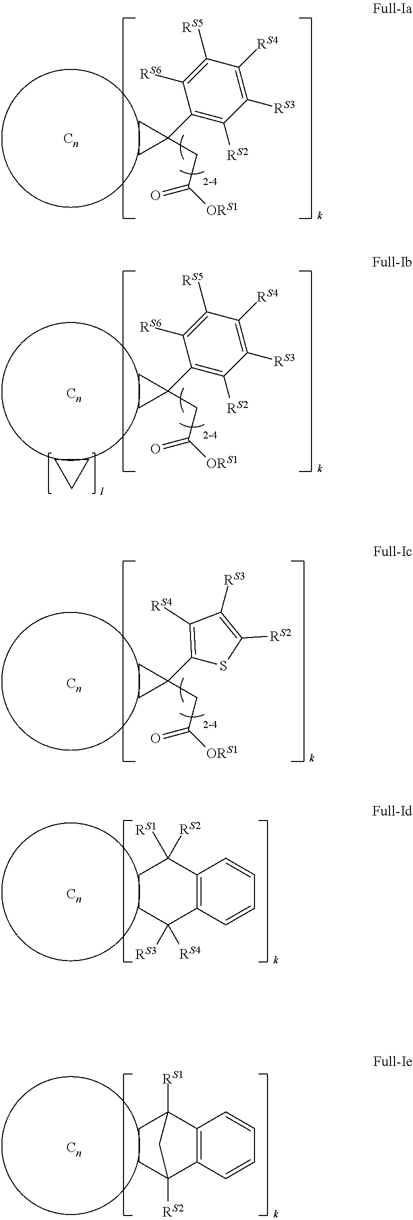

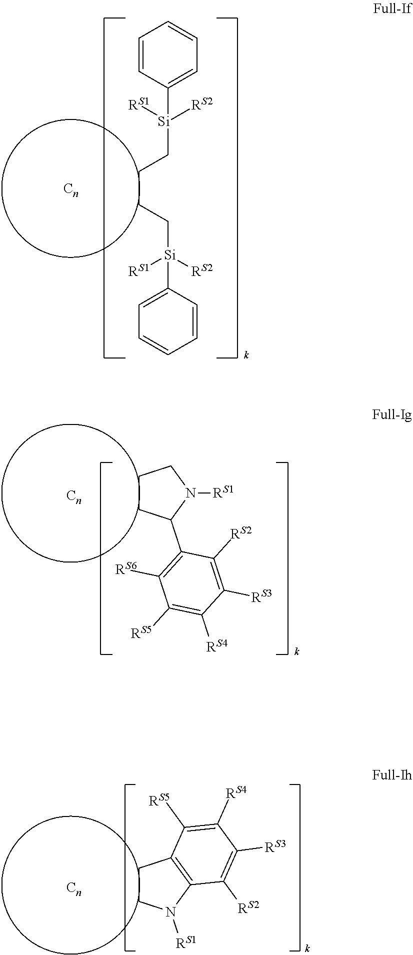

wherein
R^{S1}, R^{S2}, R^{S3}, R^{S4} R^{S5} and R^{S6} independently of each other denote H or have one of the meanings of R^{S} as defined above and below.

Most preferably the fullerene is PCBM-060, PCBM-070, bis-PCBM-060, bis-PCBM-070, ICMA-c60 (1′,4′-dihydro-naphtho[2′,3′:1,2][5,6]fullerene-060), ICBA, oQDM-C60 (1′,4′-dihydro-naphtho[2′,3′:1,9][5,6]fullerene-C60-Ih), or bis-oQDM-060.

The OPV or OPD device preferably further comprises a first transparent or semi-transparent electrode on a transparent or semi-transparent substrate on one side of the photoactive layer, and a second metallic or semi transparent electrode on the other side of the photoactive layer.

Further preferably the OPV or OPD device comprises, between the photoactive layer and the first or second electrode, one or more additional buffer layers acting as hole transporting layer and/or electron blocking layer, which comprise a material such as metal oxide, like for example, ZTO, MoOₓ, NiOₓ, a conjugated polymer electrolyte, like for example PEDOT:PSS, a conjugated polymer, like for example polytriarylamine (PTAA), an insulating polymer, like for example nation, polyethyleneimine or polystyrenesulphonate, an organic compound, like for example N,N′-diphenyl-N,N′-bis(1-naphthyl)(1,1′-biphenyl)-4,4′diamine (NPB), N,N′-diphenyl-N,N′-(3-methylphenyl)-1,1′-biphenyl-4,4′-diamine (TPD), or alternatively as hole blocking layer and/or electron transporting layer, which comprise a material such as metal oxide, like for example, ZnOₓ, TiOₓ, a salt, like for example LiF, NaF, CsF, a conjugated polymer electrolyte, like for example poly[3-(6-trimethylammoniumhexyl)thiophene], poly(9,9-bis(2-ethylhexyl)-fluorene]-b-poly[3-(6-trimethylammoniumhexyl)thiophene], or poly [(9,9-bis(3′-(N,N-dimethylamino)propyl)-2,7-fluorene)-alt-2,7-(9,9-dioctylfluorene)] or an organic compound, like for example tris(8-quinolinolato)-aluminium(III) (Alg3), 4,7-diphenyl-1,10-phenanthroline.

In a composition according to the present invention comprising a compound of formula I and a conjugated polymer, the ratio polymer:compound of formula I is preferably from 5:1 to 1:5, more preferably from 3:1 to 1:3, most preferably 2:1 to 1:2 by weight.

The composition according to the present invention may also comprise a polymeric binder, preferably from 0.001 to 95% by weight. Examples of binder include polystyrene (PS), polydimethylsilane (PDMS), polypropylene (PP) and polymethylmethacrylate (PMMA).

A binder to be used in the formulation as described before, which is preferably a polymer, may comprise either an insulating binder or a semiconducting binder, or mixtures thereof, may be referred to herein as the organic binder, the polymeric binder or simply the binder.

Preferably, the polymeric binder comprises a weight average molecular weight in the range of 1000 to 5,000,000 g/mol, especially 1500 to 1,000,000 g/mol and more preferable 2000 to 500,000 g/mol. Surprising effects can be achieved with polymers having a weight average molecular weight of at least 10000 g/mol, more preferably at least 100000 g/mol.

In particular, the polymer can have a polydispersity index M_{w}/Mₙ in the range of 1.0 to 10.0, more preferably in the range of 1.1 to 5.0 and most preferably in the range of 1.2 to 3.

Preferably, the inert binder is a polymer having a glass transition temperature in the range of −70 to 160° C., preferably 0 to 150° C., more preferably 50 to 140° C. and most preferably 70 to 130° C. The glass transition temperature can be determined by measuring the DSC of the polymer (DIN EN ISO 11357, heating rate 10° C. per minute).

The weight ratio of the polymeric binder to the OSC compound, like that of formula I, is preferably in the range of 30:1 to 1:30, particularly in the range of 5:1 to 1:20 and more preferably in the range of 1:2 to 1:10.

According to a preferred embodiment the binder preferably comprises repeating units derived from styrene monomers and/or olefin monomers. Preferred polymeric binders can comprise at least 80%, preferably 90% and more preferably 99% by weight of repeating units derived from styrene monomers and/or olefins.

Styrene monomers are well known in the art. These monomers include styrene, substituted styrenes with an alkyl substituent in the side chain, such as α-methylstyrene and α-ethylstyrene, substituted styrenes with an alkyl substituent on the ring such as vinyltoluene and p-methylstyrene, halogenated styrenes such as monochlorostyrenes, dichlorostyrenes, tribromostyrenes and tetrabromostyrenes.

Olefin monomers consist of hydrogen and carbon atoms. These monomers include ethylene, propylene, butylenes, isoprene and 1,3-butadiene.

According to a preferred embodiment of the present invention, the polymeric binder is polystyrene having a weight average molecular weight in the range of 50,000 to 2,000,000 g/mol, preferably 100,000 to 750,000 g/mol, more preferably in the range of 150,000 to 600,000 g/mol and most preferably in the range of 200,000 to 500,000 g/mol.

Further examples of suitable binders are disclosed for example in US 2007/0102696 A1. Especially suitable and preferred binders are described in the following.

The binder should preferably be capable of forming a film, more preferably a flexible film.

Suitable polymers as binders include poly(1,3-butadiene), polyphenylene, polystyrene, poly(α-methylstyrene), poly(α-vinylnaphtalene), poly(vinyltoluene), polyethylene, cis-polybutadiene, polypropylene, polyisoprene, poly(4-methyl-1-pentene), poly (4-methylstyrene), poly(chorotrifluoroethylene), poly(2-methyl-1,3-butadiene), poly(p-xylylene), poly(α-α-α′-α′ tetrafluoro-p-xylylene), poly[1,1-(2-methyl propane)bis(4-phenyl)carbonate], poly(cyclohexyl methacrylate), poly(chlorostyrene), poly(2,6-dimethyl-1,4-phenylene ether), polyisobutylene, poly(vinyl cyclohexane), poly(vinylcinnamate), poly(4-vinylbiphenyl), 1,4-polyisoprene, polynorbornene, poly(styrene-block-butadiene); 31% wt styrene, poly(styrene-block-butadiene-block-styrene); 30% wt styrene, poly(styrene-co-maleic anhydride) (and ethylene/butylene) 1-1.7% maleic anhydride, poly(styrene-block-ethylene/butylene-block-styrene) triblock polymer 13% styrene, poly(styrene-block-ethylene-propylene-block-styrene) triblock polymer 37% wt styrene, poly(styrene-block-ethylene/butylene-block-styrene) triblock polymer 29% wt styrene, poly(1-vinylnaphthalene), poly(1-vinylpyrrolidone-co-styrene) 64% styrene, poly(1-vinylpyrrolidone-co-vinyl acetate) 1.3:1, poly(2-chlorostyrene), poly(2-vinylnaphthalene), poly(2-vinylpyridine-co-styrene) 1:1, poly(4,5-Difluoro-2,2-bis(CF₃)-1,3-dioxole-co-tetrafluoroethylene) Teflon, poly(4-chlorostyrene), poly(4-methyl-1-pentene), poly(4-methylstyrene), poly(4-vinylpyridine-co-styrene) 1:1, poly(alpha-methylstyrene), poly(butadiene-graft-poly(methyl acrylate-co-acrylonitrile)) 1:1:1, poly(butyl methacrylate-co-isobutyl methacrylate) 1:1, poly(butyl methacrylate-co-methyl methacrylate) 1:1, poly(cyclohexylmethacrylate), poly(ethylene-co-1-butene-co-1-hexene) 1:1:1, poly(ethylene-co-ethylacrylate-co-maleic anhydride); 2% anhydride, 32% ethyl acrylate, poly(ethylene-co-glycidyl methacrylate) 8% glycidyl methacrylate, poly(ethylene-co-methyl acrylate-co-glycidyl meth-acrylate) 8% glycidyl metha-crylate 25% methyl acrylate, poly(ethylene-co-octene) 1:1, poly(ethylene-co-propylene-co-5-methylene-2-norbornene) 50% ethylene, poly(ethylene-co-tetrafluoroethylene) 1:1, poly(isobutyl methacrylate), poly(isobutylene), poly(methyl methacrylate)-co-(fluorescein 0-methacrylate) 80% methyl methacrylate, poly(methyl methacrylate-co-butyl methacrylate) 85% methyl methacrylate, poly(methyl methacrylate-co-ethyl acrylate) 5% ethyl acrylate, poly(propylene-co-butene) 12% 1-butene, poly(styrene-co-ally) alcohol) 40% ally) alcohol, poly(styrene-co-maleic anhydride) 7% maleic anhydride, poly(styrene-co-maleic anhydride) cumene terminated (1.3:1), poly(styrene-co-methyl methacrylate) 40% styrene, poly(vinyltoluene-co-alpha-methylstyrene) 1:1, poly-2-vinylpyridine, poly-4-vinylpyridine, poly-alpha-pinene, polymethylmethacrylate, polybenzylmethacrylate, polyethylmethacrylate, polyethylene, polyethylene terephthalate, polyethylene-co-ethylacrylate 18% ethyl acrylate, polyethylene-co-vinylacetate 12% vinyl acetate, polyethylene-graft-maleic anhydride 0.5% maleic anhydride, polypropylene, polypropylene-graft-maleic anhydride 8-10% maleic anhydride, polystyrene poly(styrene-block-ethylene/butylene-block-styrene) graft maleic anhydride 2% maleic anhydride 1:1:1 others, poly(styrene-block-butadiene) branched 1:1, poly(styrene-block-butadiene-block-styrene), 30% styrene, poly(styrene-block-isoprene) 10% wt styrene, poly(styrene-block-isoprene-block-styrene) 17% wt styrene, poly(styrene-co-4-chloromethylstyrene-co-4-methoxymethylstyrene 2:1:1, polystyrene-co-acrylonitrile 25% acrylonitrile, polystyrene-co-alpha-methylstyrene 1:1, polystyrene-co-butadiene 4% butadiene, polystyrene-co-butadiene 45% styrene, polystyrene-co-chloromethylstyrene 1:1, polyvinylchloride, polyvinylcinnamate, polyvinylcyclohexane, polyvinylidenefluoride, polyvinylidenefluoride-co-hexafluoropropylene assume 1:1, poly(styrene-block-ethylene/propylene-block-styrene) 30% styrene, poly(styrene-block-ethylene/propylene-block-styrene) 18% styrene, poly(styrene-block-ethylene/propylene-block-styrene) 13% styrene, poly(styrene-block ethylene block-ethylene/propylene-block styrene) 32% styrene, poly(styrene-block ethylene block-ethylene/propylene-block styrene) 30% styrene, poly(styrene-block-ethylene/butylene-block-styrene) 31% styrene, poly(styrene-block-ethylene/butylene-block-styrene) 34% styrene, poly(styrene-block-ethylene/butylene-block-styrene) 30% styrene, poly(styrene-block-ethylene/butylene-block-styrene) 60%, styrene, branched or non-branched polystyrene-block-polybutadiene, polystyrene-block(polyethylene-ran-butylene)-block-polystyrene, polystyrene-block-polybutadiene-block-polystyrene, polystyrene-(ethylene-propylene)-diblock-copolymers (e.g. KRATON®-G1701E, Shell), poly(propylene-co-ethylene) and poly(styrene-co-methylmethacrylate).

Preferred insulating binders to be used in the formulations as described before are polystryrene, poly(α-methylstyrene), polyvinylcinnamate, poly(4-vinylbiphenyl), poly(4-methylstyrene), and polymethyl methacrylate. Most preferred insulating binders are polystyrene and polymethyl methacrylate.

The binder can also be selected from crosslinkable binders, like e.g. acrylates, epoxies, vinylethers, thiolenes etc. The binder can also be mesogenic or liquid crystalline.

The organic binder may itself be a semiconductor, in which case it will be referred to herein as a semiconducting binder. The semiconducting binder is still preferably a binder of low permittivity as herein defined. Semiconducting binders for use in the present invention preferably have a number average molecular weight (Me) of at least 1500-2000, more preferably at least 3000, even more preferably at least 4000 and most preferably at least 5000. The semiconducting binder preferably has a charge carrier mobility of at least 10⁻⁵ cm²V⁻¹s⁻¹, more preferably at least 10⁻⁴ cm²V⁻¹s⁻¹.

A preferred semiconducting binder comprises a homo-polymer or copolymer (including block-copolymer) containing arylamine (preferably triarylamine).

To produce thin layers in BHJ OPV devices the compounds, compositions and formulations of the present invention may be deposited by any suitable method. Liquid coating of devices is more desirable than vacuum deposition techniques. Solution deposition methods are especially preferred. The formulations of the present invention enable the use of a number of liquid coating techniques. Preferred deposition techniques include, without limitation, dip coating, spin coating, ink jet printing, nozzle printing, letter-press printing, screen printing, gravure printing, doctor blade coating, roller printing, reverse-roller printing, offset lithography printing, dry offset lithography printing, flexographic printing, web printing, spray coating, curtain coating, brush coating, slot dye coating or pad printing. For the fabrication of OPV devices and modules area printing method compatible with flexible substrates are preferred, for example slot dye coating, spray coating and the like.

Suitable solutions or formulations containing the mixture of a compound of formula I and a polymer must be prepared. In the preparation of formulations, suitable solvent must be selected to ensure full dissolution of both component, p-type and n-type and take into account the boundary conditions (for example rheological properties) introduced by the chosen printing method.

Organic solvents are generally used for this purpose. Typical solvents can be aromatic solvents, halogenated solvents or chlorinated solvents, including chlorinated aromatic solvents. Examples include, but are not limited to chlorobenzene, 1,2-dichlorobenzene, chloroform, 1,2-dichloroethane, dichloromethane, carbon tetrachloride, toluene, cyclohexanone, ethylacetate, tetrahydrofuran, anisole, 2,4-dimethylanisole, 1-methylnaphthalene, morpholine, toluene, o-xylene, m-xylene, p-xylene, 1,4-dioxane, acetone, methylethylketone, 1,2-dichloroethane, 1,1,1-trichloroethane, 1,1,2,2-tetrachloroethane, ethyl acetate, n-butyl acetate, N,N-dimethylformamide, dimethylacetamide, dimethylsulfoxide, 1,5-dimethyltetraline, propiophenone, acetophenone, tetraline, 2-methylthiophene, 3-methylthiophene, decaline, indane, methyl benzoate, ethyl benzoate, mesitylene and combinations thereof.

The OPV device can for example be of any type known from the literature (see e.g. Waldauf et al., Appl. Phys. Lett., 2006, 89, 233517).

A first preferred OPV device according to the invention comprises the following layers (in the sequence from bottom to top):
optionally a substrate,
a high work function electrode, preferably comprising a metal oxide, like for example ITO, serving as anode,
an optional conducting polymer layer or hole transport layer, preferably comprising an organic polymer or polymer blend, for example of PEDOT:PSS (poly(3,4-ethylenedioxythiophene): poly(styrene-sulfonate), or TBD (N,N′-dyphenyl-N-N′-bis(3-methylphenyl)-1,1′biphenyl-4,4′-diamine) or NBD (N,N′-dyphenyl-N-N′-bis(1-napthylphenyl)-1,1′biphenyl-4,4′-diamine),
a layer, also referred to as “photoactive layer”, comprising a p-type and an n-type organic semiconductor, which can exist for example as a p type/n-type bilayer or as distinct p-type and n-type layers, or as blend or p-type and n-type semiconductor, forming a BHJ,
optionally a layer having electron transport properties, for example comprising LiF or PFN,
a low work function electrode, preferably comprising a metal like for example aluminium, serving as cathode, wherein at least one of the electrodes, preferably the anode, is transparent to visible light, and wherein the n-type semiconductor is a compound of formula I.

A second preferred OPV device according to the invention is an inverted OPV device and comprises the following layers (in the sequence from bottom to top):
optionally a substrate,
a high work function metal or metal oxide electrode, comprising for example ITO, serving as cathode,
a layer having hole blocking properties, preferably comprising an organic polymer, polymer blend, metal or metal oxide like TiOₓ, ZnOₓ, Ca, Mg, poly(ethyleneimine), poly(ethyleneimine) ethoxylated or poly [(9,9-bis(3′-(N,N-dimethylamino)propyl)-2,7-fluorene)-alt-2,7-(9,9-dioctylfluorene)],
a photoactive layer comprising a p-type and an n-type organic semiconductor, situated between the electrodes, which can exist for example as a p-type/n-type bilayer or as distinct p-type and n-type layers, or as blend or p-type and n-type semiconductor, forming a BHJ,
an optional conducting polymer layer or hole transport layer, preferably comprising an organic polymer or polymer blend, metal or metal oxide, for example of PEDOT:PSS, nafion or a substituted triaryl amine derivative like for example TBD or NBD, or WOₓ, MoOₓ, NiOₓ, Pd or Au,
an electrode comprising a high work function metal like for example silver, serving as anode,
wherein at least one of the electrodes, preferably the cathode, is transparent to visible light, and
wherein the n-type semiconductor is a compound of formula I.

In the OPV devices of the present invention the p-type and n-type semiconductor materials are preferably selected from the materials, like the compound/polymer/fullerene systems, as described above

When the photoactive layer is deposited on the substrate, it forms a BHJ that phase separates at nanoscale level. For discussion on nanoscale phase separation see Dennler et al, Proceedings of the IEEE, 2005, 93 (8), 1429 or Hoppe et al, Adv. Func. Mater, 2004, 14(10), 1005. An optional annealing step may be then necessary to optimize blend morphology and consequently OPV device performance.

Another method to optimize device performance is to prepare formulations for the fabrication of OPV(BHJ) devices that may include high boiling point additives to promote phase separation in the right way. 1,8-Octanedithiol, 1,8-diiodooctane, nitrobenzene, chloronaphthalene, and other additives have been used to obtain high-efficiency solar cells. Examples are disclosed in J. Peet, et al, Nat. Mater., 2007, 6, 497 or Fréchet et al. J. Am. Chem. Soc., 2010, 132, 7595-7597.

Another preferred embodiment of the present invention relates to the use of a compound or composition according to the present invention as dye, hole transport layer, hole blocking layer, electron transport layer and/or electron blocking layer in a DSSC or PSC, and to a DSSC or PSC comprising a compound composition or polymer blend according to the present invention.

DSSCs and PSCs can be manufactured as described in the literature, for example in Chem. Rev. 2010, 110, 6595-6663, Angew. Chem. Int. Ed. 2014, 53, 2-15 or in WO2013171520A1.

A preferred OE device according to the invention is a solar cell, preferably a PSC, comprising a light absorber which is at least in part inorganic as described below.

In a solar cell comprising the light absorber according to the invention there are no restrictions per se with respect to the choice of the light absorber material which is at least in part inorganic.

The term “at least in part inorganic” means that the light absorber material may be selected from metalorganic complexes or materials which are substantially inorganic and possess preferably a crystalline structure where single positions in the crystalline structure may be allocated by organic ions.

Preferably, the light absorber comprised in the solar cell according to the invention has an optical band-gap≤2.8 eV and ≥0.8 eV.

Very preferably, the light absorber in the solar cell according to the invention has an optical band-gap≤ 2.2 eV and ≥1.0 eV.

The light absorber used in the solar cell according to the invention does preferably not contain a fullerene. The chemistry of fullerenes belongs to the field of organic chemistry. Therefore fullerenes do not fulfil the definition of being “at least in part inorganic” according to the invention.

Preferably, the light absorber which is at least in part inorganic is a material having perovskite structure or a material having 2D crystalline perovskite structure.

The term “perovskite” as used above and below denotes generally a material having a perovskite crystalline structure or a 2D crystalline perovskite structure.

The term perovskite solar cell (PSC) means a solar cell comprising a light absorber which is a material having perovskite structure or a material having 2D crystalline perovskite structure.

The light absorber which is at least in part inorganic is without limitation composed of a material having perovskite crystalline structure, a material having 2D crystalline perovskite structure (e.g. CrystEngComm, 2010, 12, 2646-2662), Sb₂S₃ (stibnite), Sb₂(SₓSe₍ₓ₋₁₎)₃, PbSₓSe₍ₓ₋₁₎, CdSₓSe₍ₓ₋₁₎, ZnTe, CdTe, ZnSₓSe₍ₓ₋₁₎, InP, FeS, FeS₂, Fe₂S₃, Fe₂SiS₄, Fe₂GeS₄, Cu₂S, CuInGa, CuIn(SeₓS₍₁₋ₓ₎, Cu₃SbₓBi₍ₓ₋₁₎, (S_{y}Se_{(y-1)})₃, Cu₂SnS₃, SnSₓSe₍ₓ₋₁₎, Ag₂S, AgBiS₂, BiSI, BiSeI, Bi₂(SₓSe₍ₓ₋₁₎)₃, BiS₍₁₋ₓ₎SeₓI, WSe₂, AlSb, metal halides (e.g. BiI₃, Cs₂SnI₆), chalcopyrite (e.g. CuInxGa₍₁₋ₓ₎(S_{y}Se_{(1-y)})₂), kesterite (e.g. Cu₂ZnSnS₄, Cu₂ZnSn(SeₓS₍₁₋ₓ₎, Cu₂Zn(Sn₁₋ₓGeₓ)S₄) and metal oxide (e.g. CuO, Cu₂O) or a mixture thereof.

Preferably, the light absorber which is at least in part inorganic is a perovskite.

In the above definition for light absorber, x and y are each independently defined as follows: (0≤)(≤1) and (0≤y≤1).

Very preferably, the light absorber is a special perovskite namely a metal halide perovskite as described in detail above and below. Most preferably, the light absorber is an organic-inorganic hybrid metal halide perovskite contained in the perovskite solar cell (PSC).

In one particularly preferred embodiment of the invention, the perovskite denotes a metal halide perovskite with the formula ABX₃,

where
A is a monovalent organic cation, a metal cation or a mixture of two or more of these cations
B is a divalent cation and
X is F, Cl, Br, I, BF₄ or a combination thereof.

Preferably, the monovalent organic cation of the perovskite is selected from alkylammonium, wherein the alkyl group is straight chain or branched having 1 to 6 C atoms, formamidinium or guanidinium or wherein the metal cation is selected from K⁺, Cs⁺ or R^{b+}.

Suitable and preferred divalent cations B are Ge²⁺, Sn²⁺ or Pb²⁺.

Suitable and preferred perovskite materials are CsSnl₃, CH₃NH₃Pb(I₁₋ₓClₓ)₃, CH₃NH₃PbI₃, CH₃NH₃Pb(I₁₋ₓBrₓ)₃, CH₃NH₃Pb(I₁₋ₓ(BF₄)ₓ)₃, CH₃NH₃Sn(I₁₋ₓClₓ)₃, CH₃NH₃SnI₃ or CH₃NH₃Sn(I₁₋ₓBrₓ)₃ wherein x is each independently defined as follows: (0<x≤1).

Further suitable and preferred perovskites may comprise two halides corresponding to formula Xa₍₃₋ₓ₎Xb₍ₓ₎, wherein Xa and Xb are each independently selected from Cl, Br, or I, and x is greater than 0 and less than 3.

Suitable and preferred perovskites are also disclosed in WO 2013/171517, claims 52 to 71 and claims 72 to 79, which is entirely incorporated herein by reference. The materials are defined as mixed-anion perovskites comprising two or more different anions selected from halide anions and chalcogenide anions. Preferred perovskites are disclosed on page 18, lines 5 to 17. As described, the perovskite is usually selected from CH₃NH₃PbBrI₂, CH₃NH₃PbBrCl₂, CH₃NH₃PbIBr₂, CH₃NH₃PbIC₁₂, CH₃NH₃SnF₂Br, CH₃NH₃SnF₂I and (H₂N═CH—NH₂)PbI₃ZBr_{3(1-z)}, wherein z is greater than 0 and less than 1.

The invention further relates to a solar cell comprising the light absorber, preferably a PSC, as described above and below, wherein the compound of formula I is employed as a layer between one electrode and the light absorber layer.

The invention further relates to a solar cell comprising the light absorber, preferably a PSC, as described above and below, wherein the compound of formula I is comprised in an electron-selective layer.

The electron selective layer is defined as a layer providing a high electron conductivity and a low hole conductivity favoring electron-charge transport.

The invention further relates to a solar cell comprising the light absorber, preferably a PSC, as described above and below, wherein the compound of formula I is employed as electron transport material (ETM) or as hole blocking material as part of the electron selective layer.

Preferably, the compound of formula I is employed as electron transport material (ETM).

In an alternative preferred embodiment, the compound of formula I is employed as hole blocking material.

The device architecture of a PSC device according to the invention can be of any type known from the literature.

A first preferred device architecture of a PSC device according to the invention comprises the following layers (in the sequence from bottom to top):
optionally a substrate which, in any combination, can be flexible or rigid and transparent, semi-transparent or non-transparent and electrically conductive or non-conductive;
a high work function electrode, preferably comprising a doped metal oxide, for example fluorine-doped tin oxide (FTO), tin-doped indium oxide (ITO), or aluminium-doped zinc oxide;
an electron-selective layer which comprises one or more electron-transporting materials, at least one of which is a compound of formula I, and which, in some cases, can also be a dense layer and/or be composed of nanoparticles, and which preferably comprises a metal oxide such as TiO₂, ZnO₂, SnO₂, Y₂O₅, Ga₂O₃, SrTiO₃, BaTiO₃ or combinations thereof;
optionally a porous scaffold which can be conducting, semi-conducting or insulating, and which preferably comprises a metal oxide such as TiO₂, ZnO₂, SnO₂, Y₂O₅, Ga₂O₃, SrTiO₃, BaTiO₃, Al₂O₃, ZrO₂, SiO₂ or combinations thereof, and which is preferably composed of nanoparticles, nanorods, nanoflakes, nanotubes or nanocolumns;
a layer comprising a light absorber which is at least in part inorganic, particularly preferably a metal halide perovskite as described above which, in some cases, can also be a dense or porous layer and which optionally partly or fully infiltrates into the underlying layer;
optionally a hole selective layer, which comprises one or more hole-transporting materials, and which, in some cases, can also comprise additives such as lithium salts, for example LiY, where Y is a monovalent organic anion, preferably bis(trifluoromethylsulfonyl)imide, tertiary amines such as 4-tert-butylpyridine, or any other covalent or ionic compounds, for example tris(2-(1H-pyrazol-1-yl)-4-tert-butylpyridine)-cobalt(III) tris(bis(trifluoromethylsulfonyl)imide)), which can enhance the properties of the hole selective layer, for example the electrical conductivity, and/or facilitate its processing;

and a back electrode which can be metallic, for example made of Au, Ag, Al, Cu, Ca, Ni or combinations thereof, or non-metallic and transparent, semi-transparent or non-transparent.

A second preferred device architecture of a PSC device according to the invention comprises the following layers (in the sequence from bottom to top):
optionally a substrate which, in any combination, can be flexible or rigid and transparent, semi-transparent or non-transparent and electrically conductive or non-conductive;
a high work function electrode, preferably comprising a doped metal oxide, for example fluorine-doped tin oxide (FTO), tin-doped indium oxide (ITO), or aluminium-doped zinc oxide;
optionally a hole injection layer which, for example, changes the work function of the underlying electrode, and/or modifies the surface of the underlying layer and/or helps to planarize the rough surface of the underlying layer and which, in some cases, can also be a monolayer;
optionally a hole selective layer, which comprises one or more hole-transporting materials and which, in some cases, can also comprise additives such as lithium salts, for example LiY, where Y is a monovalent organic anion, preferably bis(trifluoromethylsulfonyl)imide, tertiary amines such as 4-tert-butylpyridine, or any other covalent or ionic compounds, for example tris(2-(1H-pyrazol-1-yl)-4-tert-butylpyridine)-cobalt(III) tris(bis(trifluoromethylsulfonyl)imide)), which can enhance the properties of the hole selective layer, for example the electrical conductivity, and/or facilitate its processing;
a layer comprising a light absorber which is at least in part inorganic, particularly preferably a metal halide perovskite as described or preferably described above;
an electron-selective layer, which comprises one or more electron-transporting materials, at least one of which is a compound of formula I and which, in some cases, can also be a dense layer and/or be composed of nanoparticles, and which, for example, can comprise a metal oxide such as TiO₂, ZnO₂, SnO₂, Y₂O₅, Ga₂O₃, SrTiO₃, BaTiO₃ or combinations thereof, and/or which can comprise a substituted fullerene, for example [6,6]-phenyl C61-butyric acid methyl ester, and/or which can comprise a molecular, oligomeric or polymeric electron-transport material, for example 2,9-Dimethyl-4,7-diphenyl-1,10-phenanthroline, or a mixture thereof;

and a back electrode which can be metallic, for example made of Au, Ag, Al, Cu, Ca, Ni or combinations thereof, or non-metallic and transparent, semi-transparent or non-transparent.

To produce electron selective layers in PSC devices according to the invention, the compounds of formula I, optionally together with other compounds or additives in the form of blends or mixtures, may be deposited by any suitable method. Liquid coating of devices is more desirable than vacuum deposition techniques. Solution deposition methods are especially preferred. Formulations comprising the compounds of formula I enable the use of a number of liquid coating techniques. Preferred deposition techniques include, without limitation, dip coating, spin coating, ink jet printing, nozzle printing, letter-press printing, screen printing, gravure printing, doctor blade coating, roller printing, reverse-roller printing, offset lithography printing, dry offset lithography printing, flexographic printing, web printing, spray coating, curtain coating, brush coating, slot die coating or pad printing. For the fabrication of PSC devices and modules, deposition techniques for large area coating are preferred, for example slot die coating or spray coating.

Formulations that can be used to produce electron selective layers in optoelectronic devices according to the invention, preferably in PSC devices comprise one or more compounds of formula I or preferred embodiments as described above in the form of blends or mixtures optionally together with one or more further electron transport materials and/or hole blocking materials and/or binders and/or other additives as described above and below, and one or more solvents.

The formulation may include or comprise, essentially consist of or consist of the said necessary or optional constituents as described above or below. All compounds or components which can be used in the formulations are either known or commercially available, or can be synthesised by known processes.

The formulation as described before may be prepared by a process which comprises:
(i) first mixing a compound of formula I, optionally a binder or a precursor of a binder as described before, optionally a further electron transport material, optionally one or more further additives as described above and below and a solvent or solvent mixture as described above and below and
(ii) applying such mixture to a substrate; and optionally evaporating the solvent(s) to form an electron selective layer according to the present invention.

In step (i) the solvent may be a single solvent for the compound of formula I and the organic binder and/or further electron transport material may each be dissolved in a separate solvent followed by mixing the resultant solutions to mix the compounds.

Alternatively, the binder may be formed in situ by mixing or dissolving a compound of formula I in a precursor of a binder, for example a liquid monomer, oligomer or crosslinkable polymer, optionally in the presence of a solvent, and depositing the mixture or solution, for example by dipping, spraying, painting or printing it, on a substrate to form a liquid layer and then curing the liquid monomer, oligomer or crosslinkable polymer, for example by exposure to radiation, heat or electron beams, to produce a solid layer. If a preformed binder is used it may be dissolved together with the compound formula I in a suitable solvent as described before, and the solution deposited for example by dipping, spraying, painting or printing it on a substrate to form a liquid layer and then removing the solvent to leave a solid layer. It will be appreciated that solvents are chosen which are able to dissolve all ingredients of the formulation, and which upon evaporation from the solution blend give a coherent defect free layer. Besides the said components, the formulation as described before may comprise further additives and processing assistants. These include, inter alia, surface-active substances (surfactants), lubricants and greases, additives which modify the viscosity, additives which increase the conductivity, dispersants, hydrophobicising agents, adhesion promoters, flow improvers, antifoams, deaerating agents, diluents, which may be reactive or unreactive, fillers, assistants, processing assistants, dyes, pigments, stabilisers, sensitisers, nanoparticles and inhibitors.

Additives can be used to enhance the properties of the electron selective layer and/or the properties of any of the neighbouring layers and/or the performance of the optoelectronic device according to the invention. Additives can also be used to facilitate the deposition, the processing or the formation of the electron selective layer and/or the deposition, the processing or the formation of any of the neighbouring layers. Preferably, one or more additives are used which enhance the electrical conductivity of the electron selective layer and/or passivate the surface of any of the neighbouring layers.

Suitable methods to incorporate one or more additives include, for example exposure to a vapor of the additive at atmospheric pressure or at reduced pressure, mixing a solution or solid containing one or more additives and a material or a formulation as described or preferably described before, bringing one or more additives into contact with a material or a formulation as described before, by thermal diffusion of one or more additives into a material or a formulation as described before, or by ion-implantation of one or more additives into a material or a formulation as described before.

Additives used for this purpose can be organic, inorganic, metallic or hybrid materials. Additives can be molecular compounds, for example organic molecules, salts, ionic liquids, coordination complexes or organometallic compounds, polymers or mixtures thereof. Additives can also be particles, for example hybrid or inorganic particles, preferably nanoparticles, or carbon based materials such as fullerenes, carbon nanotubes or graphene flakes.

Examples for additives that can enhance the electrical conductivity are for example halogens (e.g. I₂, Cl₂, Br₂, ICl, ICl₃, IBr and IF), Lewis acids (e.g. PF₅, AsF₅, SbF₅, BF₃, BCl₃, SbCl₅, BBr₃ and SO₃), protonic acids, organic acids, or amino acids (e.g. HF, HCl, HNO₃, H₂SO₄, HClO₄, FSO₃H and ClSO₃H), transition metal compounds (e.g. FeCl₃, FeOCl, Fe(ClO₄)₃, Fe(4-CH₃C6H₄SO₃)₃, TiCl₄, ZrCl₄, HfCl₄, NbF₅, NbCl₅, TaCl₅, MOF₅, MoCl₅, WF₅, WCl₆, UF₆ and LnCl₃ (wherein Ln is a lanthanoid)), anions (e.g. Cl⁻, Br, I⁻, I₃⁻, HSO₄_{^{−}}, SO₄²⁻, NO₃⁻, ClO₄_{^{−}}, BF₄_{^{−}}, PF₆_{^{−}}, AsF₆_{^{−}}, SbF₆_{^{−}}, FeCl₄_{^{−}}, Fe(CN)₆³⁻, and anions of various sulfonic acids, such as aryl-SO₃_{^{−}}), cations (e.g. H⁺, Li⁺, Na⁺, K⁺, Rb⁺, Cs⁺, Co³⁺ and Fe³⁺), O₂, redox active salts (e.g. XeOF₄, (NO₂_{⁺}) (SbF₆_{^{−}}), (NO₂_{⁺}) (SbCl₆_{^{−}}), (NO₂_{^{+) (BF}}₄_{^{−}}), NOBF₄, NOPF₆, AgClO₄, H₂IrCl₆ and La(NO₃)₃·6H₂O), strongly electron-accepting organic molecules (e.g. 2,3,5,6-tetrafluoro-7,7,8,8-tetracyanoquinodimethane (F₄-TCNQ)), transition metal oxides (e.g. WO₃, Re₂O₇ and MoO₃), metal-organic complexes of cobalt, iron, bismuth and molybdenum, (p-BrC₆H₄)₃NSbCl₆, bismuth(III) tris(trifluoroacetate), FSO₂OOSO₂F, acetylcholine, R₄N⁺, (R is an alkyl group), R₄P⁺ (R is a straight-chain or branched alkyl group 1 to 20), R₆As⁺ (R is an alkyl group), R₃S⁺ (R is an alkyl group) and ionic liquids (e.g. 1-Ethyl-3-methylimidazolium bis(trifluoromethylsulfonyl)imide). Suitable cobalt complexes beside of tris(2-(1H-pyrazol-1-yl)-4-tert-butylpyridine)-cobalt(III) tris(bis(trifluoromethylsulfonyl)imide)) are cobalt complex salts as described in WO 2012/114315, WO 2012/114316, WO 2014/082706, WO 2014/082704, EP 2883881 or JP 2013-131477.

Suitable lithium salts are beside of lithium bis(trifluoromethylsulfonyl)imide, lithium tris(pentafluoroethyl)trifluorophosphate, lithium dicyanamide, lithium methylsulfate, lithium trifluormethanesulfonate, lithium tetracyanoborate, lithium dicyanamide, lithium tricyanomethide, lithium thiocyanate, lithium chloride, lithium bromide, lithium iodide, lithium hexafluoroposphate, lithium tetrafluoroborate, lithium perchlorate, lithium hexafluoroantimonate, lithium hexafluoroarsenate or a combination of two or more. A preferred lithium salt is lithium bis(trifluoromethylsulfonyl)imide.

Preferably, the formulation comprises from 0.1 mM to 50 mM, preferably from 5 to 20 mM of the lithium salt.

Suitable device structures for PSCs comprising a compound formula I and a mixed halide perovskite are described in WO 2013/171517, claims 52 to 71 and claims 72 to 79, which is entirely incorporated herein by reference.

Suitable device structures for PSCs comprising a compound formula and a dielectric scaffold together with a perovskite are described in WO 2013/171518, claims 1 to 90 or WO 2013/171520, claims 1 to 94 which are entirely incorporated herein by reference.

Suitable device structures for PSCs comprising a compound of formula I, a semiconductor and a perovskite are described in WO 2014/020499, claims 1 and 3 to 14, which is entirely incorporated herein by reference The surface-increasing scaffold structure described therein comprises nanoparticles which are applied and/or fixed on a support layer, e.g. porous TiO₂.

Suitable device structures for PSCs comprising a compounds of formula and comprising a planar heterojunction are described in WO 2014/045021, claims 1 to 39, which is entirely incorporated herein by reference. Such a device is characterized in having a thin film of a light-absorbing or light-emitting perovskite disposed between n-type (electron conducting) and p-type (hole-conducting) layers. Preferably, the thin film is a compact thin film.

The invention further relates to a method of preparing a PSC as described above or below, the method comprising the steps of:
providing a first and a second electrode;
providing an electron selective layer comprising a compound of formula I.

The invention relates furthermore to a tandem device comprising at least one device according to the invention as described above and below. Preferably, the tandem device is a tandem solar cell.

The tandem device or tandem solar cell according to the invention may have two semi-cells wherein one of the semi cells comprises the compounds, oligomers or polymers in the active layer as described or preferably described above. There exists no restriction for the choice of the other type of semi cell which may be any other type of device or solar cell known in the art.

There are two different types of tandem solar cells known in the art. The so called 2-terminal or monolithic tandem solar cells have only two connections. The two subcells (or synonymously semi cells) are connected in series. Therefore, the current generated in both subcells is identical (current matching). The gain in power conversion efficiency is due to an increase in voltage as the voltages of the two subcells add up. The other type of tandem solar cells is the so called 4-terminal or stacked tandem solar cell. In this case, both subcells are operated independently. Therefore, both subcells can be operated at different voltages and can also generate different currents. The power conversion efficiency of the tandem solar cell is the sum of the power conversion efficiencies of the two subcells.

The invention furthermore relates to a module comprising a device according to the invention as described above.

The compounds and compositions of the present invention can also be used as dye or pigment in other applications, for example as an ink dye, laser dye, fluorescent marker, solvent dye, food dye, contrast dye or pigment in coloring paints, inks, plastics, fabrics, cosmetics, food and other materials.

The compounds and compositions of the present invention are also suitable for use in the semiconducting channel of an OFET. Accordingly, the invention also provides an OFET comprising a gate electrode, an insulating (or gate insulator) layer, a source electrode, a drain electrode and an organic semiconducting channel connecting the source and drain electrodes, wherein the organic semiconducting channel comprises a compound and compositions according to the present invention. Other features of the OFET are well known to those skilled in the art. OFETs where an OSC material is arranged as a thin film between a gate dielectric and a drain and a source electrode, are generally known, and are described for example in U.S. Pat. Nos. 5,892,244, 5,998,804, 6,723,394 and in the references cited in the background section. Due to the advantages, like low cost production using the solubility properties of the compounds according to the invention and thus the processability of large surfaces, preferred applications of these OFETs are such as integrated circuitry, TFT displays and security applications.

The gate, source and drain electrodes and the insulating and semiconducting layer in the OFET device may be arranged in any sequence, provided that the source and drain electrode are separated from the gate electrode by the insulating layer, the gate electrode and the semiconductor layer both contact the insulating layer, and the source electrode and the drain electrode both contact the semiconducting layer.

An OFET device according to the present invention preferably comprises:
a source electrode,
a drain electrode,
a gate electrode,
a semiconducting layer,
one or more gate insulator layers,
optionally a substrate,

wherein the semiconductor layer preferably comprises a compound of formula I.

The OFET device can be a top gate device or a bottom gate device. Suitable structures and manufacturing methods of an OFET device are known to the skilled in the art and are described in the literature, for example in US 2007/0102696 A1.

The gate insulator layer preferably comprises a fluoropolymer, like e.g. the commercially available Cytop 809M® or Cytop 107M® (from Asahi Glass). Preferably the gate insulator layer is deposited, e.g. by spin-coating, doctor blading, wire bar coating, spray or dip coating or other known methods, from a formulation comprising an insulator material and one or more solvents with one or more fluoro atoms (fluorosolvents), preferably a perfluorosolvent. A suitable perfluorosolvent is e.g. FC75® (available from Acros, catalogue number 12380). Other suitable fluoropolymers and fluorosolvents are known in prior art, like for example the perfluoropolymers Teflon AF® 1600 or 2400 (from DuPont) or Fluoropel® (from Cytonix) or the perfluorosolvent FC 43® (Acros, No. 12377). Especially preferred are organic dielectric materials having a low permittivity (or dielectric content) from 1.0 to 5.0, very preferably from 1.8 to 4.0 (“low k materials”), as disclosed for example in US 2007/0102696 A1 or U.S. Pat. No. 7,095,044.

In security applications, OFETs and other devices with semiconducting materials according to the present invention, like transistors or diodes, can be used for RFID tags or security markings to authenticate and prevent counterfeiting of documents of value like banknotes, credit cards or ID cards, national ID documents, licenses or any product with monetary value, like stamps, tickets, shares, cheques etc.

Alternatively, the compounds and compositions (hereinafter referred to as “materials”) according to the present invention can be used in OLEDs, e.g. as the active display material in a flat panel display applications, or as backlight of a flat panel display like e.g. a liquid crystal display. Common OLEDs are realized using multilayer structures. An emission layer is generally sandwiched between one or more electron-transport and/or hole-transport layers. By applying an electric voltage electrons and holes as charge carriers move towards the emission layer where their recombination leads to the excitation and hence luminescence of the lumophor units contained in the emission layer. The materials according to the present invention may be employed in one or more of the charge transport layers and/or in the emission layer, corresponding to their electrical and/or optical properties. Furthermore their use within the emission layer is especially advantageous, if the materials according to the present invention show electroluminescent properties themselves or comprise electroluminescent groups or compounds. The selection, characterization as well as the processing of suitable monomeric, oligomeric and polymeric compounds or materials for the use in OLEDs is generally known by a person skilled in the art, see, e.g., Müller et al, Synth. Metals, 2000, 111-112, 31-34, Alcala, J. Appl. Phys., 2000, 88, 7124-7128 and the literature cited therein.

According to another use, the materials according to the present invention, especially those showing photoluminescent properties, may be employed as materials of light sources, e.g. in display devices, as described in EP 0 889 350 A1 or by C. Weder et al., Science, 1998, 279, 835-837.

A further aspect of the invention relates to both the oxidised and reduced form of the materials according to the present invention. Either loss or gain of electrons results in formation of a highly delocalised ionic form, which is of high conductivity. This can occur on exposure to common dopants. Suitable dopants and methods of doping are known to those skilled in the art, e.g. from EP 0 528 662, U.S. Pat. No. 5,198,153 or WO 96/21659.

The doping process typically implies treatment of the semiconductor material with an oxidating or reducing agent in a redox reaction to form delocalised ionic centres in the material, with the corresponding counterions derived from the applied dopants. Suitable doping methods comprise for example exposure to a doping vapor in the atmospheric pressure or at a reduced pressure, electrochemical doping in a solution containing a dopant, bringing a dopant into contact with the semiconductor material to be thermally diffused, and ion-implantation of the dopant into the semiconductor material.

When electrons are used as carriers, suitable dopants are for example halogens (e.g., I₂, Cl₂, Br₂, ICl, ICl₃, IBr and IF), Lewis acids (e.g., PF₅, AsF₅, SbF₅, BF₃, BCl₃, SbCl₅, BBr₃ and SO₃), protonic acids, organic acids, or amino acids (e.g., HF, HCl, HNO₃, H₂SO₄, HClO₄, FSO₃H and ClSO₃H), transition metal compounds (e.g., FeCl₃, FeOCl, Fe(ClO₄)₃, Fe(4-CH₃C₆H₄SO₃)₃, TiCl₄, ZrCl₄, HfCl₄, NbF₅, NbCl₅, TaCl₅, MoF₅, MoCl₅, WF₅, WCl₆, UF₆ and LnCl₃ (wherein Ln is a lanthanoid), anions (e.g., Cl⁻, Br, I⁻, I₃⁻, HSO₄⁻, SO₄²⁻, NO₃⁻, ClO₄⁻, BF₄, PF₆, AsF₆⁻, SbF₆⁻, FeCl₄⁻, Fe(CN)₆³⁻, and anions of various sulfonic acids, such as aryl-SO₃⁻). When holes are used as carriers, examples of dopants are cations (e.g., H⁺, Li⁺, Na⁺, K⁺, Rb⁺ and Cs⁺), alkali metals (e.g., Li, Na, K, Rb, and Cs), alkaline-earth metals (e.g., Ca, Sr, and Ba), O₂, XeOF₄, (NO₂+) (SbF₆), (NO₂+) (SbCl₆⁻), (NO₂⁺) (BF₄⁻), AgClO₄, H₂IrCl₆, La(NO₃)₃·6H₂O, FSO₂OOSO₂F, Eu, acetylcholine, R₄N⁺, (R is an alkyl group), R₄P⁺ (R is an alkyl group), R₆As⁺ (R is an alkyl group), and R₃S⁺ (R is an alkyl group).

The conducting form of the materials according to the present invention can be used as an organic “metal” in applications including, but not limited to, charge injection layers and ITO planarising layers in OLED applications, films for flat panel displays and touch screens, antistatic films, printed conductive substrates, patterns or tracts in electronic applications such as printed circuit boards and condensers.

The materials according to the present invention may also be suitable for use in organic plasmon-emitting diodes (OPEDs), as described for example in Koller et al., Nat. Photonics, 2008, 2, 684.

According to another use, the materials according to the present invention can be used alone or together with other materials in or as alignment layers in LCD or OLED devices, as described for example in US 2003/0021913. The use of charge transport compounds according to the present invention can increase the electrical conductivity of the alignment layer. When used in an LCD, this increased electrical conductivity can reduce adverse residual dc effects in the switchable LCD cell and suppress image sticking or, for example in ferroelectric LCDs, reduce the residual charge produced by the switching of the spontaneous polarisation charge of the ferroelectric LCs. When used in an OLED device comprising a light emitting material provided onto the alignment layer, this increased electrical conductivity can enhance the electroluminescence of the light emitting material.

The materials according to the present invention having mesogenic or liquid crystalline properties can form oriented anisotropic films as described above, which are especially useful as alignment layers to induce or enhance alignment in a liquid crystal medium provided onto said anisotropic film.

According to another use, the materials according to the present invention are suitable for use in liquid crystal (LC) windows, also known as smart windows.

The materials according to the present invention may also be combined with photoisomerisable compounds and/or chromophores for use in or as photoalignment layers, as described in US 2003/0021913 A1.

According to another use, the materials according to the present invention, especially their water-soluble derivatives (for example with polar or ionic side groups) or ionically doped forms, can be employed as chemical sensors or materials for detecting and discriminating DNA sequences. Such uses are described for example in L. Chen, D. W. McBranch, H. Wang, R. Helgeson, F. Wudl and D. G. Whitten, Proc. Natl. Acad. Sci. U.S.A., 1999, 96, 12287; D. Wang, X. Gong, P. S. Heeger, F. Rininsland, G. C. Bazan and A. J. Heeger, Proc. Natl. Acad. Sci. U.S.A., 2002, 99, 49; N. DiCesare, M. R. Pinot, K. S. Schanze and J. R. Lakowicz, Langmuir, 2002, 18, 7785; D. T. McQuade, A. E. Pullen, T. M. Swager, Chem. Rev., 2000, 100, 2537.

Unless the context clearly indicates otherwise, as used herein plural forms of the terms herein are to be construed as including the singular form and vice versa.

Throughout the description and claims of this specification, the words “comprise” and “contain” and variations of the words, for example “comprising” and “comprises”, mean “including but not limited to”, and are not intended to (and do not) exclude other components.

It will be appreciated that variations to the foregoing embodiments of the invention can be made while still falling within the scope of the invention. Each feature disclosed in this specification, unless stated otherwise, may be replaced by alternative features serving the same, equivalent or similar purpose. Thus, unless stated otherwise, each feature disclosed is one example only of a generic series of equivalent or similar features.

All of the features disclosed in this specification may be combined in any combination, except combinations where at least some of such features and/or steps are mutually exclusive. In particular, the preferred features of the invention are applicable to all aspects of the invention and may be used in any combination. Likewise, features described in non-essential combinations may be used separately (not in combination).

Above and below, unless stated otherwise percentages are percent by weight and temperatures are given in degrees Celsius.

The invention will now be described in more detail by reference to the following examples, which are illustrative only and do not limit the scope of the invention.

### EXAMPLES

Molecular structures were optimized at B3LYP/6-31 G* level using Firefly QC package (see Alex A. Granovsky, Firefly version 8, www http://classic.chem.msu.su/gran/firefly/index.html), which is partially based on the GAMESS (US) source code (see M. W. Schmidt, K. K. Baldridge, J. A. Boatz, S. T. Elbert, M. S. Gordon, J. H. Jensen, S. Koseki, N. Matsunaga, K. A. Nguyen, S. Su, T. L. Windus, M. Dupuis, J. A. Montgomery J. Comput. Chem. 14, 1347-1363 (1993)).

E_{HOMO} and E_{LUMO} are defined as the eigenvalues of, respectively, the highest occupied and lowest unoccupied Kohn-Sham molecular orbitals, and are used as approximations of, respectively, ionisation potential (IP) and electron affinity (EA). E_{g} is defined as |E_{LUMO}-E_{HOMO}| and is the transport band gap of the material. S₀-S₁ is the vertical excitation energy from the ground state S₀ to the first singlet excited state S₁, and is used as the measure of the optical band gap E_{g}(opt).

An approximate relation between E_{HOMO}, E_{LUMO} and E_{g} of donor and acceptor materials in a bulk-heterojunction and device performance is known as the Scharber model [M. C. Scharber, D. Mühlbacher, M. Koppe, P. Denk, C. Waldauf, A. J. Heeger, C. J. Brabec, Adv. Mater. 2006, 18, 789-794]. It is widely accepted that when the donor material of the donor-acceptor blend absorbs light and forms an excited state, the excited electron must hop onto the neighbouring acceptor site in order for the free carriers to be formed. The driving force of this process is the energetic difference between the excited state of the donor material and the electron affinity (approximated by E_{LUMO}) of the acceptor material and has been empirically found to be at least ca. 0.35 eV for charge generation to be efficient [D. Veldman, S. C. J. Meskers, R. A. J. Janssen, Adv. Funct. Mater. 2009, 19, 1939-1948; M. C. Scharber, N. S. Sariciftci, Progr. Polym. Sci. 38 (2013) 1929-1940]. Therefore, tuning of acceptor's E_{LUMO} is of paramount importance, lowering its value will increase the driving force for charge generation and may allow using lower-bandgap donor material, whilst increasing E_{LUMO} may hinder charge generation. For the present OSC materials, owing to their small optical band gap, another mechanism is also possible: light absorption by the acceptor followed by hole injection to the donor material, driven by the energy difference between E_{HOMO} of donor and acceptor, respectively [W. Zhao, D. Qian, S. Zhang, S. Li, O. Inganäs, F. Gao, J. Hou, Adv. Mater. 2016, DOI: 10.1002/adma.201600281]. This mechanism is responsible for non-negligible external quantum efficiency beyond the absorption edge of the donor material, and retaining of this advantage of the acceptor material requires careful tuning of HOMO energy.

### Comparative Example C1

Compound C1 as shown below is calculated as a reference.

| | | E_{HOMO}/ | E_{LUMO}/ | | S₀-S₁/ |
|---|---|---|---|---|---|
| No. | Structure | eV | eV | Eg/eV | eV |
| C1 | 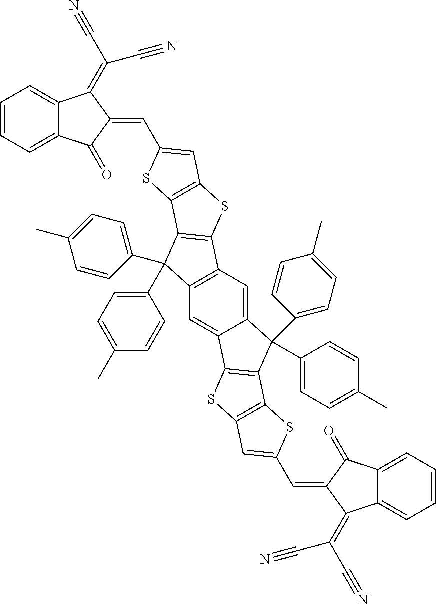 [SMILES: Cc1ccc(C2(c3ccc(C)cc3)c3cc4c(cc3-c3sc5cc(/C=C6\C(=O)c7ccccc7C6=C(C#N)C#N)sc5c32)C(c2ccc(C)cc2)(c2ccc(C)cc2)c2c-4sc3cc(/C=C4\C(=O)c5ccccc5C4=C(C#N)C#N)sc23)cc1] | −5.46 | −3.34 | 2.12 | 1.91 |

### Examples 1-8

The computed values of E_{HOMO}, E_{LUMO}, E_{g} and S₀-S₁ of compound C1 (whilst being different from experimentally determined IP, EA and E_{g}) are compared with the computed values of compounds 1-8.

| | | | | | S₀- |
|---|---|---|---|---|---|
| | | E_{HOMO}/ | E_{LUMO}/ | Eg/ | S₁/ |
| No. | Structure | eV | eV | eV | eV |
| 1 | 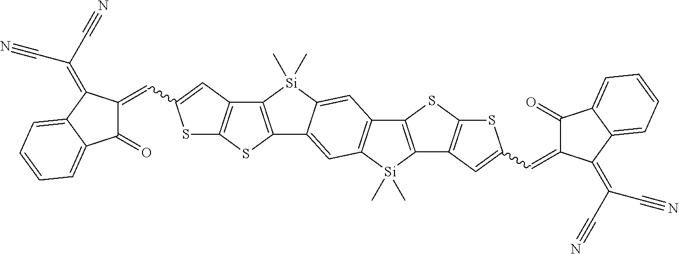 [SMILES: C[Si]1(C)c2cc3c(cc2-c2sc4sc(C=C5C(=O)c6ccccc6C5=C(C#N)C#N)cc4c21)[Si](C)(C)c1c-3sc2sc(C=C3C(=O)c4ccccc4C3=C(C#N)C#N)cc12] | −5.62 | −3.21 | 2.41 | 1.99 |
| 2 | 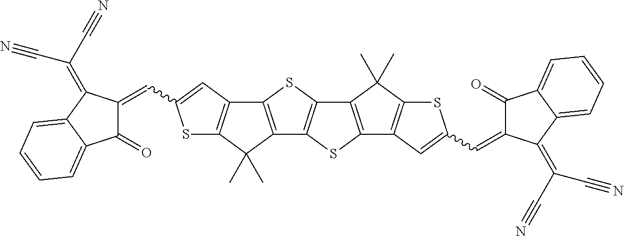 [SMILES: CC1(C)c2sc(C=C3C(=O)c4ccccc4C3=C(C#N)C#N)cc2-c2sc3c4c(sc3c21)-c1cc(C=C2C(=O)c3ccccc3C2=C(C#N)C#N)sc1C4(C)C] | −5.41 | −3.23 | 2.18 | 1.98 |
| 3 | 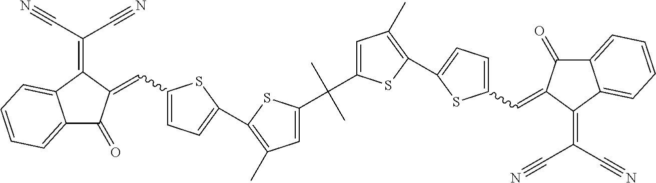 [SMILES: Cc1cc(C(C)(C)c2cc(C)c(-c3ccc(C=C4C(=O)c5ccccc5C4=C(C#N)C#N)s3)s2)sc1-c1ccc(C=C2C(=O)c3ccccc3C2=C(C#N)C#N)s1] | −5.75 | −3.22 | 2.53 | 2.37 |
| 4 | 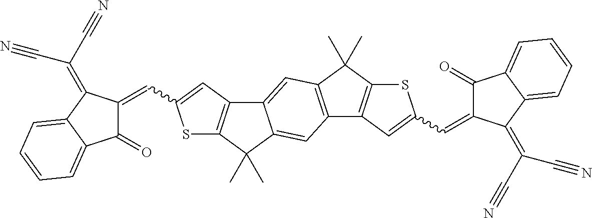 [SMILES: CC1(C)c2cc3c(cc2-c2cc(C=C4C(=O)c5ccccc5C4=C(C#N)C#N)sc21)C(C)(C)c1sc(C=C2C(=O)c4ccccc4C2=C(C#N)C#N)cc1-3] | −5.77 | −3.21 | 2.56 | 2.24 |
| 5 | 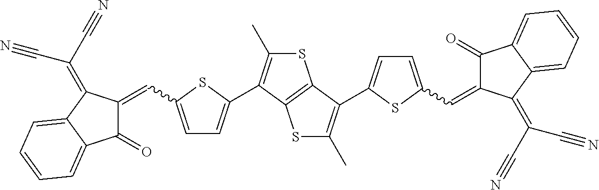 [SMILES: Cc1sc2c(-c3ccc(C=C4C(=O)c5ccccc5C4=C(C#N)C#N)s3)c(C)sc2c1-c1ccc(C=C2C(=O)c3ccccc3C2=C(C#N)C#N)s1] | −5.86 | −3.28 | 2.58 | 2.16 |
| 6 | 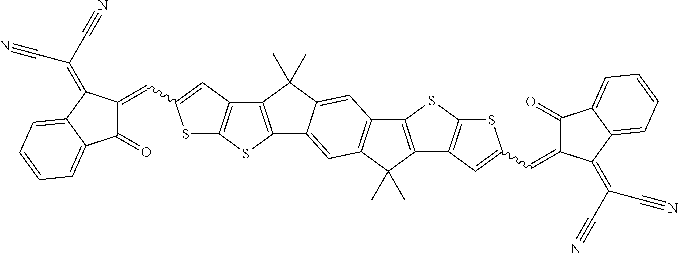 [SMILES: CC1(C)c2cc3c(cc2-c2sc4sc(C=C5C(=O)c6ccccc6C5=C(C#N)C#N)cc4c21)C(C)(C)c1c-3sc2sc(C=C3C(=O)c4ccccc4C3=C(C#N)C#N)cc12] | −5.52 | −3.24 | 2.18 | 1.86 |
| 7 | 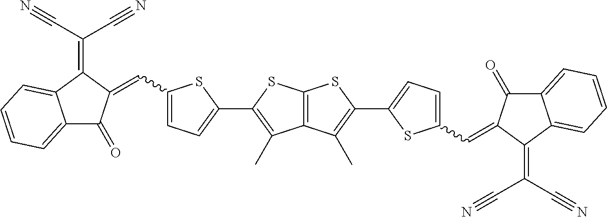 [SMILES: Cc1c(-c2ccc(C=C3C(=O)c4ccccc4C3=C(C#N)C#N)s2)sc2sc(-c3ccc(C=C4C(=O)c5ccccc5C4=C(C#N)C#N)s3)c(C)c12] | −5.84 | −3.36 | 2.48 | 2.26 |
| 8 | 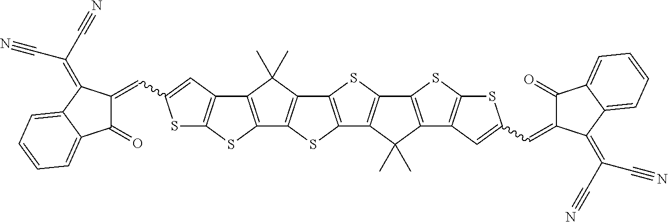 [SMILES: CC1(C)c2c(sc3sc(C=C4C(=O)c5ccccc5C4=C(C#N)C#N)cc23)-c2sc3c4c(sc3c21)-c1sc2sc(C=C3C(=O)c5ccccc5C3=C(C#N)C#N)cc2c1C4(C)C] | −5.18 | −3.25 | 1.93 | 1.53 |

### Example 9

### 2,5-Bis-thieno[2,3-b]thiophen-2-yl-terephthalic acid diethyl ester

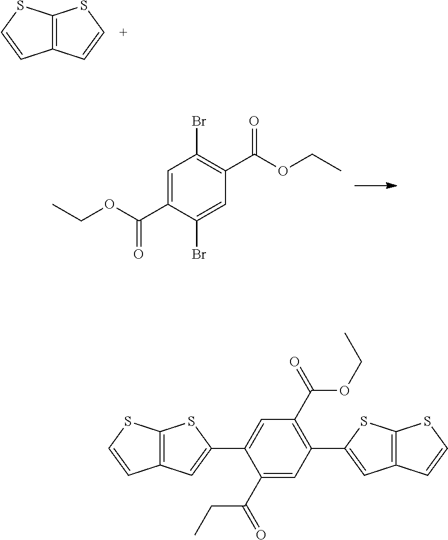 [SMILES: c1cc2ccsc2s1 ; CCOC(=O)c1cc(Br)c(C(=O)OCC)cc1Br ; CCOC(=O)c1cc(-c2cc3ccsc3s2)c(C(=O)CC)cc1-c1cc2ccsc2s1]

To a solution of thieno[2,3-b]thiophene (10.0 g, 71 mmol) in anhydrous tetrahydrofuran (100 cm³) at −78° C. was added dropwise n-butyllithium (31.4 cm³, 78 mmol) and the mixture stirred at −78° C. for 30 minutes. Tributyltin chloride (23.2 cm³, 85.6 mmol) was added in one go and the mixture stirred in the ice bath while warming to 23° C. for 17 hours. Water (500 cm³) was added and the product extracted with ether (2×200 cm³). The combined organics dried over anhydrous magnesium sulfate, filtered and the solvent removed in vacuo to give crude tributyl-thieno[2,3-b]thiophen-2-yl-stannane as a yellow oil. To the crude was added anhydrous N,N-dimethylformamide (100 cm³), 2,5-dibromo-terephthalic acid diethyl ester (11.0 g, 29 mmol) and bis(triphenylphosphine)palladium(II) dichloride (612 mg, 0.87 mmol) and the mixture heated at 100° C. for 2 hours. The mixture allowed to cool to 23° C., concentrated in vacuo and passed through a plug of silica (dichloromethane) to give a solid which was triturated in methanol. The solid collected by filtration to give 2,5-bis-thieno[2,3-b]thiophen-2-yl-terephthalic acid diethyl ester (11.68 g, 81%) as a yellow solid. ¹H-NMR (400 MHz, CDCl₃) 1.13 (6H, t, J 7.1), 4.24 (4H, q, J 7.1), 7.24 (2H, d, J 5.2), 7.26 (2H, s), 7.37 (2H, d, J 5.2), 7.88 (2H, s).

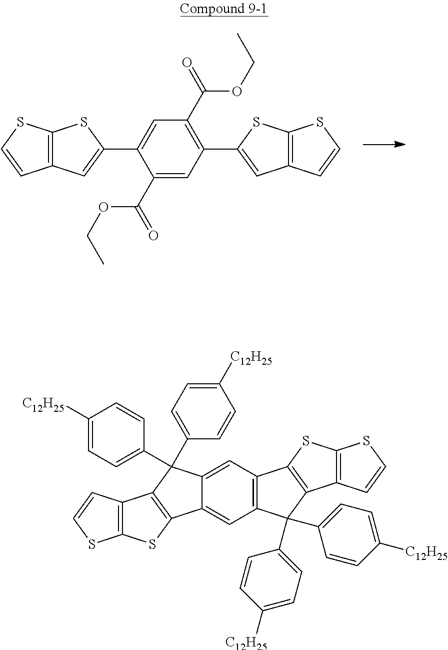

To magnesium (1.58 g, 65 mmol), anhydrous tetrahydrofuran (100 cm³) and a crystal of iodine at reflux was added dropwise 1-bromo-4-dodecylbenzene (16.3 g, 50 mmol). The reaction mixture was then heated at reflux for 2 hours. The mixture was allowed to cool to 23° C. and 2,5-bis-thieno[2,3-b]thiophen-2-yl-terephthalic acid diethyl ester (5.0 g, 2.6 mmol) was added in in go. The mixture was then heated at reflux for 17 hours. The mixture was allowed to cool to 23° C. and water (50 cm³) added followed by stirring for 15 minutes. The volatile organics removed in vacuo. Methanol (200 cm³) was added and the yellow solid was collected by filtration, washed with methanol then air-dried on the filter. The solid was dissolved in warm anhydrous dichloromethane (200 cm³) and p-toluenesulfonic acid monohydrate (3.34 g, 17.5 mmol). The solution was stirred at 23° C. for 90 minutes. The mixture was concentrated in vacuo and methanol (200 cm³) added. The solid was collected by filtration and washed with methanol (100 cm³). Further purification by column chromatography (40-60 petrol) gave compound 9-1 (3.55 g, 26%) as a yellow solid. ¹H-NMR (400 MHz, CDCl₃) 0.82-0.91 (12H, m), 1.17-1.37 (72H, m), 1.51-1.63 (8H, m), 2.49-2.59 (8H, m), 7.02-7.09 (10H, m), 7.16-7.22 (8H, m), 7.25 (2H, d, J 5.3), 7.46 (2H, s).

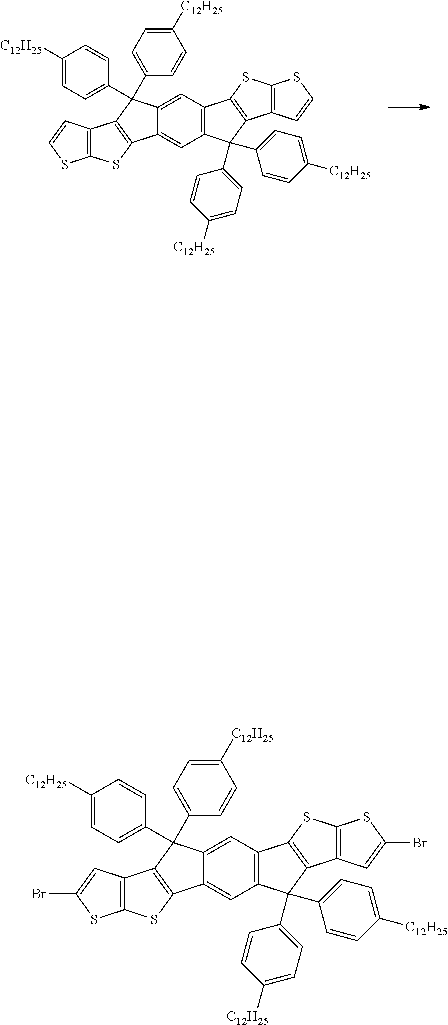

To a solution of compound 9-1 (2.0 g, 1.5 mmol) in chloroform (100 cm³) and acetic acid (20 cm³) was added in one go N-bromosuccinimide (583 mg, 3.2 mmol) and the solution stirred at 23° C. for 3 hours. The mixture was concentrated in vacuo, methanol (200 cm³) was added and the precipitate collected by filtration. The crude was purified by silica plug (40-60 petrol:dichloromethane; 2:1) followed by recrystallisation (cyclohexanes) to give compound 9-2 (1.63 g, 73%) as an off-white solid.

¹H-NMR (400 MHz, CDCl₃) 0.83-0.92 (12H, m), 1.18-1.38 (72H, m), 1.48-1.68 (8H, m), 2.50-2.59 (8H, m), 7.03-7.17 (18H, m), 7.43 (2H, s).

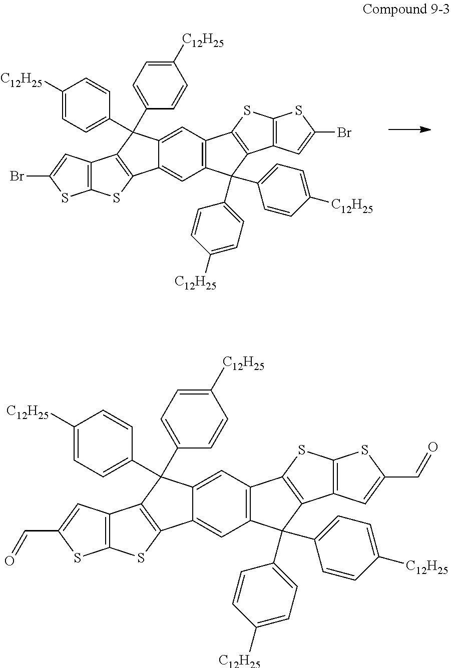

Compound 9-2 (914 mg, 0.60 mmol) was taken up in anhydrous tetrahydrofuran (60 cm³) and then cooled to −78° C. To the mixture was added n-butyllithium (1.21 cm³, 3.02 mmol, 2.5 M in hexane) dropwise over 15 minutes. The reaction mixture was then stirred for 1 hour followed by addition of a solution of anhydrous N,N-dimethylformamide (1.16 cm³, 15.1 mmol) in anhydrous ether (30 cm³) in two portions. The resulting solution was allowed to warm to 23° C. and stirred f or 24 hours. Water (100 cm³) and ethyl acetate (50 cm³) were added and the organic layer separated. Further ethyl acetate (15 cm³) was added and the organic layer separated. The combined organic extracts were then washed with water (75 cm³) and brine (100 cm³) and dried over anhydrous magnesium sulfate, filtered and the solvent removed in vacuo. The crude was purified by column chromatography using a graded solvent system (40-60 petrol:dichloromethane; 7:3 to 1:1) to give compound 9-3 (213 mg, 25%) as an orange/yellow solid. ¹H-NMR (400 MHz, CDCl₃) 0.83-0.92 (12H, m), 1.20-1.38 (72H, m), 1.54-1.64 (8H, m), 2.52-2.59 (8H, m), 7.05-7.23 (16H, m), 7.52 (2H, s), 7.70 (2H, s), 9.84 (2H, s).

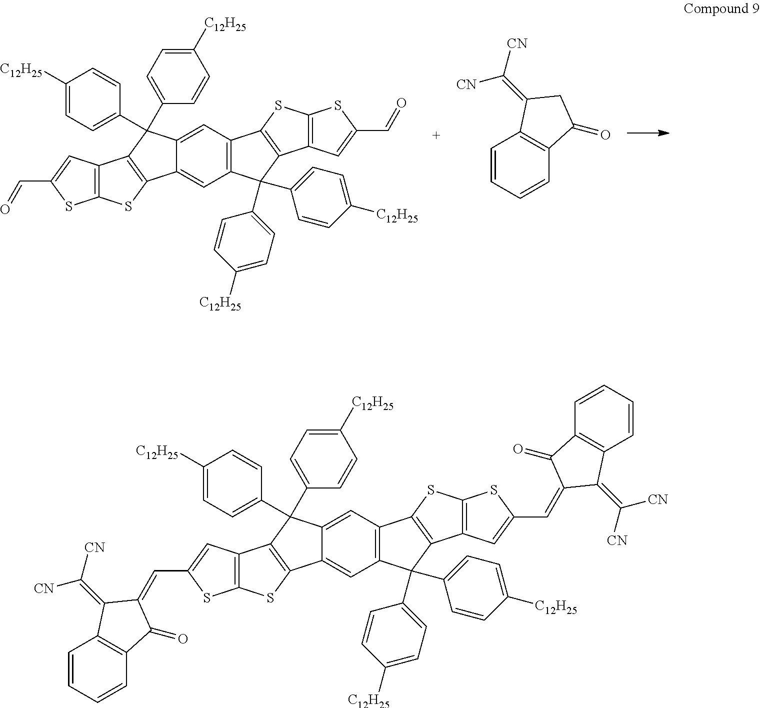

To a degassed mixture of compound 9-3 (192 mg, 0.14 mmol), anhydrous chloroform (15 cm³) and pyridine (0.77 cm³) was added 2-(3-oxo-indan-1-ylidene)-malononitrile (185 mg, 0.95 mmol). The solution was then degassed for a further 10 minutes and then stirred at 23° C. for 160 minutes. Methanol (300 cm³) was added and after stirring for 20 minutes the solid was collected by filtration. The solid was then washed with methanol (3×10 cm³), acetone (3×10 cm³) and diethyl ether (2×10 cm³) to give compound 9 (179 mg, 75%) as a solid. ¹H-NMR (400 MHz, CDCl₃) 0.80-0.90 (12H, m), 1.18-1.43 (72H, m), 1.49-1.64 (8H, m), 2.48-2.60 (8H, m), 7.06-7.19 (16H, m), 7.53 (2H, s), 7.70 (2H, s), 7.73-7.83 (4H, m), 7.90-7.95 (2H, m), 8.65-8.70 (2H, m), 8.81 (2H, s).

### Example 10

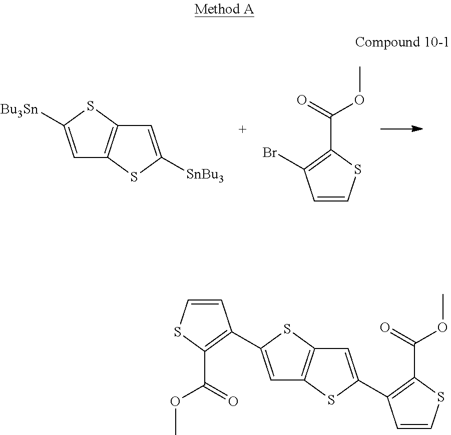

To a degassed solution of 3-bromothiophene-2-carboxylic acid methyl ester (0.68 g, 3.1 mmol), N,N-dimethylformamide (20 cm³) and 2,5-bis-tributylstannanyl-thieno[3,2-b]thiophene (1.00 g, 1.39 mmol) was added palladium tetrakis(triphenylphosphine) (80 mg, 0.07 mmol) and the mixture degassed for a further 10 minutes. The reaction mixture was then heated at 90° C. for 17 hours and then allowed to cool to 23° C. The mixture was poured into water (25 cm³) and the solid collected by filtration. The solid was washed with heptane (100 cm³) and methyl t-butyl ether (100 cm³). The crude was then heated at reflux in toluene (100 cm³) for 2 hours, cooled and the solid collected by filtration and washed with heptane (100 cm³) and methyl t-butyl ether (100 cm³) to give compound 10-1 (220 mg, 37%) as a solid. ¹H-NMR (400 MHz, d-THF) 3.83 (6H, s), 7.39 (2H, d, J 5.3), 7.71 (2H, 8.05 (2H, s).

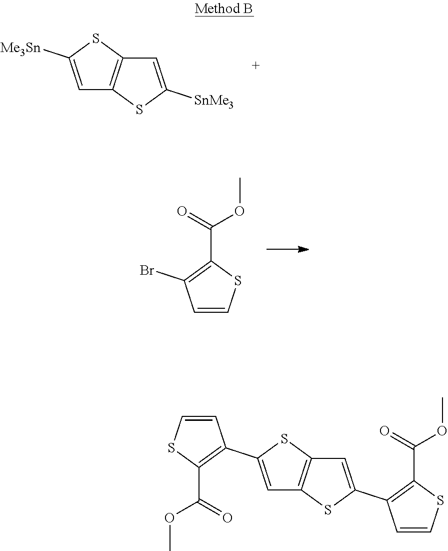

To a degassed solution of 3-bromothiophene-2-carboxylate (10.4 g, 47.2 mmol), toluene (500 cm³) and trimethyl[5-(trimethylstannyl)thieno[3,2-b]thiophen-2-yl]stannane (10.0 g, 21.5 mmol) was added tris(dibenzylideneacetone)dipalladium(0) (983 mg, 1.07 mmol) and 2-dicyclohexylphosphino-2′,4′,6′-triisopropylbiphenyl (2.05 g, 4.29 mmol). The reaction was then degassed for a further 15 minutes before heating to 80° C. for 45 minutes. The reaction was then heated to 110° C. for a further 15 minutes before cooling to 23° C. and stirring for 17 hours. The solid was collected by filtration and then washed with diethyl ether (4×50 cm³) and 40-60 petrol (3×70 cm³) to give compound 10-1 (8.76 g, 97%) as a beige solid. ¹H-NMR (400 MHz, CDCl₃) 4.02 (6H, s), 7.47 (2H, d, J 5.2), 7.56 (2H, d, J 5.2), 8.19 (2H, s).

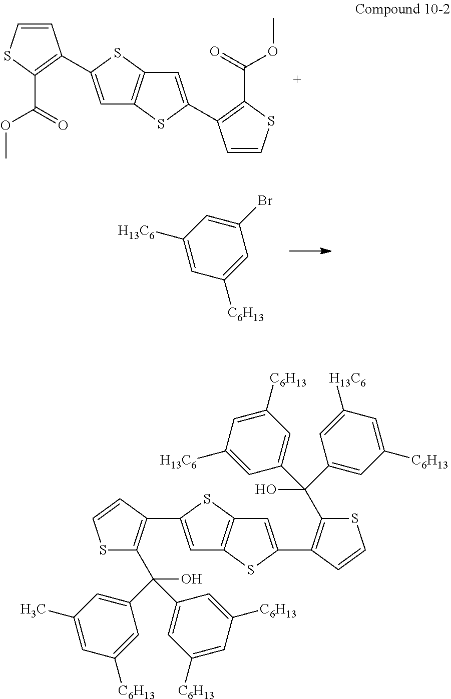

To a solution of 1-bromo-3,5-dihexyl-benzene (24.2 g, 74.3 mmol) and anhydrous tetrahydrofuran (75 cm³) at −78° C. was added dropwise n-butyllithium (32 cm³, 79 mmol, 2.5 M in hexane). After addition, the mixture was stirred for 90 minutes and then compound 10-1 (6.25 g, 14.9 mmol) was added and the mixture stirred for 17 hours while warming to 23° C. The mixture was then cooled to 0° C., water (100 cm³) added followed by dichloromethane (100 cm³). The mixture was then stirred for 30 minutes and the organic layer separated. The aqueous layer was re-extracted with dichloromethane (2×100 cm³) and the combined organics washed with water (100 cm³), brine (100 cm³), dried over anhydrous magnesium sulphate, filtered and the solvent removed in vacuo. Purification by column chromatography (heptane:dichloromethane; 7:3) gave compound 10-2 (9.0 g, 45%) as a brown oil. ¹H-NMR (400 MHz, CDCl₃) 0.75-0.98 (24H, m), 1.13-1.40 (48H, m), 1.46-1.71 (16H, m), 2.53-2.49 (16H, m), 3.47 (2H, s), 6.43 (2H, s), 6.87-7.02 (12H, m), 7.08 (2H, s), 7.14 (2H, s).

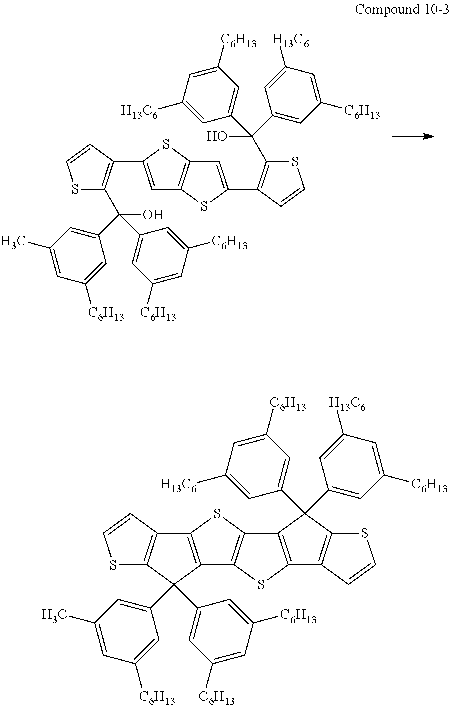

To a solution of compound 10-2 (0.24 g, 0.18 mmol) and dichloromethane (10 cm³) was added p-toluene sulfonic acid monohydrate (68 mg, 0.36 mmol) and the mixture heated at reflux for 2 hours. The mixture was then allowed to cool to 23° C. and the solvent removed in vacuo. The crude was purified by column chromatography (heptane:dichloromethane; 99.5:0.5 then 49:1) to give compound 10-3 (70 mg, 30%) as a colourless oil. ¹H-NMR (400 MHz, CDCl₃) 0.79-0.93 (24H, m), 1.19-1.38 (48H, m) 1.48-1.73 (16H, m), 2.49 (16H, t, J 7.6), 6.86 (12H, s), 7.00 (2H, d, J 5.0), 7.36 (2H, d, J 5.0).

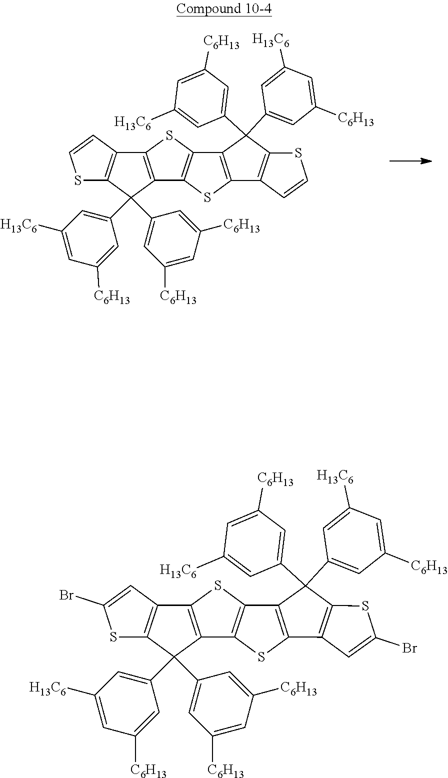

To a solution of compound 10-3 (3.00 g, 2.30 mmol) in dichloromethane (30 cm³) at −3° C. was added portion-wise N-bromosuccinimide (0.86 g, 4.8 mmol) over 20 minutes. The reaction was stirred and allowed to warm to 23° C. over 65 hours.

The reaction mixture was poured into water (50 cm³) and extracted with ethyl acetate (3×50 cm³). The combined organics were washed with brine (50 cm³) then dried over anhydrous magnesium sulphate, filtered and the solvent removed in vacuo. The crude was purified by column chromatography (heptane) to give compound 10-4 (2.90 g, 86%) as a yellow viscous oil. ¹H-NMR (400 MHz, CDCl₃) 0.76-0.94 (24H, m), 1.16-1.38 (48H, m), 1.44-1.59 (16H, m), 2.49 (16H, t, J 7.6), 6.81 (8H, s), 6.87 (4H, s), 7.01 (2H, s).

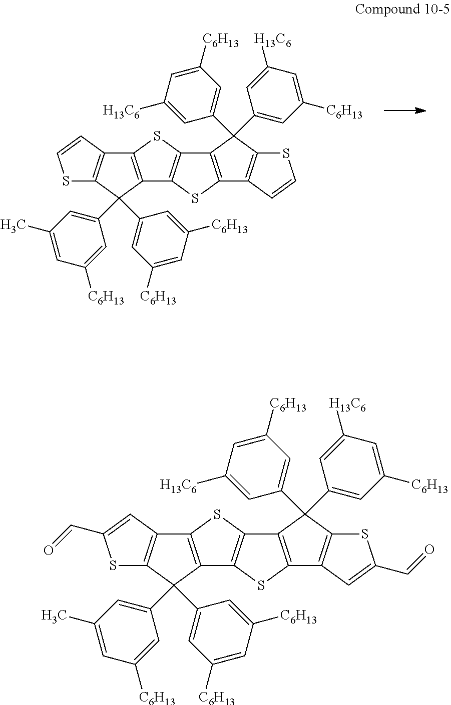

To a solution of compound 10-4 (2.90 g, 1.98 mmol) in anhydrous tetrahydrofuran (30 cm³) at −78° C. was added n-butyllithium (1.82 cm³, 4.56 mmol, 2.5 M in hexane) over 5 minutes. The reaction mixture was then stirred for 1 hour before anhydrous N,N-dimethylformamide (0.38 cm³, 5.0 mmol) was added. The reaction mixture was then stirred for 17 hours and allowed to warm to 23° C. Water (50 cm³) was slowly added and the organic layer separated. The aqueous layer was extracted with dichloromethane (3×100 cm³) and then the combined organic layer was washed with brine (50 cm³), dried over anhydrous magnesium sulfate, filtered and the solvent removed in vacuo. The crude was purified by column chromatography using a graded solvent system (heptane:dichloromethane; 1:0 to 7:3) to give compound 10-5 (1.90 g, 70%) as an orange oil. ¹H-NMR (400 MHz, CDCl₃) 0.75-0.94 (24H, m), 1.25 (48H, br. s.), 1.45-1.63 (16H, m), 2.49 (16H, t, J 7.7), 6.76-6.87 (8H, m), 6.91 (4H, s), 7.64 (2H, s), 9.89 (2H, s).

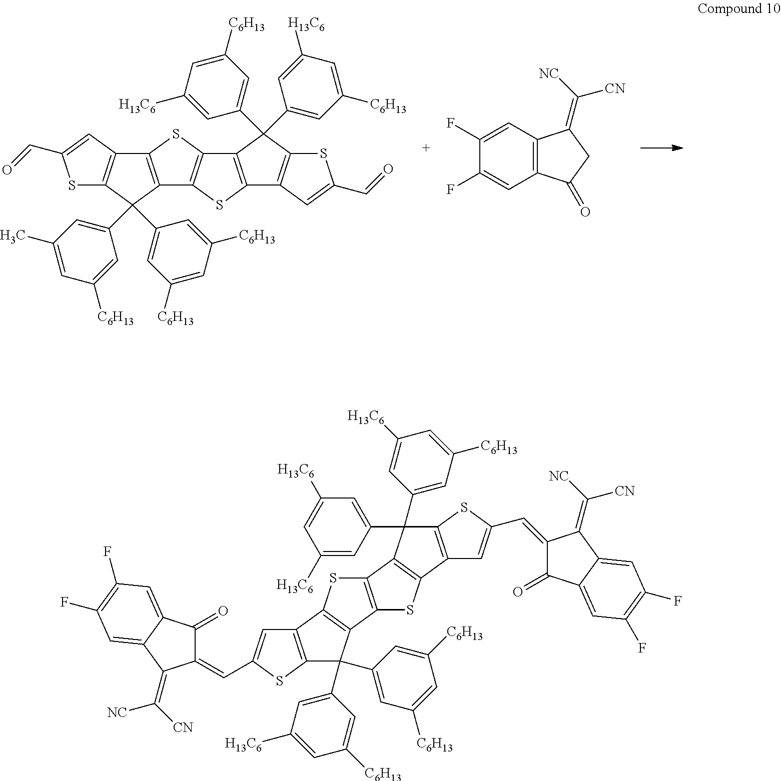

To a degassed solution of compound 10-5 (150 mg, 0.11 mmol) and pyridine (0.6 cm³) in anhydrous chloroform (40 cm³) at −10° C. was added a degassed solution of 2-(5,6-difluoro-3-oxo-2,3-dihydro-1H-inden-1-ylidene)propanedinitrile (101 mg, 0.440 mmol) in anhydrous chloroform (8 cm³) over 10 minutes. The resulting solution was then degassed for a further 30 minutes. The ice bath was removed and the reaction mixture warmed to 23° C. and stirred for 2 hours. The reaction mixture was diluted with acetonitrile (500 cm³) and stirred for 1 hour. The solid collected by filtration and washed with acetonitrile (100 cm³) and methanol (100 cm³). The solid was then triturated in n-pentane (50 cm³) and the solid collected by filtration and washed with n-pentane (20 cm³) to give compound 10 (115 mg, 59%) as a dark solid. ¹H-NMR (400 MHz, CD₂Cl₂) 0.65-0.80 (24H, m), 1.09-1.29 (48H, m), 1.37-1.56 (16H, m), 2.43 (16H, t, J 7.6), 6.75-6.83 (8H, m), 6.87 (4H, s), 7.59 (2H, t, J 7.7), 7.82 (2H, s), 8.39-8.49 (2H, m), 8.80 (2H, s).

### Example 11

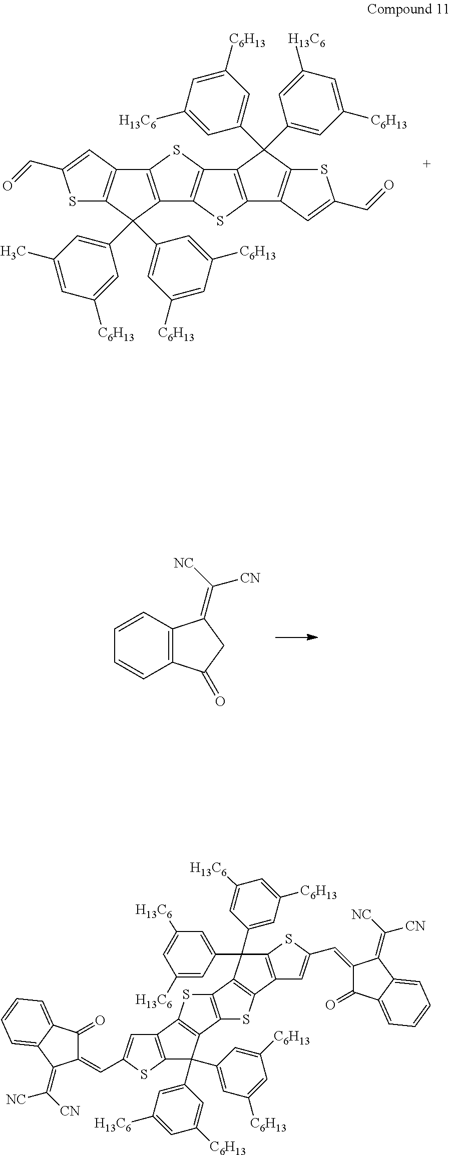

To a degassed solution of compound 10-5 (150 mg, 0.11 mmol) and pyridine (0.6 cm³) in anhydrous chloroform (40 cm³) at 0° C. was added a degassed solution of 2-(3-oxo-indan-1-ylidene)-malononitrile (86 mg, 0.440 mmol) in anhydrous chloroform (8 cm³). The resulting solution was then degassed for a further 30 minutes. The ice bath was removed and the reaction mixture warmed to 23° C. and stirred for 18 hours. The reaction mixture was diluted with acetonitrile (150 cm³) and stirred for 1 hour. The solid collected by filtration and washed with acetonitrile (100 cm³) and methanol (100 cm³). The crude was then purified by column chromatography (dichloromethane) followed by trituration in acetonitrile (100 cm³) to give compound 11 (183 mg, 97%) as a dark green solid. ¹H-NMR (400 MHz, CD₂Cl₂) 0.67-0.80 (24H, m), 1.11-1.23 (48H, m), 1.36-1.55 (16H, m), 2.43 (16H, t, J 7.7), 6.78-6.92 (12H, m), 7.63-7.74 (4H, m), 7.78-7.87 (4H, m), 8.60 (2H, d, J 7.3), 8.80 (2H, s).

### Example 12

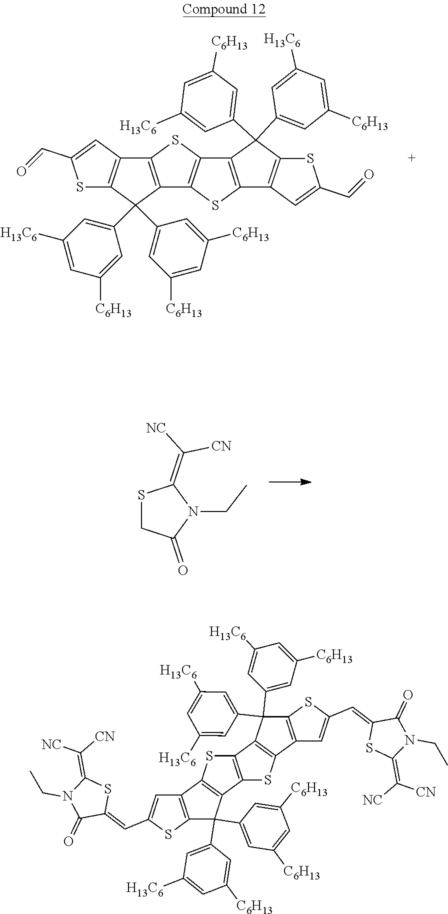

To a degassed solution of compound 10-5 (150 mg, 0.11 mmol) and pyridine (0.7 cm³) in anhydrous chloroform (40 cm³) at 0° C. was added a degassed solution of 2-(3-ethyl-4-oxo-thiazolidin-2-ylidene)-malononitrile (85 mg, 0.440 mmol) in anhydrous chloroform (8 cm³) in one portion. The resulting solution was then degassed for a further 30 minutes. The ice bath was removed and the reaction mixture warmed to 23° C. and stirred for 18 hours. The reaction mixture was diluted with acetonitrile (100 cm³) and stirred for 1 hour. The solid collected by filtration and washed with acetonitrile (100 cm³) and methanol (100 cm³) to give compound 12 (32 mg, 17%) as a dark green solid. ¹H-NMR (400 MHz, CD₂Cl₂) 0.69-0.79 (24H, m), 1.11-1.22 (48H, m), 1.26-1.33 (6H, m), 1.35-1.54 (16H, m), 2.41 (16H, t, J 7.5), 4.21 (4H, d, J 7.1), 6.71-6.87 (12H, m), 7.32 (2H, s), 7.98 (2H, s).

### Example 13

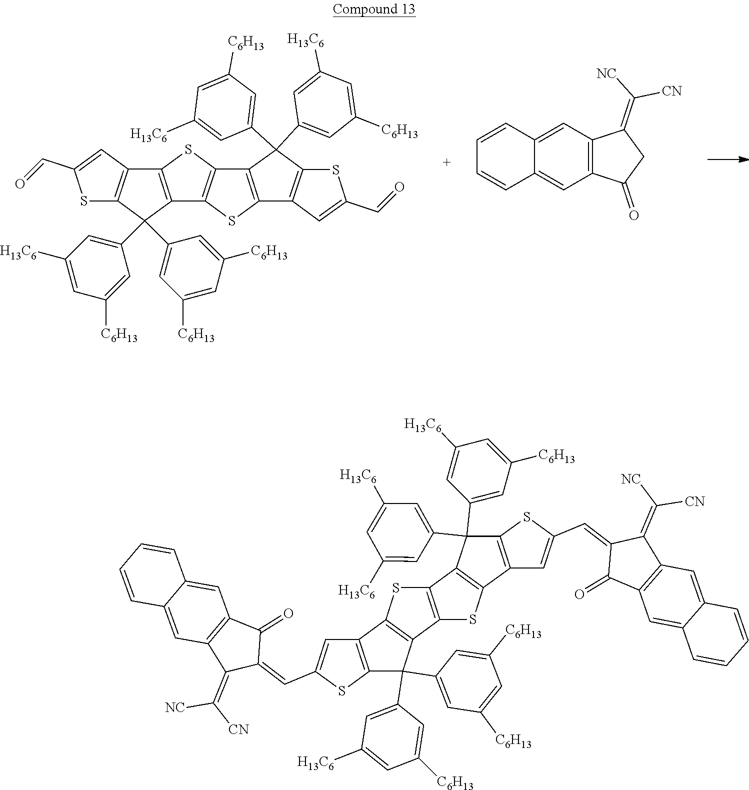

To a degassed solution of compound 10-5 (150 mg, 0.11 mmol) and pyridine (0.6 cm³) in anhydrous chloroform (24 cm³) at 0° C. was added 2-(3-oxo-2,3-dihydro-cyclopenta[b]naphthalen-1-ylidene)-malononitrile (134 mg, 0.551 mmol) in one portion. The resulting solution was then degassed for a further 30 minutes. The ice bath was removed and the reaction mixture warmed to 23° C. and stirred for 18 hours. The reaction mixture was diluted with acetonitrile (150 cm³) and stirred for 1 hour. The solid collected by filtration and washed with acetonitrile (100 cm³) and methanol (100 cm³) to give compound 13 (164 mg, 82%) as a dark solid. ¹H-NMR (400 MHz, CDCl₃) 0.66-0.83 (24H, m), 1.18 (48H, d, J 3.4), 1.37-1.58 (16H, m), 2.45 (16H, t, J 7.6), 6.77-6.89 (12H, m), 7.56-7.65 (4H, m), 7.78-7.88 (2H, m), 7.91-8.06 (4H, m), 8.27 (2H, s), 8.89 (2H, s), 9.08-9.16 (2H, m).

### Example 14

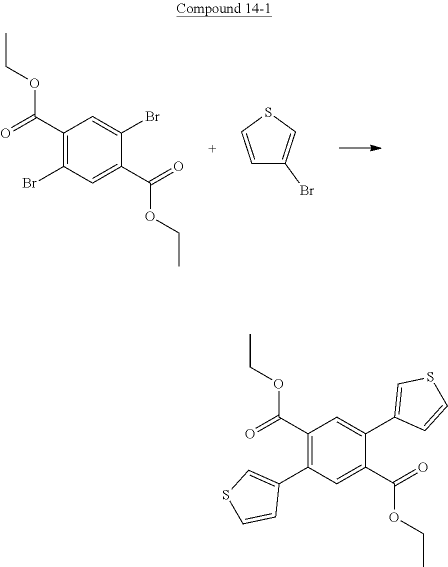 [SMILES: CCOC(=O)c1cc(Br)c(C(=O)OCC)cc1Br ; Brc1ccsc1 ; CCOC(=O)c1cc(-c2ccsc2)c(C(=O)OCC)cc1-c1ccsc1]

To a solution of 3-bromo-thiophene (30.9 g, 189 mmol) and anhydrous diethyl ether (310 cm³) at −78° C. was added n-butyllithium (83.4 cm³, 208 mmol, 2.5 M in hexanes) dropwise over 50 minutes and then stirred for 1 hour. Zinc chloride (116 cm³, 221 mmol, 1.9 M in 2-methyltetrahydrofuran) was then added dropwise over 20 minutes and the reaction mixture warmed to 0° C. and stirred for a further 1 hour. 2, 5-Dibromo-terephthalic acid diethyl ester (24.0 g, 63.2 mmol) and [1,1′-bis(diphenylphosphino)ferrocene]dichloropalladium(II) (2.77 g, 3.79 mmol) were then added. The resulting reaction mixture was then heated at reflux for 17 hours. After allowing to cool to 23° C., water (250 cm³) was added and the organics extracted with ethyl acetate (4×200 cm³). The combined organics were washed with brine (100 cm³), dried over anhydrous magnesium sulphate, filtered and the solvent removed in vacuo. The crude was purified by silica plug (dichloromethane) followed by recrystallisation (heptane) to give compound 14-1 (19.8 g, 81%) as an off white solid. ¹H-NMR (400 MHz, CDCl₃) 1.14 (6H, t, J 7.1), 4.20 (4H, q, J 7.1), 7.11-7.17 (2H, m), 7.32 (2H, dd, J 2.9, 1.2), 7.37 (2H, dd, J 4.9, 2.9), 7.81 (2H, s).

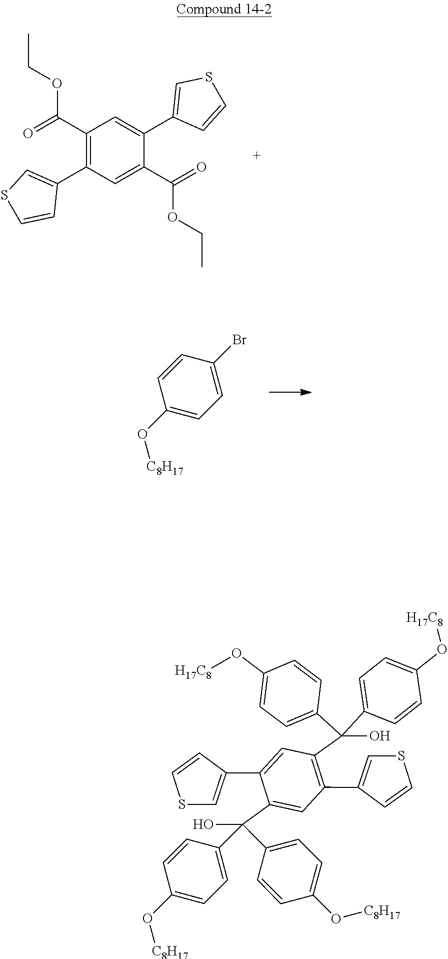

To a mixture of 1-bromo-4-octyloxy-benzene (70.1 g, 246 mmol) and anhydrous tetrahydrofuran (190 cm³) at −40° C. was added n-butyllithium (104 cm³, 261 mmol, 2.5 M in hexane) dropwise over 1 hour. Once the addition was complete, the reaction mixture was stirred at −50° C. for 1 hour. Compound 14-1 (19.0 g, 49.2 mmol) was then added and the reaction mixture stirred for 17 hours and allowed to warm to 23° C. Water (100 cm³) was then added and the mixture stirred for 20 minutes before the organics were extracted with dichloromethane (2×200 cm³). The organics combined, washed with brine (100 cm³) and dried over anhydrous magnesium sulphate, filtered and the solvent removed in vacuo. Trituration in acetonitrile followed by recrystallisation (acetonitrile/ethyl acetate) then gave compound 14-2 (32.7 g, 59%) as a beige solid. ¹H-NMR (400 MHz, CDCl₃) 0.84-0.96 (12H, m) 1.22-1.40 (32H, m) 1.40-1.52 (8H, m) 1.73-1.84 (8H, m) 3.20 (2H, s), 3.95 (8H, t, J 6.6), 6.43-6.52 (4H, m), 6.67 (2H, s), 6.80 (8H, d, J 8.8), 7.07 (8H, d, J 8.8), 7.13 (2H, dd, J 4.9, 3.1).

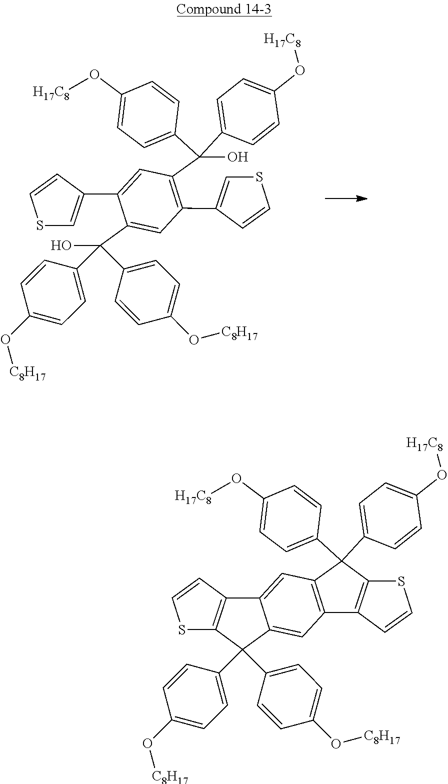

To a solution of compound 14-2 (32.0 g, 28.6 mmol) in dichloromethane (385 cm³) was added p-toluene sulfonic acid hydrate (10.9 g, 57.2 mmol) and the reaction mixture heated at reflux for 4 hours. The mixture was allowed to cool to 23° C., the solid removed by filtration and the solvent removed in vacuo. The crude was purified by silica plug using a graded solvent system (heptane:dichloromethane; 1:0 to 1:1) to give compound 14-3 (30.4 g, 98%) as a viscous yellow oil. ¹H-NMR (400 MHz, CDCl₃) 0.66-0.77 (12H, m), 1.03-1.20 (32H, m), 1.20-1.31 (8H, m), 1.52-1.63 (8H, m), 3.73 (8H, t, J 6.5), 6.61 (8H, d, J 8.8), 6.96 (2H, d, J 5.0), 7.01 (8H, d, J 8.7), 7.10 (2H, s), 7.18 (2H, d, J 5.0).

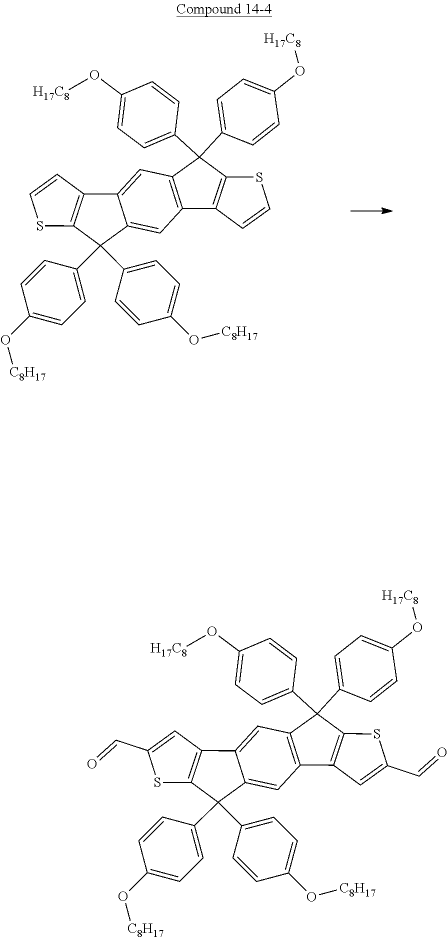

To a solution of anhydrous N,N-dimethylformamide (5.1 cm³, 66 mmol) and anhydrous chloroform (100 cm³) at 0° C. was added dropwise phosphorous oxychloride (5.2 cm³, 55 mmol) and then the reaction mixture stirred at 0° C. for 10 minutes and then allowed to warm to 23° C. A solution of compound 14-3 (8.00 g, 7.38 mmol) in chloroform (60 cm³) was then added dropwise over 40 minutes and the resulting solution stirred for 1 hour and then heated at 70° C. for 17 hours.

Further anhydrous N,N-dimethylformamide (5.1 cm³, 66 mmol) and phosphorous oxychloride (5.2 cm³, 55 mmol) were added and the mixture heated at 70° C. for 17 hours. The reaction mixture was allowed to cool to 50° C., an aqueous saturated solution of sodium acetate (100 cm³) was added and the reaction mixture stirred at 50° C. for 1 hour. The mixture was then cooled to 23° C. and poured into water (100 cm³). The aqueous layer was extracted with dichloromethane (2×100 cm³) and the combined organics washed with brine (100 cm³), dried over anhydrous magnesium sulfate, filtered and the solvent removed in vacuo. The crude was triturated (n-pentane) and recrystallised (heptane/dichloromethane) to give compound 14-4 (6.0 g, 71%) as pale-yellow crystals. ¹H-NMR (400 MHz, CDCl₃) 0.74-0.98 (12H, m), 1.18-1.37 (32H, m), 1.37-1.51 (8H, m), 1.66-1.88 (8H, m), 3.91 (8H, t, J 6.5), 6.80 (8H, d, J 8.9), 7.15 (8H, d, J 8.8), 7.52 (2H, s). 7.82 (2H, s), 9.89 (2H, s).

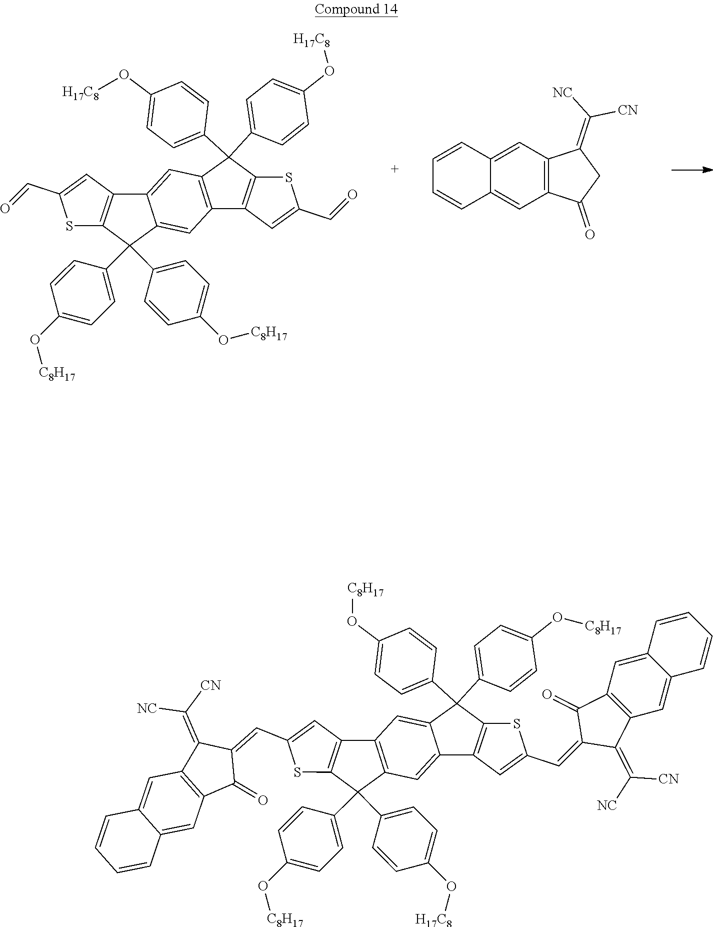

To a degassed solution of compound 14-4 (200 mg, 0.18 mmol) and pyridine (1.0 cm³) in anhydrous chloroform (24 cm³) at 0° C. was added 2-(3-oxo-2,3-dihydro-cyclopenta[b]naphthalen-1-ylidene)-malononitrile (214 mg, 0.877 mmol) in one portion. The resulting solution was then degassed for a further 30 minutes. The ice bath was removed and the reaction mixture warmed to 23° C. and stirred for 18 hours. The reaction mixture was diluted with acetone (150 cm³) and stirred for 1 hour. The solid collected by filtration and washed with diethyl ether (100 cm³) and dichloromethane (100 cm³) to give compound 14 (220 mg, 79%) as a poorly soluble dark solid.

### Example 15

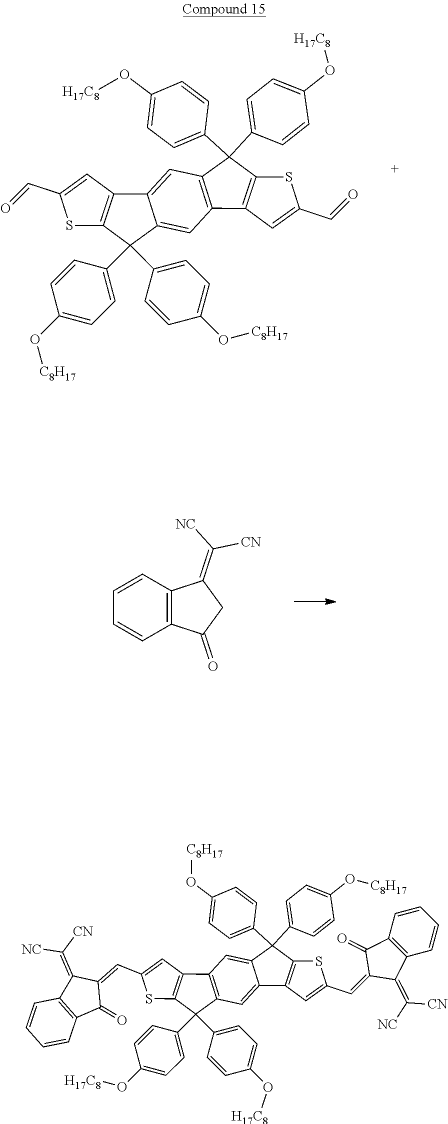

To a degassed solution of compound 14-4 (200 mg, 0.18 mmol) and pyridine (1.0 cm³) in anhydrous chloroform (24 cm³) at 0° C. was added 2-(3-oxo-2,3-dihydro-1H-inden-1-ylidene)propanedinitrile (170 mg, 0.877 mmol) in one portion. The resulting solution was then degassed for a further 30 minutes. The ice bath was removed and the reaction mixture warmed to 23° C. and stirred for 18 hours. The react ion mixture was diluted with acetonitrile (150 cm³) and stirred for 1 hour. The solid collected by filtration and washed with acetonitrile (100 cm³) and methanol (100 cm³) to give compound 15 (252 mg, 96%) as a dark solid. ¹H-NMR (400 MHz, CDCl₃) 0.82-0.96 (12H, m), 1.18-1.52 (40H, m), 1.69-1.86 (8H, m), 3.94 (8H, t, J 6.5), 6.78-6.92 (8H, m), 7.17-7.26 (8H, m), 7.55-7.64 (2H, m), 7.69-7.85 (4H, m), 7.88-7.97 (2H, m), 8.04 (2H, s), 8.66-8.77 (2H, m), 8.95 (2H, s).

### Example 16

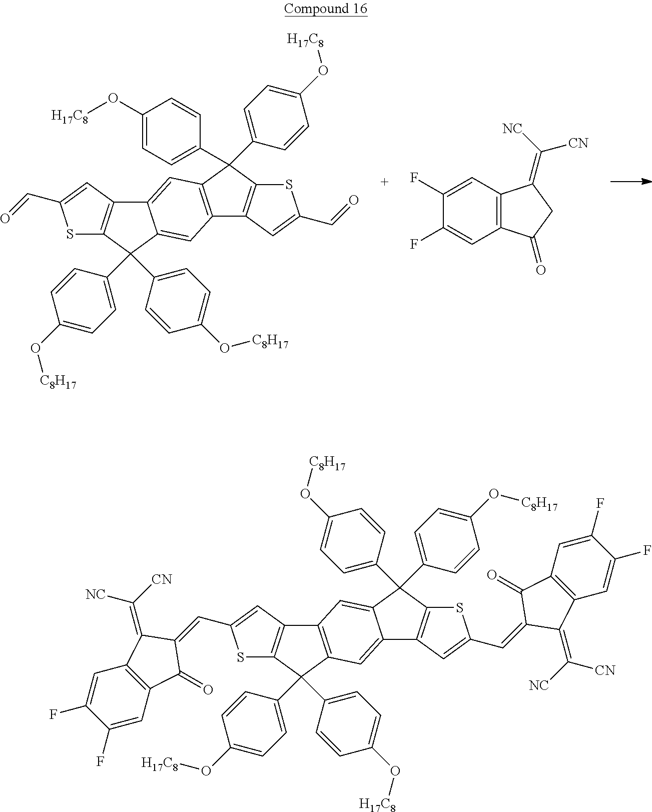

To a degassed solution of compound 14-4 (200 mg, 0.18 mmol) and pyridine (1.0 cm³) in anhydrous chloroform (24 cm³) at 0° C. was added 2-(5,6-difluoro-3-oxo-2,3-dihydro-1H-inden-1-ylidene)propanedinitrile (202 mg, 0.877 mmol) in one portion. The resulting solution was then degassed for a further 30 minutes. The ice bath was removed and the reaction mixture warmed to 23° C. and stirred for 18 hours. The reaction mixture was diluted with acetonitrile (150 cm³) and stirred for 1 hour. The solid collected by filtration and washed with acetonitrile (100 cm³) and methanol (100 cm³). The solid was then triturated in 40-60 petrol (100 cm³) and the solid collected by filtration to give compound 16 (273 mg, 99%) as a dark solid. ¹H-NMR (400 MHz, CDCl₃) 0.81-0.96 (12H, m), 1.18-1.52 (40H, m), 1.68-1.85 (8H, m), 3.94 (8H, t, J 6.5), 6.78-6.91 (8H, m), 7.14-7.26 (8H, m), 7.55-7.62 (2H, m), 7.69 (2H, t, J 7.5), 8.04 (2H, s), 8.56 (2H, dd, J 9.8, 6.6), 8.94 (2H, s).

### Example 17

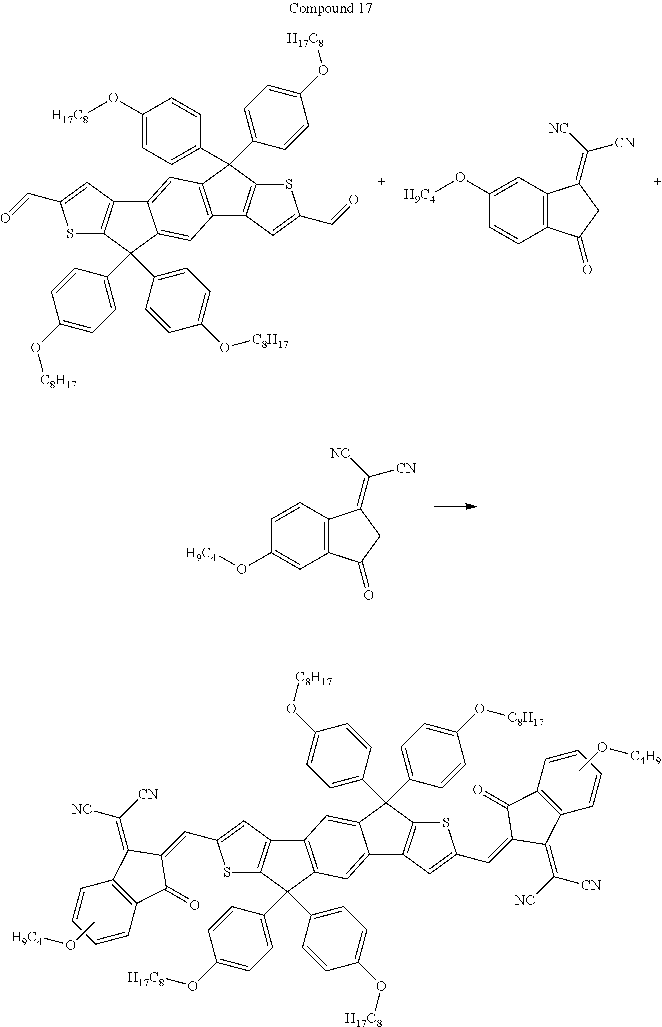

To a degassed solution of compound 14-4 (200 mg, 0.18 mmol) and pyridine (1.0 cm³) in anhydrous chloroform (24 cm³) at 0° C. was added a 4:1 mix of 2-(6-butoxy-3-oxo-2,3-dihydro-1H-inden-1-ylidene)propanedinitrile and 2-(5-butoxy-3-oxo-2,3-dihydro-1H-inden-1-ylidene)propanedinitrile (234 mg, 0.877 mmol) in one portion. The resulting solution was then degassed for a further 30 minutes. The ice bath was removed and the reaction mixture warmed to 23° C. and stirred for 18 hours. The reaction mixture was diluted with acetonitrile (150 cm³) and stirred for 1 hour. The solid collected by filtration and washed with acetonitrile (100 cm³) and methanol (100 cm³). The crude was purified by column chromatography using a graded solvent system (40-60 petrol:dichloromethane; 1:1 to 0:1) followed by trituration in acetone:acetonitrile (1:1, 25 cm³) and the solid collected by filtration, washed with n-pentane (50 cm³) to give compound 17 (124 mg, 43%) as a dark solid. ¹H-NMR (400 MHz, CDCl₃) 0.82-0.94 (12H, m), 1.02 (6H, t, J 7.5), 1.19-1.50 (44H, m), 1.70-1.96 (12H, m), 3.94 (8H, t, J 6.5), 4.08-4.21 (4H, m), 6.77-6.89 (8H, m), 7.14-7.27 (10H, m), 7.51-7.62 (2H, m), 7.78-7.88 (2H, m), 7.93-8.05 (2H, m), 8.16 (2H, d, J 2.0), 8.85 (2H, s).

### Example 18

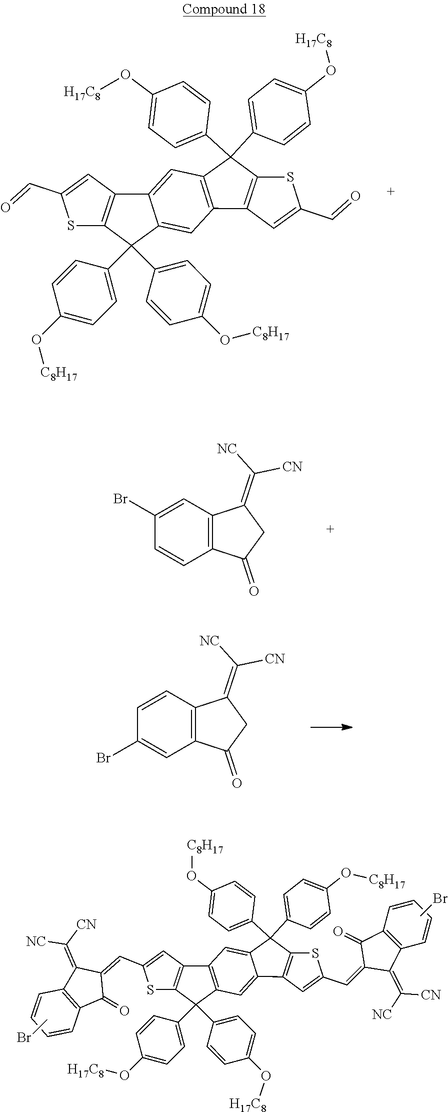

To a degassed solution of compound 14-4 (400 mg, 0.35 mmol) and pyridine (2.0 cm³) in anhydrous chloroform (24 cm³) at 0° C. was added a 2:3 mix of 2-(5-bromo-3-oxo-2,3-dihydro-1H-inden-1-ylidene)propanedinitrile and 2-(6-bromo-3-oxo-2,3-dihydro-1H-inden-1-ylidene)propanedinitrile (383 mg, 1.40 mmol) in one portion. The resulting solution was then degassed for a further 30 minutes. The ice bath was removed and the reaction mixture warmed to 23° C. an d stirred for 18 hours. The reaction mixture was diluted with acetonitrile (150 cm³) and stirred for 1 hour. The solid collected by filtration and washed with acetonitrile (100 cm³) and methanol (100 cm³) to give compound 18 (425 mg, 73%) as a dark solid. ¹H-NMR (400 MHz, CDCl₃) 0.82-1.00 (12H, m), 1.19-1.52 (40H, m), 1.68-1.89 (8H, m), 3.94 (8H, t, J 6.6), 6.79-6.93 (8H, m), 7.17-7.26 (8H, m), 7.60 (2H, s), 7.77 (1H, d, J 8.1), 7.85-7.92 (2H, m), 7.99-8.07 (3H, m), 8.56 (1H, d, J 8.6), 8.84 (1H, d, J 1.5), 8.95 (2H, s).

### Example 19

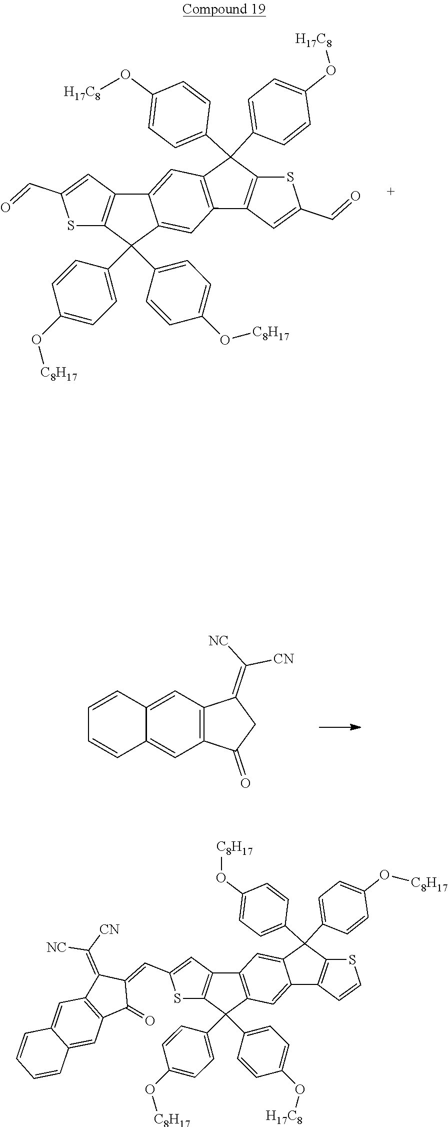

To a degassed solution of compound 14-4 (300 mg, 0.27 mmol) and pyridine (1.5 cm³) in anhydrous chloroform (24 cm³) at 0° C. was added 2-(3-oxo-2,3-dihydro-cyclopenta[b]naphthalen-1-ylidene)-malononitrile (132 mg, 0.540 mmol) in one portion. The resulting solution was then degassed for a further 30 minutes. The ice bath was removed and the reaction mixture warmed to 23° C. and stirred for 18 hours. The reaction mixture was diluted with acetone (150 cm³) and stirred for 1 hour. The solid collected by filtration and washed with acetonitrile (100 cm³) and methanol (100 cm³) to give compound 19 (325 mg, 90%) as a dark solid. ¹H-NMR (400 MHz, CDCl₃) 0.74-0.83 (12H, m), 1.11-1.44 (40H, m), 1.61-1.73 (8H, m), 3.84 (8H, t, J 6.1), 6.64-6.75 (8H, m), 7.02-7.25 (9H, m), 7.25-7.32 (1H, m), 7.35-7.42 (1H, m), 7.44-7.52 (1H, m), 7.53-7.65 (2H, m), 7.88-8.00 (3H, m), 8.22-8.32 (1H, m), 8.85-8.95 (1H, m), 9.06-9.17 (1H, m).

### Example 20

### Example 20-1

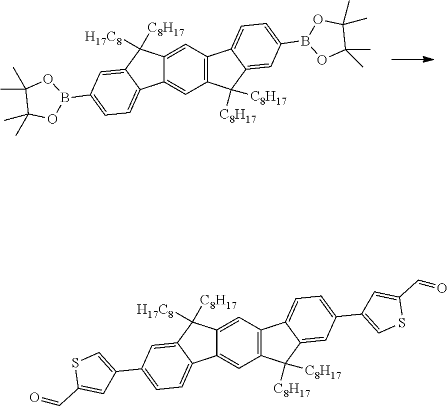

To a degassed solution of 2,2′-(6,12-dihydro-6,6,12,12-tetraoctylindeno[1,2-b]fluorene-2,8-diyl)bis[4,4,5,5-tetramethyl-1,3,2-dioxaborolane (1.00 g, 1.05 mmol), toluene (13 cm³), [1,4]-dioxane (13 cm³) and 4-bromo-thiophene-2-carbaldehyde (420 mg, 2.20 mmol) was added potassium phosphate tribasic (964 mg, 4.54 mmol) and tetrakis(triphenylphosphine)palladium(0) (30 mg, 0.026 mmol) and the solution further degassed for 1 hour. The reaction was then heated at 80° C. for 20 hours. The mixture was allowed to cool, filtered through cotton wool and the mixture concentrated in vacuo. The crude was then purified by column chromatography (40-60 petrol:ethyl acetate; 4:1) to give compound 20-1 (595 mg, 62%) as a light yellow oily solid. ¹H-NMR (400 MHz, CDCl₃) 0.62-0.73 (8H, m), 0.77 (12H, t, J 7.0), 0.96-1.21 (40H, m), 2.02-2.13 (8H, m), 7.55 (2H, d, J 1.2), 7.61 (2H, dd, J 7.9, 1.6), 7.64 (2H, s), 7.81 (2H, d, J 7.9), 7.92-7.95 (2H, m), 8.15 (2H, d, J 1.5), 10.02 (2H, d, J 1.2).

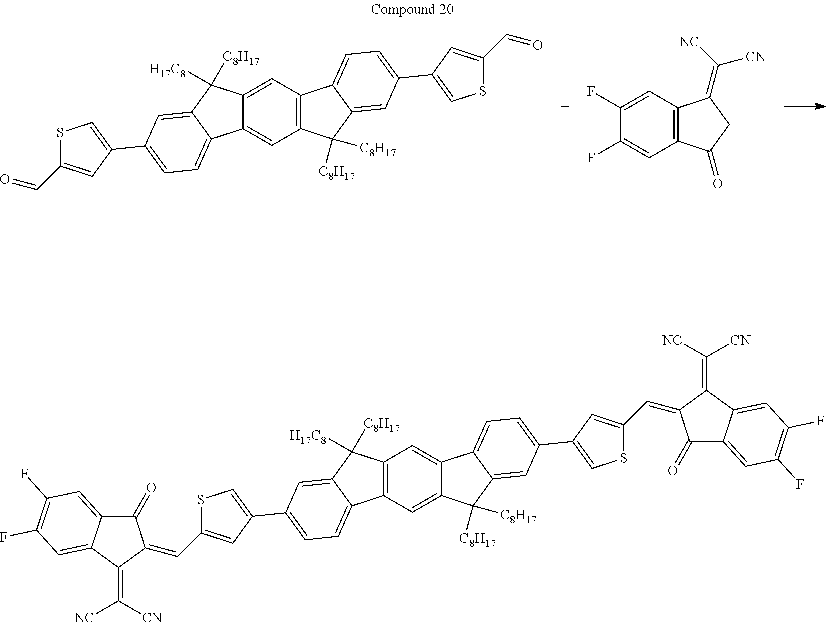

To a degassed solution of compound 20-1 (250 mg, 0.27 mmol), anhydrous chloroform (29 cm³) and pyridine (1.5 cm³) at 0° C. was added 2-(5,6-difluoro-3-oxo-indan-1-ylidene)-malononitrile (249 mg, 1.08 mmol). The reaction was stirred at 0° C. for 1.75 hours before it was added to stirred methanol (150 cm³) washing in with methanol (2×10 cm³). The suspension was stirred for 20 minutes and the precipitate collected by filtration. The collected solid was then washed with acetone (3×10 cm³), acetonitrile (3×10 cm³), 40-60 petrol (3×10 cm³) and cyclohexane (3×10 cm³) to give compound 20 (296 mg, 81%) as a dark purple/black solid.

¹H-NMR (400 MHz, CD₂Cl₂) 0.61-0.72 (8H, m), 0.76 (12H, t, J 6.9), 0.97-1.20 (40H, m), 2.05-2.17 (8H, m), 7.65 (2H, s), 7.69 (2H, dd, J 7.9, 1.4), 7.72 (2H, s), 7.74-7.80 (2H, m), 7.84 (2H, d, J 7.9), 8.26 (2H, s), 8.29 (2H, s), 8.58 (2H, dd, J 10.0, 6.6), 9.00 (2H, s).

### Example 21

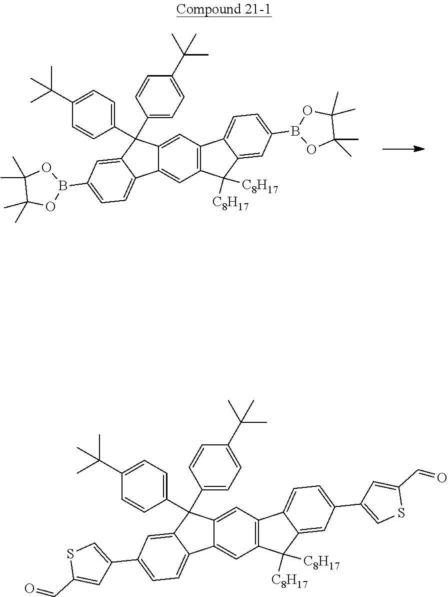

To a degassed solution of 2,2′-[6,6-bis[4-(1,1-dimethylethyl)phenyl]-6,12-dihydro-12,12-dioctylindeno[1,2-b]fluorene-2,8-diyl]bis[4,4,5,5-tetramethyl-1,3,2-dioxaborolane (1.50 g, 1.51 mmol), toluene (19 cm³), [1,4]-dioxane (19 cm³) and 4-bromo-thiophene-2-carbaldehyde (605 mg, 3.17 mmol) was added potassium phosphate tribasic (1.28 g, 6.03 mmol) and tetrakis(triphenylphosphine)palladium(0) (44 mg, 0.038 mmol) and the solution further degassed for 20 minutes. The reaction was then heated at 100° C. for 18 hours. The mixture was allowed to cool and the mixture concentrated in vacuo. Diethyl ether (100 cm³) and water (100 cm³) were then added. The aqueous layer was extracted with diethyl ether (50 cm³) and the combined organic extracts washed with water (50 cm³), brine (50 cm³) before drying over anhydrous magnesium sulfate, filtered and the solvent removed in vacuo. The crude was then purified by column chromatography using a graded solvent system (40-60 petrol:dichloromethane; 1:0 to 3:7) to give compound 21-1 (1.03 g, 71%) as a light brown solid. ¹H-NMR (400 MHz, CD₂Cl₂) 0.64-0.82 (10H, m), 1.01-1.21 (20H, m), 1.29 (18H, s), 2.04-2.19 (4H, m), 7.19-7.25 (4H, m), 7.26-7.33 (4H, m), 7.56-7.63 (2H, m), 7.64-7.72 (3H, m), 7.74 (1H, s), 7.81 (1H, s), 7.87-7.93 (2H, m), 7.96 (1H, t, J 1.3), 8.08 (1H, d, J 1.5), 8.15 (1H, d, J 1.5), 9.95 (1H, d, J 1.2), 9.99 (1H, d, J 1.2).

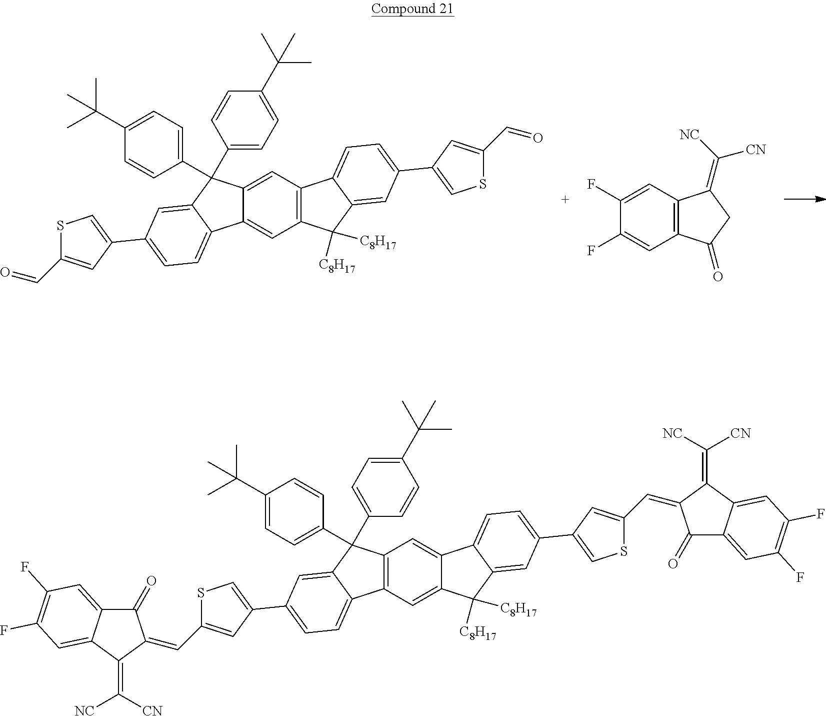

To a degassed solution of compound 21-1 (250 mg, 0.26 mmol), anhydrous chloroform (28 cm³) and pyridine (1.5 cm³) at 0° C. is added 2-(5,6-difluoro-3-oxo-indan-1-ylidene)-malononitrile (239 mg, 1.04 mmol). The reaction was stirred at 0° C. for 3.5 hours before it was added to stirred methanol (150 cm³) washing in with methanol (3×10 cm³). The suspension was stirred for 45 minutes and then allowed to stand for a further 45 minutes before the precipitate was collected by filtration. The solid was then washed with methanol (4×10 cm³). The solid was then triturated, stirring in boiling 2-butanone (50 cm³) and the solid collected by filtration of the hot suspension. The collected solid was then washed with acetone (4×10 cm³), diethyl ether (4×10 cm³), cyclohexane (4×10 cm³), 40-60 petrol (4×10 cm³) and acetonitrile (4×10 cm³) to give compound 21 (132 mg, 37%) as a dark red solid. ¹H-NMR (400 MHz, CD₂Cl₂) 0.65-0.82 (10H, m), 1.01-1.22 (20H, m), 1.29 (18H, s), 2.07-2.21 (4H, m), 7.20-7.27 (4H, m), 7.28-7.34 (4H, m), 7.61-7.66 (2H, m), 7.66-7.79 (6H, m), 7.82 (1H, s), 7.92 (1H, d, J 7.9), 8.12-8.15 (1H, m), 8.17-8.20 (1H, m), 8.22-8.25 (1H, m), 8.25-8.28 (1H, m), 8.50-8.61 (2H, m), 8.92 (1H, s), 8.98 (1H, s).

### Example 22

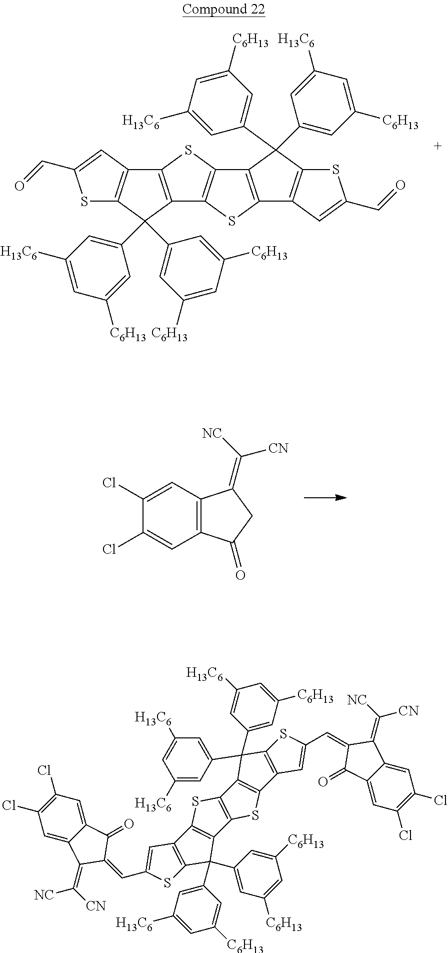

To a degassed solution of compound 10-5 (200 mg, 0.15 mmol) and pyridine (0.8 cm³) in anhydrous chloroform (8 cm³) at 0° C. was added 2-(5,6-dichloro-3-oxo-2,3-dihydro-1H-inden-1-ylidene)propanedinitrile (101 mg, 0.440 mmol). The resulting solution was then degassed for a further 30 minutes. The ice bath was removed and the reaction mixture warmed to 23° C. and stirred for 4 hours. The reaction mixture was diluted with acetonitrile (150 cm³) and stirred for 17 hours. The solid collected by filtration and washed with acetonitrile (100 cm³) and methanol (100 cm³) to give compound 22 (162 mg, 60%) as a dark solid. ¹H-NMR (400 MHz, CDCl₃) 0.75-0.92 (24H, m), 1.19-1.37 (48H, m), 1.49-1.65 (16H, m), 2.54 (16H, t, J 7.7), 6.83-7.00 (12H, m), 7.85-7.99 (4H, m), 8.80 (2H, s), 8.93 (2H, s).

### Example 23

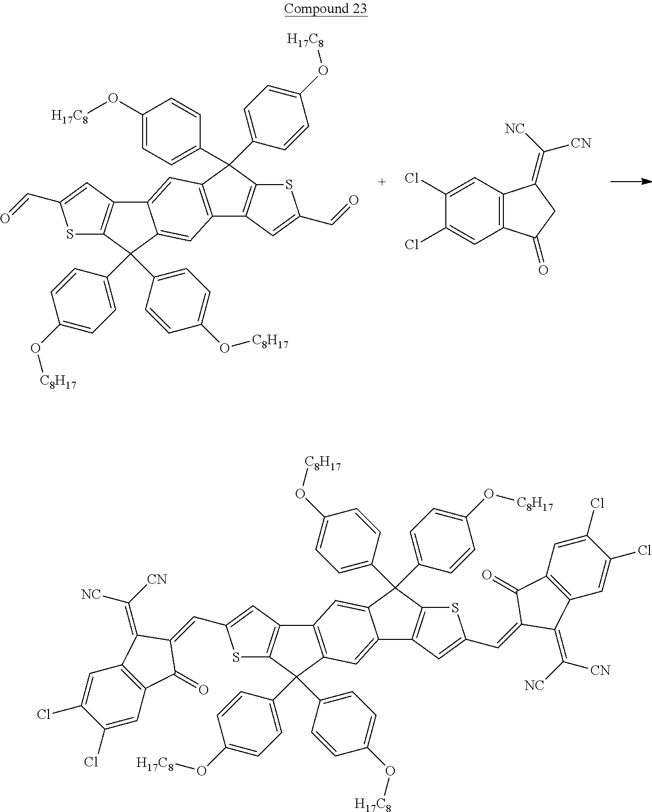

To a degassed solution of compound 14-4 (200 mg, 0.175 mmol) and pyridine (1.0 cm³) in anhydrous chloroform (24 cm³) at 0° C. was added 2-(5,6-dichloro-3-oxo-2,3-dihydro-1H-inden-1-ylidene)propanedinitrile (185 mg, 0.702 mmol) in one portion. The resulting solution was then degassed for a further 30 minutes. The ice bath was removed and the reaction mixture warmed to 23° C. and stirred for 4 hours. The reaction mixture was diluted with acetonitrile (150 cm³) and stirred for 17 hours. The solid collected by filtration and washed with acetonitrile (100 cm³) and methanol (100 cm³). The solid was then triturated in n-pentane (100 cm³) and the solid collected by filtration to give compound 23 (153 mg, 54%) as a dark solid. ¹H-NMR (400 MHz, CDCl₃) 0.80-0.97 (12H, m), 1.19-1.51 (40H, m), 1.70-1.84 (8H, m), 3.94 (8H, t, J 6.5), 6.77-6.92 (8H, m), 7.14-7.26 (8H, m), 7.55-7.64 (2H, m), 7.91-8.00 (2H, m), 8.05 (2H, s), 8.79 (2H, s), 8.95 (2H, s).

### Example 24

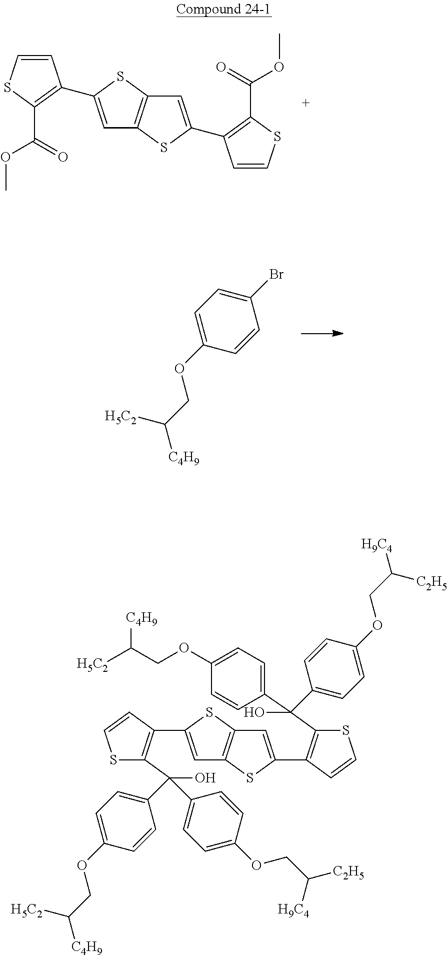

To a solution of 1-bromo-4-[(2-ethylhexyl)oxy]benzene (10.17 g, 35.67 mmol) in anhydrous tetrahydrofuran (127 cm³) at −78° C. was added dropwise tert-butyllithium (35.0 cm³, 59.4 mmol, 1.7 M in pentane) over 35 minutes keeping the reaction temperature below −50° C. Once the addition was finished the reaction mixture was stirred for 2 hours. The ice bath was then removed and the reaction was allowed to warm to about −40° C. and stirred for 15 minutes. The reaction mixture was then cooled to −78° C., compound 10-1 (2.50 g, 5.95 mmol) added and the reaction mixture stirred for 1 hour. The cooling was then removed and the resulting suspension was stirred at 23° C. for 17 hours. Water (100 cm³) was added and the mixture stirred for 1 hour. The organics were extracted with diethyl ether (3×100 cm³) and the combined organics washed with water (100 cm³), brine (100 cm³), dried over anhydrous magnesium sulfate, filtered and the solvent removed in vacuo.

The crude product was suspended in acetonitrile (150 cm³) stirred for 1 hour. The mixture was then let stand for 10 minutes and the upper solution was decanted. The crude was further purified by column chromatography using a graded solvent system (40-60 petrol:dichloromethane; 9:1 to 1:1) to give compound 24-1 (3.72 g, 53%) as a pale brown oil. ¹H-NMR (400 MHz, CDCl₃) 0.87-0.99 (24H, m), 1.25-1.55 (32H, m), 1.73 (4H, quin, J 6.1), 3.54 (2H, s), 3.85 (8H, d, J 5.9), 6.61 (2H, s), 6.79-6.86 (8H, m), 7.09 (2H, d, J 5.1), 7.15 (2H, d, J 5.1), 7.17-7.25 (8H, m).

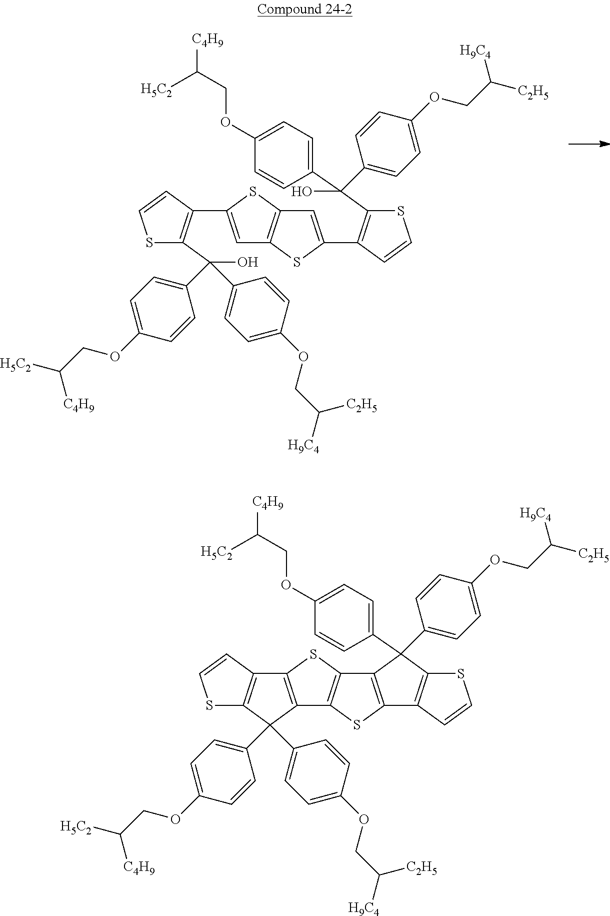

To a solution of p-toluene sulfonic acid monohydrate (1.32 g, 6.96 mmol) in chloroform (75 cm³) at 70° C. was added a solution of compound 24-1 (1.50 g, 1.16 mmol) in chloroform (75 cm³). The reaction mixture was heated at 70° C. for 65 hours. The reaction mixture was then cooled to 23° C. and the solvent removed in vacuo before water (50 cm³) was added. The organics were then extracted with ether (3×50 cm³). The combined organics layers were combined washed with brine, dried over anhydrous magnesium sulfate, filtered and the solvent removed in vacuo. Purification by column chromatography using a graded solvent system (40-60 petrol:dichloromethane: 9:1 to 7:3) to give compound 24-2 (585 mg, 44%) as a pale cream solid. ¹H-NMR (400 MHz, CDCl₃) 0.81-0.96 (24H, m), 1.20-1.57 (32H, m), 1.63-1.77 (4H, m), 3.74-3.87 (8H, m), 6.76-6.87 (8H, m), 7.01 (2H, d, J 4.9), 7.14-7.24 (8H, m), 7.36 (2H, d, J 4.9).

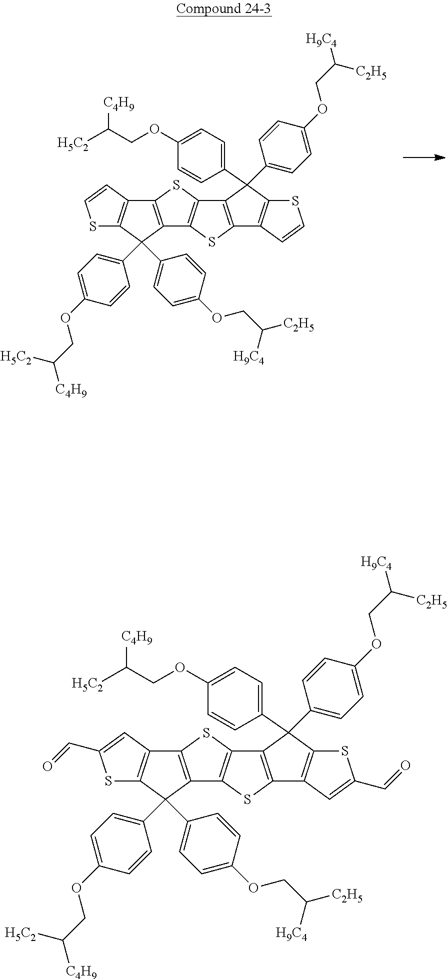

To a solution of N,N-dimethylformamide (0.22 cm³) in anhydrous chloroform (80 cm³) at 0° C. was added dropwise phosphorus(V) oxychloride (0.22 cm³, 2.40 mmol). After 15 minutes at 0° C., the reaction was stirred at 23° C. for 30 minutes.

Compound 24-2 (550 mg, 0.480 mmol) was then added to the reaction mixture and the reaction heated at reflux for 48 hours. Saturated aqueous sodium acetate solution (50 cm³) was then added and the resulting mixture was heated at 50° C. for 1 hour. The solution was then cooled to 23° C. and the solvent removed in vacuo. The organics extracted with ether (3×80 cm³) and the combined organics washed with brine (30 cm³), dried over anhydrous magnesium sulfate, filtered and the solvent removed in vacuo. The crude was purified by column chromatography using a graded solvent system (40-60 petrol:dichloromethane; 1:1 to 1:4) to give compound 24-3 (480 mg, 83%) as pale yellow solid. ¹H-NMR (400 MHz, CDCl₃) 0.71-0.92 (24H, m), 1.09-1.46 (32H, m), 1.60 (4H, dq, J 12.1, 5.8), 3.62-3.84 (8H, m), 6.66-6.82 (8H, m), 7.01-7.14 (8H, m), 7.50-7.62 (2H, m), 9.72-9.88 (2H, m).

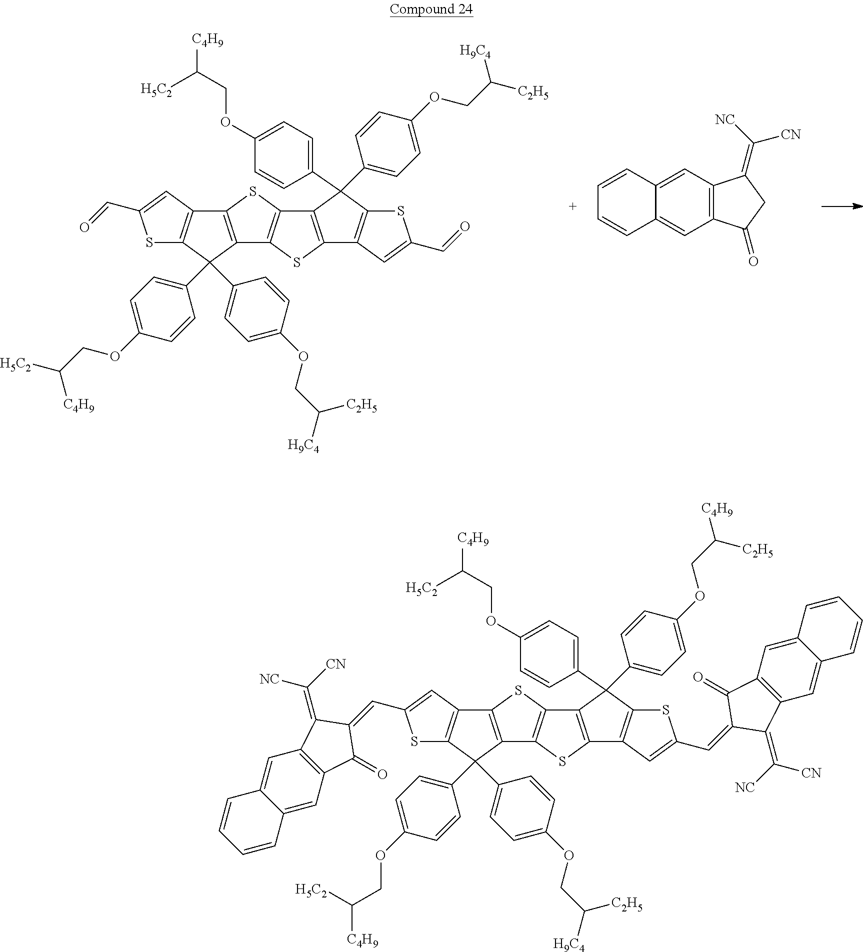

A degassed solution of compound 24-3 (150 mg, 0.13 mmol), pyridine (0.71 cm³) in anhydrous chloroform (10 cm³), 2-(3-oxo-2,3-dihydro-cyclopenta[b]naphthalen-1-ylidene)-malononitrile (152 mg, 0.624 mmol) was stirred for 17 hours. The reaction mixture was diluted with methanol (150 cm³) and stirred for 1 hour. The solid collected by filtration and washed with acetonitrile (100 cm³). The crude was triturated in ether (100 cm³) and the solid collected by filtration to give compound 24 (205 mg, 99%) as a dark solid. ¹H-NMR (400 MHz, CDCl₃) 0.76-0.87 (24H, m), 1.11-1.45 (32H, m), 1.57-1.68 (4H, m), 3.67-3.81 (8H, m), 6.75-6.86 (8H, m), 7.11-7.23 (8H, m), 7.56-7.68 (4H, m), 7.72-7.81 (2H, m), 7.97 (4H, ddd, J 12.2, 6.0, 3.6), 8.24-8.37 (2H, m), 8.88 (2H, s), 9.11 (2H, s).

### Example 25

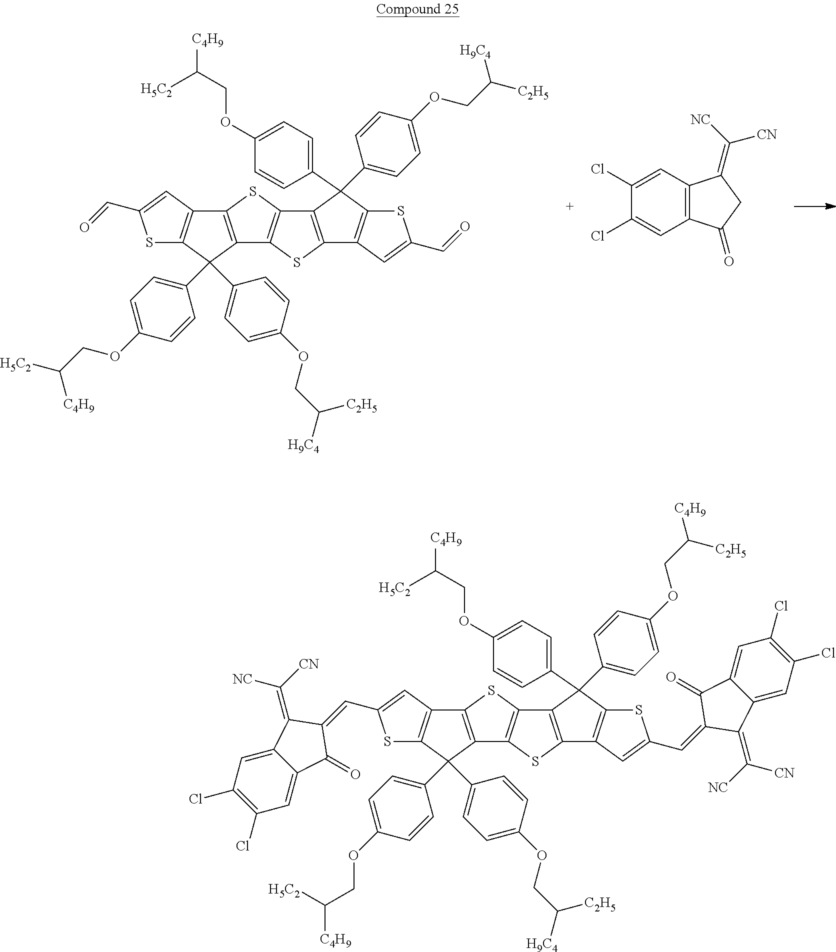

A degassed solution of compound 24-3 (150 mg, 0.13 mmol), pyridine (0.71 cm³) in anhydrous chloroform (10 cm³), 2-(5,6-dichloro-3-oxo-2,3-dihydro-1H-inden-1-ylidene)propanedinitrile (164 mg, 0.624 mmol) was stirred for 17 hours. The reaction mixture was diluted with methanol (150 cm³) and stirred for 1 hour. The solid collected by filtration and washed with acetonitrile (100 cm³). The crude was triturated in ether (100 cm³) and the solid collected by filtration to give compound 24 (199 mg, 94%) as a purple solid. ¹H-NMR (400 MHz, CDCl₃) 0.81 (24H, q, J 7.1), 1.13-1.48 (32H, m), 1.61 (4H, dt, J 11.9, 5.9), 3.66-3.80 (8H, m), 6.72-6.84 (8H, m), 7.08-7.17 (8H, m), 7.71-7.90 (4H, m), 8.70 (2H, s), 8.82 (2H, s).

### Example 26

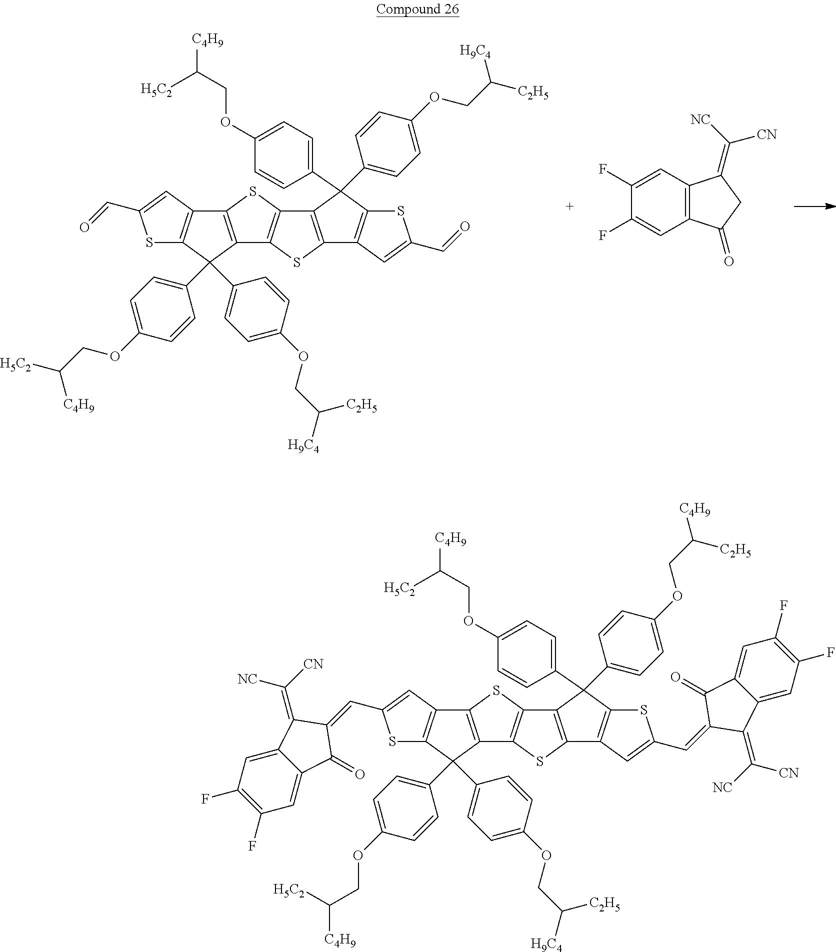

A degassed solution of compound 24-3 (150 mg, 0.13 mmol), pyridine (0.71 cm³) in anhydrous chloroform (10 cm³), 2-(5,6-difluoro-3-oxo-2,3-dihydro-1H-inden-1-ylidene)propanedinitrile (144 mg, 0.624 mmol) was stirred for 17 hours. The reaction mixture was diluted with methanol (150 cm³) and stirred for 1 hour. The solid collected by filtration and washed with acetonitrile (100 cm³) to give compound 25 (144 mg, 71%) as a blue/green solid. ¹H-NMR (400 MHz, CDCl₃) 0.82 (24H, q, J 7.0), 1.12-1.46 (32H, m), 1.61 (4H, quin, J 6.0), 3.68-3.76 (8H, m), 6.73-6.81 (8H, m), 7.08-7.17 (8H, m), 7.60 (2H, t, J 7.5), 7.77 (2H, s), 8.47 (2H, dd, J 9.8, 6.6), 8.80 (2H, s).

### Example 27

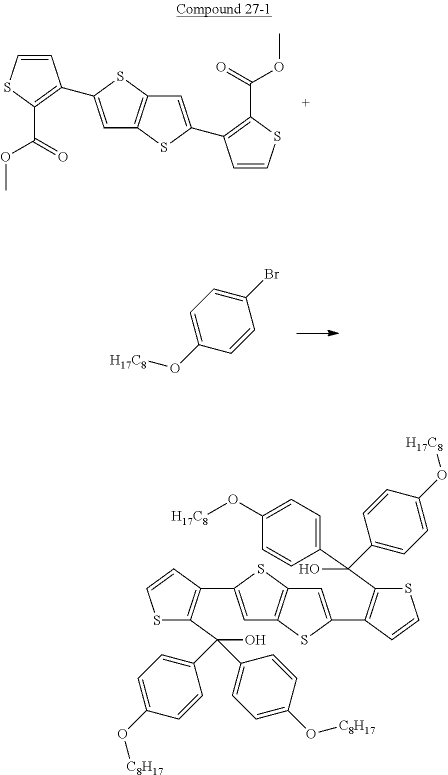

To a solution of 1-bromo-4-(octyloxy)benzene (6.06 g, 21.3 mmol) in anhydrous tetrahydrofuran (90 cm³) at −78° C. was added dropwise tert-butyllithium (25.0 cm³, 42.5 mmol, 1.7 M in pentane) keeping the reaction temperature below −60° C. After 20 minutes stirring at −78° C. the reaction was allowed to warm to −40° C. over 10 minutes before re-cooling to −78° C. After stirring at −78° C. for 15 minutes, additional 1-bromo-4-octyloxybenzene (606 mg, 2.13 mmol) was added to the reaction mixture and the reaction stirred for a further 15 minutes before the addition of compound 10-1 (1.79 g, 4.25 mmol). The reaction was stirred at −78° C. for 25 minutes before removing the cooling and allowing the reaction to warm to 23° C. over 17 hours. Water (10 cm³) was then added before the reaction mixture was added to a separating funnel with ether (100 cm³) and additional water (100 cm³). The organic layer was then washed with water (100 cm³), brine (100 cm³), dried over anhydrous magnesium sulfate, filtered and the solvent removed in vacuo. The crude purified by column chromatography using a graded solvent system (40-60 petrol:dichloromethane; 1:0 to 1:3) to give compound 27-1 (4.20 g, 84%) as a brown/green glassy oil. ¹H-NMR (400 MHz, CDCl₃) 0.91-0.85 (12H, m), 1.39-1.23 (32H, m), 1.45 (8H, p, J 6.8), 1.78 (8H, p, J 6.7), 3.52 (2H, s), 3.95 (8H, t, J 6.6), 6.60 (2H, s), 6.83-6.77 (8H, m), 7.08 (2H, d, J 5.2), 7.14 (2H, d, J 5.2), 7.22-7.17 (8H, m).

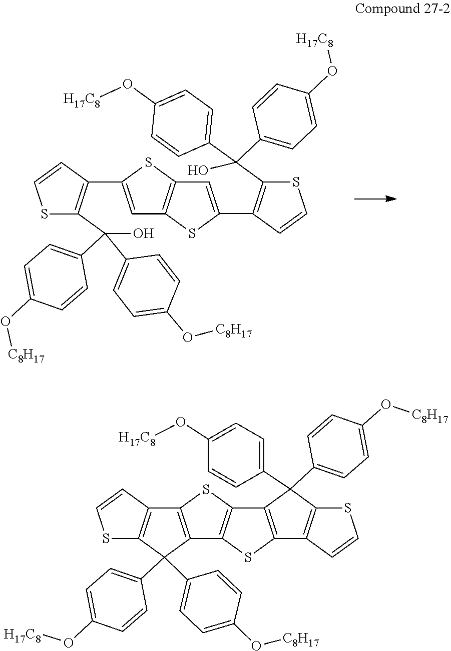

To a degassed solution of compound 27-1 (3.85 g, 3.26 mmol) in toluene (250 cm³) was added p-toluene sulfonic acid monohydrate (1.24 g, 6.51 mmol) and the mixture degassed for a further 10 minutes before stirring for 50 minutes and then heating at 50° C. for 16 hours. The reaction mixture cooled to 23° C. and concentrated in vacuo. Water (100 cm³) and ether (100 cm³) were then added. The organic layer was washed with water (100 cm³), brine (100 cm³) and dried over anhydrous magnesium sulfate, filtered and the solvent removed in vacuo. The crude was purified by column chromatography using a graded solvent system (40-60 petrol:dichloromethane; 1:0 to 7:3) to give compound 27-2 (1.03 g, 28%) as a yellow/brown oil. ¹H-NMR (400 MHz, CDCl₃) 0.92-0.84 (12H, m), 1.36-1.18 (32H, m), 1.45-1.36 (8H, m), 1.77-1.68 (8H, m), 3.89 (8H, t, J 6.5), 6.82-6.76 (8H, m), 6.98 (2H, d, J 5.0), 7.19-7.14 (8H, m), 7.34 (2H, d, J 5.0).

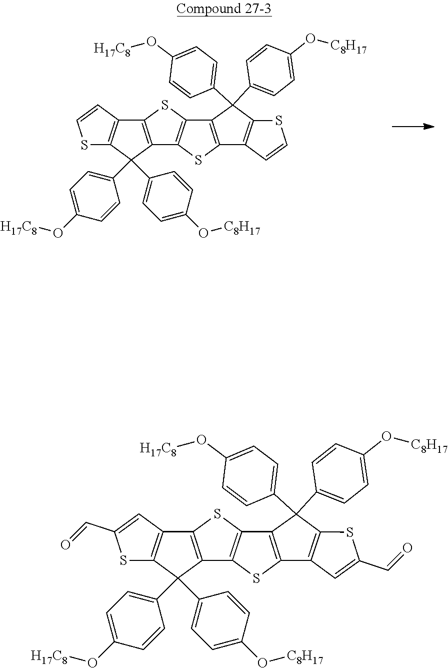

To a solution of N,N-dimethylformamide (0.41 cm³, 5.25 mmol) in anhydrous chloroform (30 cm³) at 0° C. was added dropwise phosphorous(V) oxychloride (0.41 cm³, 4.4 mmol). The mixture was then stirred for 10 minutes at 0° C. and at 23° C. for 30 minutes. A solution of compound 27-2 (1.00 g, 0.87 mmol) in chloroform (20 mL) is then added to the reaction mixture and then stirred at 23° C. f or 35 minutes before heating at 65° C. for 66 hours. The reaction was the n cooled to 23° C. before adding N,N-dimethylformamide (0.82 cm³, 10.5 mmol). After 5 minutes the reaction was cooled to 0° C. before adding phosphorous(V) oxychloride (0.82 cm³, 8.8 mmol). The reaction was stirred at 0° C. for 15 minutes before heating at reflux for 6 hours. The reaction was then cooled to 23° C. before adding N,N-dimethylformamide (0.82 cm³, 10.5 mmol). After 5 minutes the reaction was cooled to 0° C. before adding phosphorous(V) oxychloride (0.82 cm³, 8.8 mmol). The reaction was stirred at 0° C. for 10 minutes before heating at re flux for 19 hours. Saturated aqueous sodium acetate solution (20 cm³) was then added dropwise to portion wise via syringe. After a further 40 minutes at 75° C. the reaction was then cooled to 23° C. Water (100 cm³) and dichloromethane (50 cm³) were added. The organic layer was washed with water (100 cm³) and dried over anhydrous magnesium sulfate, filtered and the solvent removed in vacuo. The crude product was purified by column chromatography using a graded solvent system (40-60 petrol:dichloromethane; 1:0 to 3:7) to give compound 27-3 (763 mg, 73%) as a glassy yellow/brown solid. ¹H-NMR (400 MHz, CDCl₃) 0.89-0.84 (12H, m), 1.36-1.22 (32H, m), 1.45-1.37 (8H, m), 1.74 (8H, p, J 6.6), 3.90 (8H, t, J 6.5), 6.84-6.78 (8H, m), 7.17-7.11 (8H, m), 7.63 (2H, s), 9.87 (2H, s).

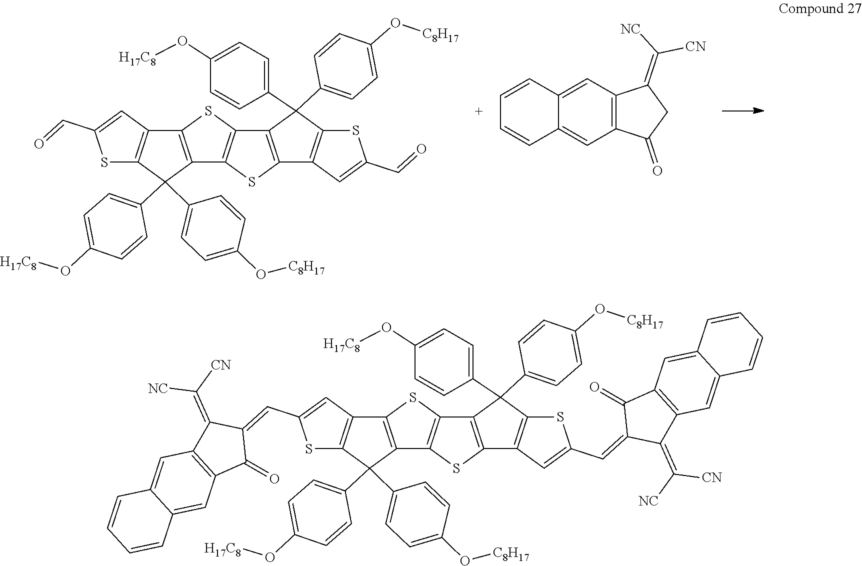

To a degassed solution of compound 27-3 (160 mg, 0.13 mmol), anhydrous chloroform (40 cm³) and pyridine (0.75 cm³) at 0° C. was added 2-(3-oxo-2,3-dihydro-cyclopenta[b]naphthalen-1-ylidene)-malononitrile (130 mg, 0.53 mmol). The reaction was stirred at 0° C. for 70 minutes, 23° C. for 85 minutes and at 40° C. for 110 minutes. The reaction mixture was then stirred at 23° C. for 10 minutes before the reaction mixture is poured into stirred methanol (150 cm³). The solid was collected by filtration and washed with methanol (4×10 cm³). The solid was then triturated in acetone (75 cm³) and the collected solid washed with acetone (2×10 cm³) and ether (3×10 cm³). The solid was then triturated in boiling acetone (50 cm³) and the collected solid washed with acetone (2×10 cm3) to give compound 27 as a (89 mg, 40%) as a black solid. ¹H-NMR (400 MHz, CDCl₃) 0.91-0.81 (12H, m), 1.37-1.19 (32H, m), 1.47-1.37 (8H, m), 1.75 (8H, p, J 6.6), 3.92 (8H, t, J 6.5), 6.90-6.82 (8H, m), 7.25-7.18 (8H, m), 7.74-7.66 (4H, m), 7.84 (2H, m), 8.11-8.00 (4H, m), 8.37 (2H, s), 8.97 (2H, s), 9.19 (2H, s).

### Example 28

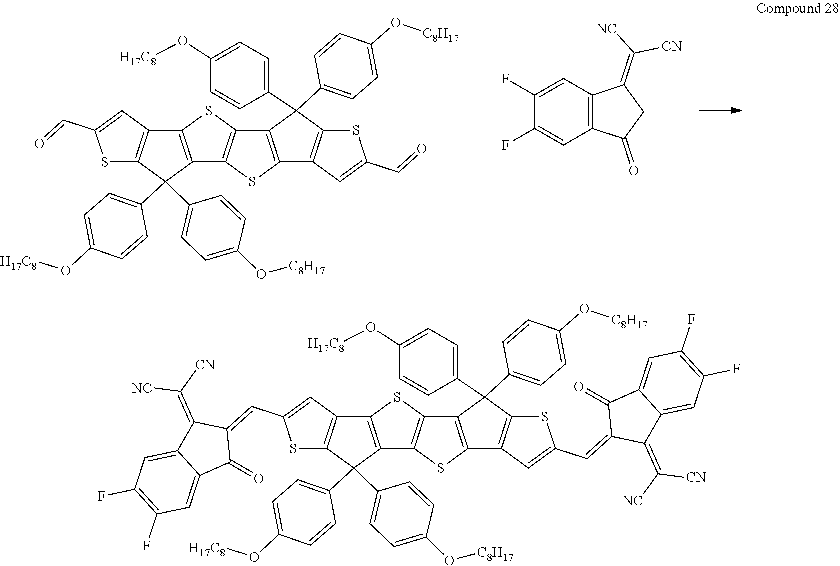

To a degassed solution of compound 27-3 (160 mg, 0.13 mmol), anhydrous chloroform (40 cm³) and pyridine (0.75 cm³) at 0° C. was added 2-(5,6-difluoro-3-oxo-indan-1-ylidene)-malononitrile (123 mg, 0.53 mmol). The reaction was stirred at 0° C. for 80 minutes, 23° C. for 85 minutes and at 40° C. for 120 minutes. The reaction mixture was then stirred at 23° C. for 5 minutes before the reaction mixture is poured into stirred methanol (150 cm³). The solid was collected by filtration and washed with methanol (4×10 cm³) to give compound 28 (173 mg, 80%) as a dark green solid.

¹H-NMR (400 MHz, CDCl₃) 0.89-0.82 (12H, m), 1.36-1.18 (32H, m), 1.46-1.37 (8H, m), 1.74 (8H, p, J 6.6), 3.91 (8H, t, J 6.5), 6.87-6.80 (8H, m), 7.23-7.16 (8H, m), 7.70-7.64 (2H, m), 7.84 (2H, s), 8.54 (2H, dd, J 9.7, 6.5), 8.87 (2H, s).

### Example 29

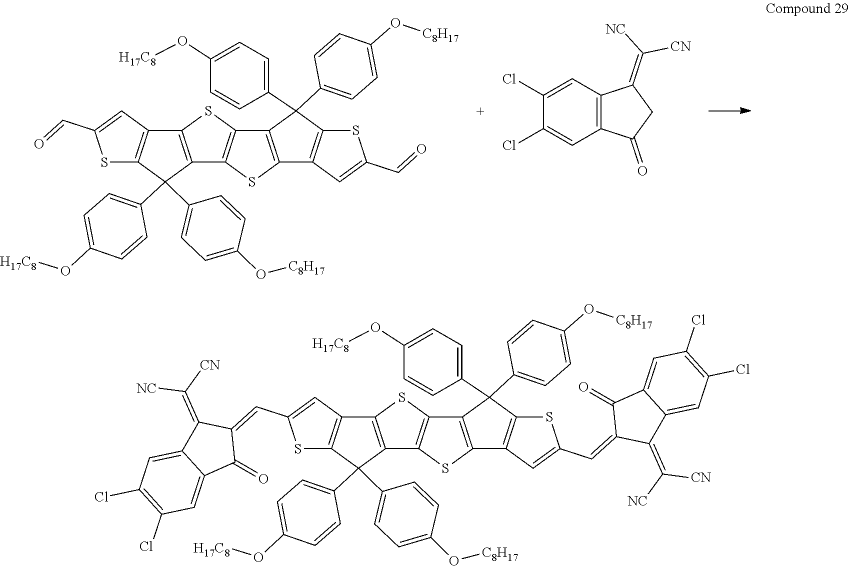

To a degassed solution of compound 27-3 (153 mg, 0.13 mmol), anhydrous chloroform (38 cm³) and pyridine (0.72 cm³) at 0° C. was added 2-(5,6-dichloro-3-oxo-indan-1-ylidene)-malononitrile (134 mg, 0.51 mmol). The reaction was stirred at slowly warmed to 23° C. over 21 hours before the reaction mixture is poured into stirred methanol (150 cm³). The solid was collected by filtration and washed with methanol (5×10 cm³) to give compound 29 (178 mg, 83%) as a black solid. ¹H-NMR (400 MHz, CDCl₃) 0.90-0.83 (12H, m), 1.36-1.19 (32H, m), 1.46-1.37 (8H, m), 1.78-1.70 (8H, m), 3.91 (8H, t, J 6.5), 6.87-6.81 (8H, m), 7.23-7.16 (8H, m), 7.84 (2H, s), 7.93 (2H, s), 8.77 (2H, s), 8.90 (2H, s).

### Example 30

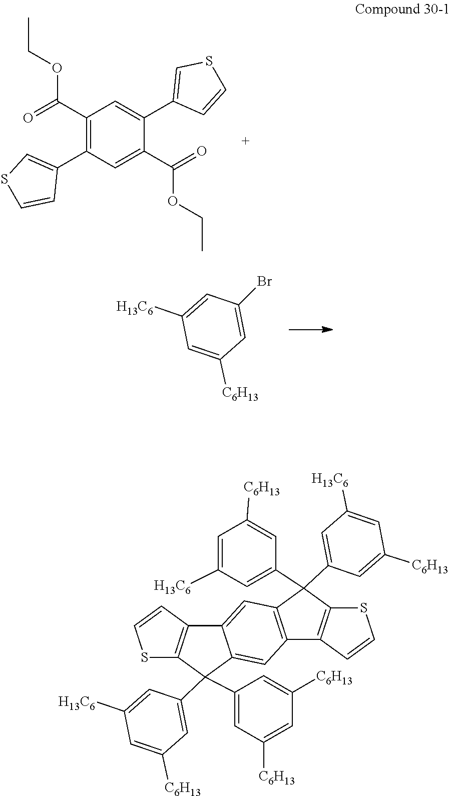

To a solution of 1-bromo-3,5-dihexylbenzene (26.5 g, 81.5 mmol) in anhydrous tetrahydrofuran (65 cm³) at −78° C. was added dropwise n-butyllithium (35.0 cm³, 86.4 mmol, 2.5 M in hexanes) and the reaction mixture stirred for 1 hour. Compound 14-1 (6.30 g, 16.3 mmol) was then added and the reaction mixture stirred at 23° C. for 17 hours. Water (50 cm³) was added, the mixture stirred for 1 hour and the organics extracted with dichloromethane (2×50 cm³). The combined organic was washed with brine (50 cm³), dried over anhydrous magnesium sulphate, filtered and the solvent removed in vacuo. The crude diol was dissolved in heptane and purified by column chromatography using a graded solvent system (heptane:dichloromethane; 19:1 to 1:4) to afford diol as a pale yellow oil (13.7 g). To a solution of diol (13.7 g, 11 mmol) in dichloromethane (165 cm³) was added p-toluene sulfonic acid monohydrate (4.1 g, 22 mmol). The mixture was heated at 40° C. for 2.5 hours before filtering and the solvent removed in vacuo. The crude is purified by column chromatography using a graded solvent system (heptane:dichloromethane; 9:1 to 1:1) to give compound 30-1 (13.0 g, 95%) as a yellow viscous oil. ¹H-NMR (400 MHz, CDCl₃) 0.77-0.96 (12H, m), 1.18-1.36 (48H, m), 1.45-1.59 (16H, m), 2.47 (16H, t, J 7.7), 6.80-6.92 (12H, m), 7.11 (2H, d, J 5.0), 7.34 (2H, m), 7.43 (2H, s).

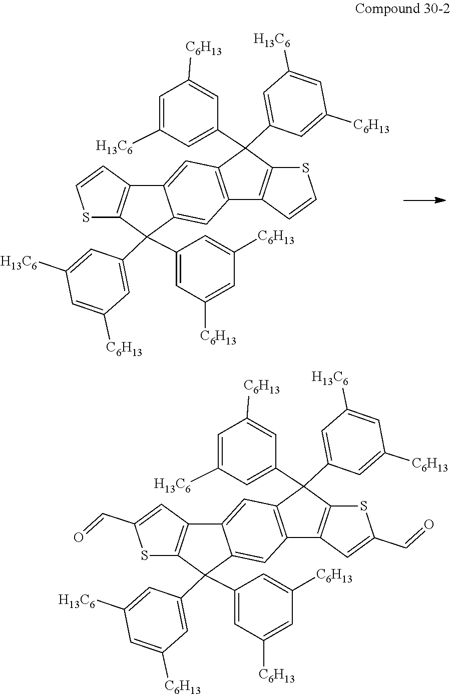

To a solution of anhydrous N,N-dimethylformamide (0.40 cm³, 4.8 mmol) in anhydrous chloroform (100 cm³) at 0° C. was added dropwise phosphorous (V) oxychloride (0.40 cm³, 4.0 mmol). The mixture was stirred for 10 minutes then warmed to 23° C. and stirred for 30 minutes. Compound 30-1 (1.00 g, 0.80 mmol) was then added and the resulting reaction mixture stirred at 23° C. for 1 hour then heated at 65° C. for 72 hours. The reaction mixture was then cooled to 45° C. and saturated aqueous sodium acetate solution (50 cm³) was added and the mixture stirred for 1 hour. The mixture was then cooled to 23° C. and the organic volatiles removed in vacuo. The residue was extracted with diethyl ether (3×50 cm³). The combined organics washed with brine (30 cm³), dried over anhydrous magnesium sulfate, filtered and the solvent removed in vacuo. The crude was crystallised from acetone and the solid washed with ice-cold acetonitrile (50 cm³) to give compound 30-2 (835 mg, 80%) as a pale yellow solid. ¹H-NMR (400 MHz, CDCl₃) 0.79-0.94 (24H, m), 1.17-1.37 (48H, m), 1.45-1.62 (16H, m), 2.40-2.59 (16H, m), 6.75-7.01 (12H, m), 7.53 (2H, s), 7.80 (2H, s), 9.90 (2H, s).

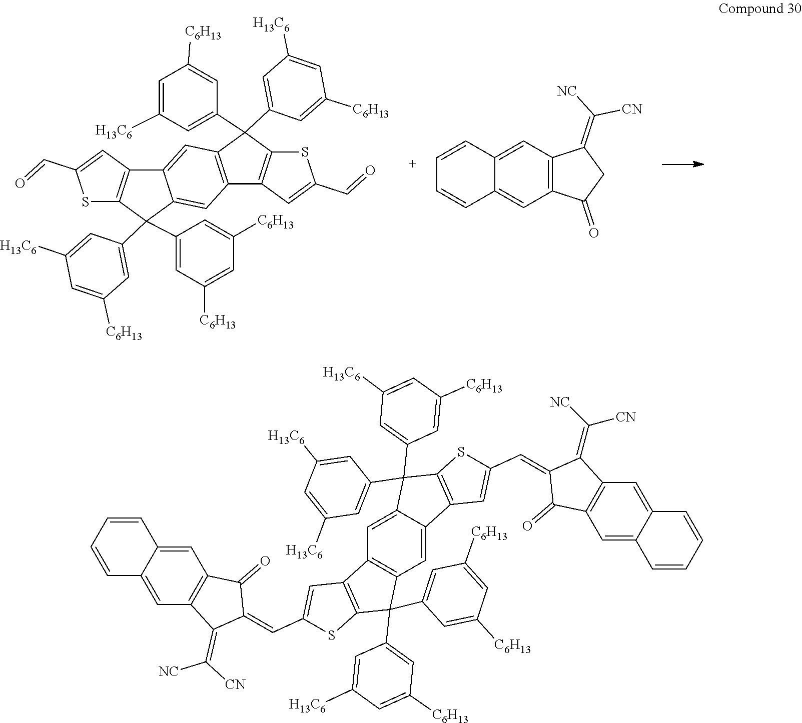

To a degassed solution of compound 30-2 (180 mg, 0.138 mmol) and anhydrous pyridine (0.78 cm³, 9.7 mmol) in anhydrous chloroform (11 cm³) at 0° C. was added 2-{3-oxo-1H,2H,3H-cyclopenta[b]naphthalen-1-ylidene}propanedinitrile (169 mg, 0.69 mmol). The resulting solution was then degassed for a further 30 minutes. The reaction mixture was warmed to 23° C. and stirred for 4 hours. Acetonitrile (150 cm³) was added and the mixture stirred for 1 hour. The solid collected by filtration and washed with acetonitrile (50 cm³) and diethyl ether (50 cm³) to give compound 30 (225 mg, 93%) as a green solid. ¹H-NMR (400 MHz, CDCl₃) 0.74-0.95 (24H, m), 1.18-1.38 (48H, m), 1.48-1.71 (16H, m), 2.55 (16H, t, J 7.7), 6.85-7.03 (12H, m), 7.58-7.65 (2H, m), 7.65-7.76 (4H, m), 7.96-8.16 (6H, m), 8.36 (2H, s), 9.00 (2H, s), 9.21 (2H, s).

### Example 31

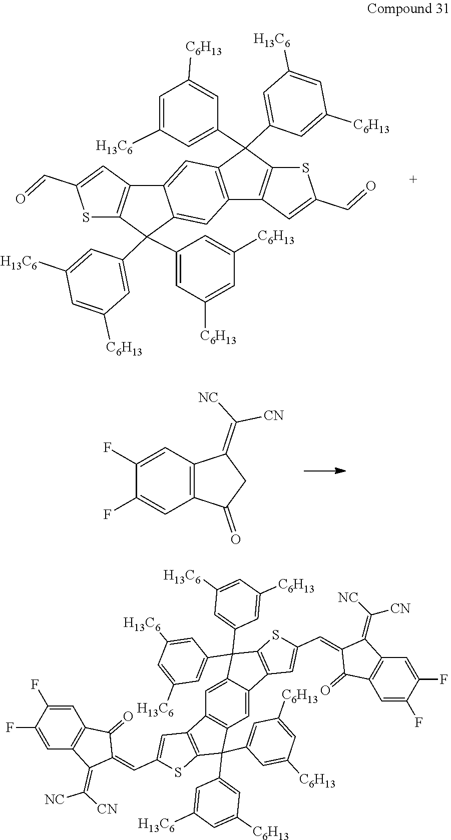

To a degassed solution of compound 30-2 (180 mg, 0.138 mmol) and anhydrous pyridine (0.78 cm³, 9.7 mmol) in anhydrous chloroform (11 cm³) at 0° C. was added 2-(5,6-difluoro-3-oxo-2,3-dihydro-1H-inden-1-ylidene)propanedinitrile (159 mg, 0.69 mmol). The resulting solution was then degassed for a further 30 minutes. The reaction mixture was warmed to 23° C. and stirred for 4 hours. Acetonitrile (150 cm³) was added and the mixture stirred for 1 hour. The solid collected by filtration and washed with acetonitrile (50 cm³) to give compound 31 (165 mg, 69%) as a dark blue/green solid. ¹H-NMR (400 MHz, CDCl₃) 0.66-0.81 (24H, m), 1.06-1.29 (48H, m), 1.37-1.58 (16H, m), 2.43 (16H, t, J 7.6), 6.73-6.92 (12H, m), 7.46-7.62 (4H, m), 7.95 (2H, s), 8.46 (2H, dd, J 9.8, 6.4).

### Example 32

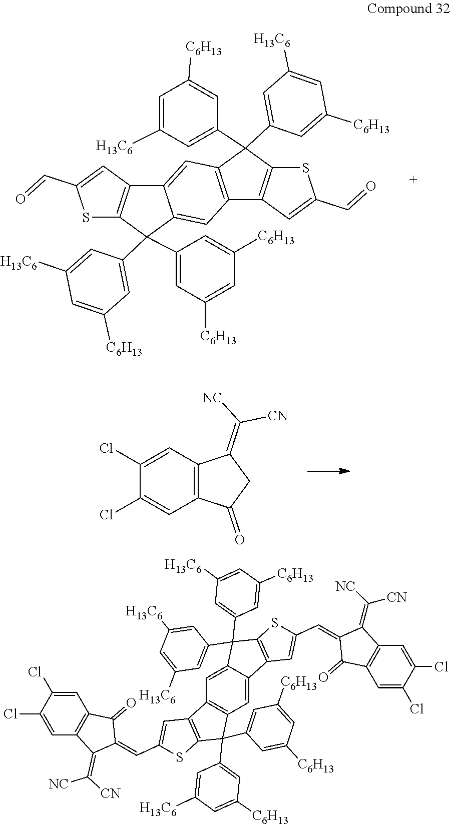

To a degassed solution of compound 30-2 (180 mg, 0.138 mmol) and anhydrous pyridine (0.78 cm³, 9.7 mmol) in anhydrous chloroform (11 cm³) at 0° C. was added 2-(5,6-dichloro-3-oxo-2,3-dihydro-1H-inden-1-ylidene)propanedinitrile (182 mg, 0.69 mmol). The resulting solution was then degassed for a further 30 minutes. The reaction mixture was warmed to 23° C. and stirred for 4 hours. Acetonitrile (150 cm³) was added and the mixture stirred for 1 hour. The solid collected by filtration and washed with acetonitrile (50 cm³) and acetone (50 cm³) to give compound 32 (110 mg, 44%) as a green solid. ¹H-NMR (400 MHz, CDCl₃) 0.74-0.93 (24H, m), 1.15-1.39 (48H, m), 1.46-1.65 (16H, m), 2.42-2.63 (16H, m), 6.82-7.04 (12H, m), 7.53-7.67 (2H, m), 7.93 (2H, s), 8.05 (2H, s), 8.79 (2H, s), 8.95 (2H, s).

### Example 33

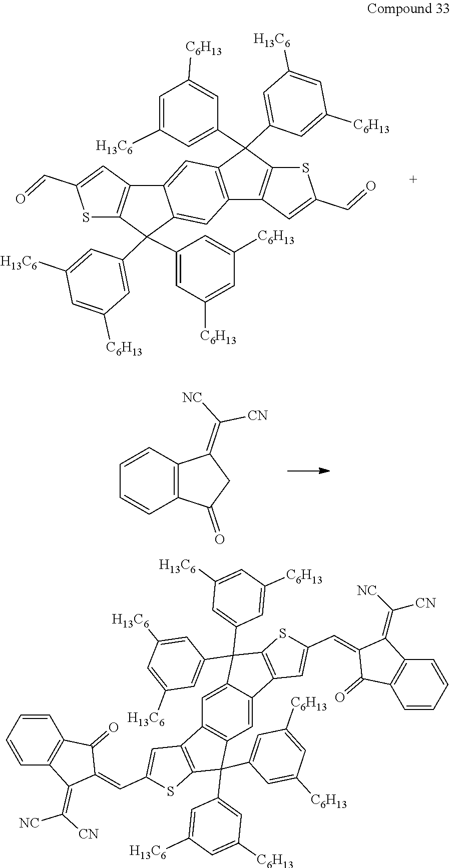

To a degassed solution of compound 30-2 (180 mg, 0.138 mmol) and anhydrous pyridine (0.78 cm³, 9.7 mmol) in anhydrous chloroform (11 cm³) at 0° C. was added 2-(3-oxo-2,3-dihydro-1H-inden-1-ylidene)propanedinitrile (134 mg, 0.69 mmol). The resulting solution was then degassed for a further 30 minutes. The reaction mixture was warmed to 23° C. and stirred for 4 hours. Acetonitrile (150 cm³) was added and the mixture stirred for 1 hour. The solid collected by filtration and washed with acetonitrile (50 cm³) to give compound 33 (219 mg, 96%) as a dark red solid. ¹H-NMR (400 MHz, CDCl₃) 0.66-0.81 (24H, m), 1.07-1.29 (48H, m), 1.38-1.55 (16H, m), 2.34-2.51 (16H, m), 6.75-6.90 (12H, m), 7.45-7.52 (2H, m), 7.67 (4H, quind, J 7.5, 7.5, 7.5, 7.5, 1.2), 7.76-7.85 (2H, m), 7.94 (2H, s), 8.55-8.67 (2H, m), 8.83 (2H, s).

### Example 34

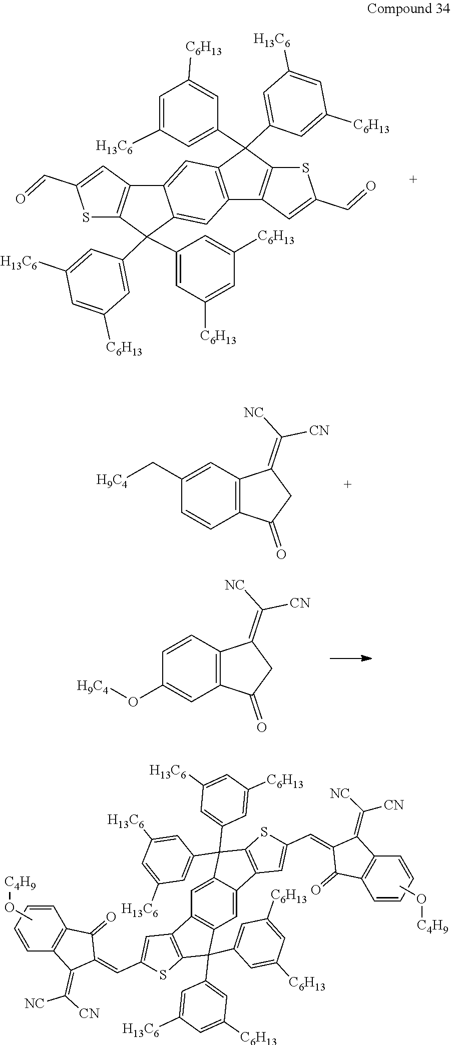

To a degassed solution of compound 30-2 (180 mg, 0.138 mmol) and anhydrous pyridine (0.78 cm³, 9.7 mmol) in anhydrous chloroform (11 cm³) at 0° C. was added a 4:1 mix of 2-(6-butoxy-3-oxo-2, 3-dihydro-1H-inden-1-ylidene)propanedinitrile and 2-(5-butoxy-3-oxo-2,3-dihydro-1H-inden-1-ylidene)propanedinitrile (184 mg, 0.69 mmol). The resulting solution was then degassed for a further 30 minutes. The reaction mixture was warmed to 23° C. and stirred for 4 hours. Acetonitrile (150 cm³) was added and the mixture stirred for 1 hour. The solid collected by filtration and washed with acetonitrile (50 cm³) to give compound 34 (240 mg, 97%) as a red solid.

¹H-NMR (400 MHz, CDCl₃) 0.67-0.82 (24H, m), 0.92 (6H, t, J 7.3), 1.09-1.30 (48H, m), 1.37-1.55 (16H, m), 1.68-1.82 (4H, m), 2.43 (16H, t, J 7.7), 4.05 (4H, t, J 6.4), 6.74-6.91 (12H, m), 7.08-7.16 (2H, m), 7.42-7.51 (2H, m), 7.71 (2H, d, J 8.3), 7.86-7.95 (2H, m), 8.06 (2H, d, J 2.2), 8.70-8.79 (2H, m).

### Example 35

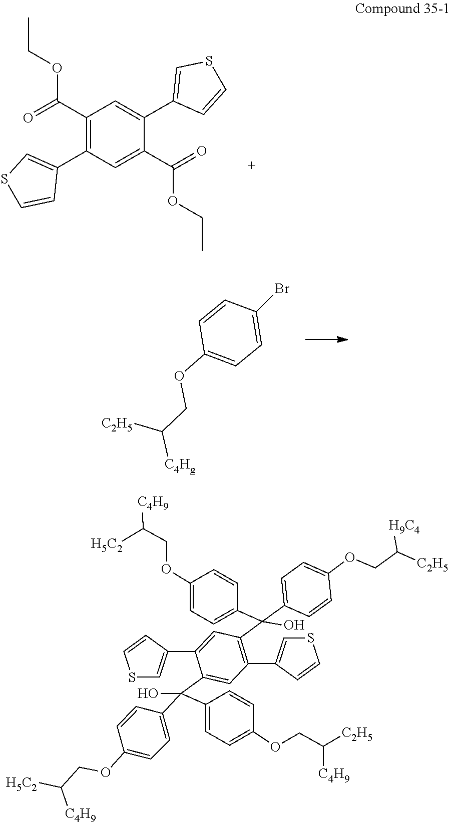

To a solution of 1-bromo-4-((2-ethylhexyl)oxy)benzene (22.1 g, 77.5 mmol) in anhydrous tetrahydrofuran (60 cm³) at −78° C. was added dropwise n-butyllithium (33.0 cm³, 83.2 mmol, 2.5 M in hexanes) and the reaction mixture stirred for 2 hours. Compound 14-1 (6.00 g, 15.5 mmol) was then added and the reaction mixture stirred at 23° C. for 17 hours. Water (50 cm³) was added, the mixture stirred for 1 hour and the organics extracted with dichloromethane (2×50 cm³). The combined organic was washed with brine (50 cm³), dried over anhydrous magnesium sulphate, filtered and the solvent removed in vacuo. The crude was triturated with acetonitrile (100 cm³). The solid collected by filtration and recrystalised (acetonitrile:ethyl acetate; 1:1) to give compound 35-1 (11.2 g, 64%) as a pale yellow solid. ¹H-NMR (400 MHz, D8-tetrahydrofuran) 0.87-0.98 (24H, m), 1.25-1.58 (32H, m), 1.68 (4H, m), 3.82 (8H, d, J 5.6), 4.36 (2H, s), 6.52 (2H, dd, J 4.9, 1.2), 6.61 (2H, dd, J 2.9, 1.1), 6.71 (8H, d, J 8.9), 6.97 (2H, s), 7.01 (2H, dd, J 4.9, 3.1), 7.07 (8H, d, J 8.8).

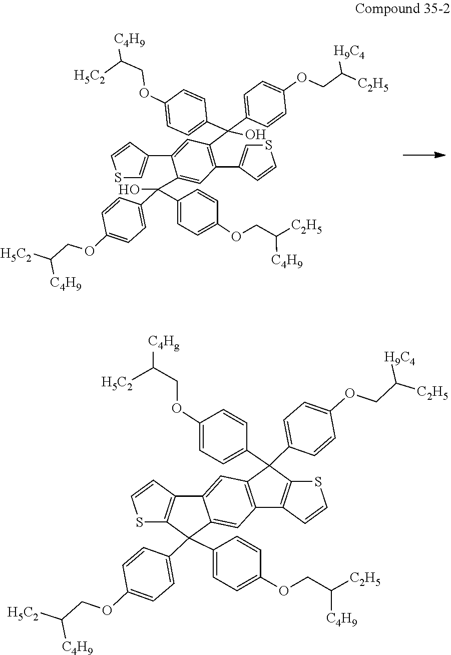

To a solution of compound 35-2 (11.0 g, 9.82 mmol) in dichloromethane (132 cm³) was added p-toluene sulfonic acid monohydrate (3.70 g, 19.7 mmol). The mixture was heated at 40° C. for 2 hours before filtering and the solvent removed in vacuo. The crude was purified by column chromatography using a graded solvent system (heptane:dichloromethane; 1:0 to 4:1) to give compound 35-2 (11.2 g, 100%) as a pale yellow viscous oil. ¹H-NMR (400 MHz, CDCl₃) 0.79-1.01 (24H, m), 1.24-1.54 (32H, m), 1.59-1.87 (4H, m), 3.79 (8H, d, J 5.6), 6.75-6.82 (8H, m), 7.11-7.23 (10H, m), 7.35 (2H, d, J 5.0), 7.44 (2H, s).

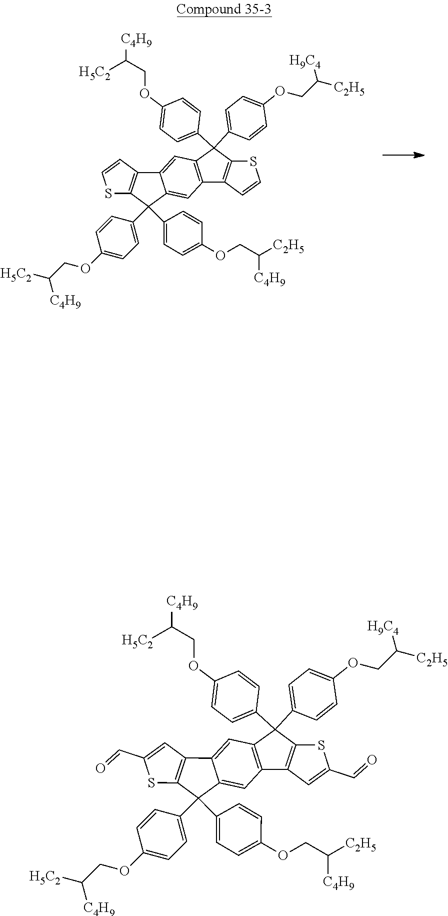

To a solution of anhydrous N,N-dimethylformamide (14.4 cm³, 186 mmol) in anhydrous chloroform (174 cm³) at 0° C. was added dropwise phosphorous (V) oxychloride (13.5 cm³, 145 mmol). The mixture was stirred for 10 minutes then warmed to 23° C. and stirred for 30 minutes. Compound 35-2 (1.00 g, 0.80 mmol) was then added. The resulting reaction mixture stirred at 23° C. for 1 hour then heated at 65° C. for 17 hours. The reaction mixture was then cooled to 50° C. and saturated aqueous sodium acetate solution (50 cm³) was added and the mixture stirred for 1 hour. The mixture was then cooled to 23° C. and extracted with dichloromethane (2×100 cm³). The combined organics washed with brine (30 cm³), dried over anhydrous magnesium sulfate, filtered and the solvent removed in vacuo. The crude was triturated with acetonitrile (200 cm³). The solid collected by filtration and recrystalised (heptane:ethyl acetate; 4:1) to give compound 35-3 (6.0 g, 51%) as a pale yellow solid. ¹H-NMR (400 MHz, CDCl₃) 0.81-1.00 (24H, m), 1.22-1.52 (32H, m), 1.70 (4H, m), 3.80 (8H, d, J 5.8), 6.81 (8H, d, J 8.8), 7.15 (8H, d, J 8.8), 7.52 (2H, s), 7.82 (2H, s), 9.89 (2H, s).

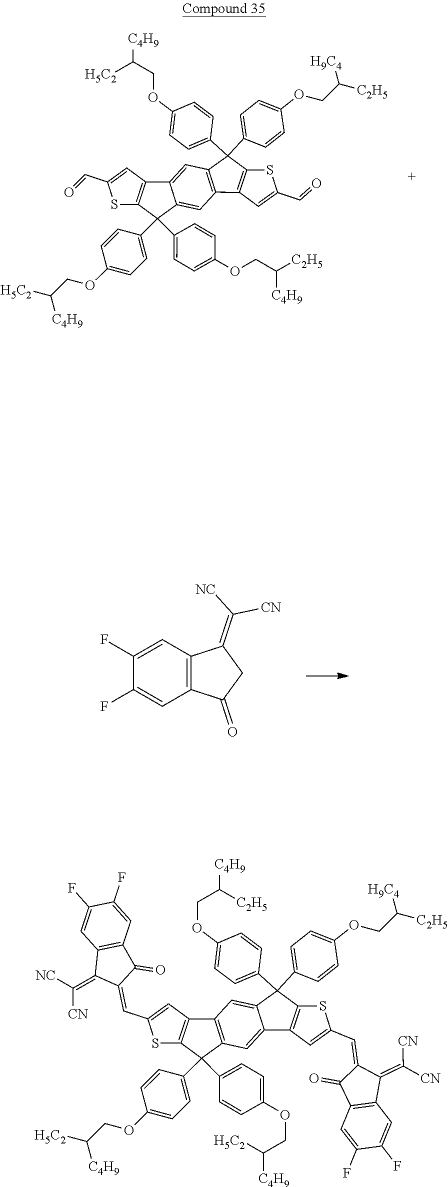

To a degassed solution of compound 35-3 (200 mg, 0.18 mmol) and anhydrous pyridine (0.99 cm³, 12 mmol) in anhydrous chloroform (14 cm³) at 0° C. was added 2-(5,6-difluoro-3-oxo-2,3-dihydro-1H-inden-1-ylidene)propanedinitrile (202 mg, 0.88 mmol). The resulting solution was then degassed for a further 30 minutes. The reaction mixture was warmed to 23° C. and stirred for 17 hours. Methanol (150 cm³) was added and the mixture stirred for 1 hour. The solid collected by filtration and washed with acetonitrile (50 cm³) to give compound 35 (270 mg, 98%) as a dark blue/green solid. ¹H-NMR (400 MHz, CDCl₃) 0.71-0.91 (24H, m), 1.12-1.50 (32H, m), 1.53-1.71 (4H, m), 3.65-3.82 (8H, m), 6.66-6.84 (8H, m), 7.04-7.17 (8H, m), 7.47-7.65 (4H, m), 7.87-8.00 (2H, m), 8.47 (2H, dd, J 9.8, 6.6), 8.75-8.90 (2H, m).

### Example 36

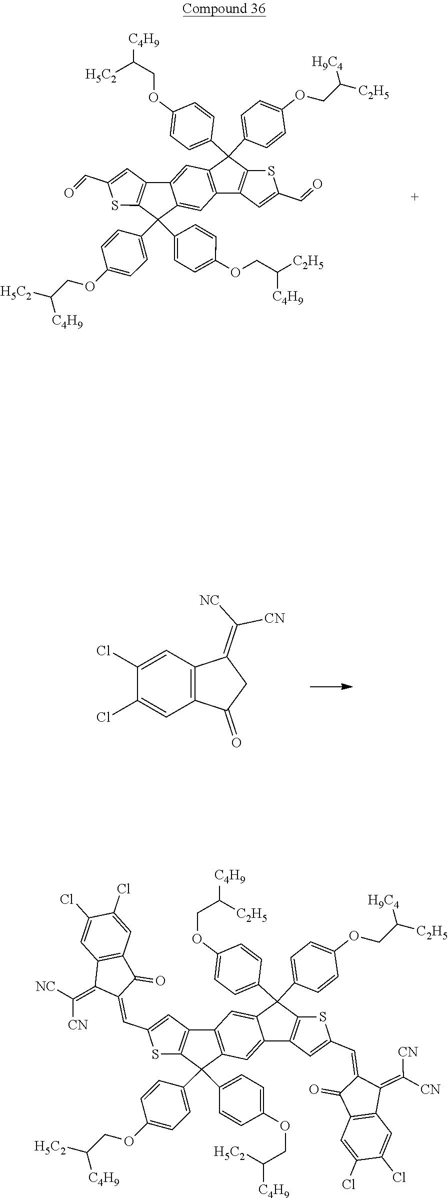

To a degassed solution of compound 35-3 (200 mg, 0.18 mmol) and anhydrous pyridine (0.99 cm³, 12 mmol) in anhydrous chloroform (14 cm³) at 0° C. was added 2-(5,6-dichloro-3-oxo-2,3-dihydro-1H-inden-1-ylidene)propanedinitrile (231 mg, 0.88 mmol). The resulting solution was then degassed for a further 30 minutes. The reaction mixture was warmed to 23° C. and stirred for 17 hours. Methanol (150 cm³) was added and the mixture stirred for 1 hour. The solid collected by filtration and washed with acetonitrile (50 cm³) to give compound 36 (270 mg, 94%) as a green solid.

¹H-NMR (400 MHz, CDCl₃) 0.73-0.92 (24H, m), 1.12-1.50 (32H, m), 1.62 (4H, quin, J 6.0), 3.66-3.84 (8H, m), 6.67-6.83 (8H, m), 7.05-7.17 (8H, m), 7.46-7.56 (2H, m), 7.79-8.02 (4H, m), 8.65-8.74 (2H, m), 8.86 (2H, m).

### Example 37

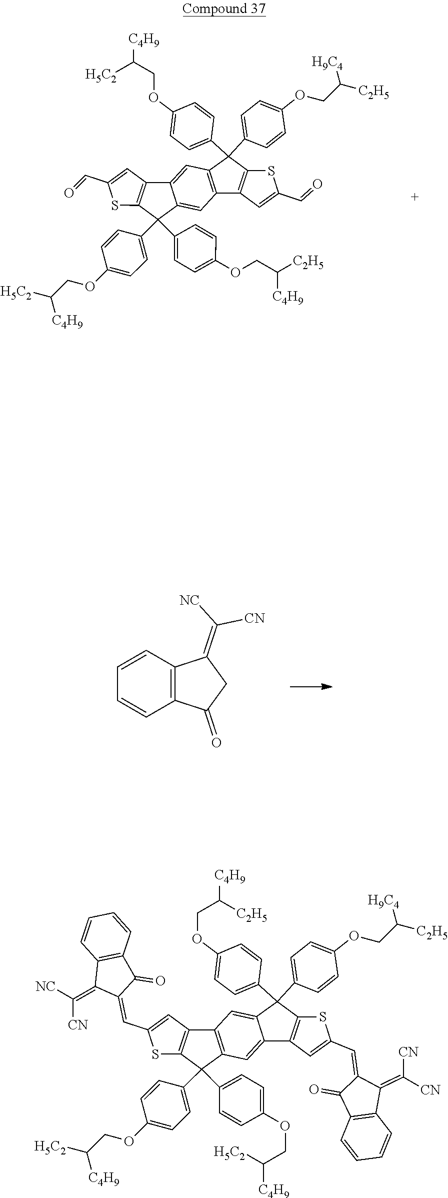

To a degassed solution of compound 35-3 (200 mg, 0.18 mmol) and anhydrous pyridine (0.99 cm³, 12 mmol) in anhydrous chloroform (14 cm³) at 0° C. was added 2-(3-oxo-2,3-dihydro-1H-inden-1-ylidene)propanedinitrile (170 mg, 0.88 mmol). The resulting solution was then degassed for a further 30 minutes. The reaction mixture was warmed to 23° C. and stirred for 17 hours. Acetonitrile (150 cm³) was added and the mixture stirred for 1 hour. The solid collected by filtration and washed with acetonitrile (100 cm³) to give compound 37 (198 mg, 76%) as a dark red/purple solid. ¹H-NMR (400 MHz, CDCl₃) 0.71-0.91 (24H, m), 1.12-1.44 (32H, m), 1.55-1.73 (4H, m), 3.65-3.82 (8H, m), 6.66-6.83 (8H, m), 7.06-7.17 (8H, m), 7.45-7.56 (2H, m), 7.61-7.78 (4H, m), 7.78-7.88 (2H, m), 7.91-7.99 (2H, m), 8.55-8.68 (2H, m), 8.85 (2H, s).

### Example 38

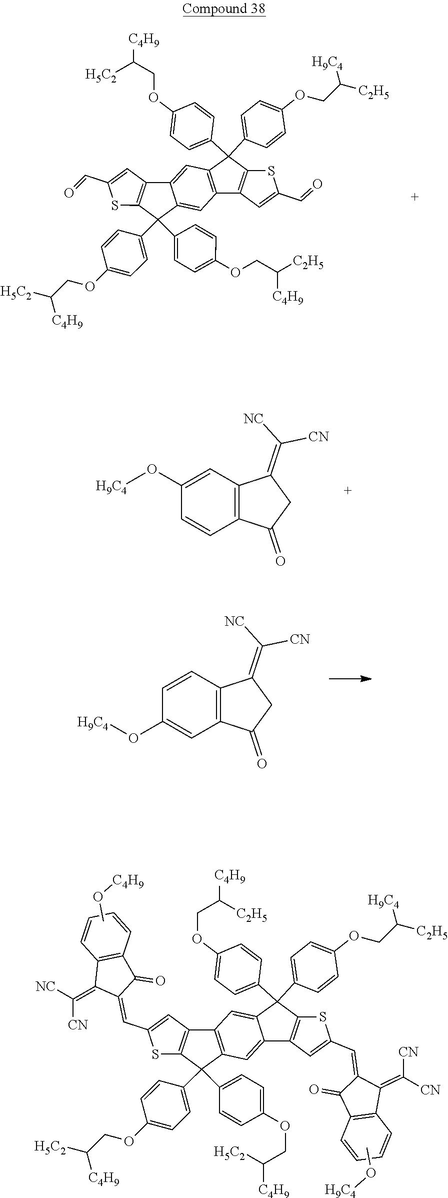

To a degassed solution of compound 35-3 (200 mg, 0.18 mmol) and anhydrous pyridine (0.99 cm³, 12 mmol) in anhydrous chloroform (14 cm³) at 0° C. was added a 4:1 mix of 2-(6-butoxy-3-oxo-2, 3-dihydro-1H-inden-1-ylidene)propanedinitrile and 2-(5-butoxy-3-oxo-2,3-dihydro-1H-inden-1-ylidene)propanedinitrile (240 mg, 1.05 mmol). The resulting solution was then degassed for a further 30 minutes. The reaction mixture was warmed to 23° C. and stirred for 17 hours. Methanol (150 cm³) was added and the mixture stirred for 1 hour. The solid collected by filtration and washed with acetonitrile (100 cm³) to give compound 38 (254 mg, 88%) as a red solid.

¹H-NMR (400 MHz, CDCl₃) 0.71-0.97 (30H, m), 1.11-1.56 (36H, m), 1.56-1.68 (4H, m), 1.69-1.88 (4H, m), 3.63-3.81 (8H, m), 3.99-4.10 (4H, m), 6.63-6.81 (8H, m), 7.08-7.17 (8H, m), 7.44-7.54 (2H, m), 7.69-7.79 (2H, m), 7.83-7.94 (2H, m), 8.06 (2H, d, J 2.0), 8.70-8.83 (2H, m).

### Use Example 1

Current-voltage characteristics are measured using a Keithley 2400 SMU while the solar cells are illuminated by a Newport Solar Simulator at 100 mW·cm⁻² white light. The solar simulator is equipped with AM1.5G filters. The illumination intensity is calibrated using a Si photodiode. All the device preparation and characterization is done in a dry-nitrogen atmosphere.

Power conversion efficiency is calculated using the following expression

$$η = \frac{{V}_{o c} \times {J}_{s c} \times F F}{{P}_{in}}$$

where FF is defined as

$$F F = \frac{{V}_{max} \times {J}_{max}}{{V}_{o c} \times {J}_{s c}}$$

OPV device characteristics for a blend which contains Polymer 1 as shown below and an acceptor compound according to the invention, and is coated from an organic solution. Details of the solution composition are shown in Table 1.

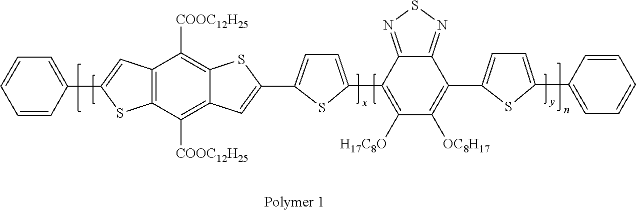

Polymer 1 and its preparation are disclosed in WO 2011/131280 A1.

A1: Inverted Bulk Heterojunction Organic Photovoltaic Devices

Organic photovoltaic (OPV) devices are fabricated on pre-patterned ITO-glass substrates (1352/sq.) purchased from LUMTEC Corporation. Substrates are cleaned using common solvents (acetone, iso-propanol, deionized-water) in an ultrasonic bath. A layer of commercially available aluminium zinc oxide (AlZnO, Nanograde) was applied as a uniform coating by doctor blade at 40° C. The AlZnO Films are then annealed at 100° C. for 10 minutes in air. Active material solutions (i.e. polymer+ acceptor) are prepared to fully dissolve the solutes at a 23 mg·cm⁻³ solution concentration. Thin films are blade-coated in air atmosphere to achieve active layer thicknesses between 50 and 800 nm as measured using a profilometer. A short drying period follows to ensure removal of any residual solvent.

Typically, blade-coated films are dried at 70° C. for 2 minutes on a hotplate. Next the devices are transferred into an air atmosphere. On top of the active layer 0.1 mL of a conducting polymer poly(ethylene dioxythiophene) doped with poly(styrene sulfonic acid) [PEDOT:PSS Clevios HTL Solar SCA 434 (Heraeus)] was spread and uniformly coated by doctor blade at 70° C. Afterwards Ag (100 nm) cathodes are thermally evaporated through a shadow mask to define the cells.

Table 1 shows the formulation characteristics of the individual photoactive material solutions, comprising a polymer as electron donor component and a compound according to the invention as electron acceptor component. Solution 1 according to the present invention contains Polymer 1 and Compound 9. The solvent is a mixture of o-xylene with 1-methylnaphthalene (oXyl:1-MN) in 95:5 ratio (v/v).

TABLE 1
Formulation characteristics

| | | Poly- | Ratio | Concen- | |
|---|---|---|---|---|---|
| No. | Acceptor | mer | Polymer:Acceptor | tration g/L | Solvent |
| 1 | Compound 9 | 1 | 1.00:1.30 | 23 | oXyl:1-MN |

A2: Inverted Device Properties

Table 2 shows the device characteristics for the individual OPV devices comprising a photoactive layer with a BHJ formed from the active material (acceptor/polymer) solutions of Table 1.

TABLE 2
Photovoltaic cell characteristics under simulated
solar irradiation at 1 sun (AM1.5G).

Average Performance

| | Voc | Jsc | FF | PCE |
|---|---|---|---|---|
| No. | mV | mA · cm⁻² | % | % |
| 1 | 945 | 3.6 | 35.5 | 1.22 |

From Table 2 it can be seen that OPV devices with a BHJ prepared from a solution of Polymer 1 and Compound 9 according to the invention, show high Voc values and functional OPV devices.

### Use Example 2

B1: Bulk Heterojunction Organic Photodetector Devices (OPDs)

Devices are fabricated onto glass substrates with six pre-patterned ITO dots of 5 mm diameter to provide the bottom electrode. The ITO substrates are cleaned using a standard process of ultrasonication in Decon90 solution (30 minutes) followed by washing with de-ionized water (×3) and ultrasonication in de-ionized water (30 minutes). The ZnO ETL layer was deposited by spin coating a ZnO nanoparticle dispersion onto the substrate and drying on a hotplate for 10 minutes at a temperature between 100 and 140° C. A formulation of Lisicon PV-D4650 (sourced from Merck KGaA) or Polymer 2 (sourced from Merck KGaA) and compound as disclosed herein was prepared at a ratio of between 1:1.3 in o-xylene at a concentration of 20 mg/ml, and stirred for 17 hours at a temperature of between 23° C. and 60° C. The active layer was deposited using blade coating (K101 Control Coater System from RK). The stage temperature was set to 30° C., the blade gap set at 200 μm and the speed set between 2-8 m/min targeting a final dry film thickness of 500-1000 nm. Following coating the active layer was annealed at 100° C. for 10 minutes. The MoO₃ HTL layer was deposited by E-beam vacuum deposition from MoO₃ pellets at a rate of 1 Å/s, targeting 15 nm thickness. Finally, the top silver electrode was deposited by thermal evaporation through a shadow mask, to achieve Ag thickness between 30-80 nm.

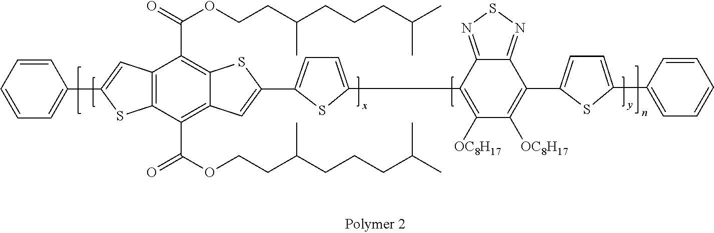

The J-V curves are measured using a Keithley 4200 system under light and dark conditions at a bias from +5 to −5 V. The light source was a 580 nm LED with power 0.5 mW/cm².

The EQE of OPD devices were characterized between 400 and 800 nm under −2V bias, using an External Quantum Efficiency (EQE) Measurement System from LOT-QuantumDesign Europe.

Table 3 shows the EQE values for the individual OPD devices comprising a photoactive layer with a BHJ formed from the photoactive acceptor/donor formulations.

TABLE 3
EQEs for the devices at 550 nm

| No. | Acceptor | Donor | EQE % (at 550 nm) |
|---|---|---|---|
| 1 | Compound 10 | PV-D4650 | 53 |
| 2 | Compound 10 | Polymer 1 | 47 |
| 3 | Compound 11 | PV-D4650 | 26 |
| 4 | Compound 11 | Polymer 1 | 27 |
| 5 | Compound 13 | PV-D4650 | 43 |
| 6 | Compound 13 | Polymer 1 | 57 |
| 7 | Compound 14 | PV-D4650 | 6 |
| 8 | Compound 14 | Polymer 1 | 13 |
| 9 | Compound 15 | PV-D4650 | 19 |
| 10 | Compound 15 | Polymer 1 | 19 |
| 11 | Compound 16 | PV-D4650 | 19 |
| 12 | Compound 16 | Polymer 1 | 30 |
| 13 | Compound 17 | PV-D4650 | 41 |
| 14 | Compound 17 | Polymer 1 | 50 |
| 15 | Compound 18 | PV-D4650 | 16 |
| 16 | Compound 18 | Polymer 1 | 30 |
| 17 | Compound 19 | PV-D4650 | 8 |
| 18 | Compound 19 | Polymer 1 | 17 |
| 19 | Compound 20 | PV-D4650 | 5 |
| 20 | Compound 20 | Polymer 1 | 18 |
| 21 | Compound 21 | PV-D4650 | 10 |
| 22 | Compound 21 | Polymer 1 | 26 |

1. A compound of formula IA 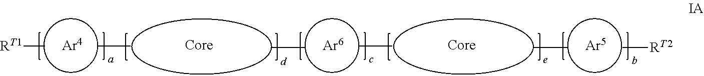
wherein
Ar⁴, Ar⁵, Ar⁶ is heteroarylene that has 5 ring atoms and contains S atom, and is unsubstituted or substituted,
a, b is 0 or 1,
c, e is 0,
d is 1,
R^{T1}, R^{T2} is selected from the following formulae 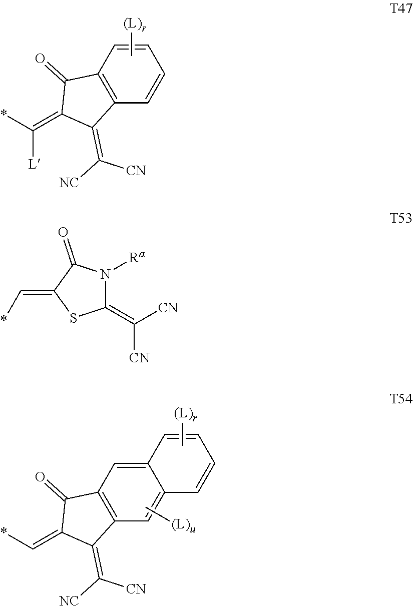
L is H, F, Cl, Br, or OR⁰,
R⁰ is straight-chain or branched alkyl with 1 to 12 C atoms,
r is 0, 1, or 2,
u is 0,
R^{a} is straight-chain or branched alkyl with 1 to 12 C atoms,
L′ is H,
“core” is a group selected from the following formulae 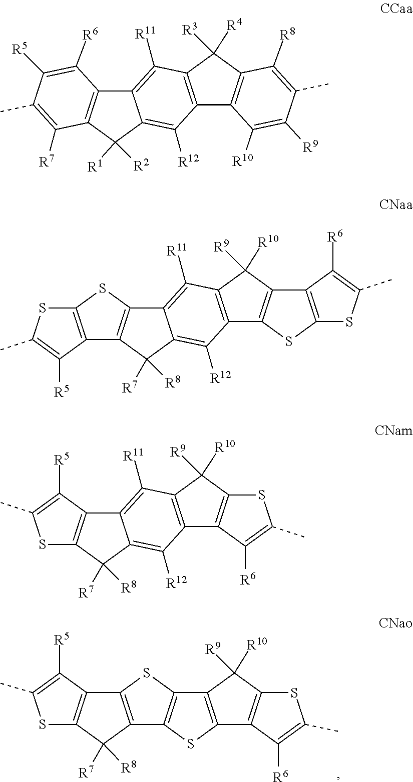
and
R¹-12 is H, unsubstituted straight-chain or branched alkyl chain with 1 to 30 C atoms, or arylalkyl, aryloxy which each of the aforementioned cyclic groups has 6 ring atoms.

2. A composition comprising one or more compounds according to claim 1, and further comprising one or more compounds having one or more of a semiconducting, hole or electron transporting, hole or electron blocking, electrically conducting, photoconducting, photoactive or light emitting property, and/or a binder.

3. The composition of claim 2, comprising one or more n-type semiconductors, at least one of which is a compound according to claim 1, and further comprising one or more p-type semiconductors.

4. The composition of claim 2, comprising one or more p-type semiconductors selected from conjugated polymers.

5. The composition according to claim 2, comprising one or more n-type semiconductors selected from fullerenes or fullerene derivatives.

6. A bulk heterojunction (BHJ) formed from a composition according to claim 2.

7. Use of a compound according to claim 1, or of a composition according to claim 2, in an electronic or optoelectronic device, or in a component of such a device or in an assembly comprising such a device.

8. A formulation comprising one or more compounds according to claim 1, or a composition according to claim 2, and further comprising one or more solvents selected from organic solvents.

9. An electronic or optoelectronic device, or a component thereof, or an assembly comprising it, which comprises a compound according to claim 1, or a composition according to claim 2.

10. The electronic or optoelectronic device according to claim 9, which is selected from organic field effect transistors (OFET), organic thin film transistors (OTFT), organic light emitting diodes (OLED), organic light emitting transistors (OLET), organic light emitting electro chemical cells (OLEC), organic photovoltaic devices (OPV), organic photodetectors (OPD), organic solar cells, dye-sensitized solar cells (DSSC), perovskite-based solar cells (PSC), organic photoelectrochemical cells (OPEC), laser diodes, Schottky diodes, photoconductors, photodetectors, thermoelectric devices and LC windows.

11. The component according to claim 9, which is selected from charge injection layers, charge transport layers, interlayers, planarising layers, antistatic films, printed polarizers, polymer electrolyte membranes (PEM), conducting substrates and conducting patterns.

12. The assembly according to claim 9, which is selected from integrated circuits (IC), radio frequency identification (RFID) tags, security markings, security devices, flat panel displays, backlights of flat panel displays, electrophotographic devices, electrophotographic recording devices, organic memory devices, sensor devices, biosensors and biochips.
